(19) United States
(12) Patent Application Publication
(10) Pub. No.: US 2025/0197856 A1
(43) Pub. Date: Jun. 19, 2025

(54) METHODS FOR THE TREATMENT OF NUCLEOTIDE REPEAT EXPANSION DISORDERS ASSOCIATED WITH MSH3 ACTIVITY

(71) Applicant: Takeda Pharmaceuticals U.S.A. Inc., Cambridge, MA (US)

(72) Inventors: Irina ANTONIJEVIC, Cambridge, MA (US); W. George LAI, Cambridge, MA (US); Pei GE, Cambridge, MA (US)

(21) Appl. No.: 18/842,923

(22) PCT Filed: Mar. 1, 2023
(86) PCT No.: PCT/US2023/063531

§ 371 Date: Aug. 30, 2024

Related U.S. Application Data
(60) Provisional application No. 63/315,707, filed on Mar. 2, 2022.

(51) Int. Cl. C12N 15/113; A61K 35/30

(52) U.S. Cl. CPC C12N 15/113; A61K 35/30; C12N 2310/11; C12N 2310/315; C12N 2310/3231; C12N 2310/3341; C12N 2310/341; C12N 2320/30

(57) ABSTRACT

The present disclosure features useful compositions and methods to treat nucleotide repeat expansion disorders, e.g., in a subject in need thereof. In some aspects, the compositions and methods described herein are useful in the treatment of disorders associated with MSH3 activity.

### CROSS-REFERENCE TO RELATED APPLICATION

This International Application claims the priority benefit of U.S. Provisional Application No. 63/315,707, filed on Mar. 2, 2022, which is hereby incorporated by reference herein, in its entirety.

### BACKGROUND

Nucleotide repeat expansion disorders (e.g., trinucleotide repeat expansion disorders) are genetic disorders caused by nucleotide repeat expansions (e.g., trinucleotide repeats). Nucleotide repeat expansions (e.g., trinucleotide repeat expansions) are a type of genetic mutation where nucleotide repeats in certain genes or introns exceed the normal, stable threshold for that gene. The nucleotide repeats (e.g., trinucleotide repeats) can result in defective or toxic gene products, impair RNA transcription, and/or cause toxic effects by forming toxic mRNA transcripts.

Nucleotide repeat expansion disorders (e.g., trinucleotide repeat expansion disorders) are generally categorized by the type of repeat expansion. For example, Type 1 disorders such as Huntington's disease are caused by CAG repeats which result in a series of glutamine residues known as a polyglutamine tract, Type 2 disorders are caused by heterogeneous expansions that are generally small in magnitude, and Type 3 disorders such as fragile X syndrome are characterized by large repeat expansions that are generally located outside of the protein coding region of the genes. Nucleotide repeat expansion disorders (e.g., trinucleotide repeat expansion disorders) are characterized by a wide variety of symptoms such as progressive degeneration of nerve cells that is common in the Type 1 disorders.

Subjects with a nucleotide repeat expansion disorder (e.g., a trinucleotide repeat expansion disorder) or those who are considered at risk for developing a nucleotide repeat expansion disorder (e.g., a trinucleotide repeat expansion disorder) have a constitutive nucleotide expansion in a gene associated with disease (i.e., the nucleotide repeat expansion is present in the gene during embryogenesis). Constitutive nucleotide repeat expansions (e.g., trinucleotide repeat expansions) can undergo expansion after embryogenesis (i.e., somatic nucleotide repeat expansion). Both constitutive nucleotide repeat expansion and somatic nucleotide repeat expansion can be associated with presence of disease, age at onset of disease, and/or rate of progression of disease.

### BRIEF DESCRIPTION OF DRAWINGS

FIG. 1A provides the MSH3 mRNA response in caudate of non-human primates after intrathecal (“IT”) administration of Antisense Oligo No. 289. FIG. 1B provides the MSH3 mRNA response in putamen after IT administration of Antisense Oligo No. 289. FIG. 1C provides the MSH3 mRNA response in cortex after IT administration of Antisense Oligo No. 289. FIG. 1D provides the MSH3 mRNA response in lumbar after IT administration of Antisense Oligo No. 289.

FIG. 2A provides the MSH3 mRNA response in caudate of non-human primates after intracerebroventricular (“ICV”) administration of Antisense Oligo No. 289. FIG. 2B provides the MSH3 mRNA response in putamen after ICV administration of Antisense Oligo No. 289. FIG. 2C provides the MSH3 mRNA response in cortex after IT administration of Antisense Oligo No. 289. FIG. 2D provides the MSH3 mRNA response in lumbar after IT administration of Antisense Oligo No. 289.

FIG. 3 provides the resulting relative expression of MSH3 following IT, ICM, and intravenous (“IV”) administration of the oligonucleotide of SEQ ID NO: 617 in non-human primates.

FIG. 4A provides the MSH3 mRNA response at t=0, t=15 days, and t=29 days after ICV administration of Antisense Oligo No. 289 in the ipsilateral caudate, ipsilateral frontal and temporal cortex, and ipsilateral nucleus accumbens.

FIG. 4B provides the MSH3 mRNA response at t=0, t=15 days, and t=29 days after ICV administration of Antisense Oligo No. 289 in the contralateral caudate, contralateral frontal and temporal cortex, and contralateral nucleus accumbens.

FIG. 5A provides the MSH3 mRNA response at t=0, t=24 h, t=48 h, t=8 days, t=15 days, and t=29 days after ICV administration of Antisense Oligo No. 289 in the ipsilateral and contralateral caudate.

FIG. 5B provides the MSH3 mRNA response at t=0, t=24 h, t=48 h, t=8 days, t=15 days, and t=29 days after ICV administration of Antisense Oligo No. 289 in the ipsilateral and contralateral putamen.

FIG. 5C provides the MSH3 mRNA response at t=0, t=24 h, t=48 h, t=8 days, t=15 days, and t=29 days after ICV administration of Antisense Oligo No. 289 in the ipsilateral and contralateral accumbens.

FIG. 5D provides the MSH3 mRNA response at t=0, t=24 h, t=48 h, t=8 days, t=15 days, and t=29 days after ICV administration of Antisense Oligo No. 289 in the ipsilateral and contralateral frontal cortex.

FIG. 5E provides the MSH3 mRNA response at t=0, t=24 h, t=48 h, t=8 days, t=15 days, and t=29 days after ICV administration of Antisense Oligo No. 289 in the ipsilateral and contralateral temporal cortex.

FIG. 6 shows MSH3 mRNA knock down (“KD”) in the frontal cortex after repeat intrathecal (“IT”) dosing in non-human primates. The X-axis shows the antisense oligos tested. From left to right: artificial CSF control, Antisense OligoNO: 1, Antisense Oligo NO: 97, Antisense Oligo NO: 193, Antisense Oligo NO: 289, and Antisense Oligo NO: 617. The Y axis shows the remaining MSH3 mRNA normalized to five house-keeping genes and compared to the aCSF group.

FIG. 7 shows MSH3 protein knockdown in the frontal cortex after repeat IT dosing in non-human primates. The X axis shows the MSH3 protein amount (normalized to beta-tubulin) using a proprietary antibody and a western blot in the cortex in the aCSF treated group vs. the ASO treated groups (treated with Antisense Oligo NO. 289), 15 days after repeat IT dosing.

FIG. 8 shows QRT-PCR results of mRNA of the mouse MSH3 gene in the retina from 3 experimental groups at the 50 μg and 100 μg dosages as compared to phosphate buffered saline (PBS).

FIGS. 9A-9D show MSH3 mRNA knockdown in the cortex for up to 12 weeks following a single 10 mg ICV dose of Antisense Oligo No. 289. FIG. 9A shows the results achieved in the ipsilateral frontal cortex. FIG. 9B shows the results achieved in the contralateral frontal cortex. FIG. 9C shows the results achieved in the ipsilateral temporal cortex. FIG. 9D shows the results achieved in the contralateral temporal cortex.

FIGS. 10A-10B show MSH3 knockdown in the caudate for up to 12 weeks following a single 10 mg ICV dose of Antisense Oligo No. 289. FIG. 10A shows the results achieved in the ipsilateral caudate. FIG. 10B shows the results achieved in the contralateral caudate.

FIGS. 11A-11B show MSH3 knockdown in the n. accumbens for up to 12 weeks following a single 10 mg ICV dose of Antisense Oligo No. 289. FIG. 11A shows the results achieved in the ipsilateral n. accumbens. FIG. 11B shows the results achieved in the contralateral n. accumbens.

FIGS. 12A-12B show MSH3 knockdown in the putamen at 8 and 12 weeks following a single 10 mg ICV dose of Antisense Oligo No. 289. FIG. 12A shows the results achieved in the ipsilateral putamen. FIG. 12B shows the results achieved in the contralateral putamen.

FIGS. 13A-13B show MSH3 knockdown in the cortex up to 4 weeks following a single 10 mg ICV dose of Antisense Oligo No. 289. FIG. 13A shows the results achieved in the ipsilateral and contralateral motor cortex. FIG. 13B shows the results achieved in the ipsilateral and contralateral occipital cortex.

FIGS. 14A-14I show MSH3 knockdown in the ipsilateral body of caudate, amygdala, and thalamus up to 4 weeks following a single 10 mg ICV dose of Antisense Oligo No. 289. FIG. 14A shows the results achieved in the ipsilateral and contralateral body of caudate. FIG. 14B shows the results achieved in the ipsilateral and contralateral body of globus pallidus. FIG. 14C shows the results achieved in the ipsilateral and contralateral amygdala. FIG. 14D shows the results achieved in the ipsilateral and contralateral hypothalamus. FIG. 14E shows the results achieved in the ipsilateral and contralateral hypothalamus. FIG. 14F shows the results achieved in the ipsilateral and contralateral thalamus. FIG. 14G shows the results achieved in the ipsilateral and contralateral substantia nigra. FIG. 14H shows the results achieved in the ipsilateral and contralateral pons. FIG. 14I shows the results achieved in the ipsilateral and contralateral medulla oblongata.

FIGS. 15A-15B show MSH3 knockdown in the ipsilateral white matter around the injection site up to 4 weeks following a single 10 mg ICV dose of Antisense Oligo No. 289. FIG. 15A shows the results achieved in the white matter at the level of the amydgala. FIG. 15B shows the results achieved in the white matter close to the injection area.

FIGS. 16A-16D show sustained bilateral MSH3 mRNA knockdown in NHPs over 12 weeks after a single ICV injection of artificial cerebrospinal fluid (aCSF) or Antisense Oligo No. 289, at the dosages shown. FIG. 16A shows the results achieved in the caudate. FIG. 16B shows the results achieved in the putamen. FIG. 16C shows the results achieved in the accumbens. FIG. 16D shows the results achieved in the frontal cortex.

FIGS. 17A-17D show sustained bilateral MSH3 mRNA knockdown in NHPs over 24 weeks after a single ICV injection of aCSF or Antisense Oligo No. 289, at the dosages shown. FIG. 17A shows the results achieved in the caudate. FIG. 17B shows the results achieved in the putamen. FIG. 17C shows the results achieved in the frontal cortex. FIG. 17D shows the results achieved in the accumbens.

### SUMMARY OF THE DISCLOSURE

The present disclosure features useful methods to treat nucleotide repeat expansion disorders (e.g., trinucleotide repeat expansion disorders), e.g., in a subject in need thereof. In some aspects, the methods described herein are useful in the treatment of disorders associated with MSH3 activity.

### Intracerebroventricular Administration and Dosages

Some aspects of the disclosure relate to a method of treating, preventing, or delaying the onset and/or progression of a nucleotide repeat expansion disorder in a subject in need thereof, the method comprising intracerebroventricularly administering a single-stranded oligonucleotide that targets MSH3, or a pharmaceutically acceptable salt thereof, wherein the single-stranded oligonucleotide, or a pharmaceutically acceptable salt thereof, is administered at a dose of about 2 mg to about 300 mg. In some aspects, the oligonucleotide, or a portion thereof, is at least 95% complementary to at least 15 contiguous nucleobase at positions 2543-2573 of SEQ ID NO: 614, or a pharmaceutically acceptable salt thereof, wherein the single-stranded oligonucleotide, or a pharmaceutically acceptable salt thereof, is administered at a dose of about 2 mg to about 300 mg. In some aspects, the oligonucleotide, or a portion thereof, is at least 98% complementary to at least 15 contiguous nucleobase at positions 2543-2573 of SEQ ID NO: 614, or a pharmaceutically acceptable salt thereof. In some aspects, the oligonucleotide, or a portion thereof, is at least 99% complementary to at least 15 contiguous nucleobase at positions 2543-2573 of SEQ ID NO: 614, or a pharmaceutically acceptable salt thereof. In some aspects, the oligonucleotide, or a portion thereof, is 100% complementary to at least 15 contiguous nucleobase at positions 2543-2573 of SEQ ID NO: 614, or a pharmaceutically acceptable salt thereof. In some aspects, the oligonucleotide, or a portion thereof, is complementary to 17-23 contiguous nucleobase at positions 2543-2573 of SEQ ID NO: 614, or a pharmaceutically acceptable salt thereof. In some aspects, the oligonucleotide is complementary to 17-20 contiguous nucleobase at positions 2543-2573 of SEQ ID NO: 614, or a pharmaceutically acceptable salt thereof. In some aspects, the 17-20 contiguous nucleobase begin at position 2543, 2544, 2545, 2546, 2547, 2548, 2549, 2550, 2551, 2552, 2553, 2554, 2555, 2556, or 2557 of SEQ ID NO: 614, or a pharmaceutically acceptable salt thereof. In some aspects, the oligonucleotide is 17-20 linked nucleotide in length, or a pharmaceutically acceptable salt thereof. In some aspects, the oligonucleotide, or a portion thereof, is complementary to 20-23 contiguous nucleobase at positions 2543-2573 of SEQ ID NO: 614, or a pharmaceutically acceptable salt thereof. In some aspects, the 20-23 contiguous nucleobase begin at position 2543, 2544, 2545, 2546, 2547, 2548, 2549, 2550, 2551, 2552, 2553, or 2554 of SEQ ID NO: 614, or a pharmaceutically acceptable salt thereof. In some aspects, the oligonucleotide is 20-23 linked nucleotide in length, or a pharmaceutically acceptable salt thereof. In some aspects, the oligonucleotide, or a portion thereof, is complementary to positions 2543-2570 of SEQ ID NO: 614, or a pharmaceutically acceptable salt thereof.

Some aspects of the disclosure are related to a method of treating, preventing, or delaying the onset and/or progression of a nucleotide repeat expansion disorder in a subject in need thereof, the method comprising intracerebroventricularly administering a single-stranded oligonucleotide of 15-30 linked nucleotide in length, wherein the oligonucleotide, or a portion thereof, is at least 95% complementary to at least 15 contiguous nucleobase at positions 2685-2714 of SEQ ID NO: 614, or a pharmaceutically acceptable salt thereof, wherein the single-stranded oligonucleotide, or a pharmaceutically acceptable salt thereof, is administered at a dose of about 2 mg to about 300 mg. In some aspects, the oligonucleotide, or a portion thereof, is at least 98% complementary to at least 15 contiguous nucleobase at positions 2685-2714 of SEQ ID NO: 614, or a pharmaceutically acceptable salt thereof. In some aspects, the oligonucleotide, or a portion thereof, is at least 99% complementary to at least 15 contiguous nucleobase at positions 2685-2714 of SEQ ID NO: 614, or a pharmaceutically acceptable salt thereof. In some aspects, the oligonucleotide or a portion thereof, is 100% complementary to at least 15 contiguous nucleobase at positions 2685-2714 of SEQ ID NO: 614, or a pharmaceutically acceptable salt thereof. In some aspects, the oligonucleotide, or a portion thereof is complementary to 17-23 contiguous nucleobase at positions 2685-2714 of SEQ ID NO: 614, or a pharmaceutically acceptable salt thereof. In some aspects, the oligonucleotide, or a portion thereof, is complementary to 17-20 contiguous nucleobase at positions 2685-2714 of SEQ ID NO: 614, or a pharmaceutically acceptable salt thereof. In some aspects, the oligonucleotide, or a portion thereof, is complementary to 17-20 contiguous nucleobase beginning at position 2685, 2686, 2687, 2688, 2689, 2690, 2691, 2692, 2693, 2694, 2695, 2696, 2697, or 2698 of SEQ ID NO: 614, or a pharmaceutically acceptable salt thereof. In some aspects, the oligonucleotide is 17-20 linked nucleotide in length, the oligonucleotide, or a portion thereof, is complementary to 20-23 contiguous nucleobase at positions 2685-2714 of SEQ ID NO: 614, or a pharmaceutically acceptable salt thereof. In some aspects, the oligonucleotide is complementary to 20-23 contiguous nucleobase beginning at position 2685, 2686, 2687, 2688, 2689, 2690, 2691, 2692, 2693, 2694, or 2695 of SEQ ID NO: 614, or a pharmaceutically acceptable salt thereof. In some aspects, the oligonucleotide is 20-23 linked nucleotide in length, or a pharmaceutically acceptable salt thereof. In some aspects, the oligonucleotide, or a portion thereof, is complementary to positions 2685-2714 of SEQ ID NO: 614, or a pharmaceutically acceptable salt thereof. In some aspects, the oligonucleotide is not any one of Antisense Oligo Nos. 1, 97, 193, or 289 of Table 3. In some aspects, the oligonucleotide does not have a nucleobase sequence consisting of any one of SEQ ID NOs: 1, 97, 193, or 289.

In some aspects, the oligonucleotide consists of a nucleobase sequence selected from the group consisting of any of SEQ ID NOs: 1-384 and 390-613, or a pharmaceutically acceptable salt thereof. In some aspects, the oligonucleotide consists of a nucleobase sequence selected from the group consisting of any of SEQ ID NOs: 2-96, 98-192, 194-288, 290-384, and 390-613, or a pharmaceutically acceptable salt thereof. In some aspects, the oligonucleotide consists of a nucleobase sequence selected from the group consisting of any of SEQ ID NOs: 1-384, or a pharmaceutically acceptable salt thereof. In some aspects, the oligonucleotide consists of a nucleobase sequence selected from the group consisting of any of SEQ ID NOs: 2-96, 98-192, 194-288, and 290-384, or a pharmaceutically acceptable salt thereof. In some aspects, the oligonucleotide consists of a nucleobase sequence selected from the group consisting of any of SEQ ID NOs: 1-96, or a pharmaceutically acceptable salt thereof. In some aspects, the oligonucleotide consists of a nucleobase sequence selected from the group consisting of any of SEQ ID NOs: 2-96, or a pharmaceutically acceptable salt thereof. In some aspects, the oligonucleotide consists of a nucleobase sequence selected from the group consisting of any of SEQ ID NOs: 97-192, or a pharmaceutically acceptable salt thereof. In some aspects, the oligonucleotide consists of a nucleobase sequence selected from the group consisting of any of SEQ ID NOs: 98-192, or a pharmaceutically acceptable salt thereof. In some aspects, the oligonucleotide consists of a nucleobase sequence selected from the group consisting of any of SEQ ID NOs: 193-288, or a pharmaceutically acceptable salt thereof. In some aspects, the oligonucleotide consists of a nucleobase sequence selected from the group consisting of any of SEQ ID NOs: 194-288, or a pharmaceutically acceptable salt thereof. In some aspects, the oligonucleotide consists of a nucleobase sequence selected from the group consisting of any of SEQ ID NOs: 289-384, or a pharmaceutically acceptable salt thereof. In some aspects, the oligonucleotide consists of a nucleobase sequence selected from the group consisting of any of SEQ ID NOs: 288-384, or a pharmaceutically acceptable salt thereof. In some aspects, the oligonucleotide consists of a nucleobase sequence selected from the group consisting of any of SEQ ID NOs: 390-613, or a pharmaceutically acceptable salt thereof. In some aspects, the oligonucleotide consists of a nucleobase sequence selected from the group consisting of any of SEQ ID NOs: 390-480, or a pharmaceutically acceptable salt thereof. In some aspects, the oligonucleotide consists of a nucleobase sequence selected from the group consisting of any of SEQ ID NOs: 481-571, or a pharmaceutically acceptable salt thereof. In some aspects, the oligonucleotide consists of a nucleobase sequence selected from the group consisting of any of SEQ ID NOs: 572-662, or a pharmaceutically acceptable salt thereof. In some aspects, the oligonucleotide consists of a nucleobase sequence selected from the group consisting of any of SEQ ID NOs: 663-613, or a pharmaceutically acceptable salt thereof. In some aspects, the oligonucleotide consists of a nucleobase sequence selected from the group consisting of any of SEQ ID NOs: 1, 6, 13, 17, 21, 24, 26, 29, 33-34, 37, 44, 49-55, 57, 60-73, 75-76, 79-82, 84-86, 88-92, or 94-96, or a pharmaceutically acceptable salt thereof. In some aspects, the oligonucleotide consists of a nucleobase sequence selected from the group consisting of any of SEQ ID NOs: 6, 13, 17, 21, 24, 26, 29, 33-34, 37, 44, 49-55, 57, 60-73, 75-76, 79-82, 84-86, 88-92, or 94-96, or a pharmaceutically acceptable salt thereof. In some aspects, the oligonucleotide consists of a nucleobase sequence that is SEQ ID NO: 1, or a pharmaceutically acceptable salt thereof. In some aspects, the oligonucleotide consists of a nucleobase sequence that is SEQ ID NO: 6, or a pharmaceutically acceptable salt thereof. In some aspects, the oligonucleotide consists of a nucleobase sequence selected from the group consisting of any of SEQ ID NOs: 97, 100, 103, 105, 108, 110-111, 113-117, 122-123, 127, 129-130, 133-136, 138-139, 141, 143-145, 147-148, 154-155, 157-165, 168-170, 172, 174-180, 184, 187, or 191, or a pharmaceutically acceptable salt thereof. In some aspects, the oligonucleotide consists of a nucleobase sequence selected from the group consisting of any of SEQ ID NOs: 100, 103, 105, 108, 110-111, 113-117, 122-123, 127, 129-130, 133-136, 138-139, 141, 143-145, 147-148, 154-155, 157-165, 168-170, 172, 174-180, 184, 187, or 191, or a pharmaceutically acceptable salt thereof. In some aspects, the oligonucleotide consists of a nucleobase sequence that is SEQ ID NO: 97, or a pharmaceutically acceptable salt thereof. In some aspects, the oligonucleotide consists of a nucleobase sequence that is SEQ ID NO: 145, or a pharmaceutically acceptable salt thereof. In some aspects, the oligonucleotide consists of a nucleobase sequence that is SEQ ID NO: 163, or a pharmaceutically acceptable salt thereof. In some aspects, the oligonucleotide consists of a nucleobase sequence selected from the group consisting of any of SEQ ID NOs: 193-200, 202-230, 232-246, 248-253, 255, 258-261, 265, 270, 274-276, or 285-286, or a pharmaceutically acceptable salt thereof. In some aspects, the oligonucleotide consists of a nucleobase sequence selected from the group consisting of any of SEQ ID NOs: 194-200, 202-230, 232-246, 248-253, 255, 258-261, 265, 270, 274-276, or 285-286, or a pharmaceutically acceptable salt thereof. In some aspects, the oligonucleotide consists of a nucleobase sequence that is SEQ ID NO: 193, or a pharmaceutically acceptable salt thereof. In some aspects, the oligonucleotide consists of a nucleobase sequence selected from the group consisting of any of SEQ ID NOs: 226-227, 234, 240, or 243-244, or a pharmaceutically acceptable salt thereof. In some aspects, the oligonucleotide consists of a nucleobase sequence selected from the group consisting of any of SEQ ID NOs: 227, 234, 240, or 243-244, or a pharmaceutically acceptable salt thereof. In some aspects, the oligonucleotide consists of the nucleobase sequence that is SEQ ID NO: 226, or a pharmaceutically acceptable salt thereof. In some aspects, the oligonucleotide consists of a nucleobase sequence selected from the group consisting of any of SEQ ID NOs: 289-290, 292, 305, 307, 313, 318, 323-324, 326, 329-330, 332, 338-339, 341, 344, or 346, or a pharmaceutically acceptable salt thereof. In some aspects, the oligonucleotide consists of a nucleobase sequence selected from the group consisting of any of SEQ ID NOs: 290, 292, 305, 307, 313, 318, 323-324, 326, 329-330, 332, 338-339, 341, 344, or 346, or a pharmaceutically acceptable salt thereof. In some aspects, the oligonucleotide consists of a nucleobase sequence that is SEQ ID NO: 289, or a pharmaceutically acceptable salt thereof. In some aspects, the oligonucleotide consists of a nucleobase sequence that is SEQ ID NO: 329, or a pharmaceutically acceptable salt thereof. In some aspects, the oligonucleotide consists of a nucleobase sequence that is SEQ ID NO: 346, or a pharmaceutically acceptable salt thereof.

In some aspects, the disclosure relates to a method of treating, preventing, or delaying the onset and/or progression of a nucleotide repeat expansion disorder in a subject in need thereof, the method comprising intracerebroventricularly administering a single-stranded oligonucleotide, wherein the nucleobase sequence of the oligonucleotide consists of any one of SEQ ID NOs: 1-384 and 390-613, or a pharmaceutically acceptable salt thereof, wherein the single-stranded oligonucleotide, or a pharmaceutically acceptable salt thereof, is administered at a dose of about 2 mg to about 300 mg. In some aspects, the nucleobase sequence of the oligonucleotide consists of any one of SEQ ID NOs: 2-96, 98-192, 194-288, 290-384, and 390-613, or a pharmaceutically acceptable salt thereof. In some aspects, the nucleobase sequence of the oligonucleotide consists of any one of SEQ ID NOs: 1-384, or a pharmaceutically acceptable salt thereof. In some aspects, the nucleobase sequence of the oligonucleotide consists of any one of SEQ ID NOs: 2-96, 98-192, 194-288, or 290-384, or a pharmaceutically acceptable salt thereof. In some aspects, the oligonucleotide consists of the nucleobase sequence of any one of SEQ ID NOs: 1-96, or a pharmaceutically acceptable salt thereof. In some aspects, the oligonucleotide consists of the nucleobase sequence of any one of SEQ ID NOs: 2-96, or a pharmaceutically acceptable salt thereof. In some aspects, the oligonucleotide consists of the nucleobase sequence of any one of SEQ ID NOs: 97-192, or a pharmaceutically acceptable salt thereof. In some aspects, the oligonucleotide consists of the nucleobase sequence of any one of SEQ ID NOs: 96-192, or a pharmaceutically acceptable salt thereof. In some aspects, the oligonucleotide consists of the nucleobase sequence of any one of SEQ ID NOs: 193-288, or a pharmaceutically acceptable salt thereof. In some aspects, the oligonucleotide consists of the nucleobase sequence of any one of SEQ ID NOs: 194-288, or a pharmaceutically acceptable salt thereof. In some aspects, the oligonucleotide consists of the nucleobase sequence of any one of SEQ ID NOs: 289-384, or a pharmaceutically acceptable salt thereof. In some aspects, the oligonucleotide consists of the nucleobase sequence of any one of SEQ ID NOs: 290-384, or a pharmaceutically acceptable salt thereof. In some aspects, the oligonucleotide consists of the nucleobase sequence of any one of SEQ ID NOs: 390-613, or a pharmaceutically acceptable salt thereof. In some aspects, the oligonucleotide consists of the nucleobase sequence of any one of SEQ ID NOs: 390-480, or a pharmaceutically acceptable salt thereof. In some aspects, the oligonucleotide consists of the nucleobase sequence of any one of SEQ ID NOs: 481-571, or a pharmaceutically acceptable salt thereof. In some aspects, the oligonucleotide consists of the nucleobase sequence of any one of SEQ ID NOs: 572-662, or a pharmaceutically acceptable salt thereof. In some aspects, the oligonucleotide consists of the nucleobase sequence of any one of SEQ ID NOs: 663-613, or a pharmaceutically acceptable salt thereof. In some aspects, the oligonucleotide consists of the nucleobase sequence of any one of SEQ ID NOs: 1, 6, 13, 17, 21, 24, 26, 29, 33-34, 37, 44, 49-55, 57, 60-73, 75-76, 79-82, 84-86, 88-92, or 94-96, or a pharmaceutically acceptable salt thereof. In some aspects, the oligonucleotide consists of the nucleobase sequence of any one of SEQ ID NOs: 6, 13, 17, 21, 24, 26, 29, 33-34, 37, 44, 49-55, 57, 60-73, 75-76, 79-82, 84-86, 88-92, or 94-96, or a pharmaceutically acceptable salt thereof. In some aspects, the oligonucleotide consists of the nucleobase sequence of SEQ ID NO: 1, or a pharmaceutically acceptable salt thereof. In some aspects, the oligonucleotide consists of the nucleobase sequence of SEQ ID NO: 6, or a pharmaceutically acceptable salt thereof. In some aspects, the oligonucleotide consists of the nucleobase sequence of any one of SEQ ID NOs: 97, 100, 103, 105, 108, 110-111, 113-117, 122-123, 127, 129-130, 133-136, 138-139, 141, 143-145, 147-148, 154-155, 157-165, 168-170, 172, 174-180, 184, 187, or 191, or a pharmaceutically acceptable salt thereof. In some aspects, the oligonucleotide consists of the nucleobase sequence of any one of SEQ TD NOs: 100, 103, 105, 108, 110-111, 113-117, 122-123, 127, 129-130, 133-136, 138-139, 141, 143-145, 147-148, 154-155, 157-165, 168-170, 172, 174-180, 184, 187, or 191, or a pharmaceutically acceptable salt thereof. In some aspects, the oligonucleotide consists of the nucleobase sequence of SEQ ID NO: 97, or a pharmaceutically acceptable salt thereof.

In some aspects, the oligonucleotide consists of a nucleobase sequence that is SEQ ID NO: 145, or a pharmaceutically acceptable salt thereof. In some aspects, the oligonucleotide consists of a nucleobase sequence that is SEQ ID NO: 163, or a pharmaceutically acceptable salt thereof. In some aspects, the oligonucleotide consists of the nucleobase sequence of any one of SEQ ID NOs: 193-200, 202-230, 232-246, 248-253, 255, 258-261, 265, 270, 274-276, or 285-286, or a pharmaceutically acceptable salt thereof. In some aspects, the oligonucleotide consists of the nucleobase sequence of any one of SEQ ID NOs: 194-200, 202-230, 232-246, 248-253, 255, 258-261, 265, 270, 274-276, or 285-286, or a pharmaceutically acceptable salt thereof. In some aspects, the oligonucleotide consists of the nucleobase sequence of any one of SEQ ID NO: 193, or a pharmaceutically acceptable salt thereof. In some aspects, the oligonucleotide consists of the nucleobase sequence of any one of SEQ ID NOs: 226-227, 234, 240, or 243-244, or a pharmaceutically acceptable salt thereof. In some aspects, the oligonucleotide consists of the nucleobase sequence of any one of SEQ ID NOs: 227, 234, 240, or 243-244, or a pharmaceutically acceptable salt thereof. In some aspects, the oligonucleotide consists of the nucleobase sequence of SEQ ID NO: 226, or a pharmaceutically acceptable salt thereof. In some aspects, the oligonucleotide consists of the nucleobase sequence of any one of SEQ ID NOs: 289-290, 292, 305, 307, 313, 318, 323-324, 326, 329-330, 332, 338-339, 341, 344, or 346, or a pharmaceutically acceptable salt thereof. In some aspects, the oligonucleotide consists of the nucleobase sequence of any one of SEQ ID NOs: 290, 292, 305, 307, 313, 318, 323-324, 326, 329-330, 332, 338-339, 341, 344, or 346, or a pharmaceutically acceptable salt thereof. In some aspects, the oligonucleotide consists of the nucleobase sequence of SEQ ID NO: 289, or a pharmaceutically acceptable salt thereof. In some aspects, the oligonucleotide consists of the nucleobase sequence of SEQ ID NO: 329, or a pharmaceutically acceptable salt thereof. In some aspects, the oligonucleotide consists of the nucleobase sequence of SEQ ID NO: 346, or a pharmaceutically acceptable salt thereof.

In some aspects, the disclosure relates to a method of treating, preventing, or delaying the onset and/or progression of a nucleotide repeat expansion disorder in a subject in need thereof, the method comprising intracerebroventricularly administering an oligonucleotide selected from the group consisting of Antisense Oligo Nos. 1-384 of Table 3 or 390-613 of Table 4, or a pharmaceutically acceptable salt thereof, wherein the single-stranded oligonucleotide, or a pharmaceutically acceptable salt thereof, is administered at a dose of about 2 mg to about 300 mg. In some aspects, the oligonucleotide is selected from the group consisting of Antisense Oligo Nos. 2-96, 98-192, 194-288, 290-384 of Table 3 and 390-613 of Table 4, or a pharmaceutically acceptable salt thereof. In some aspects, the oligonucleotide is selected from the group consisting of Antisense Oligo Nos. 1-384 of Table 3, or a pharmaceutically acceptable salt thereof. In some aspects, the oligonucleotide is selected from the group consisting of Antisense Oligo Nos. 2-96, 98-192, 194-288, and 290-384 of Table 3, or a pharmaceutically acceptable salt thereof. In some aspects, the oligonucleotide is selected from the group consisting of Antisense Oligo Nos. 1-96 of Table 3, or a pharmaceutically acceptable salt thereof. In some aspects, the oligonucleotide is selected from the group consisting of Antisense Oligo Nos. 2-96 of Table 3, or a pharmaceutically acceptable salt thereof. In some aspects, the oligonucleotide is selected from the group consisting of Antisense Oligo Nos. 97-192 of Table 3, or a pharmaceutically acceptable salt thereof. In some aspects, the oligonucleotide is selected from the group consisting of Antisense Oligo Nos. 98-192 of Table 3, or a pharmaceutically acceptable salt thereof. In some aspects, the oligonucleotide is selected from the group consisting of Antisense Oligo Nos. 193-288 of Table 3, or a pharmaceutically acceptable salt thereof. In some aspects, the oligonucleotide is selected from the group consisting of Antisense Oligo Nos. 194-288 of Table 3, or a pharmaceutically acceptable salt thereof. In some aspects, the oligonucleotide is selected from the group consisting of Antisense Oligo Nos. 289-384 of Table 3, or a pharmaceutically acceptable salt thereof. In some aspects, the oligonucleotide is selected from the group consisting of Antisense Oligo Nos. 290-384 of Table 3, or a pharmaceutically acceptable salt thereof. In some aspects, the oligonucleotide is selected from the group consisting of Antisense Oligo Nos. 390-613 of Table 4, or a pharmaceutically acceptable salt thereof. In some aspects, the oligonucleotide is selected from the group consisting of Antisense Oligo Nos. 390-480 of Table 4, or a pharmaceutically acceptable salt thereof. In some aspects, the oligonucleotide is selected from the group consisting of Antisense Oligo Nos. 481-571 of Table 4, or a pharmaceutically acceptable salt thereof. In some aspects, the oligonucleotide is selected from the group consisting of Antisense Oligo Nos. 1, 6, 13, 17, 21, 24, 26, 29, 33-34, 37, 44, 49-55, 57, 60-73, 75-76, 79-82, 84-86, 88-92, or 94-96 of Table 3, or a pharmaceutically acceptable salt thereof. In some aspects, the oligonucleotide is selected from the group consisting of Antisense Oligo Nos. 6, 13, 17, 21, 24, 26, 29, 33-34, 37, 44, 49-55, 57, 60-73, 75-76, 79-82, 84-86, 88-92, or 94-96 of Table 3, or a pharmaceutically acceptable salt thereof. In some aspects, the oligonucleotide is Antisense Oligo No. 1 of Table 3, or a pharmaceutically acceptable salt thereof. In some aspects, the oligonucleotide is Antisense Oligo No. 6 of Table 3, or a pharmaceutically acceptable salt thereof. In some aspects, the oligonucleotide is selected from the group consisting of Antisense Oligo Nos. 97, 100, 103, 105, 108, 110-111, 113-117, 122-123, 127, 129-130, 133-136, 138-139, 141, 143-145, 147-148, 154-155, 157-165, 168-170, 172, 174-180, 184, 187, or 191 of Table 3, or a pharmaceutically acceptable salt thereof. In some aspects, the oligonucleotide is selected from the group consisting of Antisense Oligo Nos. 100, 103, 105, 108, 110-111, 113-117, 122-123, 127, 129-130, 133-136, 138-139, 141, 143-145, 147-148, 154-155, 157-165, 168-170, 172, 174-180, 184, 187, or 191 of Table 3, or a pharmaceutically acceptable salt thereof. In some aspects, the oligonucleotide is Antisense Oligo No. 97 of Table 3, or a pharmaceutically acceptable salt thereof. In some aspects, the oligonucleotide is Antisense Oligo No. 145 of Table 3, or a pharmaceutically acceptable salt thereof. In some aspects, the oligonucleotide is Antisense Oligo No. 163 of Table 3, or a pharmaceutically acceptable salt thereof. In some aspects, the oligonucleotide is selected from the group consisting of Antisense Oligo Nos. 193-200, 202-230, 232-246, 248-253, 255, 258-261, 265, 270, 274-276, or 285-286 of Table 3, or a pharmaceutically acceptable salt thereof. In some aspects, the oligonucleotide is selected from the group consisting of Antisense Oligo Nos. 194-200, 202-230, 232-246, 248-253, 255, 258-261, 265, 270, 274-276, or 285-286 of Table 3, or a pharmaceutically acceptable salt thereof. In some aspects, the oligonucleotide is Antisense Oligo No. 193 of Table 3, or a pharmaceutically acceptable salt thereof. In some aspects, the oligonucleotide is selected from the group consisting of Antisense Oligo Nos. 226-227, 234, 240, or 243-244 of Table 3, or a pharmaceutically acceptable salt thereof. In some aspects, the oligonucleotide is selected from the group consisting of Antisense Oligo Nos. 227, 234, 240, or 243-244 of Table 3, or a pharmaceutically acceptable salt thereof. In some aspects, the oligonucleotide is Antisense Oligo No. 226 of Table 3, or a pharmaceutically acceptable salt thereof. In some aspects, the oligonucleotide is selected from the group consisting of Antisense Oligo Nos. 289-290, 292, 305, 307, 313, 318, 323-324, 326, 329-330, 332, 338-339, 341, 344, or 346 of Table 3, or a pharmaceutically acceptable salt thereof. In some aspects, the oligonucleotide is selected from the group consisting of Antisense Oligo Nos. 290, 292, 305, 307, 313, 318, 323-324, 326, 329-330, 332, 338-339, 341, 344, or 346 of Table 3, or a pharmaceutically acceptable salt thereof. In some aspects, the oligonucleotide is Antisense Oligo No. 289 of Table 3, or a pharmaceutically acceptable salt thereof. In some aspects, the oligonucleotide is Antisense Oligo No. 329 of Table 3, or a pharmaceutically acceptable salt thereof. In some aspects, the oligonucleotide is Antisense Oligo No. 346 of Table 3, or a pharmaceutically acceptable salt thereof.

In some aspects, the oligonucleotide, or a pharmaceutically acceptable salt thereof, causes at least a 50% reduction in MSH3 mRNA expression at an oligonucleotide concentration of 10 nM. In some aspects, the oligonucleotide, or a pharmaceutically acceptable salt thereof, causes at least a 60% reduction in MSH3 mRNA expression at an oligonucleotide concentration of 10 nM. In some aspects, the oligonucleotide, or a pharmaceutically acceptable salt thereof, causes at least a 70% reduction in MSH3 mRNA expression at an oligonucleotide concentration of 10 nM. In some aspects, the oligonucleotide, or a pharmaceutically acceptable salt thereof, causes at least an 80% reduction in MSH3 mRNA expression at an oligonucleotide concentration of 10 nM.

In some aspects, the oligonucleotide, or a pharmaceutically acceptable salt thereof, causes at least a 50% reduction in MSH3 mRNA expression at an oligonucleotide concentration of 1 nM. In some aspects, the oligonucleotide, or a pharmaceutically acceptable salt thereof, causes at least a 60% reduction in MSH3 mRNA expression at an oligonucleotide concentration of 1 nM. In some aspects, the oligonucleotide, or a pharmaceutically acceptable salt thereof, causes at least a 70% reduction in MSH3 mRNA expression at an oligonucleotide concentration of 1 nM.

In some aspects, the oligonucleotide comprises: (a) a DNA core sequence comprising linked deoxyribonucleoside; (b) a 5′ flanking sequence comprising linked nucleoside; and (c) a 3′ flanking sequence comprising linked nucleoside; wherein the DNA core comprises a region of at least 10 contiguous nucleobase positioned between the 5′ flanking sequence and the 3′ flanking sequence; wherein the 5′ flanking sequence and the 3′ flanking sequence each comprise at least two linked nucleosides; and wherein at least one nucleoside of each flanking sequence comprises an alternative nucleoside, or a pharmaceutically acceptable salt thereof.

In some aspects, the oligonucleotide comprises at least one alternative internucleoside linkage, or a pharmaceutically acceptable salt thereof. In some aspects, the at least one alternative internucleoside linkage is a phosphorothioate internucleoside linkage. In some aspects, the at least one alternative internucleoside linkage is a 2′-alkoxy internucleoside linkage. In some aspects, the at least one alternative internucleoside linkage is an alkyl phosphate internucleoside linkage. In some aspects, the oligonucleotide comprises at least one alternative nucleobase, or a pharmaceutically acceptable salt thereof. In some aspects, the alternative nucleobase is 5′-methylcytosine, pseudouridine, or 5-methoxyuridine. In some aspects, the oligonucleotide comprises at least one alternative sugar moiety, or a pharmaceutically acceptable salt thereof. In some aspects, the alternative sugar moiety is 2′-OMe or a bicyclic nucleic acid. In some aspects, the oligonucleotide further comprises a ligand conjugated to the 5′ end or the 3′ end of the oligonucleotide through a monovalent or branched bivalent or trivalent linker, or a pharmaceutically acceptable salt thereof.

In some aspects of the disclosure, the MSH3 mRNA expression is evaluated in vitro. In some aspects, the MSH3 mRNA expression is evaluated in a cell based assay. In some aspects, the MSH3 mRNA expression is evaluated in HeLa cells. In some aspects, the MSH3 mRNA expression is determined by the quantitative reverse transcription polymerase chain reaction (RT-qPCR). In some aspects, the MSH3 mRNA is expression is normalized to the mRNA expression of a reference gene. In some aspects, the MSH3 mRNA expression is normalized to the mRNA expression of beta-glucuronidase (GUSB). In some aspects, the reduction in MSH3 mRNA expression is relative to a control. In some aspects, the control is the MSH3 mRNA expression in the absence of the oligonucleotide, or pharmaceutically acceptable salt thereof. In some aspects, the control is the MSH3 mRNA expression in the absence of the oligonucleotide, or pharmaceutically acceptable salt thereof, but in the prEence of a control oligonucleotide, or salt thereof. In some aspects, the control oligonucleotide, or salt thereof, is a scrambled luciferase targeting oligonucleotide. In some aspects, the reduction in MSH3 mRNA expression is calculated by a delta-delta Ct (ΔΔCT) method. In some aspects, the delta-delta Ct (ΔΔCT) method comprising the normalization of the MSH3 mRNA expression to the mRNA expression of a reference gene and to the MSH3 mRNA expression in the absence of the oligonucleotide, or pharmaceutically acceptable salt thereof but in the prEence of a control oligonucleotide, or salt thereof. In some aspects, the reference gene is beta-glucuronidase (GUSB) and/or the control oligonucleotide, or salt thereof, is a scrambled luciferase targeting oligonucleotide. In some aspects, the reduction in MSH3 mRNA expression is determined by the method of Example 1.

In some aspects, in the same assay, Antisense Oligo No. 1 cause approximately a 58% reduction in MSH3 mRNA expression at an oligonucleotide concentration of 10 nM. In some aspects, in the same assay, Antisense Oligo No. 1 cause approximately a 14% reduction in MSH3 mRNA expression at an oligonucleotide concentration of 1 nM. In some aspects,

In some aspects, the oligonucleotide is in the free base form. In some aspects, the oligonucleotide is a pharmaceutically acceptable salt thereof. In some aspects, the oligonucleotide is a sodium salt. In some aspects, the one or more oligonucleotide, or pharmaceutically acceptable salts thereof, are intracerebroventricularly administered as a pharmaceutical composition that comprises one or more of the oligonucleotide, or pharmaceutically acceptable salts thereof, and a pharmaceutically acceptable carrier or excipient. In some aspects, the pharmaceutical composition comprises artificial cerebrospinal fluid.

In some aspects of the disclosure, the subject is a primate. In some aspects, the primate is a human. In some aspects, the primate is a nonhuman primate.

In some aspects, the nucleotide repeat expansion disorder is spinocerebellar ataxia type 36 or frontotemporal dementia. In some aspects, the nucleotide repeat expansion disorder is a trinucleotide repeat expansion disorder. In some aspects, the trinucleotide repeat expansion disorder is a polyglutamine disease. In some aspects, the polyglutamine disease is selected from the group consisting of dentatorubropallidoluysian atrophy, Huntington's disease, spinal and bulbar muscular atrophy, spinocerebellar ataxia type 1, spinocerebellar ataxia type 2, spinocerebellar ataxia type 3, spinocerebellar ataxia type 6, spinocerebellar ataxia type 7, spinocerebellar ataxia type 17, and Huntington's disease-like 2. In some aspects, the trinucleotide repeat expansion disorder is a non-polyglutamine disease. In some aspects, the non-polyglutamine disease is selected from the group consisting of fragile X syndrome, fragile X-associated tremor/ataxia syndrome, fragile XE mental retardation, Friedreich's ataxia, myotonic dystrophy type 1, spinocerebellar ataxia type 8, spinocerebellar ataxia type 12, oculopharyngeal muscular dystrophy, Fragile X-associated premature ovarian failure, FRA2 A syndrome, FRA7 A syndrome, and early infantile epileptic encephalopathy.

In some aspects, the single-stranded oligonucleotide, or a pharmaceutically acceptable salt thereof, is administered at a dose of about 10 mg to about 250 mg. In some aspects, the single-stranded oligonucleotide, or a pharmaceutically acceptable salt thereof, is administered at a dose of about 15 mg to about 200 mg. In some aspects, the single-stranded oligonucleotide, or a pharmaceutically acceptable salt thereof, is administered at a dose of about 25 mg to about 200 mg. In some aspects, the single-stranded oligonucleotide, or a pharmaceutically acceptable salt thereof, is administered at a dose of about 50 mg to about 200 mg. In some aspects, the single-stranded oligonucleotide, or a pharmaceutically acceptable salt thereof, is administered at a dose of about 100 mg to about 150 mg.

In some aspects of the disclosure, the single-stranded oligonucleotide, or a pharmaceutically acceptable salt thereof, is administered once weekly. In some aspects, the single-stranded oligonucleotide, or a pharmaceutically acceptable salt thereof, is administered once every two weeks. In some aspects, the single-stranded oligonucleotide, or a pharmaceutically acceptable salt thereof, is administered once every three weeks. In some aspects, the single-stranded oligonucleotide, or a pharmaceutically acceptable salt thereof, is administered once every four weeks. In some aspects, the single-stranded oligonucleotide, or a pharmaceutically acceptable salt thereof, is administered once every month. In some aspects, the single-stranded oligonucleotide, or a pharmaceutically acceptable salt thereof, is administered once every six weeks. In some aspects, the single-stranded oligonucleotide, or a pharmaceutically acceptable salt thereof, is administered once every eight weeks. In some aspects, the single-stranded oligonucleotide, or a pharmaceutically acceptable salt thereof, is administered once every two months. In some aspects, the single-stranded oligonucleotide, or a pharmaceutically acceptable salt thereof, is administered once every ten weeks. In some aspects, the single-stranded oligonucleotide, or a pharmaceutically acceptable salt thereof, is administered once every twelve weeks. In some aspects, the single-stranded oligonucleotide, or a pharmaceutically acceptable salt thereof, is administered once every three months. In some aspects, the single-stranded oligonucleotide, or a pharmaceutically acceptable salt thereof, is administered once every sixteen weeks. In some aspects, the single-stranded oligonucleotide, or a pharmaceutically acceptable salt thereof, is administered once every four months. In some aspects, the single-stranded oligonucleotide, or a pharmaceutically acceptable salt thereof, is administered once every twenty weeks. In some aspects, the single-stranded oligonucleotide, or a pharmaceutically acceptable salt thereof, is administered once every five months. In some aspects, the single-stranded oligonucleotide, or a pharmaceutically acceptable salt thereof, is administered once every twenty-four weeks. In some aspects, the single-stranded oligonucleotide, or a pharmaceutically acceptable salt thereof, is administered once every six months.

In some aspects, administration of the single-stranded oligonucleotide, or a pharmaceutically acceptable salt thereof delays the onset and/or progression of the nucleotide repeat expansion disorder by at least 120 days, at least 6 months, at least 12 months, at least 2 years, at least 3 years, at least 4 years, at least 5 years, at least 10 years or more, when compared with a predicted onset and/or progression.

In some aspects the disclosure further comprises administering an additional therapeutic agent. In some aspects, the additional therapeutic agent is another oligonucleotide that hybridize to an mRNA encoding the Huntingtin gene.

In some aspects, the disclosure is related to an oligonucleotide that comprises:
(a) a DNA core sequence comprising linked deoxyribonucleosides;
(b) a 5′ flanking sequence comprising linked nucleosides; and
(c) a 3′ flanking sequence comprising linked nucleosides;
wherein the DNA core comprises a region of at least 10 contiguous nucleobases positioned between the 5′ flanking sequence and the 3′ flanking sequence; wherein the 5′ flanking sequence and the 3′ flanking sequence each comprises at least two linked nucleosides; and wherein at least one nucleoside of each flanking sequence comprises an alternative nucleoside, or a pharmaceutically acceptable salt thereof. In some aspects, the oligonucleotide comprises at least one alternative internucleoside linkage, or a pharmaceutically acceptable salt thereof. In some aspects, In some aspects, the at least one alternative internucleoside linkage is a phosphorothioate internucleoside linkage. In some aspects, the at least one alternative internucleoside linkage is a 2′-alkoxy internucleoside linkage. In some aspects, the at least one alternative internucleoside linkage is an alkyl phosphate internucleoside linkage. In some aspects, the oligonucleotide comprises at least one alternative nucleobase, or a pharmaceutically acceptable salt thereof. In some aspects, the alternative nucleobase is 5′-methylcytosine, pseudouridine, or 5-methoxyuridine. In some aspects, the oligonucleotide comprises at least one alternative sugar moiety, or a pharmaceutically acceptable salt thereof. In some aspects, the alternative sugar moiety is 2′-OMe or a bicyclic nucleic acid. In some aspects, the oligonucleotide further comprises a ligand conjugated to the 5′ end or the 3′ end of the oligonucleotide through a monovalent or branched bivalent or trivalent linker, or a pharmaceutically acceptable salt thereof.

### Definitions

For convenience, the meaning of some terms and phrases used in the specification, examples, and appended claims are provided below. Unless stated otherwise, or implicit from context, the following terms and phrases include the meanings provided below. The definitions are provided to aid in describing particular aspects, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

In this application, unless otherwise clear from context, (i) the term “a” can be understood to mean “at least one”; (ii) the term “or” can be understood to mean “and/or”; and (iii) the terms “including” and “comprising” can be understood to encompass itemized components or steps whether presented by themselves or together with one or more additional components or steps.

As used herein, the terms “about” and “approximately” refer to a value that is within 10% above or below the value being described. For example, the term “about 5 nM” indicates a range of from 4.5 to 5.5 nM.

The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range. “At least” is also not limited to integers (e.g., “at least 5% includes 5.0%, 5.1%, and 5.18% without consideration of the number of significant figures.

As used herein, “no more than” or “less than” is understood as the value adjacent to the phrase and logical lower values or integers, as logical from context, to zero. For example, an oligonucleotide with “no more than 3 mismatches to a target sequence” has 3, 2, 1, or 0 mismatches to a target sequence. When “no more than” is present before a series of numbers or a range, it is understood that “no more than” can modify each of the numbers in the series or range.

As used herein, the term “administration” refers to the administration of a composition (e.g., a compound or a preparation that includes a compound as described herein) to a subject or system. Administration to an animal subject (e.g., to a human) can be by any appropriate route, such as one described herein.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some aspects, the delivery of the two or more agents is simultaneous or concurrent and the agents can be co-formulated. In some aspects, the two or more agents are not co-formulated and are administered in a sequential manner as part of a prescribed regimen. In some aspects, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intraocular routes, subcutaneous routes, intra cisterna magna routes, intravenous routes, intramuscular routes, and direct absorption through mucous membrane tissues. The therapeutic agents can be administered by the same route or by different routes. For example, one therapeutic agent of the combination can be administered by intravenous injection while an additional therapeutic agent of the combination can be administered orally.

As used herein, the term “MSH3” refers to MutS Homolog 3, a DNA mismatch repair protein, having an amino acid sequence from any vertebrate or mammalian source, including, but not limited to, human, bovine, chicken, rodent, mouse, rat, porcine, ovine, primate, monkey, and guinea pig, unless specified otherwise. The term also refers to fragments and variants of native MSH3 that maintain at least one in vivo or in vitro activity of a native MSH3. The term encompasses full-length unprocessed precursor forms of MSH3 as well as mature forms resulting from post-translational cleavage of the signal peptide. MSH3 is encoded by the MSH3 gene. The nucleic acid sequence of an exemplary Homo sapiens (human) MSH3 gene is set forth in NCBI Reference NM_002439.4 or in SEQ ID NO: 385. The term “MSH3” also refers to natural variants of the wild-type MSH3 protein, such as proteins having at least 85% identity (e.g., 85%, 86%, 87%, 88%, 89%, 90%, 91%, 92%, 93%, 94%, 95%, 96%, 97%, 98%, 99%, 99.9% identity, or more) to the amino acid sequence of wild-type human MSH3, which is set forth in NCBI Reference No. NP_002430.3 or in SEQ ID NO: 386. The nucleic acid sequence of an exemplary Mus musculus (mouse) MSH3 gene is set forth in NCBI Reference No. NM_010829.2 or in SEQ ID NO: 387. The nucleic acid sequence of an exemplary Rattus norvegicus (rat) MSH3 gene is set forth in NCBI Reference No. NM_001191957.1 or in SEQ ID NO: 388. The nucleic acid sequence of an exemplary Macaca fascicularis (cyno) MSH3 gene is set forth in NCBI Reference No. XM_005557283.2 or in SEQ ID NO: 389.

The term “MSH3” as used herein also refers to a particular polypeptide expressed in a cell by naturally occurring DNA sequence variations of the MSH3 gene, such as a single nucleotide polymorphism in the MSH3 gene. Numerous SNPs within the MSH3 gene have been identified and can be found at, for example, NCBI dbSNP (see, e.g., www.ncbi.nlm.nih.gov/snp). Non-limiting examples of SNPs within the MSH3 gene can be found at, NCBI dbSNP Accession Nos.: rs1650697, rs70991108, rs10168, rs26279, rs26282, rs26779, rs26784, rs32989, rs33003, rs33008, rs33013, rs40139, rs181747, rs184967, rs245346, rs245397, rs249633, rs380691, rs408626, rs442767, rs836802, rs836808, rs863221, rs1105525, rs1428030, rs1478834, rs1650694, rs1650737, rs1677626, rs1677658, rs1805355, rs2897298, rs3045983, rs3797897, rs4703819, rs6151627, rs6151640, rs6151662, rs6151670, rs6151735, rs6151838, rs7709909, rs7712332, rs10079641, rs12513549, and rs12522132. As used herein, “target sequence” refers to a contiguous portion of the nucleotide sequence of an mRNA molecule formed during the transcription of an MSH3 gene, including mRNA that is a product of RNA processing of a primary transcription product. In one aspect, the target portion of the sequence will be at least long enough to serve as a substrate for oligonucleotide-directed (e.g., antisense oligonucleotide (ASO)-directed) cleavage at or near that portion of the nucleotide sequence of an mRNA molecule formed during the transcription of a MSH3 gene. The target sequence can be, for example, from about 9-36 nucleotides in length, e.g., about 15-30 nucleotides in length. For example, the target sequence can be 15, 16, 17, 18, 19, 20, 21, 22, 23, 24, 25, 26, 27, 28, 29, or 30 or from about 15-30 nucleotides, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 nucleotides in length. Ranges and lengths intermediate to the above recited ranges and lengths are also contemplated.

“G,” “C,” “A,” “T,” and “U” each generally stand for a naturally-occurring nucleotide that contains guanine, cytosine, adenine, thymidine, and uracil as a base, respectively. However, it will be understood that the term “nucleotide” can refer to an alternative nucleotide, as further detailed below, or a surrogate replacement moiety. The skilled person is well aware that guanine, cytosine, adenine, and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in the nucleotide sequences of oligonucleotides by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods featured herein.

The terms “nucleobase” and “base” include the purine (e.g. adenine and guanine) and pyrimidine (e.g. uracil, thymine, and cytosine) moiety present in nucleosides and nucleotides which form hydrogen bonds in nucleic acid hybridization. The term nucleobase also encompasses alternative nucleobases which can differ from naturally-occurring nucleobases, but are functional during nucleic acid hybridization. In this context, “nucleobase” refers to both naturally occurring nucleobases such as adenine, guanine, cytosine, thymidine, uracil, xanthine, and hypoxanthine, as well as alternative nucleobases. Such variants are for example described in Hirao et al (2012) Accounts of Chemical Research vol 45 page 2055 and Bergstrom (2009) Current Protocols in Nucleic Acid Chemistry Suppl. 37 1.4.1.

The term “nucleoside” refers to a monomeric unit of an oligonucleotide or a polynucleotide having a nucleobase and a sugar moiety. A nucleoside can include those that are naturally-occurring as well as alternative nucleosides, such as those described herein. The nucleobase of a nucleoside can be a naturally-occurring nucleobase or an alternative nucleobase. Similarly, the sugar moiety of a nucleoside can be a naturally-occurring sugar or an alternative sugar.

The term “alternative nucleoside” refers to a nucleoside having an alternative sugar or an alternative nucleobase, such as those described herein.

In some aspects, the nucleobase moiety is modified by changing the purine or pyrimidine into a modified purine or pyrimidine, such as substituted purine or substituted pyrimidine, such as an “alternative nucleobase” selected from isocytosine, pseudoisocytosine, 5-methyl cytosine, 5-thiozolo-cytosine, 5-propynyl-cytosine, 5-propynyl-uridine, 5-bromouridine 5-thiazolo-uridine, 2-thio-uridine, pseudouridine, 1-methylpseudouridine, 5-methoxyuridine, 2′-thio-thymine, inosine, diaminopurine, 6-aminopurine, 2-aminopurine, 2,6-diaminopurine, and 2-chloro-6-aminopurine.

The nucleobase moieties can be indicated by the letter code for each corresponding nucleobase, e.g. A, T, G, C, or U, wherein each letter can include alternative nucleobases of equivalent function. In some aspects, e.g., for gapmers, 5-methyl cytosine LNA nucleosides can be used.

A “sugar” or “sugar moiety,” includes naturally occurring sugars having a furanose ring. A sugar also includes an “alternative sugar,” defined as a structure that is capable of replacing the furanose ring of a nucleoside. In some aspects, alternative sugars are non-furanose (or 4′-substituted furanose) rings or ring systems or open systems. Such structures include simple changes relative to the natural furanose ring, such as a six-membered ring, or can be more complicated as is the case with the non-ring system used in peptide nucleic acid. Alternative sugars can include sugar surrogates wherein the furanose ring has been replaced with another ring system such as, for example, a morpholino or hexitol ring system. Sugar moieties useful in the preparation of oligonucleotides having motifs include, without limitation, β-D-ribose, β-D-2′-deoxyribose, substituted sugars (such as 2′, 5′ and bis substituted sugars), 4′-S-sugars (such as 4′-S-ribose, 4′-S-2′-deoxyribose and 4′-S-2′-substituted ribose), bicyclic alternative sugars (such as the 2′-O—CH₂-4′ or 2′-O(CH₂)₂-4′ bridged ribose derived bicyclic sugars) and sugar surrogates (such as when the ribose ring has been replaced with a morpholino or a hexitol ring system). The type of heterocyclic base and internucleoside linkage used at each position is variable and is not a factor in determining the motif. In most nucleosides having an alternative sugar moiety, the heterocyclic nucleobase is generally maintained to permit hybridization.

A “nucleotide,” as used herein, refers to a monomeric unit of an oligonucleotide or polynucleotide that comprises a nucleoside and an internucleosidic linkage. The internucleosidic linkage can include a phosphate linkage. Similarly, “linked nucleosides” can be linked by phosphate linkages. Many “alternative internucleosidic linkages” are known in the art, including, but not limited to, phosphate, phosphorothioate, and boronophosphate linkages. Alternative nucleosides include bicyclic nucleosides (BNAs) (e.g., locked nucleosides (LNAs (e.g., A-LNA, 5mC L-NA, G-LNA, T-LNA)) and constrained ethyl (cEt) nucleosides), peptide nucleosides (PNAs), phosphotriesters, phosphorothionates, phosphoramidates, and other variants of the phosphate backbone of native nucleoside, including those described herein.

An “alternative nucleotide,” as used herein, refers to a nucleotide having an alternative nucleoside or an alternative sugar, and an internucleoside linkage, which can include alternative nucleoside linkages.

The terms “oligonucleotide” and “polynucleotide,” as used herein, are defined as it is generally understood by the skilled person as a molecule comprising two or more covalently linked nucleosides. Such covalently bound nucleosides can be referred to as nucleic acid molecules or oligomers. Oligonucleotides are commonly made in the laboratory by solid-phase chemical synthesis followed by purification. When referring to a sequence of the oligonucleotide, reference is made to the sequence or order of nucleobase moieties, or modifications thereof, of the covalently linked nucleotides or nucleosides. The oligonucleotide can be man-made. For example, the oligonucleotide can be chemically synthesized, and be purified or isolated. Oligonucleotide is also intended to include (i) compounds that have one or more furanose moieties that are replaced by furanose derivatives or by any structure, cyclic or acyclic, that can be used as a point of covalent attachment for the base moiety, (ii) compounds that have one or more phosphodiester linkages that are either modified, as in the case of phosphoramidate or phosphorothioate linkages, or completely replaced by a suitable linking moiety as in the case of formacetal or riboacetal linkages, and/or (iii) compounds that have one or more linked furanose-phosphodiester linkage moieties replaced by any structure, cyclic or acyclic, that can be used as a point of covalent attachment for the base moiety. The oligonucleotide can comprise one or more alternative nucleosides or nucleotides (e.g., including those described herein). It is also understood that oligonucleotide includes compositions lacking a sugar moiety or nucleobase but are still capable of forming a pairing with or hybridizing to a target sequence. “Oligonucleotide” refers to a short polynucleotide (e.g., of 100 or fewer linked nucleosides).

As used herein, the term “oligonucleotide comprising a nucleobase sequence” refers to an oligonucleotide comprising a chain of nucleotides or nucleosides that is described by the sequence referred to using the standard nucleotide nomenclature.

The term “contiguous nucleobase region” refers to the region of the oligonucleotide which is complementary to the target nucleic acid. The term can be used interchangeably herein with the term “contiguous nucleotide sequence” or “contiguous nucleobase sequence.” In some aspects, all the nucleotides of the oligonucleotide are present in the contiguous nucleotide or nucleoside region. In some aspects, the oligonucleotide comprises the contiguous nucleotide region and can comprise further nucleotide(s) or nucleoside(s), for example a nucleotide linker region which can be used to attach a functional group to the contiguous nucleotide sequence. The nucleotide linker region can be complementary to the target nucleic acid. In some aspects, the internucleoside linkages present between the nucleotides of the contiguous nucleotide region are all phosphorothioate internucleoside linkages. In some aspects, the contiguous nucleotide region comprises one or more sugar-modified nucleosides.

The term “gapmer,” as used herein, refers to an oligonucleotide which comprises a region of RNase H recruiting oligonucleotides (gap or DNA core) which is flanked 5′ and 3′ by regions which comprise one or more affinity enhancing alternative nucleosides (wings or flanking sequence). Various gapmer designs are described herein. Headmers and tailmers are oligonucleotides capable of recruiting RNase H where one of the flanks is missing, i.e. only one of the ends of the oligonucleotide comprises affinity enhancing alternative nucleosides. For headmers the 3′ flanking sequence is missing (i.e. the 5′ flanking sequence comprises affinity enhancing alternative nucleosides) and for tailmers the 5′ flanking sequence is missing (i.e. the 3′ flanking sequence comprises affinity enhancing alternative nucleosides). A “mixed flanking sequence gapmer” refers to a gapmer wherein the flanking sequences comprise at least one alternative nucleoside, such as at least one DNA nucleoside or at least one 2′ substituted alternative nucleoside, such as, for example, 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, 2′-F-ANA nucleoside(s), or bicyclic nucleosides (e.g., locked nucleosides or constrained ethyl (cEt) nucleosides). In some aspects, the mixed flanking sequence gapmer has one flanking sequence which comprises alternative nucleosides (e.g. 5′ or 3′) and the other flanking sequence (3′ or 5′ respectfully) comprises 2′ substituted alternative nucleoside(s).

A “linker” or “linking group” is a connection between two atoms that links one chemical group or segment of interest to another chemical group or segment of interest via one or more covalent bonds. The oligonucleotides disclosed herein can comprise one or more linkers capable of linking one or more oligonucleotides disclosed herein to one or more other oligonucleotides disclosed herein, and/or to any other oligonucleotide, and/or to any conjugate moiety. For example, a linker could be used to link an oligonucleotide disclosed herein to an oligonucleotide that targets the Huntingtin gene.

Linkers may be susceptible to cleavage (“cleavable linker”) thereby facilitating release of the different oligonucleotides and/or different conjugate moieties disclosed herein. Such cleavable linkers may be susceptible, for example, to nuclease-induced cleavage, acid-induced cleavage, photo-induced cleavage, peptidase-induced cleavage, esterase-induced cleavage, and disulfide bond cleavage, at suitable conditions. Suitable cleavable linking groups for use in cleavable linkers include those which are sufficiently stable outside the cell, but which upon entry into a target cell is cleaved to release the two parts the linker is holding together.

Alternatively, linkers may be substantially resistant to cleavage (“non-cleavable linker”). Such non-cleavable linkers can be any chemical moiety capable of linking one or more different oligonucleotides disclosed herein to one or more other oligonucleotides disclosed herein, and/or to any conjugate moiety in a stable, covalent manner and does not fall off under the categories listed above for cleavable linkers. Thus, non-cleavable linkers are substantially resistant to acid-induced cleavage, nuclease-induced cleavage, photo-induced cleavage, peptidase-induced cleavage, esterase-induced cleavage and disulfide bond cleavage. Furthermore, non-cleavable refers to the ability of the chemical bond in the linker or adjoining to the linker to withstand cleavage induced by an acid, a nuclease, photolabile-cleaving agent, a peptidase, an esterase, or a chemical or physiological compound that cleaves a disulfide bond, at conditions under which the oligonucleotides disclosed herein do not lose their activity or intended purpose.

Conjugate moieties can be attached to the oligonucleotide directly or through a linking moiety (e.g. linker or tether). Linkers serve to covalently connect a third region, e.g. a conjugate moiety to an oligonucleotide (e.g. the termini of region A or C). In some aspects, the conjugate or oligonucleotide conjugate can, comprise a linker region which is positioned between the oligonucleotide and the conjugate moiety. In some aspects, the linker between the conjugate and oligonucleotide is biocleavable. Phosphodiester containing biocleavable linkers are described in more detail in WO 2014/076195 (herein incorporated by reference).

In some aspects, two or more linkers can be linked in tandem. When multiple linkers connect one or more oligonucleotides disclosed herein to one or more other oligonucleotides disclosed herein, and/or to any conjugate moiety, each of the linkers can be the same or different.

As used herein, and unless otherwise indicated, the term “complementary,” when used to describe a first nucleotide or nucleoside sequence in relation to a second nucleotide or nucleoside sequence, refers to the ability of an oligonucleotide or polynucleotide comprising the first nucleotide or nucleoside sequence to hybridize and form a duplex structure under certain conditions with an oligonucleotide or polynucleotide comprising the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° C., or 70° C., for 12-16 hours followed by washing (see, e.g., “Molecular Cloning: A Laboratory Manual, Sambrook, et al. (1989) Cold Spring Harbor Laboratory Press). Other conditions, such as physiologically relevant conditions as can be encountered inside an organism, can be used. The skilled person will be able to determine the set of conditions most appropriate for a test of complementarity of two sequences in accordance with the ultimate application of the hybridized nucleotides or nucleosides.

“Complementary” sequences, as used herein, can include, or be formed entirely from, non-Watson-Crick base pairs and/or base pairs formed from non-natural and alternative nucleotides or nucleosides, in so far as the above requirements with respect to their ability to hybridize are fulfilled. Such non-Watson-Crick base pairs include, but are not limited to, G:U Wobble or Hoogstein base pairing. Complementary sequences between an oligonucleotide and a target sequence as described herein, include base-pairing of the oligonucleotide or polynucleotide comprising a first nucleotide or nucleoside sequence to an oligonucleotide or polynucleotide comprising a second nucleotide or nucleoside sequence over the entire length of one or both nucleotide or nucleoside sequences. Such sequences can be referred to as “fully complementary” with respect to each other herein. However, where a first sequence is referred to as “substantially complementary” with respect to a second sequence herein, the two sequences can be fully complementary, or they can form one or more, but generally not more than 5, 4, 3 or 2 mismatched base pairs upon hybridization for a duplex up to 30 base pairs, while retaining the ability to hybridize under the conditions most relevant to their ultimate application, e.g., inhibition of gene expression via an RNase H-mediated pathway. “Substantially complementary” can refer to a polynucleotide that is substantially complementary to a contiguous portion of the mRNA of interest (e.g., an mRNA encoding MSH3). For example, a polynucleotide is complementary to at least a part of a MSH3 mRNA if the sequence is substantially complementary to a non-interrupted portion of an mRNA encoding MSH3.

As used herein, the term “region of complementarity” refers to the region on the oligonucleotide that is substantially complementary to all or a portion of a gene, primary transcript, a sequence (e.g., a target sequence, e.g., an MSH3 nucleotide sequence), or processed mRNA, so as to interfere with expression of the endogenous gene (e.g., MSH3). Where the region of complementarity is not fully complementary to the target sequence, the mismatches can be in the internal or terminal regions of the molecule. Generally, the most tolerated mismatches are in the terminal regions, e.g., within 5, 4, 3, or 2 nucleotides of the 5′- and/or 3′-terminus of the oligonucleotide.

As used herein, an “agent that reduces the level and/or activity of MSH3” refers to any polynucleotide agent (e.g., an oligonucleotide, e.g., an ASO) that reduces the level of or inhibits expression of MSH3 in a cell or subject. The phrase “inhibiting expression of MSH3,” as used herein, includes inhibition of expression of any MSH3 gene (such as, e.g., a mouse MSH3 gene, a rat MSH3 gene, a monkey MSH3 gene, or a human MSH3 gene) as well as variants or mutants of a MSH3 gene that encode a MSH3 protein. Thus, the MSH3 gene can be a wild-type MSH3 gene, a mutant MSH3 gene, or a transgenic MSH3 gene in the context of a genetically manipulated cell, group of cells, or organism.

By “reducing the activity of MSH3” is meant decreasing the level of an activity related to MSH3 (e.g., by reducing the amount of nucleotide repeats in a gene associated with a nucleotide repeat expansion disorder, e.g., a trinucleotide repeat expansion disorder, that is related to MSH3 activity). The activity level of MSH3 can be measured using any method known in the art (e.g., by directly sequencing a gene associated with a nucleotide repeat expansion disorder to measure the levels of nucleotide repeats).

By “reducing the level of MSH3” is meant decreasing the level of MSH3 in a cell or subject, e.g., by administering an oligonucleotide, or pharmaceutically acceptable salt thereof, to the cell or subject. The level of MSH3 can be measured using any method known in the art (e.g., by measuring the levels of MSH3 mRNA or levels of MSH3 protein in a cell or a subject).

By “modulating the activity of a MutSP heterodimer comprising MSH3” is meant altering the level of an activity related to a MutSP heterodimer, or a related downstream effect. The activity level of a MutSP heterodimer can be measured using any method known in the art.

As used herein, the term “inhibitor” refers to any agent which reduces the level and/or activity of a protein (e.g., MSH3). Non-limiting examples of inhibitors include polynucleotides (e.g., oligonucleotide, e.g., ASOs). The term “inhibiting,” as used herein, is used interchangeably with “reducing,” “silencing,” “downregulating,” “suppressing,” “knocking down,” and other similar terms, and includes any level of inhibition.

The phrase “contacting a cell with an oligonucleotide,” such as an oligonucleotide, as used herein, includes contacting a cell by any possible means. Contacting a cell with an oligonucleotide includes contacting a cell in vitro with the oligonucleotide or contacting a cell in vivo with the oligonucleotide. The contacting can be done directly or indirectly. Thus, for example, the oligonucleotide can be put into physical contact with the cell by the individual performing the method, or alternatively, the oligonucleotide agent can be put into a situation that will permit or cause it to subsequently come into contact with the cell.

Contacting a cell in vitro can be done, for example, by incubating the cell with the oligonucleotide. Contacting a cell in vivo can be done, for example, by injecting the oligonucleotide into or near the tissue where the cell is located, or by injecting the oligonucleotide agent into another area, e.g., the bloodstream or the subcutaneous space, such that the agent will subsequently reach the tissue where the cell to be contacted is located. For example, the oligonucleotide can contain and/or be coupled to a ligand, e.g., GalNAc3, that directs the oligonucleotide to a site of interest, e.g., the liver. Combinations of in vitro and in vivo methods of contacting are also possible. For example, a cell can be contacted in vitro with an oligonucleotide and subsequently transplanted into a subject.

In one aspect, contacting a cell with an oligonucleotide includes “introducing” or “delivering the oligonucleotide into the cell” by facilitating or effecting uptake or absorption into the cell. Absorption or uptake of an ASO can occur through unaided diffusive or active cellular processes, or by auxiliary agents or devices. Introducing an oligonucleotide into a cell can be in vitro and/or in vivo. For example, for in vivo introduction, oligonucleotides can be injected into a tissue site or administered systemically. In vitro introduction into a cell includes methods known in the art such as electroporation and lipofection. Further approaches are described herein below and/or are known in the art.

The term “antisense,” as used herein, refers to a nucleic acid comprising an oligonucleotide or polynucleotide that is sufficiently complementary to all or a portion of a gene, primary transcript, or processed mRNA, so as to interfere with expression of the endogenous gene (e.g., MSH3). “Complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. It is understood that two polynucleotides can hybridize to each other even if they are not completely complementary to each other, provided that each has at least one region that is substantially complementary to the other.

As used herein, the terms “effective amount,” “therapeutically effective amount,” and “a “sufficient amount” of an agent that reduces the level and/or activity of MSH3 (e.g., in a cell or a subject) described herein refer to a quantity sufficient to, when administered to the subject, including a human, effect beneficial or desired results, including clinical results, and, as such, an “effective amount” or synonym thereto depends on the context in which it is being applied. For example, in the context of treating a nucleotide repeat expansion disorder (e.g., a trinucleotide repeat expansion disorder), it is an amount of the agent that reduces the level and/or activity of MSH3 sufficient to achieve a treatment response as compared to the response obtained without administration of the agent that reduces the level and/or activity of MSH3. The amount of a given agent that reduces the level and/or activity of MSH3 described herein that will correspond to such an amount will vary depending upon various factors, such as the given agent, the pharmaceutical formulation, the route of administration, the type of disease or disorder, the identity of the subject (e.g., age, sex, and/or weight) or host being treated, and the like, but can nevertheless be routinely determined by one of skill in the art. Also, as used herein, a “therapeutically effective amount” of an agent that reduces the level and/or activity of MSH3 of the present disclosure is an amount which results in a beneficial or desired result in a subject as compared to a control. As defined herein, a therapeutically effective amount of an agent that reduces the level and/or activity of MSH3 of the present disclosure can be readily determined by one of ordinary skill by routine methods known in the art. Dosage regimen can be adjusted to provide the optimum therapeutic response.

“Prophylactically effective amount,” as used herein, is intended to include the amount of an oligonucleotide that, when administered to a subject having or predisposed to have a nucleotide repeat expansion disorder (e.g., a trinucleotide repeat expansion disorder), is sufficient to prevent or ameliorate the disease or one or more symptoms of the disease. Ameliorating the disease includes slowing the course of the disease or reducing the severity of later-developing disease. The “prophylactically effective amount” can vary depending on the oligonucleotide, how the agent is administered, the degree of risk of disease, and the history, age, weight, family history, genetic makeup, the types of preceding or concomitant treatments, if any, and other individual characteristics of the patient to be treated. A prophylactically effective amount can refer to, for example, an amount of the agent that reduces the level and/or activity of MSH3 (e.g., in a cell or a subject) described herein or can refer to a quantity sufficient to, when administered to the subject, including a human, delay the onset of one or more of the nucleotide repeat disorders (e.g., trinucleotide repeat expansion disorders) described herein by at least 120 days, for example, at least 6 months, at least 12 months, at least 2 years, at least 3 years, at least 4 years, at least 5 years, at least 10 years or more, when compared with the predicted onset.

A “therapeutically-effective amount” or “prophylactically effective amount” also includes an amount (either administered in a single or in multiple doses) of an oligonucleotide that produces some desired local or systemic effect at a reasonable benefit/risk ratio applicable to any treatment. Oligonucleotides employed in the methods herein can be administered in a sufficient amount to produce a reasonable benefit/risk ratio applicable to such treatment.

As used herein, the term “region of complementarity” refers to the region on the oligonucleotide that is substantially complementary to all or a portion of a gene, primary transcript, a sequence (e.g., a target sequence, e.g., an MSH3 nucleotide sequence), or processed mRNA, so as to interfere with expression of the endogenous gene (e.g., MSH3). Where the region of complementarity is not fully complementary to the target sequence, the mismatches can be in the internal or terminal regions of the molecule. Generally, the most tolerated mismatches are in the terminal regions, e.g., within 5, 4, 3, or 2 nucleotides of the 5′- and/or 3′-terminus of the oligonucleotide.

An “amount effective to reduce nucleotide repeat expansion” of a particular gene refers to an amount of the agent that reduces the level and/or activity of MSH3 (e.g., in a cell or a subject) described herein, or to a quantity sufficient to, when administered to the subject, including a human, to reduce the nucleotide repeat expansion of a particular gene (e.g., a gene associated with a nucleotide repeat expansion disorder, e.g., a trinucleotide repeat expansion disorder, described herein).

As used herein, the term “a subject identified as having a nucleotide repeat expansion disorder” refers to a subject identified as having a molecular or pathological state, disease or condition of or associated with a nucleotide repeat expansion disorder, such as the identification of a nucleotide repeat expansion disorder or symptoms thereof, or to identification of a subject having or suspected of having a nucleotide repeat expansion disorder who can benefit from a particular treatment regimen.

As used herein, “trinucleotide repeat expansion disorder” refers to a class of genetic diseases or disorders characterized by excessive trinucleotide repeats (e.g., trinucleotide repeats such as CAG) in a gene or intron in the subject which exceed the normal, stable threshold, for the gene or intron. Nucleotide repeats are common in the human genome and are not normally associated with disease. In some cases, however, the number of repeats expands beyond a stable threshold and can lead to disease, with the severity of symptoms generally correlated with the number of repeats. Nucleotide repeat expansion disorders include “polyglutamine” and “non-polyglutamine” disorders.

By “determining the level of a protein” is meant the detection of a protein, or an mRNA encoding the protein, by methods known in the art either directly or indirectly. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third-party laboratory that directly acquired the physical entity or value). Methods to measure protein level generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners. Methods to measure mRNA levels are known in the art.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps (DNA core sequences), if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values can be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:
100 multiplied by (the fraction X/Y)
   where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

By “level” is meant a level or activity of a protein, or mRNA encoding the protein (e.g., MSH3), optionally as compared to a reference. The reference can be any useful reference, as defined herein. By a “decreased level” or an “increased level” of a protein is meant a decrease or increase in protein level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than 10%, 15%, 20%, 50%, 75%, 100%, or 200%, as compared to a reference; a decrease or an increase by less than 0.01-fold, 0.02-fold, 0.1-fold, 0.3-fold, 0.5-fold, 0.8-fold, or less; or an increase by more than 1.2-fold, 1.4-fold, 1.5-fold, 1.8-fold, 2.0-fold, 3.0-fold, 3.5-fold, 4.5-fold, 5.0-fold, 10-fold, 15-fold, 20-fold, 30-fold, 40-fold, 50-fold, 100-fold, 1000-fold, or more). A level of a protein can be expressed in mass/vol (e.g., g/dL, mg/mL, g/mL, or ng/mL) or percentage relative to total protein or mRNA in a sample.

The term “pharmaceutical composition,” as used herein, represents a composition containing a compound described herein formulated with a pharmaceutically acceptable excipient, and can be manufactured or sold with the approval of a governmental regulatory agency as part of a therapeutic regimen for the treatment of disease in a mammal. Pharmaceutical compositions can be formulated, for example, for intracerebroventricular injections; or in any other pharmaceutically acceptable formulation.

In some aspects, provided herein are pharmaceutical compositions that are formulated for intracerebroventricular injection.

A “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients can include, for example: artificial cerebrospinal fluid (acsf).

As used herein, the term “pharmaceutically acceptable salt” means any pharmaceutically acceptable salt of the compound of any of the compounds described herein. For example, pharmaceutically acceptable salts of any of the compounds described herein include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid.

The compounds described herein can have ionizable groups so as to be capable of preparation as pharmaceutically acceptable salts. These salts can be acid addition salts involving inorganic or organic acids or the salts can, in the case of acidic forms of the compounds described herein, be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts can be prepared from pharmaceutically acceptable non-toxic acids and bases including inorganic and organic acids and bases. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

By a “reference” is meant any useful reference used to compare protein or mRNA levels or activity. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated with a compound described herein; a sample from a subject that has been treated by a compound described herein; or a sample of a purified protein (e.g., any described herein) at a known normal concentration. By “reference standard or level” is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having a disease or disorder (e.g., a nucleotide or trinucleotide repeat expansion disorder); a subject that has been treated with a compound described herein. In some aspects, the reference sample, standard, or level is matched to the sample subject sample by at least one of the following criteria: age, weight, sex, disease stage, and overall health. A standard curve of levels of a purified protein, e.g., any described herein, within the normal reference range can be used as a reference.

As used herein, the term “subject” refers to any organism to which a composition can be administered, e.g., for experimental, diagnostic, prophylactic, and/or therapeutic purposes. Typical subjects include any animal (e.g., mammals such as mice, rats, rabbits, non-human primates, and humans). A subject can seek or be in need of treatment, require treatment, be receiving treatment, be receiving treatment in the future, or be a human or animal who is under care by a trained professional for a particular disease or condition.

As used herein, the terms “treat,” “treated,” and “treating” mean both therapeutic treatment and prophylactic or preventative measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

As used herein, the terms “variant” and “derivative” are used interchangeably and refer to naturally-occurring, synthetic, and semi-synthetic analogues of a compound, peptide, protein, or other substance described herein. A variant or derivative of a compound, peptide, protein, or other substance described herein can retain or improve upon the biological activity of the original material.

The details of one or more aspects are set forth in the description below. Other features, objects, and advantages will be apparent from the description and from the claims.

### DETAILED DESCRIPTION

The present inventors have found that suppression, inhibition, or depletion of MSH3 level and/or activity in a cell is effective in the treatment of a nucleotide repeat expansion disorder (e.g., a trinucleotide repeat expansion disorder). Accordingly, useful compositions and methods to treat nucleotide repeat expansion disorders (e.g., a trinucleotide repeat expansion disorder), e.g., in a subject in need thereof are provided herein.

### I. Nucleotide Repeat Expansion Disorders

Nucleotide repeat expansion disorders (e.g., trinucleotide repeat expansion disorders) are a family of genetic disorders characterized by the pathogenic expansion of a repeat region within a genomic region. In such disorders, the number of repeats exceeds that of a gene's normal, stable threshold, expanding into a diseased range.

Nucleotide repeat expansion disorders (e.g., trinucleotide repeat expansion disorders) generally can be categorized as “polyglutamine” or “non-polyglutamine.” Polyglutamine disorders, including Huntington's disease (HD) and several spinocerebellar ataxias, are caused by a CAG (glutamine) repeats in the protein-coding regions of specific genes. Non-polyglutamine disorders are more heterogeneous and can be caused by CAG nucleotide repeat expansions in non-coding regions, as in Myotonic dystrophy, or by the expansion of nucleotide repeats other than CAG that can be in coding or non-coding regions such as the CGG repeat expansion responsible for Fragile X Syndrome.

Nucleotide repeat expansion disorders (e.g., trinucleotide repeat expansion disorders) are dynamic in the sense that the number of repeats can vary from generation-to-generation, or even from cell-to-cell in the same individual. Repeat expansion is believed to be caused by polymerase “slipping” during DNA replication. Tandem repeats in the DNA sequence can “loop out” while maintaining complementary base pairing between the parent strand and daughter strands. If the loop structure is formed from the daughter strand, the number of repeats will increase.

Conversely, if the loop structure is formed from the parent strand, the number of repeats will decrease. It appears that expansion is more common than reduction. In general, the length of repeat expansion is negatively correlated with prognosis; longer repeats are correlated with an earlier age of onset and worsened disease severity. Thus, nucleotide repeat expansion disorders (e.g., trinucleotide repeat expansion disorders) are subject to “anticipation,” meaning the severity of symptoms and/or age of onset worsen through successive generations of affected families due to the expansion of these repeats from one generation to the next.

Nucleotide repeat expansion disorders (e.g., trinucleotide repeat expansion disorders) are well known in the art. For example, frontotemporal dementia (FTD) is a hexanucleotide repeat string of nucleotides GGGGCC that is repeated many more times in an individual than an individual without FTD. Additionally, an individual having spinocerebellar ataxia type 36 (SCA36) has many more GGCCTG repeats than an individual without SCA36.

Exemplary trinucleotide repeat expansion disorders and the trinucleotide repeats of the genes commonly associated with them are included in Table 1.

TABLE 1
Exemplary Trinucleotide Repeat Expansion Disorders

| Disease | Gene | Nucleotide Repeat |
|---|---|---|
| ARX-nonsyndromic X-linked mental | ARX | GCG |
| retardation (XLMR) | | |
| Baratela-Scott Syndrome | XYLT1 | GGC |
| Blepharophimosis / Ptosis / Epicanthus | FOXL2 | GCG |
| inversus syndrome type II | | |
| Cleidocranial dysplasia (CCD) | RUNX2 | GCG |
| Congenital central hypoventilation | PHOX-2B | GCG |
| Congenital central hypoventilation | PHOX2B | GCG |
| syndrome (CCHS) | | |
| Creutzfeldt-Jakob disease | PRNP | |
| Dentatorubral-pallidoluysian atrophy | ATN1 | CAG |
| (DRPLA) / Haw River syndrome | | |
| Early infantile epileptic encephalopathy | ARX | GCG |
| (Ohtahara syndrome) | | |
| FRA2A syndrome | AFF3 | CGC |
| FRA7A syndrome | ZNF713 | CGG |
| Fragile X mental retardation (FRAX-E) | AFF2 / FMR2 | GCC |
| Fragile X Syndrome (FXS) | FMR1 | CGG |
| Fragile X-associated Primary Ovarian | FMR1 | CGG |
| Insufficiency (FXPOI) | | |
| Fragile X-associated Tremor Ataxia | FMR1 | CGG |
| Syndrome (FXTAS) | | |
| Friedreich ataxia (FRDA) | FXN | GAA |
| Fuchs' Corneal Endothelial Dystrophy | TCF4 | CTG |
| (FECD) | | |
| Hand-foot genital syndrome (HFGS) | HOXA13 | GCG |
| Holoprosencephaly disorder (HPE) | ZIC2 | GCG |
| Huntington disease-like 2 (HDL2) | JPH3 | CTG |
| Huntington's Disease (HD) | HTT | CAG |
| Infantile spasm syndrome / West | ARX | GCG |
| syndrome (ISS) | | |
| Jacobsen syndrome | | |
| KCNN3-associated (e.g., | KCNN3 | CAG |
| schizophrenia) | | |
| Multiple Skeletal dysplasias | COMP | GAC |
| Myotonic Dystrophy type 1 (DM1) | DMPK | CTG |
| Myotonic Dystrophy type 2 (DM2) | CNBP | CCTG |
| NCOA3-associated (e.g., increased risk | NCOA3 | CAG |
| of prostate cancer) | | |
| Neuronal intranuclear inclusion disease | NOTCH2NLC | GGC |
| (NIID) | | |
| Oculopharyngeal Muscular Dystrophy | PABPN1 | GCG |
| (OPMD) | | |
| Spastic ataxia - Charlevoix-Saguenay | | |
| Spinal Muscular Bulbar Atrophy | AR | CAG |
| (SMBA) | | |
| Spinocerebellar ataxia type 1 (SCA1) | ATXNI | CAG |
| Spinocerebellar ataxia type 10 (SCA10) | ATXN10 | ATTCT |
| Spinocerebellar ataxia type 12 (SCA12) | PPP2R2B | CAG |
| Spinocerebellar ataxia type 17 (SCA17) | TBP / ATXN17 | CAG |
| Spinocerebellar ataxia type 2 (SCA2) | ATXN2 | CAG |
| Spinocerebellar ataxia type 3 (SCA3) / | ATXN3 | CAG |
| Machado-Joseph Disease | | |
| Spinocerebellar ataxia type 45 (SCA45) | FAT2 | CAG |
| Spinocerebellar ataxia type 6 (SCA6) | CACNAIA | CAG |
| Spinocerebellar ataxia type 7 (SCA7) | ATXN7 | CAG |
| Spinocerebellar ataxia type 8 (SCA8) | ATXN8 | CTG |
| Syndromic neurodevelopmental | MAB21L1 | CAG |
| disorder with cerebellar, ocular, | | |
| craniofacial, and genital features | | |
| (COFG syndrome) | | |
| Synpolydactyly (SPD I) | HOXD13 | GCG |
| Synpolydactyly (SPD II) | HOXD12 | GCG |

The proteins associated with nucleotide repeat expansion disorders (e.g., trinucleotide repeat expansion disorders) are typically selected based on an experimental association of the protein associated with a nucleotide repeat expansion disorder (e.g., a trinucleotide repeat expansion disorder) to a nucleotide repeat expansion disorder. For example, the production rate or circulating concentration of a protein associated with a nucleotide repeat expansion disorder (e.g., trinucleotide repeat expansion disorder) can be elevated or depressed in a population having a nucleotide repeat expansion disorder (e.g., a trinucleotide repeat expansion disorder) relative to a population lacking the nucleotide repeat expansion disorder (e.g., trinucleotide repeat expansion disorder). Differences in protein levels can be assessed using proteomic techniques including but not limited to Western blot, immunohistochemical staining, enzyme linked immunosorbent assay (ELISA), and mass spectrometry. Alternatively, the proteins associated with nucleotide repeat expansion disorders (e.g., trinucleotide repeat expansion disorders) can be identified by obtaining gene expression profiles of the genes encoding the proteins using genomic techniques including, but not limited to, DNA microarray analysis, serial analysis of gene expression (SAGE), and quantitative real-time polymerase chain reaction (qPCR).

### II. Evidence for the Involvement of Mismatch Repair Pathway in Nucleotide Repeat Expansion

There is growing evidence that DNA repair pathways, particularly mismatch repair (MMR), are involved in the expansion of nucleotide repeats (e.g., trinucleotide repeats). A recent genome-wide association (GWA) analysis led to the identification of loci harboring genetic variations that alter the age at neurological onset of Huntington's disease (HD) (GEM-HD Consortium, Cell. 2015 Jul. 30; 162(3):516-26). The study identified MLH1, the human homolog of the E. coli DNA mismatch repair gene mutL. A subsequent GWA study in polyglutamine disease patients found significant association of age at onset when grouping all polyglutamine diseases (HD and SCAs) with DNA repair genes as a group, as well as significant associations for specific SNPs in FAN1 and PMS2 with the diseases (Bettencourt et al., (2016) Ann. Neurol., 79: 983-990). These results were consistent with those from an earlier study comparing differences in repeat expansion in two different mouse models of Huntington's Disease, which identified Mlh1 and Mlh3 as novel critical modifiers of CAG instability (Pinto et al., (2013) Mismatch Repair Genes Mlh1 and Mlh3 Modify CAG Instability in Huntington's Disease Mice: Genome-Wide and Candidate Approaches. PLoS Genet 9(10): e1003930). Another member of the mismatch repair pathway, 8-oxo-guanine glycosylase (OGG1) has also been implicated in expansion, as somatic expansion was found to be reduced in transgenic mice lacking OGG1 (Kovtun I. V. et al. (2007) Nature 447, 447-452). However, another study found that human subjects containing a Ser326Cys polymorphism in hOGG1, which results in reduced OGG1 activity, results in increased mutant huntingtin (Coppede et al., (2009) Toxicol., 278: 199-203). Likewise, complete inactivation of Fan1, another component of the DNA repair pathway, in a mouse HD model produces somatic CAG expansions (Long et al. (2018) J. Hum Genet., 103: 1-9). MSH3, another component of the mismatch repair pathway, has been reported to be linked to somatic expansion: polymorphisms in Msh3 was associated with somatic instability of the expanded CTG trinucleotide repeat in myotonic dystrophy type 1 (DM1) patients (Morales et al., (2016) DNA Repair 40: 57-66). Furthermore, natural polymorphisms in Msh3 and Mlh1 have been revealed as mediators of mouse strain specific differences in CTG·CAG repeat instability (Pinto et al. (2013) ibid; Tome et al., (2013) PLoS Genet. 9 e1003280). Further evidence of Msh2 and Msh3's involvement in expansion repeats was reported in a study in which short hairpin RNA (shRNA) knockdown of either MSH2 or MSH3 slowed, and ectopic expression of either MSH2 or MSH3 induced GAA trinucleotide repeat expansion of the Friedreich Ataxia (FRDA) gene in fibroblasts derived from FRDA patients (Halabi et al., (2012) J. Biol. Chem. 287, 29958-29967). In spite of some inconsistent results provided above, there is strong evidence that the MMR pathway plays some role in the expansion of trinucleotide repeats in various disorders. Moreover, they are the first to recognize that the inhibition of the MMR pathway provides for the treatment or prevention of these repeat expansion disorders; however, no therapy is currently available or in development which modulates MMR for purposes of treating or preventing these repeat expansion disorders.

### III. Oligonucleotide Agents

Agents described herein that reduce the level and/or activity of MSH3 in a cell can be, for example, a polynucleotide, e.g., an oligonucleotide, or pharmaceutically acceptable salt thereof, are to be utilized in the compositions and methods described herein. These agents reduce the level of an activity related to MSH3, or a related downstream effect, or reduce the level of MSH3 in a cell or subject.

In some aspects, the agent that reduces the level and/or activity of MSH3 is a polynucleotide. In some aspects, the polynucleotide is a single-stranded oligonucleotide, e.g., that acts by way of an RNase H-mediated pathway. Oligonucleotides include DNA and DNA/RNA chimeric molecules, typically about 10 to 30 nucleotides in length, which recognize polynucleotide target sequences or sequence portions through hydrogen bonding interactions with the nucleotide bases of the target sequence (e.g., MSH3). An oligonucleotide molecule can decrease the expression level (e.g., protein level or mRNA level) of MSH3. For example, an oligonucleotide includes oligonucleotides that targets full-length MSH3. In some aspects, the oligonucleotide molecule recruits an RNase H enzyme, leading to target mRNA degradation.

In some aspects, the oligonucleotide, or pharmaceutically acceptable salt thereof, decreases the level and/or activity of a positive regulator of function. In other aspects, the oligonucleotide, or pharmaceutically acceptable salt thereof, increases the level and/or activity of an inhibitor of a positive regulator of function. In some aspects, the oligonucleotide, or pharmaceutically acceptable salt thereof, increases the level and/or activity of a negative regulator of function.

In some aspects, the oligonucleotide, or pharmaceutically acceptable salt thereof, decreases the level and/or activity or function of MSH3. In some aspects, the oligonucleotide, or pharmaceutically acceptable salt thereof, inhibits expression of MSH3. In other aspects, the oligonucleotide, or pharmaceutically acceptable salt thereof, increases degradation of MSH3 and/or decreases the stability (i.e., half-life) of MSH3. The oligonucleotide, or pharmaceutically acceptable salt thereof, can be chemically synthesized.

An oligonucleotide, or pharmaceutically acceptable salt thereof, can be synthesized by standard methods known in the art as further discussed below, e.g., by use of an automated DNA synthesizer, such as are commercially available from, for example, Biosearch, Applied Biosystems, Inc.

The oligonucleotide, or pharmaceutically acceptable salt thereof, compound can be prepared using solution-phase or solid-phase organic synthesis or both. Organic synthesis offers the advantage that the oligonucleotide, or pharmaceutically acceptable salt thereof, comprising unnatural or alternative nucleotides can be easily prepared. A single-stranded oligonucleotide, or pharmaceutically acceptable salt thereof, can be prepared using solution-phase or solid-phase organic synthesis or both.

Some aspects of the disclosure are related to a single-stranded oligonucleotide of 15-30 linked nucleotides in length, wherein the oligonucleotide, or a portion thereof, is at least 95% complementary to at least 15 contiguous nucleobases at positions 2543-2573 of SEQ ID NO: 614, or a pharmaceutically acceptable salt thereof. In some aspects, the oligonucleotide, or a portion thereof, is at least 98% complementary to at least 15 contiguous nucleobases at positions 2543-2573 of SEQ ID NO: 614, or a pharmaceutically acceptable salt thereof. In some aspects, the oligonucleotide, or a portion thereof, is at least 99% complementary to at least 15 contiguous nucleobases at positions 2543-2573 of SEQ ID NO: 614, or a pharmaceutically acceptable salt thereof. In some aspects, the oligonucleotide, or a portion thereof, is 100% complementary to at least 15 contiguous nucleobases at positions 2543-2573 of SEQ ID NO: 614, or a pharmaceutically acceptable salt thereof. In some aspects, the oligonucleotide, or a portion thereof, is complementary to 17-23 contiguous nucleobases at positions 2543-2573 of SEQ ID NO: 614, or a pharmaceutically acceptable salt thereof. In some aspects, the oligonucleotide is complementary to 17-20 contiguous nucleobases at positions 2543-2573 of SEQ ID NO: 614, or a pharmaceutically acceptable salt thereof. In some aspects, the 17-20 contiguous nucleobases begin at position 2543, 2544, 2545, 2546, 2547, 2548, 2549, 2550, 2551, 2552, 2553, 2554, 2555, 2556, or 2557 of SEQ ID NO: 614, or a pharmaceutically acceptable salt thereof. In some aspects, the oligonucleotide is 17-20 linked nucleotides in length, or a pharmaceutically acceptable salt thereof.

In some aspects, the oligonucleotide, or a portion thereof, is complementary to 20-23 contiguous nucleobases at positions 2543-2573 of SEQ ID NO: 614, or a pharmaceutically acceptable salt thereof. In some aspects, the 20-23 contiguous nucleobases begin at position 2543, 2544, 2545, 2546, 2547, 2548, 2549, 2550, 2551, 2552, 2553, or 2554 of SEQ ID NO: 614, or a pharmaceutically acceptable salt thereof. In some aspects, the oligonucleotide is 20-23 linked nucleotides in length, or a pharmaceutically acceptable salt thereof.

In some aspects, the oligonucleotide, or a portion thereof, is complementary to positions 2543-2570 of SEQ ID NO: 614, or a pharmaceutically acceptable salt thereof.

The disclosure also relates to single-stranded oligonucleotides of 15-30 linked nucleotides in length, wherein the oligonucleotide, or a portion thereof, is at least 95% complementary to at least 15 contiguous nucleobases at positions 2685-2714 of SEQ ID NO: 614, or a pharmaceutically acceptable salt thereof. In some aspects, the oligonucleotide, or a portion thereof, is at least 98% complementary to at least 15 contiguous nucleobases at positions 2685-2714 of SEQ ID NO: 614, or a pharmaceutically acceptable salt thereof. In some aspects, the oligonucleotide, or a portion thereof, is at least 99% complementary to at least 15 contiguous nucleobases at positions 2685-2714 of SEQ ID NO: 614, or a pharmaceutically acceptable salt thereof. In some aspects, the oligonucleotide or a portion thereof, is 100% complementary to at least 15 contiguous nucleobases at positions 2685-2714 of SEQ ID NO: 614, or a pharmaceutically acceptable salt thereof. In some aspects, the oligonucleotide, or a portion thereof is complementary to 17-23 contiguous nucleobases at positions 2685-2714 of SEQ ID NO: 614, or a pharmaceutically acceptable salt thereof. In some aspects, the oligonucleotide, or a portion thereof, is complementary to 17-20 contiguous nucleobases at positions 2685-2714 of SEQ ID NO: 614, or a pharmaceutically acceptable salt thereof. In some aspects, the oligonucleotide, or a portion thereof, is complementary to 17-20 contiguous nucleobases beginning at position 2685, 2686, 2687, 2688, 2689, 2690, 2691, 2692, 2693, 2694, 2695, 2696, 2697, or 2698 of SEQ ID NO: 614, or a pharmaceutically acceptable salt thereof. In some aspects, the oligonucleotide is 17-20 linked nucleotides in length, or a pharmaceutically acceptable salt thereof.

In some aspects, the oligonucleotide, or a portion thereof, is complementary to 20-23 contiguous nucleobases at positions 2685-2714 of SEQ ID NO: 614, or a pharmaceutically acceptable salt thereof. In some aspects, the oligonucleotide is complementary to 20-23 contiguous nucleobases beginning at position 2685, 2686, 2687, 2688, 2689, 2690, 2691, 2692, 2693, 2694, or 2695 of SEQ ID NO: 614, or a pharmaceutically acceptable salt thereof. In some aspects, the oligonucleotide is 20-23 linked nucleotides in length, or a pharmaceutically acceptable salt thereof. In some aspects, the oligonucleotide, or a portion thereof, is complementary to positions 2685-2714 of SEQ ID NO: 614, or a pharmaceutically acceptable salt thereof.

In some aspects of the above, the oligonucleotide is not any one of Antisense Oligo Nos. 1, 97, 193, or 289 of Table 3. In some aspects of the above, the oligonucleotide does not have a nucleobase sequence consisting of any one of SEQ ID NOs: 1, 97, 193, or 289.

In some aspects of the above disclosures, the oligonucleotide consists of a nucleobase sequence selected from the group consisting of any of SEQ ID NOs: 1-384 and 390-613, or a pharmaceutically acceptable salt thereof. In some aspects, the oligonucleotide consists of a nucleobase sequence selected from the group consisting of any of SEQ ID NOs: 2-96, 98-192, 194-288, 290-384, and 390-613, or a pharmaceutically acceptable salt thereof. In some aspects, the oligonucleotide consists of a nucleobase sequence selected from the group consisting of any of SEQ ID NOs: 1-384, or a pharmaceutically acceptable salt thereof. In some aspects, the oligonucleotide consists of a nucleobase sequence selected from the group consisting of any of SEQ ID NOs: 2-96, 98-192, 194-288, and 290-384, or a pharmaceutically acceptable salt thereof. In some aspects, the oligonucleotide consists of a nucleobase sequence selected from the group consisting of any of SEQ ID NOs: 1-96, or a pharmaceutically acceptable salt thereof. In some aspects, the oligonucleotide consists of a nucleobase sequence selected from the group consisting of any of SEQ ID NOs: 2-96, or a pharmaceutically acceptable salt thereof. In some aspects, the oligonucleotide consists of a nucleobase sequence selected from the group consisting of any of SEQ ID NOs: 97-192, or a pharmaceutically acceptable salt thereof. In some aspects, the oligonucleotide consists of a nucleobase sequence selected from the group consisting of any of SEQ ID NOs: 98-192, or a pharmaceutically acceptable salt thereof. In some aspects, the oligonucleotide consists of a nucleobase sequence selected from the group consisting of any of SEQ ID NOs: 193-288, or a pharmaceutically acceptable salt thereof. In some aspects, the oligonucleotide consists of a nucleobase sequence selected from the group consisting of any of SEQ ID NOs: 194-288, or a pharmaceutically acceptable salt thereof. In some aspects, the oligonucleotide consists of a nucleobase sequence selected from the group consisting of any of SEQ ID NOs: 289-384, or a pharmaceutically acceptable salt thereof. In some aspects, the oligonucleotide consists of a nucleobase sequence selected from the group consisting of any of SEQ ID NOs: 288-384, or a pharmaceutically acceptable salt thereof. In some aspects, the oligonucleotide consists of a nucleobase sequence selected from the group consisting of any of SEQ ID NOs: 390-613, or a pharmaceutically acceptable salt thereof. In some aspects, the oligonucleotide consists of a nucleobase sequence selected from the group consisting of any of SEQ ID NOs: 390-480, or a pharmaceutically acceptable salt thereof. In some aspects, the oligonucleotide consists of a nucleobase sequence selected from the group consisting of any of SEQ ID NOs: 481-571, or a pharmaceutically acceptable salt thereof. In some aspects, the oligonucleotide consists of a nucleobase sequence selected from the group consisting of any of SEQ ID NOs: 572-662, or a pharmaceutically acceptable salt thereof. In some aspects, the oligonucleotide consists of a nucleobase sequence selected from the group consisting of any of SEQ ID NOs: 663-613, or a pharmaceutically acceptable salt thereof. In some aspects, the oligonucleotide consists of a nucleobase sequence selected from the group consisting of any of SEQ ID NOs: 1, 6, 13, 17, 21, 24, 26, 29, 33-34, 37, 44, 49-55, 57, 60-73, 75-76, 79-82, 84-86, 88-92, or 94-96, or a pharmaceutically acceptable salt thereof. In some aspects, the oligonucleotide consists of a nucleobase sequence selected from the group consisting of any of SEQ ID NOs: 6, 13, 17, 21, 24, 26, 29, 33-34, 37, 44, 49-55, 57, 60-73, 75-76, 79-82, 84-86, 88-92, or 94-96, or a pharmaceutically acceptable salt thereof. In some aspects, the oligonucleotide consists of a nucleobase sequence that is SEQ ID NO: 1, or a pharmaceutically acceptable salt thereof. In some aspects, the oligonucleotide consists of a nucleobase sequence that is SEQ ID NO: 6, or a pharmaceutically acceptable salt thereof. In some aspects, the oligonucleotide consists of a nucleobase sequence selected from the group consisting of any of SEQ ID NOs: 97, 100, 103, 105, 108, 110-111, 113-117, 122-123, 127, 129-130, 133-136, 138-139, 141, 143-145, 147-148, 154-155, 157-165, 168-170, 172, 174-180, 184, 187, or 191, or a pharmaceutically acceptable salt thereof. In some aspects, the oligonucleotide consists of a nucleobase sequence selected from the group consisting of any of SEQ ID NOs: 100, 103, 105, 108, 110-111, 113-117, 122-123, 127, 129-130, 133-136, 138-139, 141, 143-145, 147-148, 154-155, 157-165, 168-170, 172, 174-180, 184, 187, or 191, or a pharmaceutically acceptable salt thereof. In some aspects, the oligonucleotide consists of a nucleobase sequence that is SEQ ID NO: 97, or a pharmaceutically acceptable salt thereof. In some aspects, the oligonucleotide consists of a nucleobase sequence that is wherein the oligonucleotide consists of a nucleobase sequence that is SEQ ID NO: 145, or a pharmaceutically acceptable salt thereof. In some aspects, the oligonucleotide consists of a nucleobase sequence that is wherein the oligonucleotide consists of a nucleobase sequence that is SEQ ID NO: 163, or a pharmaceutically acceptable salt thereof. In some aspects, the oligonucleotide consists of a nucleobase sequence selected from the group consisting of any of SEQ ID NOs: 193-200, 202-230, 232-246, 248-253, 255, 258-261, 265, 270, 274-276, or 285-286, or a pharmaceutically acceptable salt thereof. In some aspects, the oligonucleotide consists of a nucleobase sequence selected from the group consisting of any of SEQ ID NOs: 194-200, 202-230, 232-246, 248-253, 255, 258-261, 265, 270, 274-276, or 285-286, or a pharmaceutically acceptable salt thereof. In some aspects, the oligonucleotide consists of a nucleobase sequence that is SEQ ID NO: 193, or a pharmaceutically acceptable salt thereof. In some aspects, the oligonucleotide consists of a nucleobase sequence selected from the group consisting of any of SEQ ID NOs: 226-227, 234, 240, or 243-244, or a pharmaceutically acceptable salt thereof. In some aspects, the oligonucleotide consists of a nucleobase sequence selected from the group consisting of any of SEQ ID NOs: 227, 234, 240, or 243-244, or a pharmaceutically acceptable salt thereof. In some aspects, the oligonucleotide consists of the nucleobase sequence that is SEQ ID NO: 226, or a pharmaceutically acceptable salt thereof. In some aspects, the oligonucleotide consists of a nucleobase sequence selected from the group consisting of any of SEQ ID NOs: 289-290, 292, 305, 307, 313, 318, 323-324, 326, 329-330, 332, 338-339, 341, 344, or 346, or a pharmaceutically acceptable salt thereof. In some aspects, the oligonucleotide consists of a nucleobase sequence selected from the group consisting of any of SEQ ID NOs: 290, 292, 305, 307, 313, 318, 323-324, 326, 329-330, 332, 338-339, 341, 344, or 346, or a pharmaceutically acceptable salt thereof. In some aspects, the oligonucleotide consists of a nucleobase sequence that is SEQ ID NO: 289, or a pharmaceutically acceptable salt thereof. In some aspects, the oligonucleotide consists of a nucleobase sequence that is SEQ ID NO: 329, or a pharmaceutically acceptable salt thereof. In some aspects, the oligonucleotide consists of a nucleobase sequence that is SEQ ID NO: 346, or a pharmaceutically acceptable salt thereof.

Some aspects of the disclosure are directed to single-stranded oligonucleotides, wherein the nucleobase sequence of the oligonucleotide consists of any one of SEQ ID NOs: 1-384 and 390-613, or a pharmaceutically acceptable salt thereof. In some aspects, the nucleobase sequence of the oligonucleotide consists of any one of SEQ ID NOs: 2-96, 98-192, 194-288, 290-384, and 390-613, or a pharmaceutically acceptable salt thereof. In some aspects, the nucleobase sequence of the oligonucleotide consists of any one of SEQ ID NOs: 1-384, or a pharmaceutically acceptable salt thereof. In some aspects, the nucleobase sequence of the oligonucleotide consists of any one of SEQ ID NOs: 2-96, 98-192, 194-288, or 290-384, or a pharmaceutically acceptable salt thereof. In some aspects, the oligonucleotide consists of the nucleobase sequence of any one of SEQ ID NOs: 1-96, or a pharmaceutically acceptable salt thereof. In some aspects, the oligonucleotide consists of the nucleobase sequence of any one of SEQ ID NOs: 2-96, or a pharmaceutically acceptable salt thereof. In some aspects, the oligonucleotide consists of the nucleobase sequence of any one of SEQ ID NOs: 97-192, or a pharmaceutically acceptable salt thereof. In some aspects, the oligonucleotide consists of the nucleobase sequence of any one of SEQ ID NOs: 96-192, or a pharmaceutically acceptable salt thereof. In some aspects, the oligonucleotide consists of the nucleobase sequence of any one of SEQ ID NOs: 193-288, or a pharmaceutically acceptable salt thereof. In some aspects, the oligonucleotide consists of the nucleobase sequence of any one of SEQ ID NOs: 194-288, or a pharmaceutically acceptable salt thereof. In some aspects, the oligonucleotide consists of the nucleobase sequence of any one of SEQ ID NOs: 289-384, or a pharmaceutically acceptable salt thereof. In some aspects, the oligonucleotide consists of the nucleobase sequence of any one of SEQ ID NOs: 290-384, or a pharmaceutically acceptable salt thereof. In some aspects, the oligonucleotide consists of the nucleobase sequence of any one of SEQ ID NOs: 390-613, or a pharmaceutically acceptable salt thereof. In some aspects, the oligonucleotide consists of the nucleobase sequence of any one of SEQ ID NOs: 390-480, or a pharmaceutically acceptable salt thereof. In some aspects, the oligonucleotide consists of the nucleobase sequence of any one of SEQ ID NOs: 481-571, or a pharmaceutically acceptable salt thereof. In some aspects, the oligonucleotide consists of the nucleobase sequence of any one of SEQ ID NOs: 572-662, or a pharmaceutically acceptable salt thereof. In some aspects, the oligonucleotide consists of the nucleobase sequence of any one of SEQ ID NOs: 663-613, or a pharmaceutically acceptable salt thereof. In some aspects, the oligonucleotide consists of the nucleobase sequence of any one of SEQ ID NOs: 1, 6, 13, 17, 21, 24, 26, 29, 33-34, 37, 44, 49-55, 57, 60-73, 75-76, 79-82, 84-86, 88-92, or 94-96, or a pharmaceutically acceptable salt thereof. In some aspects, the oligonucleotide consists of the nucleobase sequence of any one of SEQ ID NOs: 6, 13, 17, 21, 24, 26, 29, 33-34, 37, 44, 49-55, 57, 60-73, 75-76, 79-82, 84-86, 88-92, or 94-96, or a pharmaceutically acceptable salt thereof. In some aspects, the oligonucleotide consists of the nucleobase sequence of SEQ ID NO: 1, or a pharmaceutically acceptable salt thereof. In some aspects, the oligonucleotide consists of the nucleobase sequence of SEQ ID NO: 6, or a pharmaceutically acceptable salt thereof. In some aspects, the oligonucleotide consists of the nucleobase sequence of any one of SEQ ID NOs: 97, 100, 103, 105, 108, 110-111, 113-117, 122-123, 127, 129-130, 133-136, 138-139, 141, 143-145, 147-148, 154-155, 157-165, 168-170, 172, 174-180, 184, 187, or 191, or a pharmaceutically acceptable salt thereof. In some aspects, the oligonucleotide consists of the nucleobase sequence of any one of SEQ ID NOs: 100, 103, 105, 108, 110-111, 113-117, 122-123, 127, 129-130, 133-136, 138-139, 141, 143-145, 147-148, 154-155, 157-165, 168-170, 172, 174-180, 184, 187, or 191, or a pharmaceutically acceptable salt thereof. In some aspects, the oligonucleotide consists of the nucleobase sequence of SEQ ID NO: 97, or a pharmaceutically acceptable salt thereof. In some aspects, the oligonucleotide consists of the nucleobase sequence of SEQ ID NO: 145, or a pharmaceutically acceptable salt thereof. In some aspects, the oligonucleotide consists of the nucleobase sequence of SEQ ID NO: 163, or a pharmaceutically acceptable salt thereof. In some aspects, the oligonucleotide consists of the nucleobase sequence of any one of SEQ ID NOs: 193-200, 202-230, 232-246, 248-253, 255, 258-261, 265, 270, 274-276, or 285-286, or a pharmaceutically acceptable salt thereof. In some aspects, the oligonucleotide consists of the nucleobase sequence of any one of SEQ ID NOs: 194-200, 202-230, 232-246, 248-253, 255, 258-261, 265, 270, 274-276, or 285-286, or a pharmaceutically acceptable salt thereof. In some aspects, the oligonucleotide consists of the nucleobase sequence of any one of SEQ ID NO: 193, or a pharmaceutically acceptable salt thereof. In some aspects, the oligonucleotide consists of the nucleobase sequence of any one of SEQ ID NOs: 226-227, 234, 240, or 243-244, or a pharmaceutically acceptable salt thereof. In some aspects, the oligonucleotide consists of the nucleobase sequence of any one of SEQ ID NOs: 227, 234, 240, or 243-244, or a pharmaceutically acceptable salt thereof. In some aspects, the oligonucleotide consists of the nucleobase sequence of SEQ ID NO: 226, or a pharmaceutically acceptable salt thereof. In some aspects, the oligonucleotide consists of the nucleobase sequence of any one of SEQ ID NOs: 289-290, 292, 305, 307, 313, 318, 323-324, 326, 329-330, 332, 338-339, 341, 344, or 346, or a pharmaceutically acceptable salt thereof. In some aspects, the oligonucleotide consists of the nucleobase sequence of any one of SEQ ID NOs: 290, 292, 305, 307, 313, 318, 323-324, 326, 329-330, 332, 338-339, 341, 344, or 346, or a pharmaceutically acceptable salt thereof. In some aspects, the oligonucleotide consists of the nucleobase sequence of SEQ ID NO: 289, or a pharmaceutically acceptable salt thereof. In some aspects, the oligonucleotide consists of a nucleobase sequence that is SEQ ID NO: 329, or a pharmaceutically acceptable salt thereof. In some aspects, the oligonucleotide consists of a nucleobase sequence that is SEQ ID NO: 346, or a pharmaceutically acceptable salt thereof.

Some aspects of the disclosure are directed to nn oligonucleotide selected from the group consisting of Antisense Oligo Nos. 1-384 of Table 3 or 390-613 of Table 4, or a pharmaceutically acceptable salt thereof. In some aspects, the oligonucleotide is selected from the group consisting of Antisense Oligo Nos. 2-96, 98-192, 194-288, 290-384 of Table 3 and 390-613 of Table 4, or a pharmaceutically acceptable salt thereof. In some aspects, the oligonucleotide is selected from the group consisting of Antisense Oligo Nos. 1-384 of Table 3, or a pharmaceutically acceptable salt thereof. In some aspects, the oligonucleotide is selected from the group consisting of Antisense Oligo Nos. 2-96, 98-192, 194-288, and 290-384 of Table 3, or a pharmaceutically acceptable salt thereof. In some aspects, the oligonucleotide is selected from the group consisting of Antisense Oligo Nos. 1-96 of Table 3, or a pharmaceutically acceptable salt thereof. In some aspects, the oligonucleotide is selected from the group consisting of Antisense Oligo Nos. 2-96 of Table 3, or a pharmaceutically acceptable salt thereof. In some aspects, the oligonucleotide is selected from the group consisting of Antisense Oligo Nos. 97-192 of Table 3, or a pharmaceutically acceptable salt thereof. In some aspects, the oligonucleotide is selected from the group consisting of Antisense Oligo Nos. 98-192 of Table 3, or a pharmaceutically acceptable salt thereof. In some aspects, the oligonucleotide is selected from the group consisting of Antisense Oligo Nos. 193-288 of Table 3, or a pharmaceutically acceptable salt thereof. In some aspects, the oligonucleotide is selected from the group consisting of Antisense Oligo Nos. 194-288 of Table 3, or a pharmaceutically acceptable salt thereof. In some aspects, the oligonucleotide is selected from the group consisting of Antisense Oligo Nos. 289-384 of Table 3, or a pharmaceutically acceptable salt thereof. In some aspects, the oligonucleotide is selected from the group consisting of Antisense Oligo Nos. 290-384 of Table 3, or a pharmaceutically acceptable salt thereof. In some aspects, the oligonucleotide is selected from the group consisting of Antisense Oligo Nos. 390-613 of Table 4, or a pharmaceutically acceptable salt thereof. In some aspects, the oligonucleotide is selected from the group consisting of Antisense Oligo Nos. 390-480 of Table 4, or a pharmaceutically acceptable salt thereof. In some aspects, the oligonucleotide is selected from the group consisting of Antisense Oligo Nos. 481-571 of Table 4, or a pharmaceutically acceptable salt thereof. In some aspects, the oligonucleotide is selected from the group consisting of Antisense Oligo Nos. 1, 6, 13, 17, 21, 24, 26, 29, 33-34, 37, 44, 49-55, 57, 60-73, 75-76, 79-82, 84-86, 88-92, or 94-96 of Table 3, or a pharmaceutically acceptable salt thereof. In some aspects, the oligonucleotide is selected from the group consisting of Antisense Oligo Nos. 6, 13, 17, 21, 24, 26, 29, 33-34, 37, 44, 49-55, 57, 60-73, 75-76, 79-82, 84-86, 88-92, or 94-96 of Table 3, or a pharmaceutically acceptable salt thereof. In some aspects, the oligonucleotide is Antisense Oligo No. 1 of Table 3, or a pharmaceutically acceptable salt thereof. In some aspects, the oligonucleotide is Antisense Oligo No. 6 of Table 3, or a pharmaceutically acceptable salt thereof. In some aspects, the oligonucleotide is selected from the group consisting of Antisense Oligo Nos. 97, 100, 103, 105, 108, 110-111, 113-117, 122-123, 127, 129-130, 133-136, 138-139, 141, 143-145, 147-148, 154-155, 157-165, 168-170, 172, 174-180, 184, 187, or 191 of Table 3, or a pharmaceutically acceptable salt thereof. In some aspects, the oligonucleotide is selected from the group consisting of Antisense Oligo Nos. 100, 103, 105, 108, 110-111, 113-117, 122-123, 127, 129-130, 133-136, 138-139, 141, 143-145, 147-148, 154-155, 157-165, 168-170, 172, 174-180, 184, 187, or 191 of Table 3, or a pharmaceutically acceptable salt thereof. In some aspects, the oligonucleotide is Antisense Oligo No. 97 of Table 3, or a pharmaceutically acceptable salt thereof. In some aspects, the oligonucleotide is Antisense Oligo No. 145 of Table 3, or a pharmaceutically acceptable salt thereof. In some aspects, the oligonucleotide is Antisense Oligo No. 163 of Table 3, or a pharmaceutically acceptable salt thereof. In some aspects, the oligonucleotide is selected from the group consisting of Antisense Oligo Nos. 193-200, 202-230, 232-246, 248-253, 255, 258-261, 265, 270, 274-276, or 285-286 of Table 3, or a pharmaceutically acceptable salt thereof. In some aspects, the oligonucleotide is selected from the group consisting of Antisense Oligo Nos. 194-200, 202-230, 232-246, 248-253, 255, 258-261, 265, 270, 274-276, or 285-286 of Table 3, or a pharmaceutically acceptable salt thereof. In some aspects, the oligonucleotide is Antisense Oligo No. 193 of Table 3. In some aspects, the oligonucleotide is selected from the group consisting of Antisense Oligo Nos. 226-227, 234, 240, or 243-244 of Table 3, or a pharmaceutically acceptable salt thereof. In some aspects, the oligonucleotide is selected from the group consisting of Antisense Oligo Nos. 227, 234, 240, or 243-244 of Table 3, or a pharmaceutically acceptable salt thereof. In some aspects, the oligonucleotide is Antisense Oligo No. 226 of Table 3, or a pharmaceutically acceptable salt thereof. In some aspects, the oligonucleotide is selected from the group consisting of Antisense Oligo Nos. 289-290, 292, 305, 307, 313, 318, 323-324, 326, 329-330, 332, 338-339, 341, 344, or 346 of Table 3, or a pharmaceutically acceptable salt thereof. In some aspects, the oligonucleotide is selected from the group consisting of Antisense Oligo Nos. 290, 292, 305, 307, 313, 318, 323-324, 326, 329-330, 332, 338-339, 341, 344, or 346 of Table 3, or a pharmaceutically acceptable salt thereof. In some aspects, the oligonucleotide is Antisense Oligo No. 289 of Table 3, or a pharmaceutically acceptable salt thereof. In some aspects, the oligonucleotide is Antisense Oligo No. 329 of Table 3, or a pharmaceutically acceptable salt thereof. In some aspects, the oligonucleotide is Antisense Oligo No. 346 of Table 3, or a pharmaceutically acceptable salt thereof.

In some aspects, the oligonucleotide, or a pharmaceutically acceptable salt thereof, described herein causes at least a 50% reduction in MSH3 mRNA expression at an oligonucleotide concentration of 10 nM. In some aspects, the oligonucleotide, or a pharmaceutically acceptable salt thereof, described herein causes at least a 60% reduction in MSH3 mRNA expression at an oligonucleotide concentration of 10 nM. In some aspects, the oligonucleotide, or a pharmaceutically acceptable salt thereof, described herein causes at least a 70% reduction in MSH3 mRNA expression at an oligonucleotide concentration of 10 nM. In some aspects, the oligonucleotide, or a pharmaceutically acceptable salt thereof, described herein causes at least an 80% reduction in MSH3 mRNA expression at an oligonucleotide concentration of 10 nM.

In some aspects, the oligonucleotide, or a pharmaceutically acceptable salt thereof, described herein causes at least a 50% reduction in MSH3 mRNA expression at an oligonucleotide concentration of 1 nM. In some aspects, the oligonucleotide, or a pharmaceutically acceptable salt thereof, described herein causes at least a 60% reduction in MSH3 mRNA expression at an oligonucleotide concentration of 1 nM. In some aspects, the oligonucleotide, or a pharmaceutically acceptable salt thereof, described herein causes at least a 70% reduction in MSH3 mRNA expression at an oligonucleotide concentration of 1 nM.

The cell assay can comprise transfecting mammalian cells, such as HEK293, NIH3T3, or HeLa cells, with the desired a concentration of oligonucleotide (e.g., 1 nM or 10 nM) using Lipofectamine 2000 (Invitrogen) and comparing levels of transfected cells to levels of control cells, e.g., comparing MSH3 mRNA levels of transfected cells to MSH3 levels of control cells. Control cells can be transfected with oligonucleotides not specific to MSH3 or mock transfected. mRNA levels can be determined using RT-qPCR and MSH3 mRNA levels can be normalized to GAPDH mRNA levels. The percent inhibition can be calculated as the percent of MSH3 mRNA concentration relative to the MSH3 concentration of the control cells.

In some aspects, the MSH3 mRNA expression is evaluated in vitro. In some aspects, the MSH3 mRNA expression is evaluated in a cell based assay. In some aspects, the MSH3 mRNA expression is evaluated in HeLa cells. In some aspects, the MSH3 mRNA expression is determined by the quantitative reverse transcription polymerase chain reaction (RT-qPCR). In some aspects, the MSH3 mRNA is expression is normalized to the mRNA expression of a reference gene. In some aspects, the MSH3 mRNA expression is normalized to the mRNA expression of beta-glucuronidase (GUSB). In some aspects, the reduction in MSH3 mRNA expression is relative to a control. In some aspects, the control is the MSH3 mRNA expression in the absence of the oligonucleotide, or pharmaceutically acceptable salt thereof. In some aspects, the control is the MSH3 mRNA expression in the absence of the oligonucleotide, or pharmaceutically acceptable salt thereof, but in the presence of a control oligonucleotide, or salt thereof. In some aspects, the control oligonucleotide, or salt thereof, is a scrambled luciferase targeting oligonucleotide. In some aspects, the reduction in MSH3 mRNA expression is calculated by a delta-delta Ct (ΔΔCT) method. In some aspects, the delta-delta Ct (ΔΔCT) method comprises the normalization of the MSH3 mRNA expression to the mRNA expression of a reference gene and to the MSH3 mRNA expression in the absence of the oligonucleotide, or pharmaceutically acceptable salt thereof but in the presence of a control oligonucleotide, or salt thereof. In some aspects, the reference gene is beta-glucuronidase (GUSB) and/or the control oligonucleotide, or salt thereof, is a scrambled luciferase targeting oligonucleotide. In some aspects, the reduction in MSH3 mRNA expression is determined by the method of Example 1. In some aspects, in the same assay, Antisense Oligo No. 1 causes approximately a 58% reduction in MSH3 mRNA expression at an oligonucleotide concentration of 10 nM. In some aspects, in the same assay, Antisense Oligo No. 1 causes approximately a 14% reduction in MSH3 mRNA expression at an oligonucleotide concentration of 1 nM.

In some aspects, the oligonucleotide, or contiguous nucleotide region thereof, has a gapmer design or structure also referred herein merely as “gapmer.” In a gapmer structure the oligonucleotide comprises at least three distinct structural regions a 5′-flanking sequence (also known as a 5′-wing), a DNA core sequence (also known as a gap) and a 3′-flanking sequence (also known as a 3′-wing), in ‘5->3’ orientation. In this design, the 5′ and 3′ flanking sequences comprise at least one alternative nucleoside which is adjacent to a DNA core sequence, and can, in some aspects, comprise a contiguous stretch of 2-7 alternative nucleosides, or a contiguous stretch of alternative and DNA nucleosides (mixed flanking sequences comprising both alternative and DNA nucleosides).

The length of the 5′-flanking sequence region can be at least two nucleosides in length (e.g., at least at least 2, at least 3, at least 4, at least 5, at least 6, or more nucleosides in length). The length of the 3′-flanking sequence region can be at least two nucleosides in length (e.g., at least 2, at least 3, at least at least 4, at least 5, at least 6, or more nucleosides in length). The 5′ and 3′ flanking sequences can be symmetrical or asymmetrical with respect to the number of nucleosides they comprise. In some aspects, the DNA core sequence comprises about 10 nucleosides flanked by a 5′ and a 3′ flanking sequence each comprising about 5 nucleosides. In some aspects, the DNA core sequence comprises about 11 nucleosides flanked by a 5′ and a 3′ flanking sequence each comprising about 5 or about 6 nucleosides. In some aspects, the DNA core sequence comprises about 12 nucleosides flanked by a 5′ sequence comprising about 5 nucleosides, and a 3′ flanking sequence comprising about 6 nucleosides. In some aspects, the DNA core sequence comprises about 12 nucleosides flanked by a 5′ sequence comprising about 6 nucleosides, and a 3′ flanking sequence comprising about 5 nucleosides. In some aspects, the DNA core sequence comprises about 12 nucleosides flanked by a 5′ and a 3′ flanking sequence each comprising about 6 nucleosides.

Consequently, the nucleosides of the 5′ flanking sequence and the 3′ flanking sequence which are adjacent to the DNA core sequence are alternative nucleosides, such as 2′ alternative nucleosides. The DNA core sequence comprises a contiguous stretch of nucleotides which are capable of recruiting RNase H, when the oligonucleotide is in duplex with the MSH3 target nucleic acid. In some aspects, the DNA core sequence comprises a contiguous stretch of 5-16 DNA nucleosides. In other aspects, the DNA core sequence comprises a region of at least 10 contiguous nucleobases having at least 80% (e.g., at least 85%, at least 90%, at least 95%, or at least 99%) complementarity to an MSH3 gene. In some aspects, the gapmer comprises a region complementary to at least 17 contiguous nucleotides, 19-23 contiguous nucleotides, or 19 contiguous nucleotides of a MSH3 gene. The gapmer is complementary to the MSH3 target nucleic acid, and can therefore be the contiguous nucleoside region of the oligonucleotide. In some aspects, the gapmer comprises a region complementary to at least 21 contiguous nucleotides, 20-25 contiguous nucleotides, or 23 contiguous nucleotides of a MSH3 gene. The gapmer is complementary to the MSH3 target nucleic acid, and can therefore be the contiguous nucleoside region of the oligonucleotide.

The 5′ and 3′ flanking sequences, flanking the 5′ and 3′ ends of the DNA core sequence, can comprise one or more affinity enhancing alternative nucleosides. In some aspects, the 5′ and/or 3′ flanking sequence comprises at least one 2′-O-methoxyethyl (MOE) nucleoside. In some aspects, the 5′ and/or 3′ flanking sequences, contain at least two MOE nucleosides. In some aspects, the 5′ flanking sequence comprises at least one, at least two, at least three, at least four, at least five, or at least six or more MOE nucleosides. In some aspects, the 5′ flanking sequence comprises at least one, at least two, at least three, at least four, at least five, or at least six or more MOE nucleosides. In some aspects, both the 5′ and 3′ flanking sequence comprise a MOE nucleoside. In some aspects, all the nucleosides in the flanking sequences are MOE nucleosides. In other aspects, the flanking sequence can comprise both MOE nucleosides and other nucleosides (mixed flanking sequence), such as DNA nucleosides and/or non-MOE alternative nucleosides, such as bicyclic nucleosides (BNAs) (e.g., LNA nucleosides (e.g., A-LNA, 5mC L-NA, G-LNA, T-LNA) or cET nucleosides), or other 2′ substituted nucleosides. In this case the DNA core sequence is defined as a contiguous sequence of at least 5 RNase H recruiting nucleosides (such as 5-16 DNA nucleosides) flanked at the 5′ and 3′ end by an affinity enhancing alternative nucleoside, such as an MOE nucleoside.

In other aspects, the 5′ and/or 3′ flanking sequence comprises at least one BNA (e.g., at least one LNA nucleoside (e.g., A-LNA, 5mC L-NA, G-LNA, T-LNA) or cET nucleoside). In some aspects, 5′ and/or 3′ flanking sequence comprises at least 2 bicyclic nucleosides. In some aspects, the 5′ flanking sequence comprises at least one BNA. In some aspects, both the 5′ and 3′ flanking sequence comprise a BNA. In some aspects, all the nucleosides in the flanking sequences are BNAs. In other aspects, the flanking sequence can comprise both BNAs and other nucleosides (mixed flanking sequences), such as DNA nucleosides and/or non-BNA alternative nucleosides, such as 2′ substituted nucleosides. In this case the DNA core sequence is defined as a contiguous sequence of at least five RNase H recruiting nucleosides (such as 5-16 DNA nucleosides) flanked at the 5′ and 3′ end by an affinity enhancing alternative nucleoside, such as a BNA, such as an LNA, such as beta-D-oxy-LNA.

The 5′ flank attached to the 5′ end of the DNA core sequence comprises, contains, or consists of at least one alternative sugar moiety (e.g., at least three, at least four, at least five, at least six, at least seven, or more alternative sugar moieties). In some aspects, the flanking sequence comprises or consists of from 1 to 7 alternative nucleobases, such as from 2 to 6 alternative nucleobases, such as from 2 to 5 alternative nucleobases, such as from 2 to 4 alternative nucleobases, such as from 1 to 3 alternative nucleobases, such as one, two, three or four alternative nucleobases. In some aspects, the flanking sequence comprises or consists of at least one alternative internucleoside linkage (e.g., at least three, at least four, at least five, at least six, at least seven, or more alternative internucleoside linkages).

The 3′ flank attached to the 3′ end of the DNA core sequence comprises, contains, or consists of at least one alternative sugar moiety (e.g., at least three, at least four, at least five, at least six, at least seven, or more alternative sugar moieties). In some aspects, the flanking sequence comprises or consists of from 1 to 7 alternative nucleobases, such as from 2 to 6 alternative nucleobases, such as from 2 to 5 alternative nucleobases, such as from 2 to 4 alternative nucleobases, such as from 1 to 3 alternative nucleobases, such as one, two, three, or four alternative nucleobases. In some aspects, the flanking sequence comprises or consists of at least one alternative internucleoside linkage (e.g., at least three, at least four, at least five, at least six, at least seven, or more alternative internucleoside linkages).

In an aspect, one or more or all of the alternative sugar moieties in the flanking sequence are 2′ alternative sugar moieties.

In a further aspect, one or more of the 2′ alternative sugar moieties in the wing regions are selected from 2′-O-alkyl-sugar moieties, 2′-O-methyl-sugar moieties, 2′-amino-sugar moieties, 2′-fluoro-sugar moieties, 2′-alkoxy-sugar moieties, MOE sugar moieties, LNA sugar moieties, arabino nucleic acid (ANA) sugar moieties, and 2′-fluoro-ANA sugar moieties.

In one aspect, all the alternative nucleosides in the flanking sequences are bicyclic nucleosides. In a further aspect, the bicyclic nucleosides in the flanking sequences are independently selected from the group consisting of oxy-LNA, thio-LNA, amino-LNA, cET, and/or ENA, in either the beta-D or alpha-L configurations or combinations thereof.

In some aspects, the one or more alternative internucleoside linkages in the flanking sequences are phosphorothioate internucleoside linkages. In some aspects, the phosphorothioate linkages are stereochemically pure phosphorothioate linkages. In some aspects, the phosphorothioate linkages are Sp phosphorothioate linkages. In other aspects, the phosphorothioate linkages are Rp phosphorothioate linkages. In some aspects, the alternative internucleoside linkages are 2′-alkoxy internucleoside linkages. In other aspects, the alternative internucleoside linkages are alkyl phosphate internucleoside linkages.

The DNA core sequence can comprise, contain, or consist of at least 5-16 consecutive DNA nucleosides capable of recruiting RNase H. In some aspects, all of the nucleosides of the DNA core sequence are DNA units. In further aspects, the DNA core region can consist of a mixture of DNA and other nucleosides capable of mediating RNase H cleavage. In some aspects, at least 50% of the nucleosides of the DNA core sequence are DNA, such as at least 60%, at least 70% or at least 80%, or at least 90% DNA. In some aspects, all of the nucleosides of the DNA core sequence are RNA units.

The oligonucleotide comprises a contiguous region which is complementary to the target nucleic acid. In some aspects, the oligonucleotide can further comprise additional linked nucleosides positioned 5′ and/or 3′ to either the 5′ and 3′ flanking sequences. These additional linked nucleosides can be attached to the 5′ end of the 5′ flanking sequence or the 3′ end of the 3′ flanking sequence, respectively. The additional nucleosides can, in some aspects, form part of the contiguous sequence which is complementary to the target nucleic acid, or in other aspects, can be non-complementary to the target nucleic acid.

The inclusion of the additional nucleosides at either, or both of the 5′ and 3′ flanking sequences can independently comprise one, two, three, four, or five additional nucleotides, which can be complementary or non-complementary to the target nucleic acid. In this respect the oligonucleotide, can in some aspects comprise a contiguous sequence capable of modulating the target which is flanked at the 5′ and/or 3′ end by additional nucleotides. Such additional nucleosides can serve as a nuclease susceptible biocleavable linker, and can therefore be used to attach a functional group such as a conjugate moiety to the oligonucleotide. In some aspects, the additional 5′ and/or 3′ end nucleosides are linked with phosphodiester linkages, and can be DNA or RNA. In another aspect, the additional 5′ and/or 3′ end nucleosides are alternative nucleosides which can for example be included to enhance nuclease stability or for ease of synthesis.

In other aspects, the oligonucleotides utilize “altimer” design and comprise alternating 2′-fluoro-ANA and DNA regions that are alternated every three nucleosides. Altimer oligonucleotides are discussed in more detail in Min. et al., Bioorganic & Medicinal Chemistry Letters, 2002, 12(18): 2651-2654 and Kalota, et al., Nuc. Acid Res. 2006, 34(2): 451-61 (herein incorporated by reference).

In other aspects, the oligonucleotides utilize “hemimer” design and comprise a single 2′-modified flanking sequence adjacent to (on either side of the 5′ or the 3′ side of) a DNA core sequence. Hemimer oligonucleotides are discussed in more detail in Geary et al., 2001, J. Pharm. Exp. Therap., 296: 898-904 (herein incorporated by reference).

In some aspects, an oligonucleotide has a nucleic acid sequence with at least 50% (e.g., at least 50%, at least 60%, at least 70%, at least 80%, at least 85%, at least 90%, at least 95%, at least 96%, at least 97%, at least 98%, at least 99%, or 100%) sequence identity to the nucleic acid sequence of any one of SEQ ID NOs: 1-384 and 390-613. In some aspects, an oligonucleotide has a nucleic acid sequence with at least 85% sequence identity to the nucleic acid sequence of any one of SEQ ID NOs: 1-384 and 390-613.

It will be understood that the nucleosides of the oligonucleotide e.g., an oligonucleotide, can comprise any one of the sequences set forth in any one of SEQ ID NOs: 1-384 that is an alternative nucleoside and/or conjugated or linked as described in detail below.

In some aspects, the oligonucleotide is an oligonucleotide having at least 15 contiguous bases of the nucleobase sequence selected from the group consisting of Antisense Oligo Nos. 1-384 of Table 3 or 390-613 of Table 4. In some aspects, the oligonucleotide is an oligonucleotide having at least 15 contiguous bases of the nucleobase sequence selected from the group consisting of Antisense Oligo Nos. 1-384 of Table 3. In some aspects, the oligonucleotide is an oligonucleotide having at least 15 contiguous bases of the nucleobase sequence selected from the group consisting one of Antisense Oligo Nos. 1-96 of Table 3. In some aspects, the oligonucleotide is an oligonucleotide having at least 15 contiguous bases of the nucleobase sequence selected from the group consisting of Antisense Oligo Nos. 97-192 of Table 3. In some aspects, the oligonucleotide is an oligonucleotide having at least 15 contiguous bases of the nucleobase sequence selected from the group consisting of Antisense Oligo Nos. 193-288 of Table 3. In some aspects, the oligonucleotide is an oligonucleotide having at least 15 contiguous bases of the nucleobase sequence selected from the group consisting of Antisense Oligo Nos. 289-384 of Table 3. In some aspects, the oligonucleotide is an oligonucleotide having at least 15 contiguous bases of the nucleobase sequence selected from the group consisting of Antisense Oligo Nos. 390-613 of Table 4. In some aspects, the oligonucleotide is an oligonucleotide having at least 15 contiguous bases of the nucleobase sequence selected from the group consisting of Antisense Oligo Nos. 390-480 of Table 4. In some aspects, the oligonucleotide is an oligonucleotide having at least 15 contiguous bases of the nucleobase sequence selected from the group consisting of Antisense Oligo Nos. 481-501 of Table 4. In some aspects, the oligonucleotide is an oligonucleotide having at least 15 contiguous bases of the nucleobase sequence selected from the group consisting of Antisense Oligo Nos. 502-592 of Table 4. In some aspects, the oligonucleotide is an oligonucleotide having at least 15 contiguous bases of the nucleobase sequence selected from the group consisting of Antisense Oligo Nos. 593-613 of Table 4.

In some aspects, the oligonucleotide is an oligonucleotide having at least 15 contiguous bases of the nucleobase sequence selected from the group consisting of Antisense Oligo Nos. 1, 6, 13, 17, 21, 24, 26, 29, 33-34, 37, 44, 49-55, 57, 60-73, 75-76, 79-82, 84-86, 88-92, or 94-96 of Table 3. In some aspects, the oligonucleotide is an oligonucleotide having at least 15 contiguous bases of the nucleobase sequence selected from the group consisting of Antisense Oligo No. 1 of Table 3. In some aspects, the oligonucleotide an oligonucleotide having at least 15 contiguous bases of the nucleobase sequence of Antisense Oligo No. 6 of Table 3.

In some aspects, the oligonucleotide is an oligonucleotide having at least 15 contiguous bases of the nucleobase sequence selected from the group consisting of Antisense Oligo Nos. 97, 100, 103, 105, 108, 110-111, 113-117, 122-123, 127, 129-130, 133-136, 138-139, 141, 143-145, 147-148, 154-155, 157-165, 168-170, 172, 174-180, 184, 187, or 191 of Table 3. In some aspects, the oligonucleotide is an oligonucleotide having at least 15 contiguous bases of the nucleobase sequence selected from the group consisting of Antisense Oligo No. 97 of Table 3. In some aspects, the oligonucleotide is an oligonucleotide having at least 15 contiguous bases of the nucleobase sequence selected from the group consisting of Antisense Oligo No. 145 of Table 3. In some aspects, the oligonucleotide is an oligonucleotide having at least 15 contiguous bases of the nucleobase sequence selected from the group consisting of Antisense Oligo No. 163 of Table 3.

In some aspects, the oligonucleotide is an oligonucleotide having at least 15 contiguous bases of the nucleobase sequence selected from the group consisting of Antisense Oligo Nos. 193-200, 202-230, 232-246, 248-253, 255, 258-261, 265, 270, 274-276, or 285-286 of Table 3. In some aspects, the oligonucleotide is an oligonucleotide having at least 15 contiguous bases of the nucleobase sequence selected from the group consisting of Antisense Oligo No. 193 of Table 3.

In some aspects, the oligonucleotide is an oligonucleotide having at least 15 contiguous bases of the nucleobase sequence selected from the group consisting of Antisense Oligo Nos. 226-227, 234, 240, or 243-244 of Table 3. In some aspects, the oligonucleotide is an oligonucleotide having at least 15 contiguous bases of the nucleobase sequence selected from the group consisting of Antisense Oligo No. 226 of Table 3.

In some aspects, the oligonucleotide is an oligonucleotide having at least 15 contiguous bases of the nucleobase sequence selected from the group consisting of Antisense Oligo Nos. 289-290, 292, 305, 307, 313, 318, 323-324, 326, 329-330, 332, 338-339, 341, 344, or 346 of Table 3. In some aspects, the oligonucleotide is an oligonucleotide having at least 15 contiguous bases of the nucleobase sequence Antisense Oligo No. 289 of Table 3. In some aspects, the oligonucleotide is an oligonucleotide having at least 15 contiguous bases of the nucleobase sequence of Antisense Oligo No. 329 of Table 3. In some aspects, the oligonucleotide is an oligonucleotide having at least 15 contiguous bases of the nucleobase sequence of Antisense Oligo No. 346 of Table 3.

An oligonucleotide agent as described herein can contain one or more mismatches to the target sequence. In one aspect, an oligonucleotide as described herein contains no more than 3 mismatches. If the oligonucleotide contains mismatches to a target sequence, in some aspects, the area of mismatch is not located in the center of the region of complementarity. If the oligonucleotide contains mismatches to the target sequence, in some aspects, the mismatch should be restricted to be within the last 5 nucleotides from either the 5′- or 3′-end of the region of complementarity. For example, for a 30-linked nucleoside oligonucleotide agent, the contiguous nucleobase region which is complementary to a region of a MSH3 gene, generally does not contain any mismatch within the central 5-10 linked nucleosides. The methods described herein or methods known in the art can be used to determine whether an oligonucleotide containing a mismatch to a target sequence is effective in inhibiting the expression of a MSH3 gene. Consideration of the efficacy of oligonucleotides with mismatches in inhibiting expression of a MSH3 gene is important, especially if the particular region of complementarity in a MSH3 gene is known to have polymorphic sequence variation within the population.

Construction of vectors for expression of polynucleotides can be accomplished using conventional techniques which do not require detailed explanation to one of ordinary skill in the art. For generation of efficient expression vectors, it is necessary to have regulatory sequences that control the expression of the polynucleotide. These regulatory sequences include promoter and enhancer sequences and are influenced by specific cellular factors that interact with these sequences, and are well known in the art.

### A. Alternative Oligonucleosides

In one aspect, one or more of the linked nucleosides or internucleosidic linkages of the oligonucleotide, is naturally occurring, and does not comprise, e.g., chemical modifications and/or conjugations known in the art and described herein. In another aspect, one or more of the linked nucleosides or internucleosidic linkages of an oligonucleotide, is chemically modified to enhance stability or other beneficial characteristics. Without being bound by theory, it is believed that certain modifications can increase nuclease resistance and/or serum stability, or decrease immunogenicity. For example, oligonucleotides can contain nucleotides found to occur naturally in DNA or RNA (e.g., adenine, thymidine, guanosine, cytidine, uridine, or inosine) or can contain alternative nucleosides or internucleosidic linkages which have one or more chemical modifications to one or more components of the nucleotide (e.g., the nucleobase, sugar, or phospho-linker moiety). Oligonucleotides can be linked to one another through naturally occurring phosphodiester bonds, or can contain alternative linkages (e.g., covalently linked through phosphorothioate (e.g., Sp phosphorothioate or Rp phosphorothioate), 3′-methylenephosphonate, 5′-methylenephosphonate, 3′-phosphoamidate, 2′-5′ phosphodiester, guanidinium, S-methylthiourea, 2′-alkoxy, alkyl phosphate, or peptide bonds).

In some aspects, substantially all of the nucleosides or internucleosidic linkages of an oligonucleotide are alternative nucleosides. In other aspects, all of the nucleosides or internucleosidic linkages of an oligonucleotide are alternative nucleosides. Oligonucleotides in which “substantially all of the nucleosides are alternative nucleosides” are largely but not wholly modified and can include not more than five, four, three, two, or one naturally-occurring nucleosides. In still other aspects, oligonucleotides can include not more than five, four, three, two, or one alternative nucleosides.

The nucleic acids can be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. Alternative nucleotides and nucleosides include those with modifications including, for example, end modifications, e.g., 5′-end modifications (phosphorylation, conjugation, inverted linkages) or 3′-end modifications (conjugation, DNA nucleotides, inverted linkages, etc.); base modifications, e.g., replacement with stabilizing bases, destabilizing bases, or bases that base pair with an expanded repertoire of partners, removal of bases (abasic nucleotides), or conjugated bases; sugar modifications (e.g., at the 2′-position or 4′-position) or replacement of the sugar; and/or backbone modifications, including modification or replacement of the phosphodiester linkages. The nucleobase can be an isonucleoside in which the nucleobase is moved from the C1 position of the sugar moiety to a different position (e.g. C2, C3, C4, or C5). Specific examples of oligonucleotide compounds useful in the aspects described herein include, but are not limited to alternative nucleosides containing modified backbones or no natural internucleoside linkages. Nucleotides and nucleosides having modified backbones include, among others, those that do not have a phosphorus atom in the backbone. For the purposes of this specification, and as sometimes referenced in the art, alternative RNAs that do not have a phosphorus atom in their internucleoside backbone can be considered to be oligonucleosides. In some aspects, an oligonucleotide will have a phosphorus atom in its internucleoside backbone.

Alternative internucleoside linkages include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boronophosphates having normal 3′-5′ linkages, 2′-5′-linked analogs of these, and those having inverted polarity wherein the adjacent pairs of nucleoside units are linked 3′-5′ to 5′-3′ or 2′-5′ to 5′-2′. Various salts, mixed salts, and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, the entire contents of each of which are hereby incorporated herein by reference.

Alternative internucleoside linkages that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, the entire contents of each of which are hereby incorporated herein by reference.

In other aspects, suitable oligonucleotides include those in which both the sugar and the internucleoside linkage, i.e., the backbone, of the nucleotide units are replaced. The base units are maintained for hybridization with an appropriate nucleic acid target compound. One such oligomeric compound, a mimetic that has been shown to have excellent hybridization properties, is referred to as a peptide nucleic acid (PNA). In PNA compounds, the sugar of a nucleoside is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, the entire contents of each of which are hereby incorporated herein by reference. Additional PNA compounds suitable for use in the oligonucleotides are described in, for example, in Nielsen et al., Science, 1991, 254, 1497-1500.

Some aspects include oligonucleotides with phosphorothioate backbones and oligonucleotides with heteroatom backbones, and in particular—CH₂—NH—CH₂—, —CH₂—N(CH₃)—O—CH₂—[known as a methylene (methylimino) or MMI backbone], —CH₂—O—N(CH₃)—CH₂—, —CH₂—N(CH₃)—N(CH₃)—CH₂— and —N(CH₃)—CH₂—CH₂—[wherein the native phosphodiester backbone is represented as —O—P—O—CH₂—] of the above-referenced U.S. Pat. No. 5,489,677, and the amide backbones of the above-referenced U.S. Pat. No. 5,602,240. In some aspects, the oligonucleotides featured herein have morpholino backbone structures of the above-referenced U.S. Pat. No. 5,034,506. In other aspects, the oligonucleotides described herein include phosphorodiamidate morpholino oligomers (PMO), in which the deoxyribose moiety is replaced by a morpholine ring, and the charged phosphodiester inter-subunit linkage is replaced by an uncharged phophorodiamidate linkage, as described in Summerton, et al., Antisense Nucleic Acid Drug Dev. 1997, 7:63-70.

Alternative nucleosides and nucleotides can contain one or more substituted sugar moieties. The oligonucleotides, e.g., oligonucleotides, featured herein can include one of the following at the 2′-position: OH; F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Exemplary suitable modifications include —O[(CH₂)ₙO]ₘCH₃, —O(CH₂)ₙOCH₃, —O(CH₂)ₙ—NH₂, —O(CH₂)ₙCH₃, —O(CH₂)ₙ—ONH₂, and —O(CH₂)ₙ—ON[(CH₂)ₙCH₃]₂, where n and m are from 1 to about 10. In other aspects, oligonucleotides include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. In some aspects, the modification includes a 2′-methoxyethoxy (2′—O—CH₂CH₂OCH₃, also known as 2′—O-(2-methoxyethyl) or 2′-MOE) (Martin et al., Helv. Chin. Acta, 1995, 78:486-504) i.e., an alkoxy-alkoxy group. MOE nucleosides confer several beneficial properties to oligonucleotides including, but not limited to, increased nuclease resistance, improved pharmacokinetics properties, reduced non-specific protein binding, reduced toxicity, reduced immunostimulatory properties, and enhanced target affinity as compared to unmodified oligonucleotides.

Another exemplary alternative contains 2′-dimethylaminooxyethoxy, i.e., a —O(CH₂)₂ON(CH₃)₂ group, also known as 2′-DMAOE, as described in examples herein below, and 2′-dimethylaminoethoxyethoxy (also known in the art as 2′—O— dimethylaminoethoxyethyl or 2′-DMAEOE), i.e., 2′—O—(CH₂)₂—O—(CH₂)₂—N(CH₃)₂. Further exemplary alternatives include: 5′-Me-2′-F nucleotides, 5′-Me-2′—OMe nucleotides, 5′-Me-2′-deoxynucleotides, (both R and S isomers in these three families); 2′-alkoxyalkyl; and 2′-NMA (N-methylacetamide).

Other alternatives include 2′-methoxy (2′—OCH₃), 2′-aminopropoxy (2′—OCH₂CH₂CH₂NH₂) and 2′-fluoro (2′-F). Similar modifications can be made at other positions on the nucleosides and nucleotides of an oligonucleotide, particularly the 3′ position of the sugar on the 3′ terminal nucleotide or in 2′-5′ linked oligonucleotides and the 5′ position of 5′ terminal nucleotide. Oligonucleotides can have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application. The entire contents of each of the foregoing are hereby incorporated herein by reference.

An oligonucleotide can include nucleobase (often referred to in the art simply as “base”) alternatives (e.g., modifications or substitutions). Unmodified or natural nucleobases include the purine bases adenine (A) and guanine (G), and the pyrimidine bases thymine (T), cytosine (C) and uracil (U). Alternative nucleobases include other synthetic and natural nucleobases such as 5-methylcytidine, 5-hydroxymethylcytidine, 5-formylcytidine, 5-carboxycytidine, pyrrolocytidine, dideoxycytidine, uridine, 5-methoxyuridine, 5-hydroxydeoxyuridine, dihydrouridine, 4-thiourdine, pseudouridine, 1-methyl-pseudouridine, deoxyuridine, 5-hydroxybutynl-2′-deoxyuridine, xanthine, hypoxanthine, 7-deaza-xanthine, thienoguanine, 8-aza-7-deazaguanosine, 7-methylguanosine, 7-deazaguanosine, 6-aminomethyl-7-deazaguanosine, 8-aminoguanine, 2,2,7-trimethylguanosine, 8-methyladenine, 8-azidoadenine, 7-methyladenine, 7-deazaadenine, 3-deazaadenine, 2,6-diaminopurine, 2-aminopurine, 7-deaza-8-azaadenine, 8-amino-adenine, thymine, dideoxythymine, 5-nitroindole, 2-aminoadenine, 6-methyl and other alkyl derivatives of adenine and guanine, 2-propyl and other alkyl derivatives of adenine and guanine, 2-thiouridine, 2-thiothymine and 2-thiocytosine, 5-halouracil and cytosine, 5-propynyl uridine and cytidine, 6-azo uridine, cytidine and thymine, 4-thiouridine, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uridines and cytidines, 8-azaguanine and 8-azaadenine, and 3-deazaguanine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., (1991) Angewandte Chemie, International Edition, 30:613, and those disclosed by Sanghvi, Y S., Chapter 15, Antisense Research and Applications, pages 289-302, Crooke, S. T. and Lebleu, B., Ed., CRC Press, 1993. Certain of these nucleobases are particularly useful for increasing the binding affinity of the oligonucleotide. These include 5-substituted pyrimidines, 6-azapyrimidines, and N-2, N-6 and 0-6 substituted purines, including 2-aminopropyladenine, 5-propynyluracil, and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., Antisense Research and Applications, CRC Press, Boca Raton, 1993, pp. 276-278) and are exemplary base substitutions, even more particularly when combined with 2′—O— methoxyethyl sugar modifications.

Representative U.S. patents that teach the preparation of certain of the above noted alternative nucleobases as well as other alternative nucleobases include, but are not limited to, the above noted U.S. Pat. Nos. 3,687,808, 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088, the entire contents of each of which are hereby incorporated herein by reference.

In other aspects, the sugar moiety in the nucleotide can be a ribose molecule, optionally having a 2′—O-methyl, 2′—O-MOE, 2′-F, 2′-amino, 2′—O-propyl, 2′-aminopropyl, or 2′—OH modification.

An oligonucleotide can include one or more bicyclic sugar moieties. A “bicyclic sugar” is a furanosyl ring modified by the bridging of two atoms. A “bicyclic nucleoside” (“BNA”) is a nucleoside having a sugar moiety comprising a bridge connecting two carbon atoms of the sugar ring, thereby forming a bicyclic ring system. In some aspects, the bridge connects the 4′-carbon and the 2′-carbon of the sugar ring. Thus, in some aspects, an oligonucleotide can include one or more locked nucleosides. A locked nucleoside is a nucleoside having a modified ribose moiety in which the ribose moiety comprises an extra bridge connecting the 2′ and 4′ carbons. In other words, a locked nucleoside is a nucleoside comprising a bicyclic sugar moiety comprising a 4′—CH₂—O-2′ bridge. This structure effectively “locks” the ribose in the 3′-endo structural conformation. The addition of locked nucleosides to oligonucleotides has been shown to increase oligonucleotide stability in serum, and to reduce off-target effects (Grunweller, A. et al., (2003) Nucleic Acids Research 31(12):3185-3193). Examples of bicyclic nucleosides for use in the polynucleotides include without limitation nucleosides comprising a bridge between the 4′ and the 2′ ribosyl ring atoms. In some aspects, the polynucleotide agents include one or more bicyclic nucleosides comprising a 4′ to 2′ bridge. Examples of such 4′ to 2′ bridged bicyclic nucleosides, include but are not limited to 4′-(CH₂)—O-2′ (LNA); 4′-(CH₂)—S-2′; 4′-(CH₂)₂—O-2′ (ENA); 4′—CH(CH₃)—O-2′ (also referred to as “constrained ethyl” or “cEt”) and 4′—CH(CH₂OCH₃)—O-2′ (and analogs thereof; see, e.g., U.S. Pat. No. 7,399,845); 4′-C(CH₃)(CH₃)—O-2′ (and analogs thereof, see e.g., U.S. Pat. No. 8,278,283); 4′—CH₂—N(OCH₃)-2′ (and analogs thereof, see e.g., U.S. Pat. No. 8,278,425); 4′—CH₂—O—N(CH₃)₂-2′ (see, e.g., U.S. Patent Publication No. 2004/0171570); 4′—CH₂—N(R)—O-2′, wherein R is H, C₁-C₁₂ alkyl, or a protecting group (see, e.g., U.S. Pat. No. 7,427,672); 4′—CH₂—C(H)(CH₃)-2′ (see, e.g., Chattopadhyaya et al., J. Org. Chem., 2009, 74, 118-134); and 4′—CH₂—C(═CH₂)-2′ (and analogs thereof; see, e.g., U.S. Pat. No. 8,278,426). The entire contents of each of the foregoing are hereby incorporated herein by reference.

Additional representative U.S. Patents and US Patent Publications that teach the preparation of locked nucleic acid nucleotides include, but are not limited to, the following: U.S. Pat. Nos. 6,268,490; 6,525,191; 6,670,461; 6,770,748; 6,794,499; 6,998,484; 7,053,207; 7,034,133; 7,084,125; 7,399,845; 7,427,672; 7,569,686; 7,741,457; 8,022,193; 8,030,467; 8,278,425; 8,278,426; 8,278,283; US 2008/0039618; and US 2009/0012281, the entire contents of each of which are hereby incorporated herein by reference.

Any of the foregoing bicyclic nucleosides can be prepared having one or more stereochemical sugar configurations including for example α-L-ribofuranose and β-D-ribofuranose (see WO 99/14226).

An oligonucleotide can be modified to include one or more constrained ethyl nucleosides. As used herein, a “constrained ethyl nucleoside” or “cEt” is a locked nucleoside comprising a bicyclic sugar moiety comprising a 4′—CH(CH₃)—O-2′ bridge. In one aspect, a constrained ethyl nucleoside is in the S conformation referred to herein as “S-cEt.”

An oligonucleotide can include one or more “conformationally restricted nucleosides” (“CRN”). CRN are nucleoside analogs with a linker connecting the C2′ and C4′ carbons of ribose or the C3 and —C5′ carbons of ribose. CRN lock the ribose ring into a stable conformation and increase the hybridization affinity to mRNA. The linker is of sufficient length to place the oxygen in an optimal position for stability and affinity resulting in less ribose ring puckering.

Representative publications that teach the preparation of certain of the above noted CRN include, but are not limited to, US Patent Publication No. 2013/0190383; and PCT publication WO 2013/036868, the entire contents of each of which are hereby incorporated herein by reference.

In some aspects, an oligonucleotide comprises one or more monomers that are UNA (unlocked nucleoside) nucleosides. UNA is unlocked acyclic nucleoside, wherein any of the bonds of the sugar has been removed, forming an unlocked “sugar” residue. In one example, UNA also encompasses monomer with bonds between C1′-C4′ have been removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar has been removed (see Nuc. Acids Symp. Series, 52, 133-134 (2008) and Fluiter et al., Mol. Biosyst., 2009, 10, 1039 hereby incorporated by reference).

Representative U.S. publications that teach the preparation of UNA include, but are not limited to, U.S. Pat. No. 8,314,227; and US Patent Publication Nos. 2013/0096289; 2013/0011922; and 2011/0313020, the entire contents of each of which are hereby incorporated herein by reference.

The ribose molecule can be modified with a cyclopropane ring to produce a tricyclodeoxynucleic acid (tricyclo DNA). The ribose moiety can be substituted for another sugar such as 1,5,-anhydrohexitol, threose to produce a threose nucleoside (TNA), or arabinose to produce an arabino nucleoside. The ribose molecule can be replaced with non-sugars such as cyclohexene to produce cyclohexene nucleoside or glycol to produce glycol nucleosides.

Potentially stabilizing modifications to the ends of nucleoside molecules can include N-(acetylaminocaproyl)-4-hydroxyprolinol (Hyp-C6-NHAc), N-(caproyl-4-hydroxyprolinol (Hyp-C6), N-(acetyl-4-hydroxyprolinol (Hyp-NHAc), thymidine-2′—O— deoxythymidine (ether), N-(aminocaproyl)-4-hydroxyprolinol (Hyp-C6-amino), 2-docosanoyl-uridine-3″-phosphate, inverted base dT(idT) and others. Disclosure of this modification can be found in PCT Publication No. WO 2011/005861.

Other alternatives chemistries of an oligonucleotide include a 5′ phosphate or 5′ phosphate mimic, e.g., a 5′-terminal phosphate or phosphate mimic of an oligonucleotide. Suitable phosphate mimics are disclosed in, for example US Patent Publication No. 2012/0157511, the entire contents of which are incorporated herein by reference.

Exemplary oligonucleotides comprise nucleosides with alternative sugar moieties and can comprise DNA or RNA nucleosides. In some aspects, the oligonucleotide comprises nucleosides comprising alternative sugar moieties and DNA nucleosides. Incorporation of alternative nucleosides into the oligonucleotide can enhance the affinity of the oligonucleotide for the target nucleic acid. In that case, the alternative nucleosides can be referred to as affinity enhancing alternative nucleotides.

In some aspects, the oligonucleotide comprises at least 1 alternative nucleoside, such as at least 2, at least 3, at least 4, at least 5, at least 6, at least 7, at least 8, at least 9, at least 10, at least 11, at least 12, at least 13, at least 14, at least 15 or at least 16 alternative nucleosides. In other aspects, the oligonucleotides comprise from 1 to 10 alternative nucleosides, such as from 2 to 9 alternative nucleosides, such as from 3 to 8 alternative
A-LNA 5mC-LNA G-LNA T-LNA

nucleosides, such as from 4 to 7 alternative nucleosides, such as 6 or 7 alternative nucleosides. In an aspect, the oligonucleotide can comprise alternatives, which are independently selected from these three types of alternatives (alternative sugar moiety, alternative nucleobase, and alternative internucleoside linkage), or a combination thereof. In one aspect, the oligonucleotide comprises one or more nucleosides comprising alternative sugar moieties, e.g., 2′ sugar alternative nucleosides. In some aspect, the oligonucleotide comprises the one or more 2′ sugar alternative nucleoside independently selected from the group consisting of 2′—O-alkyl-RNA, 2′—O-methyl-RNA, 2′-alkoxy-RNA, 2′—O-methoxyethyl-RNA, 2′-amino-DNA, 2′-fluoro-DNA, arabino nucleic acid (ANA), 2′-fluoro-ANA, and BNA (e.g., LNA) nucleosides. Exemplary structures of the LNAs are as follows.

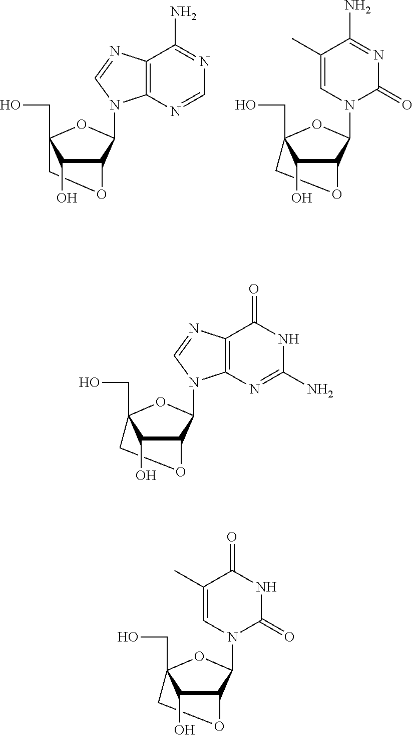 [SMILES: Nc1ncnc2c1ncn2[C@@H]1O[C@@]2(CO)CO[C@H]1C2O ; Cc1cn([C@@H]2O[C@@]3(CO)CO[C@H]2C3O)c(=O)nc1N ; Nc1nc2c(ncn2[C@@H]2O[C@@]3(CO)CO[C@H]2C3O)c(=O)[nH]1 ; Cc1cn([C@@H]2O[C@@]3(CO)CO[C@H]2C3O)c(=O)[nH]c1=O]

In some aspects, the one or more alternative nucleoside is a BNA.

In some aspects, at least 1 of the alternative nucleosides is a BNA (e.g., an LNA (e.g., A-LNA, 5mC L-NA, G-LNA, T-LNA)), such as at least 2, such as at least 3, at least 4, at least 5, at least 6, at least 7, or at least 8 of the alternative nucleosides are BNAs. In a still further aspect, all the alternative nucleosides are BNAs.

In a further aspect, the oligonucleotide comprises at least one alternative internucleoside linkage. In some aspects, the internucleoside linkages within the contiguous nucleotide sequence are phosphorothioate or boronophosphate internucleoside linkages. In some aspects, all the internucleotide linkages in the contiguous sequence of the oligonucleotide are phosphorothioate linkages. In some aspects, the phosphorothioate linkages are stereochemically pure phosphorothioate linkages. In some aspects, the phosphorothioate linkages are Sp phosphorothioate linkages. In other aspects, the phosphorothioate linkages are Rp phosphorothioate linkages.

In some aspects, the oligonucleotide comprises at least one alternative nucleoside which is a 2′-MOE-RNA, such as 2, 3, 4, 5, 6, 7, 8, 9, or 10 2′-MOE-RNA nucleoside units. In some aspects, the 2′-MOE-RNA nucleoside units are connected by phosphorothioate linkages. In some aspects, at least one of said alternative nucleoside is 2′-fluoro DNA (also known as 2′-deoxy, 2′-fluoro-DNA), such as 2, 3, 4, 5, 6, 7, 8, 9, or 10 2′-fluoro-DNA nucleoside units. In some aspects, the oligonucleotide comprises at least one BNA unit and at least one 2′ substituted modified nucleoside. In some aspects, the oligonucleotide comprises both 2′ sugar modified nucleosides and DNA units. In some aspects, the oligonucleotide or contiguous nucleotide region thereof is a gapmer oligonucleotide.

### B. Oligonucleotides Conjugated to Ligands

Oligonucleotides can be chemically linked to one or more ligands, moieties, or conjugates that enhance the activity, cellular distribution, or cellular uptake of the oligonucleotide. Such moieties include but are not limited to lipid moieties such as a cholesterol moiety (Letsinger et al., (1989) Proc. Natl. Acid. Sci. USA, 86: 6553-6556), cholic acid (Manoharan et al., (1994) Biorg. Med. Chem. Let., 4:1053-1060), a thioether, e.g., beryl-S-tritylthiol (Manoharan et al., (1992) Ann. N.Y. Acad. Sci., 660:306-309; Manoharan et al., (1993) Biorg. Med. Chem. Let., 3:2765-2770), a thiocholesterol (Oberhauser et al., (1992) Nucl. Acids Res., 20:533-538), an aliphatic chain, e.g., dodecandiol or undecyl residues (Saison-Behmoaras et al., (1991) EMBO J, 10:1111-1118; Kabanov et al., (1990) FEBS Lett., 259:327-330; Svinarchuk et al., (1993) Biochimie, 75:49-54), a phospholipid, e.g., di-hexadecyl-rac-glycerol or triethylammonium 1,2-di-O-hexadecyl-rac-glycero-3-phosphonate (Manoharan et al., (1995) Tetrahedron Lett., 36:3651-3654; Shea et al., (1990) Nucl. Acids Res., 18:3777-3783), a polyamine or a polyethylene glycol chain (Manoharan et al., (1995) Nucleosides & Nucleotides, 14:969-973), or adamantane acetic acid (Manoharan et al., (1995) Tetrahedron Lett., 36:3651-3654), a palmityl moiety (Mishra et al., (1995) Biochim. Biophys. Acta, 1264:229-237), or an octadecylamine or hexylamino-carbonyloxycholesterol moiety (Crooke et al., (1996) J. Pharmacol. Exp. Ther., 277:923-937).

In one aspect, a ligand alters the distribution, targeting, or lifetime of an oligonucleotide agent into which it is incorporated. In some aspects, a ligand provides an enhanced affinity for a selected target, e.g., molecule, cell or cell type, compartment, e.g., a cellular or organ compartment, tissue, organ, or region of the body, as, e.g., compared to a species absent such a ligand.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin, N-acetylglucosamine, N-acetylgalactosamine, or hyaluronic acid); or a lipid. The ligand can be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can include targeting groups, e.g., a cell or tissue targeting agent, e.g., a lectin, glycoprotein, lipid or protein, e.g., an antibody, that bind to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, vitamin A, biotin, or an RGD peptide or RGD peptide mimetic.

Other examples of ligands include dyes, intercalating agents (e.g. acridines), cross-linkers (e.g. psoralen, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid*, O3-(oleoyl)lithocholic acid, 03-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a hepatic cell. Ligands can include hormones and hormone receptors. They can include non-peptidic species, such as lipids, lectins, carbohydrates, vitamins, cofactors, multivalent lactose, multivalent galactose, N-acetyl-galactosamine, N-acetyl-gulucosamine multivalent mannose, or multivalent fucose.

The ligand can be a substance, e.g., a drug, which can increase the uptake of the oligonucleotide agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin.

In some aspects, a ligand attached to an oligonucleotide as described herein acts as a pharmacokinetic modulator (PK modulator). PK modulators include lipophiles, bile acids, steroids, phospholipid analogues, peptides, protein binding agents, PEG, vitamins etc. Exemplary PK modulators include, but are not limited to, cholesterol, fatty acids, cholic acid, lithocholic acid, dialkylglycerides, diacylglyceride, phospholipids, sphingolipids, naproxen, ibuprofen, vitamin E, biotin etc. Oligonucleotides that comprise a number of phosphorothioate linkages are also known to bind to serum protein, thus short oligonucleotides, e.g., oligonucleotides of about 5 bases, 10 bases, 15 bases, or 20 bases, comprising multiple of phosphorothioate linkages in the backbone are also amenable as ligands (e.g. as PK modulating ligands). In addition, aptamers that bind serum components (e.g. serum proteins) are also suitable for use as PK modulating ligands in the aspects described herein.

Ligand-conjugated oligonucleotides can be synthesized by the use of an oligonucleotide that bears a pendant reactive functionality, such as that derived from the attachment of a linking molecule onto the oligonucleotide (described below). This reactive oligonucleotide can be reacted directly with commercially-available ligands, ligands that are synthesized bearing any of a variety of protecting groups, or ligands that have a linking moiety attached thereto.

The oligonucleotides used in the conjugates can be conveniently and routinely made through the well-known technique of solid-phase synthesis. Equipment for such synthesis is sold by several vendors including, for example, Applied Biosystems (Foster City, Calif.). Any other means for such synthesis known in the art can additionally or alternatively be employed. It is also known to use similar techniques to prepare other oligonucleotides, such as the phosphorothioates and alkylated derivatives.

In the ligand-conjugated oligonucleotides, such as the ligand-molecule bearing sequence-specific linked nucleosides, the oligonucleotides and oligonucleosides can be assembled on a suitable DNA synthesizer utilizing standard nucleotide or nucleoside precursors, or nucleotide or nucleoside conjugate precursors that already bear the linking moiety, ligand-nucleotide or nucleoside-conjugate precursors that already bear the ligand molecule, or non-nucleoside ligand-bearing building blocks.

When using conjugate precursors that already bear a linking moiety, the synthesis of the sequence-specific linked nucleosides is typically completed, and the ligand molecule is then reacted with the linking moiety to form the ligand-conjugated oligonucleotide. In some aspects, the oligonucleotides or linked nucleosides are synthesized by an automated synthesizer using phosphoramidites derived from ligand-nucleoside conjugates in addition to the standard phosphoramidites and non-standard phosphoramidites that are commercially available and routinely used in oligonucleotide synthesis.

i. Lipid Conjugates

In one aspect, the ligand or conjugate is a lipid or lipid-based molecule. Such a lipid or lipid-based molecule can bind a serum protein, e.g., human serum albumin (HSA). An HSA binding ligand allows for distribution of the conjugate to a target tissue, e.g., a non-kidney target tissue of the body. A lipid or lipid-based ligand can (a) increase resistance to degradation of the conjugate, (b) increase targeting or transport into a target cell or cell membrane, and/or (c) be used to adjust binding to a serum protein, e.g., HSA.

In another aspect, the ligand is a moiety, e.g., a vitamin, which is taken up by a target cell, e.g., a proliferating cell. Exemplary vitamins include vitamin A, E, and K.

ii. Cell Permeation Agents

In another aspect, the ligand is a cell-permeation agent, such as a helical cell-permeation agent. In one aspect, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. In one aspect, the helical agent is an alpha-helical agent, which can have a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to oligonucleotide agents can affect pharmacokinetic distribution of the oligonucleotide, such as by enhancing cellular recognition and absorption. The peptide or peptidomimetic moiety can be about 5-50 amino acids long, e.g., about 5, 10, 15, 20, 25, 30, 35, 40, 45, or 50 amino acids long.

A peptide or peptidomimetic can be, for example, a cell permeation peptide, cationic peptide, amphipathic peptide, or hydrophobic peptide (e.g., consisting primarily of Tyr, Trp, or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF having the amino acid sequence AAVALLPAVLLALLAP. An RFGF analogue (e.g., amino acid sequence AALLPVLLAAP containing a hydrophobic MTS can be a targeting moiety. The peptide moiety can be a “delivery” peptide, which can carry large polar molecules including peptides, oligonucleotides, and protein across cell membranes. For example, sequences from the HIV Tat protein (GRKKRRQRRRPPQ and the Drosophila Antennapedia protein (RQIKIWFQNRRMKWKK have been found to be capable of functioning as delivery peptides. A peptide or peptidomimetic can be encoded by a random sequence of DNA, such as a peptide identified from a phage-display library, or one-bead-one-compound (OBOC) combinatorial library (Lam et al., Nature, 354:82-84, 1991). Examples of a peptide or peptidomimetic tethered to an oligonucleotide agent via an incorporated monomer unit for cell targeting purposes is an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

An RGD peptide for use in the compositions and methods can be linear or cyclic, and can be modified, e.g., glycosylated or methylated, to facilitate targeting to a specific tissue(s). RGD-containing peptides and peptidiomimemtics can include D-amino acids, as well as synthetic RGD mimics. In addition to RGD, one can use other moieties that target the integrin ligand. Some conjugates of this ligand target PECAM-1 or VEGF.

A cell permeation peptide is capable of permeating a cell, e.g., a microbial cell, such as a bacterial or fungal cell, or a mammalian cell, such as a human cell. A microbial cell-permeating peptide can be, for example, an α-helical linear peptide (e.g., LL-37 or Ceropin P1), a disulfide bond-containing peptide (e.g., α-defensin, β-defensin, or bactenecin), or a peptide containing only one or two dominating amino acids (e.g., PR-39 or indolicidin). A cell permeation peptide can include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni et al., Nucl. Acids Res. 31:2717-2724, 2003).

iii. Carbohydrate Conjugates

In some aspects of the compositions and methods described herein, an oligonucleotide further comprises a carbohydrate. The carbohydrate conjugated oligonucleotides are advantageous for the in vivo delivery of nucleic acids, as well as compositions suitable for in vivo therapeutic use, as described herein. As used herein, “carbohydrate” refers to a compound which is either a carbohydrate per se made up of one or more monosaccharide units having at least 6 carbon atoms (which can be linear, branched or cyclic) with an oxygen, nitrogen or sulfur atom bonded to each carbon atom; or a compound having as a part thereof a carbohydrate moiety made up of one or more monosaccharide units each having at least six carbon atoms (which can be linear, branched or cyclic), with an oxygen, nitrogen or sulfur atom bonded to each carbon atom. Representative carbohydrates include the sugars (mono-, di-, tri- and oligosaccharides containing from about 4, 5, 6, 7, 8, or 9 monosaccharide units), and polysaccharides such as starches, glycogen, cellulose and polysaccharide gums. Specific monosaccharides include C5 and above (e.g., C5, C6, C7, or C8) sugars; di- and trisaccharides include sugars having two or three monosaccharide units (e.g., C5, C6, C7, or C8).

In one aspect, a carbohydrate conjugate for use in the compositions and methods described herein is a monosaccharide.

In some aspects, the carbohydrate conjugate further comprises one or more additional ligands as described above, such as, but not limited to, a PK modulator and/or a cell permeation peptide.

Additional carbohydrate conjugates (and linkers) suitable for use include those described in PCT Publication Nos. WO 2014/179620 and WO 2014/179627, the entire contents of each of which are incorporated herein by reference.

iv. Linkers

In some aspects, the conjugate or ligand described herein can be attached to an oligonucleotide with various linkers that can be cleavable or non-cleavable.

Linkers typically comprise a direct bond or an atom such as oxygen or sulfur, a unit such as NR⁸, C(O), C(O)NH, SO, SO₂, SO₂NH or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by O, S, S(O), SO₂, N(R⁸), C(O), substituted or unsubstituted aryl, substituted or unsubstituted heteroaryl, substituted or unsubstituted heterocyclic; where R⁸ is hydrogen, acyl, aliphatic or substituted aliphatic. In one aspect, the linker is between about 1-24, 2-24, 3-24, 4-24, 5-24, 6-24, 6-18, 7-18, 8-18, 7-17, 8-17, 6-16, 7-17, 8-16 or 1, 2, 3, 4, 5, 6, 7, 8, 9, 10, 11, 12, 13, 14, 15, 16, 17, 18, 19, 20, 21, 21, 22, 23, or 24 atoms.

A cleavable linking group is one which is sufficiently stable outside the cell, but which upon entry into a target cell is cleaved to release the two parts the linker is holding together. In somes aspects, the cleavable linking group is cleaved at least 10 times, 20 times, 30 times, 40 times, 50 times, 60 times, 70 times, 80 times, 90 times, or more, or at least 100 times faster in a target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linking groups are susceptible to cleavage agents, e.g., pH, redox potential, or the presence of degradative molecules. Generally, cleavage agents are more prevalent or found at higher levels or activities inside cells than in serum or blood. Examples of such degradative agents include: redox agents which are selective for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing a cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, a liver-targeting ligand can be linked to a cationic lipid through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis.

Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It will also be desirable to test the candidate cleavable linking group for the ability to resist cleavage in the blood or when in contact with other non-target tissue. Thus, one can determine the relative susceptibility to cleavage between at least two conditions, where at least one condition is selected to be indicative of cleavage in a target cell and another condition is selected to be indicative of cleavage in other tissues or biological fluids, e.g., blood or serum. The evaluations can be carried out in cell free systems, in cells, in cell culture, in organ or tissue culture, or in whole animals. It can be useful to make initial evaluations in cell-free or culture conditions and to confirm by further evaluations in whole animals. In some aspects, useful candidate compounds are cleaved at least 2, 4, 10, 20, 30, 40, 50, 60, 70, 80, 90, or 100 times faster in the cell (or under in vitro conditions selected to mimic intracellular conditions) as compared to blood or serum (or under in vitro conditions selected to mimic extracellular conditions).

a. Redox Cleavable Linking Groups

In one aspect, a cleavable linking group is a redox cleavable linking group that is cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular oligonucleotide moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can be evaluated under conditions which are selected to mimic blood or serum conditions. In one aspect, candidate compounds are cleaved by at most about 10% in the blood. In other aspects, useful candidate compounds are degraded at least 2, 4, 10, 20, 30, 40, 50, 60, 70, 80, 90, or 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

b. Phosphate-Based Cleavable Linking Groups

In another aspect, a cleavable linker comprises a phosphate-based cleavable linking group. A phosphate-based cleavable linking group is cleaved by agents that degrade or hydrolyze the phosphate group. An example of an agent that cleaves phosphate groups in cells are enzymes such as phosphatases in cells. Examples of phosphate-based linking groups are —O—P(O)(OR^{k})—O—, —O—P(S)(OR^{k})—O—, —O—P(S)(SR^{k})—O—, —S—P(O)(OR^{k})—O—, —O—P(O)(OR^{k})—S—, —S—P(O)(OR^{k})—S—, —O—P(S)(OR^{k})—S—, —S—P(S)(OR^{k})—O—, —O—P(O)(R^{k})—O—, —O—P(S)(R^{k})—O—, —S—P(O)(R^{k})—O—, —S—P(S)(R^{k})—O—, —S—P(O)(R^{k})—S—, —O—P(S)(R^{k})—S—. These candidates can be evaluated using methods analogous to those described above.

c. Acid Cleavable Linking Groups

In another aspect, a cleavable linker comprises an acid cleavable linking group. An acid cleavable linking group is a linking group that is cleaved under acidic conditions. In some aspects, acid cleavable linking groups are cleaved in an acidic environment with a pH of about 6.5 or lower (e.g., about 6.0, 5.75, 5.5, 5.25, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula —C═NN—, C(O)O, or —OC(O). In one aspect, the carbon is attached to the oxygen of the ester (the alkoxy group) is an aryl group, substituted alkyl group, or tertiary alkyl group such as dimethyl pentyl or t-butyl. These candidates can be evaluated using methods analogous to those described above.

d. Ester-Based Linking Groups

In another aspect, a cleavable linker comprises an ester-based cleavable linking group. An ester-based cleavable linking group is cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

e. Peptide-Based Cleaving Groups

In yet another aspect, a cleavable linker comprises a peptide-based cleavable linking group. A peptide-based cleavable linking group is cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene, or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linking groups have the general formula —NHCHR^{A}C(O)NHCHR^{B}C(O)—, where R^{A} and RB are the R groups of the two adjacent amino acids. These candidates can be evaluated using methods analogous to those described above.

In one aspect, an oligonucleotide is conjugated to a carbohydrate through a linker. Linkers include bivalent and trivalent branched linker groups. Linkers for oligonucleotide carbohydrate conjugates include, but are not limited to, those described in formulas 24-35 of PCT Publication No. WO 2018/195165.

Representative U.S. patents that teach the preparation of oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941; 6,294,664; 6,320,017; 6,576,752; 6,783,931; 6,900,297; 7,037,646; 8,106,022, the entire contents of each of which are hereby incorporated herein by reference.

It is not necessary for all positions in a given compound to be uniformly modified, and in fact more than one of the aforementioned modifications can be incorporated in a single compound or even at a single nucleoside within an oligonucleotide. Oligonucleotide compounds that are chimeric compounds are also contemplated. Chimeric oligonucleotides typically contain at least one region wherein the RNA is modified so as to confer upon the oligonucleotide increased resistance to nuclease degradation, increased cellular uptake, and/or increased binding affinity for the target nucleic acid. An additional region of the oligonucleotide can serve as a substrate for enzymes capable of cleaving RNA:DNA. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxy oligonucleotides hybridizing to the same target region. Cleavage of the RNA target can be routinely detected by gel electrophoresis and, if necessary, associated nucleic acid hybridization techniques known in the art.

In certain instances, the nucleotides of an oligonucleotide can be modified by a non-ligand group. A number of non-ligand molecules have been conjugated to oligonucleotides in order to enhance the activity, cellular distribution, or cellular uptake of the oligonucleotide, and procedures for performing such conjugations are available in the scientific literature. Such non-ligand moieties have included lipid moieties, such as cholesterol (Kubo, T. et al., Biochem. Biophys. Res. Comm, 2007, 365(1):54-61; Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-S-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Representative United States patents that teach the preparation of such oligonucleotide conjugates have been listed above. Typical conjugation protocols involve the synthesis of an oligonucleotide bearing an aminolinker at one or more positions of the sequence. The amino group is then reacted with the molecule being conjugated using appropriate coupling or activating reagents. The conjugation reaction can be performed either with the oligonucleotide still bound to the solid support or following cleavage of the oligonucleotide, in solution phase. Purification of the oligonucleotide conjugate by HPLC typically affords the pure conjugate.

### IV. Pharmaceutical Uses

The oligonucleotide, or pharmaceutically acceptable salt thereof, compositions described herein are useful in the methods described herein, and, while not bound by theory, are believed to exert their desirable effects through their ability to modulate the level, status, and/or activity of a MutSP heterodimer comprising MSH3, e.g., by inhibiting the activity or level of the MSH3 protein in a cell in a mammal.

An aspect relates to methods of treating disorders related to DNA mismatch repair such as nucleotide repeat expansion disorders (e.g., trinucleotide repeat expansion disorders) in a subject in need thereof. Another aspect includes reducing the level of MSH3 in a cell of a subject identified as having a nucleotide repeat expansion disorder (e.g., a trinucleotide repeat expansion disorder). Still another aspect includes a method of inhibiting expression of MSH3 in a cell in a subject. Further aspects include methods of decreasing nucleotide repeat expansion in a cell. The methods include contacting a cell with an oligonucleotide, or pharmaceutically acceptable salt thereof, in an amount effective to inhibit expression of MSH3 in the cell, thereby inhibiting expression of MSH3 in the cell.

Based on the above methods, an oligonucleotide, or pharmaceutically acceptable salt thereof, or a composition comprising such an oligonucleotide, or pharmaceutically acceptable salt thereof, for use in therapy, or for use as a medicament, or for use in treating disorders related to DNA mismatch repair such as repeat expansion disorders in a subject in need thereof, or for use in reducing the level of MSH3 in a cell of a subject identified as having a nucleotide repeat expansion disorder (e.g., a trinucleotide repeat expansion disorder), or for use in inhibiting expression of MSH3 in a cell in a subject, or for use in decreasing nucleotide repeat expansion (e.g., trinucleotide repeat expansion) in a cell is contemplated. The uses include the contacting of a cell with the oligonucleotide, or pharmaceutically acceptable salt thereof, in an amount effective to inhibit expression of MSH3 in the cell, thereby inhibiting expression of MSH3 in the cell. Aspects described below in relation to the methods described herein are also applicable to these further aspects.

Contacting of a cell with an oligonucleotide, or pharmaceutically acceptable salt thereof, can be done in vitro or in vivo. Contacting a cell in vivo with the oligonucleotide, or pharmaceutically acceptable salt thereof, includes contacting a cell or group of cells within a subject, e.g., a human subject, with the oligonucleotide, or pharmaceutically acceptable salt thereof. Combinations of in vitro and in vivo methods of contacting a cell are also possible. Contacting a cell can be direct or indirect, as discussed above. Furthermore, contacting a cell can be accomplished via a targeting ligand, including any ligand described herein or known in the art. In some aspects, the targeting ligand is a carbohydrate moiety, e.g., a GalNAc3 ligand, or any other ligand that directs the oligonucleotide to a site of interest. Cells can include those of the central nervous system, or muscle cells. Inhibiting expression of a MSH3 gene includes any level of inhibition of a MSH3 gene, e.g., at least partial suppression of the expression of a MSH3 gene, such as an inhibition by at least 20%. In some aspects, inhibition is by at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%.

The expression of a MSH3 gene can be assessed based on the level of any variable associated with MSH3 gene expression, e.g., MSH3 mRNA level or MSH3 protein level.

Inhibition can be assessed by a decrease in an absolute or relative level of one or more of these variables compared with a control level. The control level can be any type of control level that is utilized in the art, e.g., a pre-dose baseline level, or a level determined from a similar subject, cell, or sample that is untreated or treated with a control (such as, e.g., buffer only control or inactive agent control).

In some aspects, surrogate markers can be used to detect inhibition of MSH3. For example, effective treatment of a nucleotide repeat expansion disorder (e.g., a trinucleotide repeat expansion disorder), as demonstrated by acceptable diagnostic and monitoring criteria with an agent to reduce MSH3 expression can be understood to demonstrate a clinically relevant reduction in MSH3.

In some aspects of the methods, expression of a MSH3 gene is inhibited by at least 20%, 25%, 30%, 35%, 40%, 45%, 50%, 55%, 60%, 65%, 70%, 75%, 80%, 85%, 90%, or 95%, or to below the level of detection of the assay. In some aspects, the methods include a clinically relevant inhibition of expression of MSH3, e.g., as demonstrated by a clinically relevant outcome after treatment of a subject with an agent to reduce the expression of MSH3.

Inhibition of the expression of a MSH3 gene can be manifested by a reduction of the amount of mRNA expressed by a first cell or group of cells (such cells can be present, for example, in a sample derived from a subject) in which a MSH3 gene is transcribed and which has or have been treated (e.g., by contacting the cell or cells with an oligonucleotide, or pharmaceutically acceptable salt thereof, or by administering an oligonucleotide, or pharmaceutically acceptable salt thereof, to a subject in which the cells are or were present) such that the expression of a MSH3 gene is inhibited, as compared to a second cell or group of cells substantially identical to the first cell or group of cells but which has not or have not been so treated (control cell(s) not treated with an oligonucleotide, or pharmaceutically acceptable salt thereof, or not treated with an oligonucleotide, or pharmaceutically acceptable salt thereof, targeted to the gene of interest). The degree of inhibition can be expressed in terms of:
×100%

In other aspects, inhibition of the expression of a MSH3 gene can be assessed in terms of a reduction of a parameter that is functionally linked to MSH3 gene expression, e.g., MSH3 protein expression or MSH3 signaling pathways. MSH3 gene silencing can be determined in any cell expressing MSH3, either endogenous or heterologous from an expression construct, and by any assay known in the art.

Inhibition of the expression of a MSH3 protein can be manifested by a reduction in the level of the MSH3 protein that is expressed by a cell or group of cells (e.g., the level of protein expressed in a sample derived from a subject). As explained above, for the assessment of mRNA suppression, the inhibition of protein expression levels in a treated cell or group of cells can similarly be expressed as a percentage of the level of protein in a control cell or group of cells.

A control cell or group of cells that can be used to assess the inhibition of the expression of a MSH3 gene includes a cell or group of cells that has not yet been contacted with an oligonucleotide. For example, the control cell or group of cells can be derived from an individual subject (e.g., a human or animal subject) prior to treatment of the subject with an oligonucleotide.

The level of MSH3 mRNA that is expressed by a cell or group of cells can be determined using any method known in the art for assessing mRNA expression. In one aspect, the level of expression of MSH3 in a sample is determined by detecting a transcribed polynucleotide, or portion thereof, e.g., mRNA of the MSH3 gene. RNA can be extracted from cells using RNA extraction techniques including, for example, using acid phenol/guanidine isothiocyanate extraction (RNAzol B; Biogenesis), RNEASY™ RNA preparation kits (Qiagen) or PAXgene (PreAnalytix, Switzerland). Typical assay formats utilizing ribonucleic acid hybridization include nuclear run-on assays, RT-PCR, RNase protection assays, northern blotting, in situ hybridization, and microarray analysis. Circulating MSH3 mRNA can be detected using methods the described in PCT Publication WO2012/177906, the entire contents of which are hereby incorporated herein by reference. In some aspects, the level of expression of MSH3 is determined using a nucleic acid probe. The term “probe,” as used herein, refers to any molecule that is capable of selectively binding to a specific MSH3 sequence, e.g. to an mRNA or polypeptide. Probes can be synthesized by one of skill in the art, or derived from appropriate biological preparations. Probes can be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or northern analyses, polymerase chain reaction (PCR) analyses, and probe arrays. One method for the determination of mRNA levels involves contacting the isolated mRNA with a nucleic acid molecule (probe) that can hybridize to MSH3 mRNA. In one aspect, the mRNA is immobilized on a solid surface and contacted with a probe, for example by running the isolated mRNA on an agarose gel and transferring the mRNA from the gel to a membrane, such as nitrocellulose. In an alternative aspect, the probe(s) are immobilized on a solid surface and the mRNA is contacted with the probe(s), for example, in an AFFYMETRIX gene chip array. A skilled artisan can readily adapt known mRNA detection methods for use in determining the level of MSH3 mRNA.

An alternative method for determining the level of expression of MSH3 in a sample involves the process of nucleic acid amplification and/or reverse transcriptase (to prepare cDNA) of for example mRNA in the sample, e.g., by RT-PCR (the experimental aspect set forth in Mullis, 1987, U.S. Pat. No. 4,683,202), ligase chain reaction (Barany (1991) Proc. Natl. Acad. Sci. USA 88:189-193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In some aspects, the level of expression of MSH3 is determined by quantitative fluorogenic RT-PCR (i.e., the TAQMAN™ System) or the DUAL-GLO® Luciferase assay.

The expression levels of MSH3 mRNA can be monitored using a membrane blot (such as used in hybridization analysis such as northern, Southern, dot, and the like), or microwells, sample tubes, gels, beads or fibers (or any solid support comprising bound nucleic acids). See U.S. Pat. Nos. 5,770,722; 5,874,219; 5,744,305; 5,677,195; and 5,445,934, which are incorporated herein by reference. The determination of MSH3 expression level can comprise using nucleic acid probes in solution.

In some aspects, the level of mRNA expression is assessed using branched DNA (bDNA) assays or real time PCR (qPCR). The use of this PCR method is described and exemplified in the Examples presented herein. Such methods can be used for the detection of MSH3 nucleic acids.

The level of MSH3 protein expression can be determined using any method known in the art for the measurement of protein levels. Such methods include, for example, electrophoresis, capillary electrophoresis, high performance liquid chromatography (HPLC), thin layer chromatography (TLC), hyperdiffusion chromatography, fluid or gel precipitin reactions, absorption spectroscopy, a colorimetric assays, spectrophotometric assays, flow cytometry, immunodiffusion (single or double), immunoelectrophoresis, western blotting, radioimmunoassay (RIA), enzyme-linked immunosorbent assays (ELISAs), immunofluorescent assays, electrochemiluminescence assays, and the like. Such assays can be used for the detection of proteins indicative of the presence or replication of MSH3 proteins.

In some aspects of the methods described herein, the oligonucleotide, or pharmaceutically acceptable salt thereof, is administered to a subject such that the oligonucleotide, or pharmaceutically acceptable salt thereof, is delivered to a specific site within the subject. The inhibition of expression of MSH3 can be assessed using measurements of the level or change in the level of MSH3 mRNA or MSH3 protein in a sample derived from a specific site within the subject. In some aspects, the methods include a clinically relevant inhibition of expression of MSH3, e.g., as demonstrated by a clinically relevant outcome after treatment of a subject with an agent to reduce the expression of MSH3.

In other aspects, the oligonucleotide, or pharmaceutically acceptable salt thereof, is administered in an amount and for a time effective to result in one of (or more, e.g., two or more, three or more, four or more of): (a) decrease the number of repeats, (b) decrease the level of polyglutamine, (c) decreased cell death (e.g., CNS cell death and/or muscle cell death), (d) delayed onset of the disorder, (e) increased survival of subject, and (f) increased progression free survival of a subject.

Treating nucleotide repeat expansion disorders (e.g., trinucleotide repeat expansion disorders) can result in an increase in average survival time of an individual or a population of subjects treated with an oligonucleotide, or pharmaceutically acceptable salt thereof, described herein in comparison to a population of untreated subjects. For example, the survival time of an individual or average survival time of a population is increased by more than 30 days (more than 60 days, 90 days, or 120 days). An increase in survival time of an individual or in average survival time of a population can be measured by any reproducible means. An increase in survival time of an individual can be measured, for example, by calculating for an individual the length of survival time following the initiation of treatment with the compound described herein. An increase in average survival time of a population can be measured, for example, by calculating for the average length of survival time following initiation of treatment with the compound described herein. An increase in survival time of an individual can be measured, for example, by calculating for an individual length of survival time following completion of a first round of treatment with a compound or pharmaceutically acceptable salt of a compound described herein. An increase in average survival time of a population can be measured, for example, by calculating for a population the average length of survival time following completion of a first round of treatment with a compound or pharmaceutically acceptable salt of a compound described herein.

Treating nucleotide repeat expansion disorders (e.g., trinucleotide repeat expansion disorders) can result in a decrease in the mortality rate of a population of treated subjects in comparison to an untreated population. For example, the mortality rate is decreased by more than 2% (e.g., more than 5%, 10%, or 25%). A decrease in the mortality rate of a population of treated subjects can be measured by any reproducible means, for example, by calculating for a population the average number of disease-related deaths per unit time following initiation of treatment with a compound or pharmaceutically acceptable salt of a compound described herein. A decrease in the mortality rate of a population can be measured, for example, by calculating for a population the average number of disease-related deaths per unit time following completion of a first round of treatment with a compound or pharmaceutically acceptable salt of a compound described herein.

A. Delivery of anti-MSH3 Agents

The delivery of an oligonucleotide to a cell e.g., a cell within a subject, such as a human subject e.g., a subject in need thereof, such as a subject having a nucleotide repeat expansion disorder (e.g., a trinucleotide repeat expansion disorder) can be achieved via intracerebroventricular (ICV) administration.

### B. Combination Therapies

An oligonucleotide, or pharmaceutically acceptable salt thereof, can be used alone or in combination with at least one additional therapeutic agent, e.g., other agents that treat nucleotide repeat expansion disorders (e.g., trinucleotide repeat expansion disorders) or symptoms associated therewith, or in combination with other types of therapies to treat nucleotide repeat expansion disorders (e.g., trinucleotide repeat expansion disorders). In combination treatments, the dosages of one or more of the therapeutic compounds can be reduced from standard dosages when administered alone. For example, doses can be determined empirically from drug combinations and permutations or can be deduced by isobolographic analysis (e.g., Black et al., Neurology 65:S3-S6 (2005)). In this case, dosages of the compounds when combined should provide a therapeutic effect.

In some aspects, the oligonucleotide, or pharmaceutically acceptable salt thereof, agents described herein can be used in combination with at least one additional therapeutic agent to treat a nucleotide repeat expansion disorder (e.g., a trinucleotide repeat expansion disorder) associated with gene having a nucleotide repeat (e.g., any of the trinucleotide repeat expansion disorders and associated genes having a nucleotide repeat listed in Table 1). In some aspects, at least one of the additional therapeutic agents can be an oligonucleotide (e.g., an ASO) that hybridizes with the mRNA of gene associated with a nucleotide or trinucleotide repeat expansion disorder (e.g., any of the genes listed in Table 1). In some aspects, the nucleotide repeat expansion disorder (e.g., a trinucleotide repeat expansion disorder) is Huntington's disease (HD). In some aspects, the gene associated with a nucleotide repeat expansion disorder (e.g., a trinucleotide repeat expansion disorder) is Huntingtin (HTT). Several allelic variants of the Huntingtin gene have been implicated in the etiology of Huntington's disease. In some cases, these variants are identified on the basis of having unique HD-associated single nucleotide polymorphisms (SNPs). In some aspects, the oligonucleotide hybridizes to an mRNA of the Huntingtin gene containing any of the HD-associated SNPs known in the art (e.g., any of the HD-associated SNPs described in Skotte et al., PLoS One 2014, 9(9): e107434, Carroll et al., Mol. Ther. 2011, 19(12): 2178-85, Warby et al., Am. J. Hum. Gen. 2009, 84(3): 351-66 (herein incorporated by reference)). In some aspects, the oligonucleotide that is an additional therapeutic agent hybridizes to an mRNA of the Huntingtin gene lacking any of the HD-associated SNPs. In some of the aspects, the oligonucleotide, or pharmaceutically acceptable salt thereof, that is an additional therapeutic agent hybridizes to an mRNA of the Huntingtin gene having any of the SNPs selected from the group of rs362307 and rs365331. In some aspects, the oligonucleotide, or pharmaceutically acceptable salt thereof, that is an additional therapeutic agent can be a modified oligonucleotide (e.g., an oligonucleotide including any of the modifications described herein). In some aspects, the modified oligonucleotides that is an additional therapeutic agent comprise one or more phosphorothioate internucleoside linkages. In some aspects, the modified oligonucleotide comprises one or more 2′-MOE moieties. In some aspects, the oligonucleotide that is an additional therapeutic agent that hybridizes to the mRNA of the Huntingtin gene has a sequence selected from the SEQ ID NOs. 6-285 of U.S. Pat. No. 9,006,198; SEQ ID NOs. 6-8 of US Patent Application Publication No. 2017/0044539; SEQ ID NOs. 1-1565 of US Patent Application Publication 2018/0216108; and SEQ ID NOs. 1-2432 of PCT Publication WO 2017/192679, the sequences of which are hereby incorporated by reference.

In some aspects, at least one of the additional therapeutic agents is a chemotherapeutic agent (e.g., a cytotoxic agent or other chemical compound useful in the treatment of a nucleotide repeat expansion disorder, e.g., a trinucleotide repeat expansion disorder).

In some aspects, at least one of the additional therapeutic agents can be a therapeutic agent which is a non-drug treatment. For example, at least one of the additional therapeutic agents is physical therapy.

In any of the combination aspects described herein, the two or more therapeutic agents are administered simultaneously or sequentially, in either order. For example, a first therapeutic agent can be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after one or more of the additional therapeutic agents.

### V. Pharmaceutical Compositions

The oligonucleotides, or pharmaceutically acceptable salt thereof, described herein are formulated into pharmaceutical compositions for administration to human subjects in a biologically compatible form suitable for administration in vivo.

The compounds described herein can be used in the form of the free base, in the form of salts, solvates, and as prodrugs. All forms are within the methods described herein. In accordance with the methods described herein, the described oligonucleotides or salts, solvates, or prodrugs thereof can be administered to a patient in a variety of forms depending on the selected route of administration, as will be understood by those skilled in the art. The compounds described herein can be administered, for example, by intracerebroventricular administration and the pharmaceutical compositions formulated accordingly.

Solutions of a compound described herein for intracerebroventricular administration can be prepared in artificial cerebrospinal fluid or water suitably mixed with a suitable buffer and/or osmolarity agents, such as one or more of sodium chloride, potassium chloride, potassium phosphate, sodium carbonate, glucose, calcium chloride, sodium bicarbonate, and/or magnesium chloride. Conventional procedures and ingredients for the selection and preparation of suitable formulations are described, for example, in Remington's Pharmaceutical Sciences (2012, 22nd ed.) and in The United States Pharmacopeia: The National Formulary (USP 41 NF 36), published in 2018. The pharmaceutical forms suitable for injectable use include sterile aqueous solutions or dispersions and sterile powders for the extemporaneous preparation of sterile injectable solutions or dispersions. In all cases the form must be sterile and must be fluid to the extent that can be easily administered via syringe.

The compounds described herein can be administered to an animal, e.g., a human, alone or in combination with pharmaceutically acceptable carriers, as noted herein, the proportion of which is determined by the solubility and chemical nature of the compound, chosen route of administration, and standard pharmaceutical practice.

### VI. Dosages

The dosage of the compositions (e.g., a composition including an oligonucleotide, or pharmaceutically acceptable salt thereof, described herein, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. The compositions described herein can be administered initially in a suitable dosage that can be adjusted as required, depending on the clinical response. In some aspects, the dosage of a composition (e.g., a composition including an oligonucleotide, or pharmaceutically acceptable salt thereof,) is a prophylactically or a therapeutically effective amount.

In some aspects, provided herein are pharmaceutical compositions that are formulated for intracerebroventricular injection. The compositions described herein can be intracerebroventricularly administered initially in a suitable dosage that can be adjusted as required, depending on the clinical response. In some aspects, the dosage of a composition (e.g., a composition including an oligonucleotide, or pharmaceutically acceptable salt thereof,) is a prophylactically or a therapeutically effective amount.

In some aspects, provided herein is a method of treating, preventing, or delaying the onset and/or progression of a nucleotide repeat expansion disorder in a subject in need thereof, the method comprising intracerebroventricularly administering a single-stranded oligonucleotide, or pharmaceutically acceptable salt thereof, described herein. In some aspects, the single-stranded oligonucleotide, or pharmaceutically acceptable salt thereof, is administered at a dose of about 1 mg to about 300 mg. In some aspects, the single-stranded oligonucleotide, or a pharmaceutically acceptable salt thereof, is administered at a dose of about 10 mg to about 250 mg. In some aspects, the single-stranded oligonucleotide, or a pharmaceutically acceptable salt thereof, is administered at a dose of about 15 mg to about 200 mg. In some aspects, the single-stranded oligonucleotide, or a pharmaceutically acceptable salt thereof, is administered at a dose of about 25 mg to about 200 mg. In some aspects, the single-stranded oligonucleotide, or a pharmaceutically acceptable salt thereof, is administered at a dose of about 50 mg to about 200 mg. In some aspects, the single-stranded oligonucleotide, or a pharmaceutically acceptable salt thereof, is administered at a dose of about 100 mg to about 150 mg.

In some aspects, the single-stranded oligonucleotide or pharmaceutically acceptable salt thereof is administered at a dose of about 1 mg, about 2 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, or about 300 mg.

In some aspects, the single-stranded oligonucleotide, or a pharmaceutically acceptable salt thereof is administered once weekly. In some aspects, the single-stranded oligonucleotide, or a pharmaceutically acceptable salt thereof is administered once every two weeks. In some aspects, the single-stranded oligonucleotide, or a pharmaceutically acceptable salt thereof, is administered once every three weeks. In some aspects, the single-stranded oligonucleotide, or a pharmaceutically acceptable salt thereof is administered once every four weeks. In some aspects, the single-stranded oligonucleotide, or a pharmaceutically acceptable salt thereof is administered once every eight weeks. In some aspects, the single-stranded oligonucleotide, or a pharmaceutically acceptable salt thereof is administered once every sixteen weeks. In some aspects, the single-stranded oligonucleotide, or a pharmaceutically acceptable salt thereof, is administered once every twenty weeks. In some aspects, the single-stranded oligonucleotide, or a pharmaceutically acceptable salt thereof, is administered once every twenty-four weeks.

In some aspects, the single-stranded oligonucleotide, or a pharmaceutically acceptable salt thereof is administered once every twenty-eight weeks. In some aspects, the single-stranded oligonucleotide, or a pharmaceutically acceptable salt thereof is administered once every thirty-two weeks. In some aspects, the single-stranded oligonucleotide, or a pharmaceutically acceptable salt thereof, is administered once every thirty-six weeks.

In some aspects, the single-stranded oligonucleotide, or a pharmaceutically acceptable salt thereof is administered once every every month. In some aspects, the single-stranded oligonucleotide, or a pharmaceutically acceptable salt thereof is administered once every two months. In some aspects, the single-stranded oligonucleotide, or a pharmaceutically acceptable salt thereof is administered once every three months. In some aspects, the single-stranded oligonucleotide, or a pharmaceutically acceptable salt thereof is administered once every four months. In some aspects, the single-stranded oligonucleotide, or a pharmaceutically acceptable salt thereof is administered once every five months. In some aspects, the single-stranded oligonucleotide, or a pharmaceutically acceptable salt thereof is administered once every six months. In some aspects, the single-stranded oligonucleotide, or a pharmaceutically acceptable salt thereof is administered once every seven months. In some aspects, the single-stranded oligonucleotide, or a pharmaceutically acceptable salt thereof is administered once every eight months. In some aspects, the single-stranded oligonucleotide, or a pharmaceutically acceptable salt thereof is administered once every nine months.

In some aspects, the methods described herein delay the onset and/or progression of the nucleotide repeat expansion disorder by at least 120 days, at least 6 months, at least 12 months, at least 2 years, at least 3 years, at least 4 years, at least 5 years, at least 10 years or more, when compared with a predicted onset and/or progression.

### VII. Kits

Kits including (a) a pharmaceutical composition including an oligonucleotide, or pharmaceutically acceptable salt thereof, agent that reduces the level and/or activity of MSH3 in a cell or subject described herein, and (b) a package insert with instructions to perform any of the methods described herein are contemplated. In some aspects, the kit includes (a) a pharmaceutical composition including an oligonucleotide, or pharmaceutically acceptable salt thereof, agent that reduces the level and/or activity of MSH3 in a cell or subject described herein, (b) an additional therapeutic agent, and (c) a package insert with instructions to perform any of the methods described herein.

### EXAMPLES

### Example 1. Antisense Oligonucleotides

### Knock Down by ASOs

ASO screen in Hela cells to identify the top ASO in Table 3 for the MSH3 gene was performed by Horizon.

In summary: ASO knockdown activity was evaluated in HeLa by transfection at 1 nM and 10 nM. mRNA knockdown was analyzed by quantitative reverse transcription polymerase chain reaction (RT-qPCR) using TaqMan Gene Expression probes. mRNA expression was calculated via delta-delta Ct (ΔΔCT) method where target expression was normalized to expression of the reference gene beta-glucuronidase (GUSB) and to cells treated with a scrambled luciferase targeting control ASO.

Transfection in HeLa cells

ASOs were resuspended in dH2O to 1000-fold their final assay concentration (10 uM or 1 uM). ASOs were dispensed in quadruplicates and complexed with 5 ul of Lipofectamine 3000 (Invitrogen) for 20 minutes before HeLa cells were added at 2,500 cells/well. Cells were cultured under standard culturing conditions for 24 hours. Cells were processed for RT-qPCR readout using the Cells-to-CT 1-step TaqMan Kit (Invitrogen) according to manufacturer's instructions. TaqMan Gene Expression probe for MSH3 was Hs00989003_ml (Life Technologies Ltd) on a QuantStudio 6 (Applied BioSystems).

TABLE 2
Key to Chemical Modifiers in Tables 3 and 4

| | |
|---|---|
| “s” after base | phosphorothioate linkage |
| “p” after base | phosphodiester linkage |
| “o” before base | 2′-O-methoxyethyl-RNA (“moe”) |
| “d” before base | deoxy (a DNA nucleoside) |
| “L” before base | Locked nucleic acid (LNA) |
| ACTG | core DNA bases: adenine; cytosine; thymine; and guanine |
| U | uracil; all U (uracil) are 5-methyl |
| “OU” | synonymous with “oT” |
| “5m” | methyl at position 5 on the nucleobase; all C (cytosine) are 5-methyl |

In Tables 3 and 4 below, the SEQ TD No. corresponds to the nucleobase sequence of the Antisense Oligo No. However, the specific Antisense Oligo No. (e.g., Antisense Oligo No. 1) includes the specified chemical modifications. It is well known in the field that all nucleobases in the DNA core are deoxy. Therefore, in Tables 3 and 4, if the nucleobase in the DNA core of a sequence is not predicated by an “o” to represent “moe,” then the nucleobase is a DNA nucleobase (deoxy).

TABLE 3

| Antisense | | Mean % mRNA | | SEM % mRNA | |
|---|---|---|---|---|---|
| oligo No./ | | Remaining | | Remaining | |
| SEQ ID No: | Chem Mod Seq | 1 nM | 10 nM | 1 nM | 10 nM |
| 1 | [oCs\|oUs\|oAs\|oGs\|oGs\|dTs\|dGs\|dAs\|dT | 86.09785 | 41.79781 | 10.956696 | 1.978577 |
| | s\|dGs\|5mCs\|dAs\|5mCs\|dTs\|dGs\|oCs\|oU | | | | |
| | s\|oUs\|oUs\|oA] | | | | |
| 2 | [oCs\|oUs\|oAp\|oGs\|oGs\|dTs\|dGs\|dAs\|d | 102.13406 | 40.30929 | 2.5403645 | 1.368438 |
| | Ts\|dGs\|5mCs\|dAs\|5mCs\|dTs\|dGs\|oCs\|o | | | | |
| | Us\|oUs\|oUs\|oA] | | | | |
| 3 | [oCs\|oUs\|oAs\|oGp\|oGs\|dTs\|dGs\|dAs\|d | 86.89053 | 37.50409 | 4.95608 | 1.237848 |
| | Ts\|dGs\|5mCs\|dAs\|5mCs\|dTs\|dGs\|oCs\|o | | | | |
| | Us\|oUs\|oUs\|oA] | | | | |
| 4 | [oCs\|oUs\|oAp\|oGp\|oGs\|dTs\|dGs\|dAs\|d | 88.21922 | 46.14382 | 8.324711 | 2.068448 |
| | Ts\|dGs\|5mCs\|dAs\|5mCs\|dTs\|dGs\|oCs\|o | | | | |
| | Us\|oUs\|oUs\|oA] | | | | |
| 5 | [oCs\|oUs\|oAs\|oGs\|oGs\|dTs\|dGs\|dAs\|dT | 88.06973 | 44.49033 | 7.0556625 | 1.637641 |
| | s\|dGs\|5mCs\|dAs\|5mCs\|dTs\|dGs\|oCs\|oU | | | | |
| | p\|oUs\|oUs\|oA] | | | | |
| 6 | [oCs\|oUs\|oAs\|oGs\|oGs\|dTs\|dGs\|dAs\|dT | 66.27929 | 38.82585 | 7.304814 | 2.701934 |
| | sdGs\|5mCs\|dAs\|5mCs\|dTs\|dGs\|oCp\|o | | | | |
| | Us\|oUs\|oUs\|oA] | | | | |
| 7 | [oCs\|oUs\|oAs\|oGs\|oGs\|dTs\|dGs\|dAs\|dT | 87.77148 | 39.91306 | 3.7650095 | 1.899814 |
| | s\|dGs\|5mCs\|dAs\|5mCs\|dTs\|dGs\|oCp\|o | | | | |
| | Up\|oUs\|oUs\|oA] | | | | |
| 8 | [oCs\|oUs\|oAp\|oGs\|oGs\|dTs\|dGs\|dAs\|d | 93.78302 | 39.9579 | 5.800865 | 2.591389 |
| | Ts\|dGs\|5mCs\|dAs\|5mCs\|dTs\|dGs\|oCs\|o | | | | |
| | Up\|oUs\|oUs\|oA] | | | | |
| 9 | [oCs\|oUs\|oAp\|oGs\|oGs\|dTs\|dGs\|dAs\|d | 91.70171 | 46.43532 | 6.7248825 | 1.898713 |
| | Ts\|dGs\|5mCs\|dAs\|5mCs\|dTs\|dGs\|oCp\|o | | | | |
| | Us\|oUs\|oUs\|oA] | | | | |
| 10 | [oCs\|oUs\|oAs\|oGp\|oGs\|dTs\|dGs\|dAs\|d | 90.12033 | 43.42065 | 11.205575 | 2.148471 |
| | Ts\|dGs\|5mCs\|dAs\|5mCs\|dTs\|dGs\|oCs\|o | | | | |
| | Up\|oUs\|oUs\|oA] | | | | |
| 11 | [oCs\|oUs\|oAs\|oGp\|oGs\|dTs\|dGs\|dAs\|d | 99.38491 | 40.8259 | 7.1537125 | 4.453034 |
| | Ts\|dGs\|5mCs\|dAs\|5mCs\|dTs\|dGs\|oCp\|o | | | | |
| | Us\|oUs\|oUs\|oA] | | | | |
| 12 | [oCs\|oUs\|oAp\|oGs\|oGs\|dTs\|dGs\|dAs\|d | 92.85072 | 47.68991 | 9.7849105 | 0.732238 |
| | Ts\|dGs\|5mCs\|dAs\|5mCs\|dTs\|dGs\|oCp\|o | | | | |
| | Up\|oUs\|oUs\|oA] | | | | |
| 13 | [oCs\|oUs\|oAs\|oGp\|oGs\|dTs\|dGs\|dAs\|d | 82.76355 | 44.94987 | 2.3796215 | 1.213861 |
| | Ts\|dGs\|5mCs\|dAs\|5mCs\|dTs\|dGs\|oCp\|o | | | | |
| | Up\|oUs\|oUs\|oA] | | | | |
| 14 | [oCs\|oUs\|oAp\|oGp\|oGs\|dTs\|dGs\|dAs\|d | 100.30431 | 42.47324 | 7.2137325 | 3.762178 |
| | Ts\|dGs\|5mCs\|dAs\|5mCs\|dTs\|dGs\|oCs\|o | | | | |
| | Up\|oUs\|oUs\|oA] | | | | |
| 15 | [oCs\|oUs\|oAp\|oGp\|oGs\|dTs\|dGs\|dAs\|d | 90.00654 | 39.81399 | 10.431815 | 2.091189 |
| | Ts\|dGs\|5mCs\|dAs\|5mCs\|dTs\|dGs\|oCp\|o | | | | |
| | Us\|oUs\|oUs\|oA] | | | | |
| 16 | [oCs\|oUs\|oAp\|oGp\|oGs\|dTs\|dGs\|dAs\|d | 93.50777 | 41.81402 | 6.027847 | 2.177984 |
| | Ts\|dGs\|5mCs\|dAs\|5mCs\|dTs\|dGs\|oCp\|o | | | | |
| | Up\|oUs\|oUs\|oA] | | | | |
| 17 | [oCs\|oUs\|oAs\|oGs\|oGs\|dTs\|dGs\|dAs\|dT | 78.87919 | 39.04837 | 4.3289815 | 3.888626 |
| | sdGs\|5mCs\|dAs\|5mCs\|dTs\|dGs\|oCs\|oU | | | | |
| | s\|oUs\|oUs\|oAs\|oC] | | | | |
| 18 | [oCs\|oUs\|oAp\|oGs\|oGs\|dTs\|dGs\|dAs\|d | 89.8343 | 37.95063 | 6.6130055 | 2.550701 |
| | Ts\|dGs\|5mCs\|dAs\|5mCs\|dTs\|dGs\|oCs\|o | | | | |
| | Us\|oUs\|oUs\|oAs\|oC] | | | | |
| 19 | [oCs\|oUs\|oAs\|oGp\|oGs\|dTs\|dGs\|dAs\|d | 91.97128 | 45.99061 | 1.903787 | 2.837245 |
| | Ts\|dGs\|5mCs\|dAs\|5mCs\|dTs\|dGs\|oCs\|o | | | | |
| | Us\|oUs\|oUs\|oAs\|oC] | | | | |
| 20 | [oCs\|oUs\|oAp\|oGp\|oGs\|dTs\|dGs\|dAs\|d | 90.0287 | 45.36505 | 7.652128 | 3.536383 |
| | Ts\|dGs\|5mCs\|dAs\|5mCs\|dTs\|dGs\|oCs\|o | | | | |
| | Us\|oUs\|oUs\|oAs\|oC] | | | | |
| 21 | [oCs\|oUs\|oAs\|oGs\|oGs\|dTs\|dGs\|dAs\|dT | 80.24857 | 37.65808 | 8.935247 | 5.090914 |
| | s\|dGs\|5mCs\|dAs\|5mCs\|dTs\|dGs\|oCs\|oU | | | | |
| | p\|oUs\|oUs\|oAs\|oC] | | | | |
| 22 | [oCs\|oUs\|oAs\|oGs\|oGs\|dTs\|dGs\|dAs\|dT | 101.45303 | 37.27497 | 23.097582 | 3.052247 |
| | s\|dGs\|5mCs\|dAs\|5mCs\|dTs\|dGs\|oCp\|o | | | | |
| | Us\|oUs\|oUs\|oAs\|oC] | | | | |
| 23 | [oCs\|oUs\|oAs\|oGs\|oGs\|dTs\|dGs\|dAs\|dT | 97.15427 | 41.07113 | 12.915649 | 3.502463 |
| | s\|dGs\|5mCs\|dAs\|5mCs\|dTs\|dGs\|oCp\|o | | | | |
| | Up\|oUs\|oUs\|oAs\|oC] | | | | |
| 24 | [oCs\|oUs\|oAp\|oGs\|oGs\|dTs\|dGs\|dAs\|d | 80.67146 | 37.30983 | 3.1350315 | 4.460652 |
| | Ts\|dGs\|5mCs\|dAs\|5mCs\|dTs\|dGs\|oCs\|o | | | | |
| | Up\|oUs\|oUs\|oAs\|oC] | | | | |
| 25 | [oCs\|oUs\|oAp\|oGs\|oGs\|dTs\|dGs\|dAs\|d | 90.35424 | 39.93492 | 10.3572485 | 3.866769 |
| | Ts\|dGs\|5mCs\|dAs\|5mCs\|dTs\|dGs\|oCp\|o | | | | |
| | Us\|oUs\|oUs\|oAs\|oC] | | | | |
| 26 | [oCs\|oUs\|oAs\|oGp\|oGs\|dTs\|dGs\|dAs\|d | 82.80083 | 43.89603 | 2.679553 | 3.062266 |
| | Ts\|dGs\|5mCs\|dAs\|5mCs\|dTs\|dGs\|oCs\|o | | | | |
| | Up\|oUs\|oUs\|oAs\|oC] | | | | |
| 27 | [oCs\|oUs\|oAs\|oGp\|oGs\|dTs\|dGs\|dAs\|d | 88.08068 | 46.08224 | 6.516991 | 2.883583 |
| | Ts\|dGs\|5mCs\|dAs\|5mCs\|dTs\|dGs\|oCp\|o | | | | |
| | Us\|oUs\|oUs\|oAs\|oC] | | | | |
| 28 | [oCs\|oUs\|oAp\|oGs\|oGs\|dTs\|dGs\|dAs\|d | 100.38683 | 42.14532 | 7.9455835 | 2.066183 |
| | Ts\|dGs\|5mCs\|dAs\|5mCs\|dTs\|dGs\|oCp\|o | | | | |
| | Up\|oUs\|oUs\|oAs\|oC] | | | | |
| 29 | [oCs\|oUs\|oAs\|oGp\|oGs\|dTs\|dGs\|dAs\|d | 82.39148 | 42.89111 | 4.9474165 | 1.687775 |
| | Ts\|dGs\|5mCs\|dAs\|5mCs\|dTs\|dGs\|oCp\|o | | | | |
| | Up\|oUs\|oUs\|oAs\|oC] | | | | |
| 30 | [oCs\|oUs\|oAp\|oGp\|oGs\|dTs\|dGs\|dAs\|d | 92.49721 | 41.87519 | 4.286409 | 1.419928 |
| | Ts\|dGs\|5mCs\|dAs\|5mCs\|dTs\|dGs\|oCs\|o | | | | |
| | Up\|oUs\|oUs\|oAs\|oC] | | | | |
| 31 | [oCs\|oUs\|oAp\|oGp\|oGs\|dTs\|dGs\|dAs\|d | 88.1524 | 43.50525 | 4.0152095 | 1.165505 |
| | Ts\|dGs\|5mCs\|dAs\|5mCs\|dTs\|dGs\|oCp\|o | | | | |
| | Us\|oUs\|oUs\|oAs\|oC] | | | | |
| 32 | [oCs\|oUs\|oAp\|oGp\|oGs\|dTs\|dGs\|dAs\|d | 95.59035 | 44.36914 | 6.098232 | 1.947992 |
| | Ts\|dGs\|5mCs\|dAs\|5mCs\|dTs\|dGs\|oCp\|o | | | | |
| | Up\|oUs\|oUs\|oAs\|oC] | | | | |
| 33 | [oCs\|oUs\|oAs\|oGs\|oGs\|dTs\|dGs\|dAs\|dT | 81.5396 | 46.37686 | 5.051866 | 2.340369 |
| | s\|dGs\|5mCs\|dAs\|5mCs\|dTs\|dGs\|oCs\|oU | | | | |
| | s\|oUs\|oUs\|oAs\|oCs\|oA] | | | | |
| 34 | [oCs\|oUs\|oAp\|oGs\|oGs\|dTs\|dGs\|dAs\|d | 75.50977 | 37.42608 | 6.55804 | 2.427113 |
| | Ts\|dGs\|5mCs\|dAs\|5mCs\|dTs\|dGs\|oCs\|o | | | | |
| | Us\|oUs\|oUs\|oAs\|oCs\|oA] | | | | |
| 35 | [oCs\|oUs\|oAs\|oGp\|oGs\|dTs\|dGs\|dAs\|d | 86.89931 | 38.66673 | 1.6915285 | 0.403006 |
| | Ts\|dGs\|5mCs\|dAs\|5mCs\|dTs\|dGs\|oCs\|o | | | | |
| | Us\|oUs\|oUs\|oAs\|oCs\|oA] | | | | |
| 36 | [oCs\|oUs\|oAp\|oGp\|oGs\|dTs\|dGs\|dAs\|d | 92.05609 | 44.46799 | 6.699515 | 0.811156 |
| | Ts\|dGs\|5mCs\|dAs\|5mCs\|dTs\|dGs\|oCs\|o | | | | |
| | Us\|oUs\|oUs\|oAs\|oCs\|oA] | | | | |
| 37 | [oCs\|oUs\|oAs\|oGs\|oGs\|dTs\|dGs\|dAs\|dT | 82.63172 | 39.09093 | 8.238769 | 2.549669 |
| | s\|dGs\|5mCs\|dAs\|5mCs\|dTs\|dGs\|oCs\|oU | | | | |
| | p\|oUs\|oUs\|oAs\|oCs\|oA] | | | | |
| 38 | [oCs\|oUs\|oAs\|oGs\|oGs\|dTs\|dGs\|dAs\|dT | 102.24694 | 36.96398 | 14.530799 | 2.884441 |
| | s\|dGs\|5mCs\|dAs\|5mCs\|dTs\|dGs\|oCp\|o | | | | |
| | Us\|oUs\|oUs\|oAs\|oCs\|oA] | | | | |
| 39 | [oCs\|oUs\|oAs\|oGs\|oGs\|dTs\|dGs\|dAs\|dT | 87.2629 | 39.3864 | 12.0735685 | 1.500768 |
| | s\|dGs\|5mCs\|dAs\|5mCs\|dTs\|dGs\|oCp\|o | | | | |
| | Up\|oUs\|oUs\|oAs\|oCs\|oA] | | | | |
| 40 | [oCs\|oUs\|oAp\|oGs\|oGs\|dTs\|dGs\|dAs\|d | 96.78945 | 37.19026 | 9.535124 | 1.52692 |
| | Ts\|dGs\|5mCs\|dAs\|5mCs\|dTs\|dGs\|oCs\|o | | | | |
| | Up\|oUs\|oUs\|oAs\|oCs\|oA] | | | | |
| 41 | [oCs\|oUs\|oAp\|oGs\|oGs\|dTs\|dGs\|dAs\|d | 95.79925 | 45.78015 | 7.062957 | 5.396491 |
| | Ts\|dGs\|5mCs\|dAs\|5mCs\|dTs\|dGs\|oCp\|o | | | | |
| | Us\|oUs\|oUs\|oAs\|oCs\|oA] | | | | |
| 42 | [oCs\|oUs\|oAs\|oGp\|oGs\|dTs\|dGs\|dAs\|d | 87.43444 | 39.82331 | 10.1333325 | 1.186569 |
| | Ts\|dGs\|5mCs\|dAs\|5mCs\|dTs\|dGs\|oCs\|o | | | | |
| | Up\|oUs\|oUs\|oAs\|oCs\|oA] | | | | |
| 43 | [oCs\|oUs\|oAs\|oGp\|oGs\|dTs\|dGs\|dAs\|d | 93.03859 | 41.55274 | 8.5237595 | 1.542313 |
| | Ts\|dGs\|5mCs\|dAs\|5mCs\|dTs\|dGs\|oCp\|o | | | | |
| | Us\|oUs\|oUs\|oAs\|oCs\|oA] | | | | |
| 44 | [oCs\|oUs\|oAp\|oGs\|oGs\|dTs\|dGs\|dAs\|d | 79.29174 | 41.69657 | 9.4802245 | 3.7328 |
| | Ts\|dGs\|5mCs\|dAs\|5mCs\|dTs\|dGs\|oCp\|o | | | | |
| | Up\|oUs\|oUs\|oAs\|oCs\|oA] | | | | |
| 45 | [oCs\|oUs\|oAs\|oGp\|oGs\|dTs\|dGs\|dAs\|d | 97.84352 | 40.24762 | 4.024119 | 3.662828 |
| | Ts\|dGs\|5mCs\|dAs\|5mCs\|dTs\|dGs\|oCp\|o | | | | |
| | Up\|oUs\|oUs\|oAs\|oCs\|oA] | | | | |
| 46 | [oCs\|oUs\|oAp\|oGp\|oGs\|dTs\|dGs\|dAs\|d | 94.18266 | 36.69312 | 3.2339695 | 2.577563 |
| | Ts\|dGs\|5mCs\|dAs\|5mCs\|dTs\|dGs\|oCs\|o | | | | |
| | Up\|oUs\|oUs\|oAs\|oCs\|oA] | | | | |
| 47 | [oCs\|oUs\|oAp\|oGp\|oGs\|dTs\|dGs\|dAs\|d | 97.71451 | 49.27591 | 15.6053125 | 5.231299 |
| | Ts\|dGs\|5mCs\|dAs\|5mCs\|dTs\|dGs\|oCp\|o | | | | |
| | Us\|oUs\|oUs\|oAs\|oCs\|oA] | | | | |
| 48 | [oCs\|oUs\|oAp\|oGp\|oGs\|dTs\|dGs\|dAs\|d | 91.5412 | 44.77662 | 6.4424815 | 4.030398 |
| | Ts\|dGs\|5mCs\|dAs\|5mCs\|dTs\|dGs\|oCp\|o | | | | |
| | Up\|oUs\|oUs\|oAs\|oCs\|oA] | | | | |
| 49 | [oCs\|oUs\|oAs\|oGs\|oGs\|dTs\|dGs\|dAs\|dT | 85.14544 | 43.97438 | 5.329607 | 1.787627 |
| | s\|dGs\|5mCs\|dAs\|5mCs\|dTs\|dGs\|oCs\|oU | | | | |
| | s\|oUs\|oUs\|oAs\|oCs\|oAs\|oC] | | | | |
| 50 | [oCs\|oUs\|oAp\|oGs\|oGs\|dTs\|dGs\|dAs\|d | 81.85128 | 40.2583 | 5.194841 | 1.498131 |
| | Ts\|dGs\|5mCs\|dAs\|5mCs\|dTs\|dGs\|oCs\|o | | | | |
| | Us\|oUs\|oUs\|oAs\|oCs\|oAs\|oC] | | | | |
| 51 | [oCs\|oUs\|oAs\|oGp\|oGs\|dTs\|dGs\|dAs\|d | 79.48116 | 41.09427 | 2.3949705 | 1.899097 |
| | Ts\|dGs\|5mCs\|dAs\|5mCs\|dTs\|dGs\|oCs\|o | | | | |
| | Us\|oUs\|oUs\|oAs\|oCs\|oAs\|oC] | | | | |
| 52 | [oCs\|oUs\|oAp\|oGp\|oGs\|dTs\|dGs\|dAs\|d | 82.36726 | 40.83192 | 5.178712 | 3.06104 |
| | Ts\|dGs\|5mCs\|dAs\|5mCs\|dTs\|dGs\|oCs\|o | | | | |
| | Us\|oUs\|oUs\|oAs\|oCs\|oAs\|oC] | | | | |
| 53 | [oCs\|oUs\|oAs\|oGs\|oGs\|dTs\|dGs\|dAs\|dT | 73.45186 | 36.40204 | 4.508506 | 2.072795 |
| | s\|dGs\|5mCs\|dAs\|5mCs\|dTs\|dGs\|oCs\|oU | | | | |
| | p\|oUs\|oUs\|oAs\|oCs\|oAs\|oC] | | | | |
| 54 | [oCs\|oUs\|oAs\|oGs\|oGs\|dTs\|dGs\|dAs\|dT | 83.26679 | 42.28042 | 9.2488475 | 3.422593 |
| | s\|dGs\|5mCs\|dAs\|5mCs\|dTs\|dGs\|oCp\|o | | | | |
| | Us\|oUs\|oUs\|oAs\|oCs\|oAs\|oC] | | | | |
| 55 | [oCs\|oUs\|oAs\|oGs\|oGs\|dTs\|dGs\|dAs\|dT | 73.0733 | 44.80567 | 3.2515675 | 5.215558 |
| | s\|dGs\|5mCs\|dAs\|5mCs\|dTs\|dGs\|oCp\|o | | | | |
| | Up\|oUs\|oUs\|oAs\|oCs\|oAs\|oC] | | | | |
| 56 | [oCs\|oUs\|oAp\|oGs\|oGs\|dTs\|dGs\|dAs\|d | 86.11374 | 41.79482 | 6.120118 | 3.145374 |
| | Ts\|dGs\|5mCs\|dAs\|5mCs\|dTs\|dGs\|oCs\|o | | | | |
| | Up\|oUs\|oUs\|oAs\|oCs\|oAs\|oC] | | | | |
| 57 | [oCs\|oUs\|oAp\|oGs\|oGs\|dTs\|dGs\|dAs\|d | 82.10891 | 41.5037 | 10.9839625 | 2.257748 |
| | Ts\|dGs\|5mCs\|dAs\|5mCs\|dTs\|dGs\|oCp\|o | | | | |
| | Us\|oUs\|oUs\|oAs\|oCs\|oAs\|oC] | | | | |
| 58 | [oCs\|oUs\|oAs\|oGp\|oGs\|dTs\|dGs\|dAs\|d | 89.70608 | 40.60014 | 3.8721065 | 0.87326 |
| | Ts\|dGs\|5mCs\|dAs\|5mCs\|dTs\|dGs\|oCs\|o | | | | |
| | Up\|oUs\|oUs\|oAs\|oCs\|oAs\|oC] | | | | |
| 59 | [oCs\|oUs\|oAs\|oGp\|oGs\|dTs\|dGs\|dAs\|d | 93.23447 | 43.85248 | 4.0394125 | 4.050107 |
| | Ts\|dGs\|5mCs\|dAs\|5mCs\|dTs\|dGs\|oCp\|o | | | | |
| | Us\|oUs\|oUs\|oAs\|oCs\|oAs\|oC] | | | | |
| 60 | [oCs\|oUs\|oAp\|oGs\|oGs\|dTs\|dGs\|dAs\|d | 80.85138 | 43.55119 | 6.949772 | 3.486459 |
| | Ts\|dGs\|5mCs\|dAs\|5mCs\|dTs\|dGs\|oCp\|o | | | | |
| | Up\|oUs\|oUs\|oAs\|oCs\|oAs\|oC] | | | | |
| 61 | [oCs\|oUs\|oAs\|oGp\|oGs\|dTs\|dGs\|dAs\|d | 77.98907 | 37.1955 | 9.4067375 | 1.362401 |
| | Ts\|dGs\|5mCs\|dAs\|5mCs\|dTs\|dGs\|oCp\|o | | | | |
| | Up\|oUs\|oUs\|oAs\|oCs\|oAs\|oC] | | | | |
| 62 | [oCs\|oUs\|oAp\|oGp\|oGs\|dTs\|dGs\|dAs\|d | 71.11867 | 37.22296 | 7.9519255 | 2.922158 |
| | Ts\|dGs\|5mCs\|dAs\|5mCs\|dTs\|dGs\|oCs\|o | | | | |
| | Up\|oUs\|oUs\|oAs\|oCs\|oAs\|oC] | | | | |
| 63 | [oCs\|oUs\|oAp\|oGp\|oGs\|dTs\|dGs\|dAs\|d | 81.67628 | 40.47476 | 2.889408 | 2.316071 |
| | Ts\|dGs\|5mCs\|dAs\|5mCs\|dTs\|dGs\|oCp\|o | | | | |
| | Us\|oUs\|oUs\|oAs\|oCs\|oAs\|oC] | | | | |
| 64 | [oCs\|oUs\|oAp\|oGp\|oGs\|dTs\|dGs\|dAs\|d | 79.69746 | 38.81524 | 7.742031 | 0.204948 |
| | Ts\|dGs\|5mCs\|dAs\|5mCs\|dTs\|dGs\|oCp\|o | | | | |
| | Up\|oUs\|oUs\|oAs\|oCs\|oAs\|oC] | | | | |
| 65 | [oCs\|oUs\|oAs\|oGs\|oGs\|dTs\|dGs\|dAs\|dT | 69.99261 | 32.0998 | 5.4826715 | 3.037028 |
| | s\|dGs\|5mCs\|dAs\|5mCs\|dTs\|dGs\|5mCs\|o | | | | |
| | Us\|oUs\|oUs\|oAs\|oC] | | | | |
| 66 | [oCs\|oUs\|oAp\|oGs\|oGs\|dTs\|dGs\|dAs\|d | 75.98915 | 34.73835 | 3.798421 | 3.084744 |
| | Ts\|dGs\|5mCs\|dAs\|5mCs\|dTs\|dGs\|5mCs | | | | |
| | \|oUs\|oUs\|oUs\|oAs\|oC] | | | | |
| 67 | Ts\|dGs\|5mCs\|dAs\|5mCs\|dTs\|dGs\|5mCs\|d | 83.04812 | 42.68424 | 4.758469 | 4.679494 |
| | Ts\|dGs\|5mCs\|dAs\|5mCs\|dTs\|dGs\|5mCs | | | | |
| | \|oUs\|oUs\|oUs\|oAs\|oC] | | | | |
| 68 | [oCs\|oUs\|oAp\|oGp\|oGs\|dTs\|dGs\|dAs\|d | 74.06207 | 38.55632 | 9.9099225 | 7.981079 |
| | Ts\|dGs\|5mCs\|dAs\|5mCs\|dTs\|dGs\|5mCs | | | | |
| | \|oUs\|oUs\|oUs\|oAs\|oC] | | | | |
| 69 | [oCs\|oUs\|oAs\|oGs\|oGs\|dTs\|dGs\|dAs\|dT | 81.32662 | 38.51888 | 1.5401265 | 2.565575 |
| | s\|dGs\|5mCs\|dAs\|5mCs\|dTs\|dGs\|5mCs\|o | | | | |
| | Up\|oUs\|oUs\|oAs\|oC] | | | | |
| 70 | [oCs\|oUs\|oAs\|oGs\|oGs\|dTs\|dGs\|dAs\|dT | 79.67053 | 27.90351 | 2.039101 | 1.648429 |
| | s\|dGs\|5mCs\|dAs\|5mCs\|dTs\|dGs\|5mCp\| | | | | |
| | oUs\|oUs\|oUs\|oAs\|oC] | | | | |
| 71 | [oCs\|oUs\|oAs\|oGs\|oGs\|dTs\|dGs\|dAs\|dT | 72.32385 | 34.23619 | 4.994041 | 3.100586 |
| | s\|dGs\|5mCs\|dAs\|5mCs\|dTs\|dGs\|5mCp\| | | | | |
| | oUp\|oUs\|oUs\|oAs\|oC] | | | | |
| 72 | [oCs\|oUs\|oAp\|oGs\|oGs\|dTs\|dGs\|dAs\|d | 73.88174 | 35.08737 | 3.987852 | 3.319998 |
| | Ts\|dGs\|5mCs\|dAs\|5mCs\|dTs\|dGs\|5mCs | | | | |
| | \|oUp\|oUs\|oUs\|oAs\|oC] | | | | |
| 73 | [oCs\|oUs\|oAp\|oGs\|oGs\|dTs\|dGs\|dAs\|d | 73.84298 | 39.89966 | 6.744107 | 3.444095 |
| | Ts\|dGs\|5mCs\|dAs\|5mCs\|dTs\|dGs\|5mC | | | | |
| | p\|oUs\|oUs\|oUs\|oAs\|oC] | | | | |
| 74 | [oCs\|oUs\|oAs\|oGp\|oGs\|dTs\|dGs\|dAs\|d | 87.48091 | 31.76795 | 4.7688275 | 2.561686 |
| | Ts\|dGs\|5mCs\|dAs\|5mCs\|dTs\|dGs\|5mCs | | | | |
| | \|oUp\|oUs\|oUs\|oAs\|oC] | | | | |
| 75 | [oCs\|oUs\|oAs\|oGp\|oGs\|dTs\|dGs\|dAs\|d | 85.5695 | 37.95998 | 7.860177 | 4.320679 |
| | Ts\|dGs\|5mCs\|dAs\|5mCs\|dTs\|dGs\|5mC | | | | |
| | p\|oUs\|oUs\|oUs\|oAs\|oC] | | | | |
| 76 | [oCs\|oUs\|oAp\|oGs\|oGs\|dTs\|dGs\|dAs\|d | 82.15887 | 38.7112 | 6.4298045 | 2.851738 |
| | Ts\|dGs\|5mCs\|dAs\|5mCs\|dTs\|dGs\|5mC | | | | |
| | p\|oUp\|oUs\|oUs\|oAs\|oC] | | | | |
| 77 | [oCs\|oUs\|oAs\|oGp\|oGs\|dTs\|dGs\|dAs\|d | 92.45412 | 41.20665 | 7.931752 | 5.873654 |
| | Ts\|dGs\|5mCs\|dAs\|5mCs\|dTs\|dGs\|5mC | | | | |
| | p\|oUp\|oUs\|oUs\|oAs\|oC] | | | | |
| 78 | [oCs\|oUs\|oAp\|oGp\|oGs\|dTs\|dGs\|dAs\|d | 87.20785 | 34.56143 | 6.5475275 | 3.904361 |
| | Ts\|dGs\|5mCs\|dAs\|5mCs\|dTs\|dGs\|5mCs | | | | |
| | \|oUp\|oUs\|oUs\|oAs\|oC] | | | | |
| 79 | [oCs\|oUs\|oAp\|oGp\|oGs\|dTs\|dGs\|dAs\|d | 77.67484 | 47.25886 | 4.025454 | 9.140193 |
| | Ts\|dGs\|5mCs\|dAs\|5mCs\|dTs\|dGs\|5mC | | | | |
| | p\|oUs\|oUs\|oUs\|oAs\|oC] | | | | |
| 80 | [oCs\|oUs\|oAp\|oGp\|oGs\|dTs\|dGs\|dAs\|d | 84.26525 | 44.90753 | 2.9419125 | 3.931427 |
| | Ts\|dGs\|5mCs\|dAs\|5mCs\|dTs\|dGs\|5mC | | | | |
| | p\|oUp\|oUs\|oUs\|oAs\|oC] | | | | |
| 81 | [oCs\|oUs\|oAs\|oGs\|oGs\|dTs\|dGs\|dAs\|dT | 74.15488 | 28.91957 | 4.4416545 | 2.079845 |
| | s\|dGs\|5mCs\|dAs\|5mCs\|dTs\|dGs\|5mCs\|d | | | | |
| | Ts\|oUs\|oUs\|oAs\|oCs\|oA] | | | | |
| 82 | [oCs\|oUs\|oAp\|oGs\|oGs\|dTs\|dGs\|dAs\|d | 83.52985 | 34.76393 | 7.7233095 | 5.276733 |
| | Ts\|dGs\|5mCs\|dAs\|5mCs\|dTs\|dGs\|5mCs | | | | |
| | \|dTs\|oUs\|oUs\|oAs\|oCs\|oA] | | | | |
| 83 | [oCs\|oUs\|oAs\|oGp\|oGs\|dTs\|dGs\|dAs\|d | 87.34228 | 34.26426 | 2.8872435 | 3.491405 |
| | Ts\|dGs\|5mCs\|dAs\|5mCs\|dTs\|dGs\|5mCs | | | | |
| | \|dTs\|oUs\|oUs\|oAs\|oCs\|oA] | | | | |
| 84 | [oCs\|oUs\|oAp\|oGp\|oGs\|dTs\|dGs\|dAs\|d | 85.67873 | 35.6361 | 5.8302875 | 2.544952 |
| | Ts\|dGs\|5mCs\|dAs\|5mCs\|dTs\|dGs\|5mCs | | | | |
| | dTs\|oUs\|oUs\|oAs\|oCs\|oA] | | | | |
| 85 | [oCs\|oUs\|oAs\|oGs\|oGs\|dTs\|dGs\|dAs\|dT | 75.35312 | 32.86732 | 10.951265 | 3.621277 |
| | s\|dGs\|5mCs\|dAs\|5mCs\|dTs\|dGs\|5mCs\|d | | | | |
| | Tp\|oUs\|oUs\|oAs\|oCs\|oA] | | | | |
| 86 | [oCs\|oUs\|oAs\|oGs\|oGs\|dTs\|dGs\|dAs\|dT | 79.96271 | 34.14079 | 9.351093 | 1.427892 |
| | s\|dGs\|5mCs\|dAs\|5mCs\|dTs\|dGs\|5mCp\| | | | | |
| | dTs\|oUs\|oUs\|oAs\|oCs\|oA] | | | | |
| 87 | [oCs\|oUs\|oAs\|oGs\|oGs\|dTs\|dGs\|dAs\|dT | 100.34146 | 32.93465 | 7.409104 | 1.965485 |
| | s\|dGs\|5mCs\|dAs\|5mCs\|dTs\|dGs\|5mCp\| | | | | |
| | dTp\|oUs\|oUs\|oAs\|oCs\|oA] | | | | |
| 88 | [oCs\|oUs\|oAp\|oGs\|oGs\|dTs\|dGs\|dAs\|d | 76.43184 | 29.11159 | 1.9009085 | 1.712673 |
| | Ts\|dGs\|5mCs\|dAs\|5mCs\|dTs\|dGs\|5mCs | | | | |
| | \|dTp\|oUs\|oUs\|oAs\|oCs\|oA] | | | | |
| 89 | [oCs\|oUs\|oAp\|oGs\|oGs\|dTs\|dGs\|dAs\|d | 84.95746 | 33.1336 | 6.0100595 | 0.819655 |
| | Ts\|dGs\|5mCs\|dAs\|5mCs\|dTs\|dGs\|5mC | | | | |
| | p\|dTs\|oUs\|oUs\|oAs\|oCs\|oA] | | | | |
| 90 | [oCs\|oUs\|oAs\|oGp\|oGs\|dTs\|dGs\|dAs\|d | 77.40614 | 34.79199 | 2.737082 | 1.090979 |
| | Ts\|dGs\|5mCs\|dAs\|5mCs\|dTs\|dGs\|5mCs | | | | |
| | \|dTp\|oUs\|oUs\|oAs\|oCs\|oA] | | | | |
| 91 | [oCs\|oUs\|oAs\|oGp\|oGs\|dTs\|dGs\|dAs\|d | 75.61531 | 41.57976 | 2.6137425 | 2.594538 |
| | Ts\|dGs\|5mCs\|dAs\|5mCs\|dTs\|dGs\|5mC | | | | |
| | p\|dTs\|oUs\|oUs\|oAs\|oCs\|oA] | | | | |
| 92 | [oCs\|oUs\|oAp\|oGs\|oGs\|dTs\|dGs\|dAs\|d | 81.40445 | 36.37792 | 7.319196 | 0.14288 |
| | Ts\|dGs\|5mCs\|dAs\|5mCs\|dTs\|dGs\|5mC | | | | |
| | p\|dTp\|oUs\|oUs\|oAs\|oCs\|oA] | | | | |
| 93 | [oCs\|oUs\|oAs\|oGp\|oGs\|dTs\|dGs\|dAs\|d | 87.14688 | 41.27511 | 2.983684 | 3.624091 |
| | Ts\|dGs\|5mCs\|dAs\|5mCs\|dTs\|dGs\|5mC | | | | |
| | p\|dTp\|oUs\|oUs\|oAs\|oCs\|oA] | | | | |
| 94 | [oCs\|oUs\|oAp\|oGp\|oGs\|dTs\|dGs\|dAs\|d | 83.39023 | 40.63864 | 1.431887 | 1.205991 |
| | Ts\|dGs\|5mCs\|dAs\|5mCs\|dTs\|dGs\|5mCs | | | | |
| | \|dTp\|oUs\|oUs\|oAs\|oCs\|oA] | | | | |
| 95 | [oCs\|oUs\|oAp\|oGp\|oGs\|dTs\|dGs\|dAs\|d | 78.10296 | 40.32743 | 4.682538 | 1.18152 |
| | Ts\|dGs\|5mCs\|dAs\|5mCs\|dTs\|dGs\|5mC | | | | |
| | p\|dTs\|oUs\|oUs\|oAs\|oCs\|oA] | | | | |
| 96 | [oCs\|oUs\|oAp\|oGp\|oGs\|dTs\|dGs\|dAs\|d | 78.36184 | 41.53986 | 3.2471535 | 4.451705 |
| | Ts\|dGs\|5mCs\|dAs\|5mCs\|dTs\|dGs\|5mC | | | | |
| | p\|dTp\|oUs\|oUs\|oAs\|oCs\|oA] | | | | |
| 97 | [oUs\|oGs\|oCs\|oUs\|oAs\|dGs\|dGs\|dTs\|d | 73.53306 | 32.22054 | 5.7741956 | 1.828104 |
| | Gs\|dAs\|dTs\|dGs\|5mCs\|dAs\|5mCs\|oUs\|o | | | | |
| | Gs\|oCs\|oUs\|oU] | | | | |
| 98 | [oUs\|oGs\|oCp\|oUs\|oAs\|dGs\|dGs\|dTs\|d | 85.30286 | 28.26088 | 7.1938475 | 1.170224 |
| | Gs\|dAs\|dTs\|dGs\|5mCs\|dAs\|5mCs\|oUs\|o | | | | |
| | Gs\|oCs\|oUs\|oU] | | | | |
| 99 | [oUs\|oGs\|oCs\|oUp\|oAs\|dGs\|dGs\|dTs\|d | 80.72095 | 31.14705 | 6.94178635 | 0.847079 |
| | Gs\|dAs\|dTs\|dGs\|5mCs\|dAs\|5mCs\|oUs\|o | | | | |
| | Gs\|oCs\|oUs\|oU] | | | | |
| 100 | [oUs\|oGs\|oCp\|oUp\|oAs\|dGs\|dGs\|dTs\|d | 68.59723 | 30.44545 | 5.6598163 | 1.925702 |
| | Gs\|dAs\|dTs\|dGs\|5mCs\|dAs\|5mCs\|oUs\|o | | | | |
| | Gs\|oCs\|oUs\|oU] | | | | |
| 101 | [oUs\|oGs\|oCs\|oUs\|oAs\|dGs\|dGs\|dTs\|d | 78.30012 | 29.94689 | 6.2378524 | 4.046731 |
| | Gs\|dAs\|dTs\|dGs\|5mCs\|dAs\|5mCs\|oUs\|o | | | | |
| | Gp\|oCs\|oUs\|oU] | | | | |
| 102 | [oUs\|oGs\|oCs\|oUs\|oAs\|dGs\|dGs\|dTs\|d | 78.25236 | 30.06093 | 5.9663877 | 1.79938 |
| | Gs\|dAs\|dTs\|dGs\|5mCs\|dAs\|5mCs\|oUp\| | | | | |
| | oGs\|oCs\|oUs\|oU] | | | | |
| 103 | [oUs\|oGs\|oCs\|oUs\|oAs\|dGs\|dGs\|dTs\|d | 69.03511 | 35.56989 | 2.59911095 | 2.403012 |
| | Gs\|dAs\|dTs\|dGs\|5mCs\|dAs\|5mCs\|oUp\| | | | | |
| | oGp\|oCs\|oUs\|oU] | | | | |
| 104 | [oUs\|oGs\|oCp\|oUs\|oAs\|dGs\|dGs\|dTs\|d | 73.74024 | 30.77626 | 1.62019145 | 0.873961 |
| | Gs\|dAs\|dTs\|dGs\|5mCs\|dAs\|5mCs\|oUs\|o | | | | |
| | Gp\|oCs\|oUs\|oU] | | | | |
| 105 | [oUs\|oGs\|oCp\|oUs\|oAs\|dGs\|dGs\|dTs\|d | 72.2508 | 33.81869 | 4.46870435 | 3.070223 |
| | Gs\|dAs\|dTs\|dGs\|5mCs\|dAs\|5mCs\|oUp\| | | | | |
| | oGs\|oCs\|oUs\|oU | | | | |
| 106 | [oUs\|oGs\|oCs\|oUp\|oAs\|dGs\|dGs\|dTs\|d | 80.18669 | 27.94099 | 3.6700273 | 0.683295 |
| | Gs\|dAs\|dTs\|dGs\|5mCs\|dAs\|5mCs\|oUs\|o | | | | |
| | Gp\|oCs\|oUs\|oU] | | | | |
| 107 | [oUs\|oGs\|oCs\|oUp\|oAs\|dGs\|dGs\|dTs\|d | 74.09831 | 34.42556 | 6.01870395 | 1.988182 |
| | Gs\|dAs\|dTs\|dGs\|5mCs\|dAs\|5mCs\|oUp\| | | | | |
| | oGs\|oCs\|oUs\|oU] | | | | |
| 108 | [oUs\|oGs\|oCp\|oUs\|oAs\|dGs\|dGs\|dTs\|d | 70.94729 | 35.1242 | 1.85488915 | 3.987895 |
| | Gs\|dAs\|dTs\|dGs\|5mCs\|dAs\|5mCs\|oUp\| | | | | |
| | oGp\|oCs\|oUs\|oU] | | | | |
| 109 | [oUs\|oGs\|oCs\|oUp\|oAs\|dGs\|dGs\|dTs\|d | 78.33754 | 34.87067 | 4.7590703 | 1.027952 |
| | Gs\|dAs\|dTs\|dGs\|5mCs\|dAs\|5mCs\|oUp\| | | | | |
| | oGp\|oCs\|oUs\|oU] | | | | |
| 110 | [oUs\|oGs\|oCp\|oUp\|oAs\|dGs\|dGs\|dTs\|d | 62.32599 | 33.30946 | 3.7307496 | 2.867815 |
| | Gs\|dAs\|dTs\|dGs\|5mCs\|dAs\|5mCs\|oUs\|o | | | | |
| | Gp\|oCs\|oUs\|oU] | | | | |
| 111 | [oUs\|oGs\|oCp\|oUp\|oAs\|dGs\|dGs\|dTs\|d | 70.12462 | 35.35927 | 3.28310255 | 2.413138 |
| | Gs\|dAs\|dTs\|dGs\|5mCs\|dAs\|5mCs\|oUp\| | | | | |
| | oGs\|oCs\|oUs\|oU] | | | | |
| 112 | [oUs\|oGs\|oCp\|oUp\|oAs\|dGs\|dGs\|dTs\|d | 76.05292 | 32.20627 | 7.8049074 | 3.219622 |
| | Gs\|dAs\|dTs\|dGs\|5mCs\|dAs\|5mCs\|oUp\| | | | | |
| | oGp\|oCs\|oUs\|oU] | | | | |
| 113 | [oUs\|oGs\|oCs\|oUs\|oAs\|dGs\|dGs\|dTs\|d | 65.23498 | 32.17334 | 3.72245235 | 1.859401 |
| | Gs\|dAs\|dTs\|dGs\|5mCs\|dAs\|5mCs\|oUs\|o | | | | |
| | Gs\|oCs\|oUs\|oUs\|oU] | | | | |
| 114 | [oUs\|oGs\|oCp\|oUs\|oAs\|dGs\|dGs\|dTs\|d | 69.29675 | 32.68384 | 2.39326015 | 4.127576 |
| | Gs\|dAs\|dTs\|dGs\|5mCs\|dAs\|5mCs\|oUs\|o | | | | |
| | Gs\|oCs\|oUs\|oUs\|oU] | | | | |
| 115 | [oUs\|oGs\|oCs\|oUp\|oAs\|dGs\|dGs\|dTs\|d | 70.21315 | 28.19354 | 5.9917878 | 1.260233 |
| | Gs\|dAs\|dTs\|dGs\|5mCs\|dAs\|5mCs\|oUs\|o | | | | |
| | Gs\|oCs\|oUs\|oUs\|oU] | | | | |
| 116 | [oUs\|oGs\|oCp\|oUp\|oAs\|dGs\|dGs\|dTs\|d | 61.38246 | 27.89351 | 2.31272875 | 0.940955 |
| | Gs\|dAs\|dTs\|dGs\|5mCs\|dAs\|5mCs\|oUs\|o | | | | |
| | Gs\|oCs\|oUs\|oUs\|oU | | | | |
| 117 | [oUs\|oGs\|oCs\|oUs\|oAs\|dGs\|dGs\|dTs\|d | 67.38278 | 29.83921 | 4.699614 | 3.62231 |
| | Gs\|dAs\|dTs\|dGs\|5mCs\|dAs\|5mCs\|oUs\|o | | | | |
| | Gp\|oCs\|oUs\|oUs\|oU] | | | | |
| 118 | [oUs\|oGs\|oCs\|oUs\|oAs\|dGs\|dGs\|dTs\|d | 74.8669 | 33.51358 | 4.66523895 | 3.23944 |
| | Gs\|dAs\|dTs\|dGs\|5mCs\|dAs\|5mCs\|oUp\| | | | | |
| | oGs\|oCs\|oUs\|oUs\|oU] | | | | |
| 119 | [oUs\|oGs\|oCs\|oUs\|oAs\|dGs\|dGs\|dTs\|d | 75.87658 | 33.86225 | 3.36764485 | 2.561053 |
| | Gs\|dAs\|dTs\|dGs\|5mCs\|dAs\|5mCs\|oUp\| | | | | |
| | oGp\|oCs\|oUs\|oUs\|oU] | | | | |
| 120 | [oUs\|oGs\|oCp\|oUs\|oAs\|dGs\|dGs\|dTs\|d | 74.89974 | 30.42127 | 5.3692428 | 1.390075 |
| | Gs\|dAs\|dTs\|dGs\|5mCs\|dAs\|5mCs\|oUs\|o | | | | |
| | Gp\|oCs\|oUs\|oUs\|oU] | | | | |
| 121 | [oUs\|oGs\|oCp\|oUs\|oAs\|dGs\|dGs\|dTs\|d | 77.25051 | 35.01204 | 10.42313865 | 2.358081 |
| | Gs\|dAs\|dTs\|dGs\|5mCs\|dAs\|5mCs\|oUp\| | | | | |
| | oGs\|oCs\|oUs\|oUs\|oU] | | | | |
| 122 | [oUs\|oGs\|oCs\|oUp\|oAs\|dGs\|dGs\|dTs\|d | 68.7315 | 29.79101 | 4.2400331 | 3.545545 |
| | Gs\|dAs\|dTs\|dGs\|5mCs\|dAs\|5mCs\|oUs\|o | | | | |
| | Gp\|oCs\|oUs\|oUs\|oU] | | | | |
| 123 | [oUs\|oGs\|oCs\|oUp\|oAs\|dGs\|dGs\|dTs\|d | 71.42427 | 34.17864 | 4.1743313 | 2.028014 |
| | Gs\|dAs\|dTs\|dGs\|5mCs\|dAs\|5mCs\|oUp\| | | | | |
| | oGs\|oCs\|oUs\|oUs\|oU] | | | | |
| 124 | [oUs\|oGs\|oCp\|oUs\|oAs\|dGs\|dGs\|dTs\|d | 86.14101 | 31.27527 | 4.48923135 | 2.071295 |
| | Gs\|dAs\|dTs\|dGs\|5mCs\|dAs\|5mCs\|oUp\| | | | | |
| | oGp\|oCs\|oUs\|oUs\|oU] | | | | |
| 125 | [oUs\|oGs\|oCs\|oUp\|oAs\|dGs\|dGs\|dTs\|d | 77.77647 | 31.7119 | 4.05551285 | 1.114638 |
| | Gs\|dAs\|dTs\|dGs\|5mCs\|dAs\|5mCs\|oUp\| | | | | |
| | oGp\|oCs\|oUs\|oUs\|oU] | | | | |
| 126 | [oUs\|oGs\|oCp\|oUp\|oAs\|dGs\|dGs\|dTs\|d | 78.6535 | 37.61556 | 1.55106765 | 2.362156 |
| | Gs\|dAs\|dTs\|dGs\|5mCs\|dAs\|5mCs\|oUs\|o | | | | |
| | Gp\|oCs\|oUs\|oUs\|oU] | | | | |
| 127 | [oUs\|oGs\|oCp\|oUp\|oAs\|dGs\|dGs\|dTs\|d | 67.14048 | 33.39059 | 1.7917388 | 1.42932 |
| | Gs\|dAs\|dTs\|dGs\|5mCs\|dAs\|5mCs\|oUp\| | | | | |
| | oGs\|oCs\|oUs\|oUs\|oU] | | | | |
| 128 | [oUs\|oGs\|oCp\|oUp\|oAs\|dGs\|dGs\|dTs\|d | 86.42336 | 39.03898 | 2.597663 | 2.239799 |
| | Gs\|dAs\|dTs\|dGs\|5mCs\|dAs\|5mCs\|oUp\| | | | | |
| | oGp\|oCs\|oUs\|oUs\|oU] | | | | |
| 129 | [oUs\|oGs\|oCs\|oUs\|oAs\|dGs\|dGs\|dTs\|d | 68.63405 | 35.13624 | 1.53173715 | 2.451205 |
| | Gs\|dAs\|dTs\|dGs\|5mCs\|dAs\|5mCs\|oUs\|o | | | | |
| | Gs\|oCs\|oUs\|oUs\|oUs\|oA] | | | | |
| 130 | [oUs\|oGs\|oCp\|oUs\|oAs\|dGs\|dGs\|dTs\|d | 69.38908 | 31.9245 | 4.0133836 | 3.413571 |
| | Gs\|dAs\|dTs\|dGs\|5mCs\|dAs\|5mCs\|oUs\|o | | | | |
| | Gs\|oCs\|oUs\|oUs\|oUs\|oA] | | | | |
| 131 | [oUs\|oGs\|oCs\|oUp\|oAs\|dGs\|dGs\|dTs\|d | 82.64957 | 31.6315 | 10.9520439 | 1.487167 |
| | Gs\|dAs\|dTs\|dGs\|5mCs\|dAs\|5mCs\|oUs\|o | | | | |
| | Gs\|oCs\|oUs\|oUs\|oUs\|oA] | | | | |
| 132 | [oUs\|oGs\|oCp\|oUp\|oAs\|dGs\|dGs\|dTs\|d | 76.22312 | 31.57458 | 5.88891735 | 2.253033 |
| | Gs\|dAs\|dTs\|dGs\|5mCs\|dAs\|5mCs\|oUs\|o | | | | |
| | Gs\|oCs\|oUs\|oUs\|oUs\|oA] | | | | |
| 133 | [oUs\|oGs\|oCs\|oUs\|oAs\|dGs\|dGs\|dTs\|d | 73.47365 | 27.08401 | 3.77061195 | 0.92052 |
| | Gs\|dAs\|dTs\|dGs\|5mCs\|dAs\|5mCs\|oUs\|o | | | | |
| | Gp\|oCs\|oUs\|oUs\|oUs\|oA] | | | | |
| 134 | [oUs\|oGs\|oCs\|oUs\|oAs\|dGs\|dGs\|dTs\|d | 70.30414 | 33.09977 | 4.27095005 | 3.045878 |
| | Gs\|dAs\|dTs\|dGs\|5mCs\|dAs\|5mCs\|oUp\| | | | | |
| | oGs\|oCs\|oUs\|oUs\|oUs\|oA] | | | | |
| 135 | [oUs\|oGs\|oCs\|oUs\|oAs\|dGs\|dGs\|dTs\|d | 72.20899 | 30.08256 | 6.4512196 | 1.841916 |
| | Gs\|dAs\|dTs\|dGs\|5mCs\|dAs\|5mCs\|oUp\| | | | | |
| | oGp\|oCs\|oUs\|oUs\|oUs\|oA] | | | | |
| 136 | [oUs\|oGs\|oCp\|oUs\|oAs\|dGs\|dGs\|dTs\|d | 67.44648 | 31.14491 | 2.3933433 | 2.847244 |
| | Gs\|dAs\|dTs\|dGs\|5mCs\|dAs\|5mCs\|oUs\|o | | | | |
| | Gp\|oCs\|oUs\|oUs\|oUs\|oA] | | | | |
| 137 | [oUs\|oGs\|oCp\|oUs\|oAs\|dGs\|dGs\|dTs\|d | 73.72042 | 31.65609 | 3.1864744 | 1.736871 |
| | Gs\|dAs\|dTs\|dGs\|5mCs\|dAs\|5mCs\|oUp\| | | | | |
| | oGs\|oCs\|oUs\|oUs\|oUs\|oA] | | | | |
| 138 | [oUs\|oGs\|oCs\|oUp\|oAs\|dGs\|dGs\|dTs\|d | 67.00788 | 29.57105 | 4.13148395 | 0.996282 |
| | Gs\|dAs\|dTs\|dGs\|5mCs\|dAs\|5mCs\|oUs\|o | | | | |
| | Gp\|oCs\|oUs\|oUs\|oUs\|oA] | | | | |
| 139 | [oUs\|oGs\|oCs\|oUp\|oAs\|dGs\|dGs\|dTs\|d | 67.67345 | 29.52463 | 2.84221865 | 1.821037 |
| | Gs\|dAs\|dTs\|dGs\|5mCs\|dAs\|5mCs\|oUp\| | | | | |
| | oGs\|oCs\|oUs\|oUs\|oUs\|oA] | | | | |
| 140 | [oUs\|oGs\|oCp\|oUs\|oAs\|dGs\|dGs\|dTs\|d | 74.02419 | 34.75728 | 4.05854245 | 2.515477 |
| | Gs\|dAs\|dTs\|dGs\|5mCs\|dAs\|5mCs\|oUp\| | | | | |
| | oGp\|oCs\|oUs\|oUs\|oUs\|oA] | | | | |
| 141 | [oUs\|oGs\|oCs\|oUp\|oAs\|dGs\|dGs\|dTs\|d | 70.79225 | 32.87075 | 1.72015605 | 1.818799 |
| | Gs\|dAs\|dTs\|dGs\|5mCs\|dAs\|5mCs\|oUp\| | | | | |
| | oGp\|oCs\|oUs\|oUs\|oUs\|oA] | | | | |
| 142 | [oUs\|oGs\|oCp\|oUp\|oAs\|dGs\|dGs\|dTs\|d | 80.55014 | 28.38316 | 4.20708315 | 1.230702 |
| | Gs\|dAs\|dTs\|dGs\|5mCs\|dAs\|5mCs\|oUs\|o | | | | |
| | Gp\|oCs\|oUs\|oUs\|oUs\|oA] | | | | |
| 143 | [oUs\|oGs\|oCp\|oUp\|oAs\|dGs\|dGs\|dTs\|d | 62.97423 | 33.06359 | 2.1320595 | 3.346436 |
| | Gs\|dAs\|dTs\|dGs\|5mCs\|dAs\|5mCs\|oUp\| | | | | |
| | oGs\|oCs\|oUs\|oUs\|oUs\|oA] | | | | |
| 144 | [oUs\|oGs\|oCp\|oUp\|oAs\|dGs\|dGs\|dTs\|d | 70.79163 | 33.67681 | 5.18027175 | 3.32755 |
| | Gs\|dAs\|dTs\|dGs\|5mCs\|dAs\|5mCs\|oUp\| | | | | |
| | oGp\|oCs\|oUs\|oUs\|oUs\|oA] | | | | |
| 145 | [oUs\|oGs\|oCs\|oUs\|oAs\|dGs\|dGs\|dTs\|d | 61.07007 | 32.76229 | 1.578968 | 2.337807 |
| | Gs\|dAs\|dTs\|dGs\|5mCs\|dAs\|5mCs\|oUs\|o | | | | |
| | Gs\|oCs\|oUs\|oUs\|oUs\|oAs\|oC] | | | | |
| 146 | [oUs\|oGs\|oCp\|oUs\|oAs\|dGs\|dGs\|dTs\|d | 77.42478 | 34.61478 | 7.0884636 | 3.366066 |
| | Gs\|dAs\|dTs\|dGs\|5mCs\|dAs\|5mCs\|oUs\|o | | | | |
| | Gs\|oCs\|oUs\|oUs\|oUs\|oAs\|oC] | | | | |
| 147 | [oUs\|oGs\|oCs\|oUp\|oAs\|dGs\|dGs\|dTs\|d | 67.36325 | 38.44457 | 3.158251 | 2.192247 |
| | Gs\|dAs\|dTs\|dGs\|5mCs\|dAs\|5mCs\|oUs\|o | | | | |
| | Gs\|oCs\|oUs\|oUs\|oUs\|oAs\|oC] | | | | |
| 148 | [oUs\|oGs\|oCp\|oUp\|oAs\|dGs\|dGs\|dTs\|d | 69.55673 | 34.56908 | 2.1775117 | 2.805347 |
| | Gs\|dAs\|dTs\|dGs\|5mCs\|dAs\|5mCs\|oUs\|o | | | | |
| | Gs\|oCs\|oUs\|oUs\|oUs\|oAs\|oC] | | | | |
| 149 | [oUs\|oGs\|oCs\|oUs\|oAs\|dGs\|dGs\|dTs\|d | 82.73055 | 33.15298 | 3.68306915 | 2.319368 |
| | Gs\|dAs\|dTs\|dGs\|5mCs\|dAs\|5mCs\|oUs\|o | | | | |
| | Gp\|oCs\|oUs\|oUs\|oUs\|oAs\|oC] | | | | |
| 150 | [oUs\|oGs\|oCs\|oUs\|oAs\|dGs\|dGs\|dTs\|d | 74.0629 | 34.61137 | 4.9070243 | 1.082019 |
| | Gs\|dAs\|dTs\|dGs\|5mCs\|dAs\|5mCs\|oUp\| | | | | |
| | oGs\|oCs\|oUs\|oUs\|oUs\|oAs\|oC] | | | | |
| 151 | [oUs\|oGs\|oCs\|oUs\|oAs\|dGs\|dGs\|dTs\|d | 86.07845 | 39.51103 | 3.76062145 | 3.134823 |
| | Gs\|dAs\|dTs\|dGs\|5mCs\|dAs\|5mCs\|oUp\| | | | | |
| | oGp\|oCs\|oUs\|oUs\|oUs\|oAs\|oC] | | | | |
| 152 | [oUs\|oGs\|oCp\|oUs\|oAs\|dGs\|dGs\|dTs\|d | 82.2174 | 29.35929 | 3.78661745 | 1.69256 |
| | Gs\|dAs\|dTs\|dGs\|5mCs\|dAs\|5mCs\|oUs\|o | | | | |
| | Gp\|oCs\|oUs\|oUs\|oUs\|oAs\|oC] | | | | |
| 153 | [oUs\|oGs\|oCp\|oUs\|oAs\|dGs\|dGs\|dTs\|d | 75.98421 | 35.81497 | 2.16191275 | 2.093193 |
| | Gs\|dAs\|dTs\|dGs\|5mCs\|dAs\|5mCs\|oUp\| | | | | |
| | oGs\|oCs\|oUs\|oUs\|oUs\|oAs\|oC] | | | | |
| 154 | [oUs\|oGs\|oCs\|oUp\|oAs\|dGs\|dGs\|dTs\|d | 68.75699 | 32.99567 | 4.0256092 | 1.228557 |
| | Gs\|dAs\|dTs\|dGs\|5mCs\|dAs\|5mCs\|oUs\|o | | | | |
| | Gp\|oCs\|oUs\|oUs\|oUs\|oAs\|oC] | | | | |
| 155 | [oUs\|oGs\|oCs\|oUp\|oAs\|dGs\|dGs\|dTs\|d | 65.0077 | 33.91453 | 4.8970901 | 1.843589 |
| | Gs\|dAs\|dTs\|dGs\|5mCs\|dAs\|5mCs\|oUp\| | | | | |
| | oGs\|oCs\|oUs\|oUs\|oUs\|oAs\|oC] | | | | |
| 156 | [oUs\|oGs\|oCp\|oUs\|oAs\|dGs\|dGs\|dTs\|d | 81.28814 | 36.71857 | 4.66906275 | 3.495077 |
| | Gs\|dAs\|dTs\|dGs\|5mCs\|dAs\|5mCs\|oUp\| | | | | |
| | oGp\|oCs\|oUs\|oUs\|oUs\|oAs\|oC] | | | | |
| 157 | [oUs\|oGs\|oCs\|oUp\|oAs\|dGs\|dGs\|dTs\|d | 69.80645 | 66.34306 | 2.97949155 | 17.28836 |
| | Gs\|dAs\|dTs\|dGs\|5mCs\|dAs\|5mCs\|oUp\| | | | | |
| | oGp\|oCs\|oUs\|oUs\|oUs\|oAs\|oC] | | | | |
| 158 | [oUs\|oGs\|oCp\|oUp\|oAs\|dGs\|dGs\|dTs\|d | 68.37275 | 72.33517 | 4.17040095 | 18.58276 |
| | Gs\|dAs\|dTs\|dGs\|5mCs\|dAs\|5mCs\|oUs\|o | | | | |
| | Gp\|oCs\|oUs\|oUs\|oUs\|oAs\|oC] | | | | |
| 159 | [oUs\|oGs\|oCp\|oUp\|oAs\|dGs\|dGs\|dTs\|d | 72.61681 | 47.70773 | 2.2537468 | 6.897452 |
| | Gs\|dAs\|dTs\|dGs\|5mCs\|dAs\|5mCs\|oUp\| | | | | |
| | oGs\|oCs\|oUs\|oUs\|oUs\|oAs\|oC] | | | | |
| 160 | [oUs\|oGs\|oCp\|oUp\|oAs\|dGs\|dGs\|dTs\|d | 68.78353 | 84.24789 | 3.0864697 | 31.8067 |
| | Gs\|dAs\|dTs\|dGs\|5mCs\|dAs\|5mCs\|oUp\| | | | | |
| | oGp\|oCs\|oUs\|oUs\|oUs\|oAs\|oC] | | | | |
| 161 | [oUs\|oGs\|oCs\|oUs\|oAs\|dGs\|dGs\|dTs\|d | 69.3108 | 75.14277 | 15.38158025 | 25.56809 |
| | Gs\|dAs\|dTs\|dGs\|5mCs\|dAs\|5mCs\|dTs\|o | | | | |
| | Gs\|oCs\|oUs\|oUs\|oU] | | | | |
| 162 | [oUs\|oGs\|oCp\|oUs\|oAs\|dGs\|dGs\|dTs\|d | 72.9714 | 69.75189 | 5.9076121 | 32.69531 |
| | Gs\|dAs\|dTs\|dGs\|5mCs\|dAs\|5mCs\|dTs\|o | | | | |
| | Gs\|oCs\|oUs\|oUs\|oU] | | | | |
| 163 | [oUs\|oGs\|oCs\|oUp\|oAs\|dGs\|dGs\|dTs\|d | 59.9413 | 33.69336 | 2.7716944 | 2.300999 |
| | Gs\|dAs\|dTs\|dGs\|5mCs\|dAs\|5mCs\|dTs\|o | | | | |
| | Gs\|oCs\|oUs\|oUs\|oU] | | | | |
| 164 | [oUs\|oGs\|oCp\|oUp\|oAs\|dGs\|dGs\|dTs\|d | 65.88331 | 42.9639 | 6.38280315 | 2.950032 |
| | Gs\|dAs\|dTs\|dGs\|5mCs\|dAs\|5mCs\|dTs\|o | | | | |
| | Gs\|oCs\|oUs\|oUs\|oU] | | | | |
| 165 | [oUs\|oGs\|oCs\|oUs\|oAs\|dGs\|dGs\|dTs\|d | 60.39611 | 41.07525 | 4.74049725 | 1.401234 |
| | Gs\|dAs\|dTs\|dGs\|5mCs\|dAs\|5mCs\|dTs\|o | | | | |
| | Gp\|oCs\|oUs\|oUs\|oU] | | | | |
| 166 | [oUs\|oGs\|oCs\|oUs\|oAs\|dGs\|dGs\|dTs\|d | 75.65893 | 53.6832 | 6.7614234 | 2.709928 |
| | Gs\|dAs\|dTs\|dGs\|5mCs\|dAs\|5mCs\|dTp\|o | | | | |
| | Gs\|oCs\|oUs\|oUs\|oU] | | | | |
| 167 | [oUs\|oGs\|oCs\|oUs\|oAs\|dGs\|dGs\|dTs\|d | 78.27304 | 68.71663 | 3.6412356 | 20.69837 |
| | Gs\|dAs\|dTs\|dGs\|5mCs\|dAs\|5mCs\|dTp\|o | | | | |
| | Gp\|oCs\|oUs\|oUs\|oU] | | | | |
| 168 | [oUs\|oGs\|oCp\|oUs\|oAs\|dGs\|dGs\|dTs\|d | 69.66589 | 40.46645 | 4.8223807 | 1.966849 |
| | Gs\|dAs\|dTs\|dGs\|5mCs\|dAs\|5mCs\|dTs\|o | | | | |
| | Gp\|oCs\|oUs\|oUs\|oU] | | | | |
| 169 | [oUs\|oGs\|oCp\|oUs\|oAs\|dGs\|dGs\|dTs\|d | 65.01222 | 51.18193 | 2.0901017 | 5.432837 |
| | Gs\|dAs\|dTs\|dGs\|5mCs\|dAs\|5mCs\|dTp\|o | | | | |
| | Gs\|oCs\|oUs\|oUs\|oU] | | | | |
| 170 | [oUs\|oGs\|oCs\|oUp\|oAs\|dGs\|dGs\|dTs\|d | 64.20334 | 44.9343 | 3.03968285 | 2.38631 |
| | Gs\|dAs\|dTs\|dGs\|5mCs\|dAs\|5mCs\|dTs\|o | | | | |
| | Gp\|oCs\|oUs\|oUs\|oU] | | | | |
| 171 | [oUs\|oGs\|oCs\|oUp\|oAs\|dGs\|dGs\|dTs\|d | 74.50087 | 46.7009 | 7.1516196 | 3.17865 |
| | Gs\|dAs\|dTs\|dGs\|5mCs\|dAs\|5mCs\|dTp\|o | | | | |
| | Gs\|oCs\|oUs\|oUs\|oU] | | | | |
| 172 | [oUs\|oGs\|oCp\|oUs\|oAs\|dGs\|dGs\|dTs\|d | 66.0219 | 51.29474 | 5.5452825 | 7.02709 |
| | Gs\|dAs\|dTs\|dGs\|5mCs\|dAs\|5mCs\|dTp\|o | | | | |
| | Gp\|oCs\|oUs\|oUs\|oU] | | | | |
| 173 | [oUs\|oGs\|oCs\|oUp\|oAs\|dGs\|dGs\|dTs\|d | 88.90626 | 43.66599 | 7.76517935 | 4.598041 |
| | Gs\|dAs\|dTs\|dGs\|5mCs\|dAs\|5mCs\|dTp\|o | | | | |
| | Gp\|oCs\|oUs\|oUs\|oU] | | | | |
| 174 | [oUs\|oGs\|oCp\|oUp\|oAs\|dGs\|dGs\|dTs\|d | 70.63885 | 81.73461 | 10.1429891 | 39.6005 |
| | Gs\|dAs\|dTs\|dGs\|5mCs\|dAs\|5mCs\|dTs\|o | | | | |
| | Gp\|oCs\|oUs\|oUs\|oU] | | | | |
| 175 | [oUs\|oGs\|oCp\|oUp\|oAs\|dGs\|dGs\|dTs\|d | 69.65344 | 57.02863 | 4.09660005 | 13.08032 |
| | Gs\|dAs\|dTs\|dGs\|5mCs\|dAs\|5mCs\|dTp\|o | | | | |
| | Gs\|oCs\|oUs\|oUs\|oU] | | | | |
| 176 | [oUs\|oGs\|oCp\|oUp\|oAs\|dGs\|dGs\|dTs\|d | 71.0292 | 88.44799 | 3.57082955 | 37.50085 |
| | Gs\|dAs\|dTs\|dGs\|5mCs\|dAs\|5mCs\|dTp\|o | | | | |
| | Gp\|oCs\|oUs\|oUs\|oU] | | | | |
| 177 | [oUs\|oGs\|oCs\|oUs\|oAs\|dGs\|dGs\|dTs\|d | 66.62139 | 41.51005 | 2.37850925 | 4.804473 |
| | Gs\|dAs\|dTs\|dGs\|5mCs\|dAs\|5mCs\|dTs\|d | | | | |
| | Gs\|oCs\|oUs\|oUs\|oUs\|oA] | | | | |
| 178 | [oUs\|oGs\|oCp\|oUs\|oAs\|dGs\|dGs\|dTs\|d | 72.29763 | 44.50319 | 6.87317685 | 2.677576 |
| | Gs\|dAs\|dTs\|dGs\|5mCs\|dAs\|5mCs\|dTs\|d | | | | |
| | Gs\|oCs\|oUs\|oUs\|oUs\|oA] | | | | |
| 179 | [oUs\|oGs\|oCs\|oUp\|oAs\|dGs\|dGs\|dTs\|d | 68.20743 | 47.68644 | 5.01288235 | 7.92615 |
| | Gs\|dAs\|dTs\|dGs\|5mCs\|dAs\|5mCs\|dTs\|d | | | | |
| | Gs\|oCs\|oUs\|oUs\|oUs\|oA] | | | | |
| 180 | [oUs\|oGs\|oCp\|oUp\|oAs\|dGs\|dGs\|dTs\|d | 68.99648 | 42.6724 | 0.11444645 | 1.915543 |
| | Gs\|dAs\|dTs\|dGs\|5mCs\|dAs\|5mCs\|dTs\|d | | | | |
| | Gs\|oCs\|oUs\|oUs\|oUs\|oA] | | | | |
| 181 | [oUs\|oGs\|oCs\|oUs\|oAs\|dGs\|dGs\|dTs\|d | 74.55986 | 75.31866 | 4.8690439 | 16.158 |
| | Gs\|dAs\|dTs\|dGs\|5mCs\|dAs\|5mCs\|dTs\|d | | | | |
| | Gp\|oCs\|oUs\|oUs\|oUs\|oA] | | | | |
| 182 | [oUs\|oGs\|oCs\|oUs\|oAs\|dGs\|dGs\|dTs\|d | 90.17217 | 57.85726 | 6.4500736 | 10.25964 |
| | Gs\|dAs\|dTs\|dGs\|5mCs\|dAs\|5mCs\|dTp\|d | | | | |
| | Gs\|oCs\|oUs\|oUs\|oUs\|oA] | | | | |
| 183 | [oUs\|oGs\|oCs\|oUs\|oAs\|dGs\|dGs\|dTs\|d | 79.6849 | 44.0499 | 4.4692627 | 2.659523 |
| | Gs\|dAs\|dTs\|dGs\|5mCs\|dAs\|5mCs\|dTp\|d | | | | |
| | Gp\|oCs\|oUs\|oUs\|oUs\|oA] | | | | |
| 184 | [oUs\|oGs\|oCp\|oUs\|oAs\|dGs\|dGs\|dTs\|d | 72.7416 | 46.00267 | 6.4985466 | 1.990908 |
| | Gs\|dAs\|dTs\|dGs\|5mCs\|dAs\|5mCs\|dTs\|d | | | | |
| | Gp\|oCs\|oUs\|oUs\|oUs\|oA] | | | | |
| 185 | [oUs\|oGs\|oCp\|oUs\|oAs\|dGs\|dGs\|dTs\|d | 78.36759 | 57.88874 | 8.4461547 | 7.916173 |
| | Gs\|dAs\|dTs\|dGs\|5mCs\|dAs\|5mCs\|dTp\|d | | | | |
| | Gs\|oCs\|oUs\|oUs\|oUs\|oA] | | | | |
| 186 | [oUs\|oGs\|oCs\|oUp\|oAs\|dGs\|dGs\|dTs\|d | 77.17065 | 41.09619 | 4.06900365 | 5.266146 |
| | Gs\|dAs\|dTs\|dGs\|5mCs\|dAs\|5mCs\|dTs\|d | | | | |
| | Gp\|oCs\|oUs\|oUs\|oUs\|oA] | | | | |
| 187 | [oUs\|oGs\|oCs\|oUp\|oAs\|dGs\|dGs\|dTs\|d | 71.80721 | 47.54313 | 4.0522398 | 4.300292 |
| | Gs\|dAs\|dTs\|dGs\|5mCs\|dAs\|5mCs\|dTp\|d | | | | |
| | Gs\|oCs\|oUs\|oUs\|oUs\|oA] | | | | |
| 188 | [oUs\|oGs\|oCp\|oUs\|oAs\|dGs\|dGs\|dTs\|d | 83.68087 | 47.27023 | 11.6233265 | 2.662367 |
| | Gs\|dAs\|dTs\|dGs\|5mCs\|dAs\|5mCs\|dTp\|d | | | | |
| | Gp\|oCs\|oUs\|oUs\|oUs\|oA] | | | | |
| 189 | [oUs\|oGs\|oCs\|oUp\|oAs\|dGs\|dGs\|dTs\|d | 90.45532 | 46.05918 | 4.6439005 | 3.872029 |
| | Gs\|dAs\|dTs\|dGs\|5mCs\|dAs\|5mCs\|dTp\|d | | | | |
| | Gp\|oCs\|oUs\|oUs\|oUs\|oA] | | | | |
| 190 | [oUs\|oGs\|oCp\|oUp\|oAs\|dGs\|dGs\|dTs\|d | 89.19433 | 34.62675 | 9.5661226 | 4.858138 |
| | Gs\|dAs\|dTs\|dGs\|5mCs\|dAs\|5mCs\|dTs\|d | | | | |
| | Gp\|oCs\|oUs\|oUs\|oUs\|oA] | | | | |
| 191 | [oUs\|oGs\|oCp\|oUp\|oAs\|dGs\|dGs\|dTs\|d | 64.72372 | 43.20547 | 3.7289791 | 2.557415 |
| | Gs\|dAs\|dTs\|dGs\|5mCs\|dAs\|5mCs\|dTp\|d | | | | |
| | Gs\|oCs\|oUs\|oUs\|oUs\|oA] | | | | |
| 192 | [oUs\|oGs\|oCp\|oUp\|oAs\|dGs\|dGs\|dTs\|d | 92.70258 | 41.35463 | 12.14039325 | 0.531077 |
| | Gs\|dAs\|dTs\|dGs\|5mCs\|dAs\|5mCs\|dTp\|d | | | | |
| | Gp\|oCs\|oUs\|oUs\|oUs\|oA] | | | | |
| 193 | [oUs\|oGs\|oAs\|oUs\|oCs\|5mCs\|dTs\|dGs\| | 41.82279 | 25.79387 | 2.85807925 | 1.785736 |
| | dTs\|dTs\|5mCs\|dTs\|5mCs\|5mCs\|5mCs\|o | | | | |
| | As\|oGs\|oCs\|oAs\|oA] | | | | |
| 194 | [oUs\|oGs\|oAp\|oUs\|oCs\|5mCs\|dTs\|dGs\| | 34.50835 | 24.88784 | 2.37599485 | 2.49983 |
| | dTs\|dTs\|5mCs\|dTs\|5mCs\|5mCs\|5mCs\|o | | | | |
| | As\|oGs\|oCs\|oAs\|oA] | | | | |
| 195 | [oUs\|oGs\|oAs\|oUp\|oCs\|5mCs\|dTs\|dGs\| | 34.48394 | 27.68268 | 3.0471537 | 1.050805 |
| | dTs\|dTs\|5mCs\|dTs\|5mCs\|5mCs\|5mCs\|o | | | | |
| | As\|oGs\|oCs\|oAs\|oA] | | | | |
| 196 | [oUs\|oGs\|oAp\|oUp\|oCs\|5mCs\|dTs\|dGs\| | 35.91642 | 27.09371 | 0.5861107 | 3.367908 |
| | dTs\|dTs\|5mCs\|dTs\|5mCs\|5mCs\|5mCs\|o | | | | |
| | As\|oGs\|oCs\|oAs\|oA] | | | | |
| 197 | [oUs\|oGs\|oAs\|oUs\|oCs\|5mCs\|dTs\|dGs\| | 40.41755 | 30.50893 | 3.2331104 | 1.618927 |
| | dTs\|dTs\|5mCs\|dTs\|5mCs\|5mCs\|5mCs\|o | | | | |
| | As\|oGp\|oCs\|oAs\|oA] | | | | |
| 198 | [oUs\|oGs\|oAs\|oUs\|oCs\|5mCs\|dTs\|dGs\| | 37.18947 | 26.7628 | 1.96874185 | 1.810888 |
| | dTs\|dTs\|5mCs\|dTs\|5mCs\|5mCs\|5mCs\|o | | | | |
| | Ap\|oGs\|oCs\|oAs\|oA] | | | | |
| 199 | [oUs\|oGs\|oAs\|oUs\|oCs\|5mCs\|dTs\|dGs\| | 41.58604 | 32.4533 | 2.0955341 | 3.083019 |
| | dTs\|dTs\|5mCs\|dTs\|5mCs\|5mCs\|5mCs\|o | | | | |
| | Ap\|oGp\|oCs\|oAs\|oA] | | | | |
| 200 | [oUs\|oGs\|oAp\|oUs\|oCs\|5mCs\|dTs\|dGs\| | 41.50165 | 29.4431 | 1.99516615 | 2.336989 |
| | dTs\|dTs\|5mCs\|dTs\|5mCs\|5mCs\|5mCs\|o | | | | |
| | As\|oGp\|oCs\|oAs\|oA] | | | | |
| 201 | dTs\|dTs\|5mCs\|dTs\|5mCs\|5mCs\|5mCs\|o | 42.01393 | 23.06252 | 1.3193855 | 4.944764 |
| | dTs\|dTs\|5mCs\|dTs\|5mCs\|5mCs\|5mCs\|o | | | | |
| | Apo\|Gs\|oCs\|oAs\|oA] | | | | |
| 202 | [oUs\|oGs\|oAs\|oUp\|oCs\|5mCs\|dTs\|dGs\| | 41.74463 | 27.31196 | 3.3971388 | 1.186669 |
| | dTs\|dTs\|5mCs\|dTs\|5mCs\|5mCs\|5mCs\|o | | | | |
| | As\|oGp\|oCs\|oAs\|oA] | | | | |
| 203 | [oUs\|oGs\|oAs\|oUp\|oCs\|5mCs\|dTs\|dGs\| | 40.8989 | 27.94533 | 2.5002774 | 2.019763 |
| | dTs\|dTs\|5mCs\|dTs\|5mCs\|5mCs\|5mCs\|o | | | | |
| | Ap\|oGs\|oCs\|oAs\|oA] | | | | |
| 204 | [oUs\|oGs\|oAp\|oUs\|oCs\|5mCs\|dTs\|dGs\| | 39.77508 | 30.17107 | 2.68253975 | 3.295311 |
| | dTs\|dTs\|5mCs\|dTs\|5mCs\|5mCs\|5mCs\|o | | | | |
| | Ap\|oGp\|oCs\|oAs\|oA] | | | | |
| 205 | [oUs\|oGs\|oAs\|oUp\|oCs\|5mCs\|dTs\|dGs\| | 40.56325 | 26.69679 | 1.7302717 | 0.745227 |
| | dTs\|dTs\|5mCs\|dTs\|5mCs\|5mCs\|5mCs\|o | | | | |
| | Ap\|oGp\|oCs\|oAs\|oA] | | | | |
| 206 | [oUs\|oGs\|oAp\|oUp\|oCs\|5mCs\|dTs\|dGs\| | 39.89604 | 19.42737 | 3.86707555 | 1.982879 |
| | dTs\|dTs\|5mCs\|dTs\|5mCs\|5mCs\|5mCs\|o | | | | |
| | As\|oGp\|oCs\|oAs\|oA] | | | | |
| 207 | [oUs\|oGs\|oAp\|oUp\|oCs\|5mCs\|dTs\|dGs\| | 40.47438 | 25.35393 | 2.3847392 | 1.251586 |
| | dTs\|dTs\|5mCs\|dTs\|5mCs\|5mCs\|5mCs\|o | | | | |
| | Ap\|oGs\|oCs\|oAs\|oA] | | | | |
| 208 | [oUs\|oGs\|oAp\|oUp\|oCs\|5mCs\|dTs\|dGs\| | 39.84144 | 26.46836 | 2.76645765 | 1.184643 |
| | dTs\|dTs\|5mCs\|dTs\|5mCs\|5mCs\|5mCs\|o | | | | |
| | Ap\|oGp\|oCs\|oAs\|oA] | | | | |
| 209 | [oUs\|oGs\|oAs\|oUs\|oCs\|5mCs\|dTs\|dGs\| | 36.38624 | 24.02288 | 1.92817845 | 1.780277 |
| | dTs\|dTs\|5mCs\|dTs\|5mCs\|5mCs\|5mCs\|o | | | | |
| | As\|oGs\|oCs\|oAs\|oAs\|oC] | | | | |
| 210 | [oUs\|oGs\|oAp\|oUs\|oCs\|5mCs\|dTs\|dGs\| | 36.27817 | 23.59247 | 1.9107749 | 1.785808 |
| | dTs\|dTs\|5mCs\|dTs\|5mCs\|5mCs\|5mCs\|o | | | | |
| | As\|oGs\|oCs\|oAs\|oAs\|oC] | | | | |
| 211 | [oUs\|oGs\|oAs\|oUp\|oCs\|5mCs\|dTs\|dGs\| | 40.23379 | 26.02175 | 3.7752339 | 0.579919 |
| | dTs\|dTs\|5mCs\|dTs\|5mCs\|5mCs\|5mCs\|o | | | | |
| | As\|oGs\|oCs\|oAs\|oAs\|oC] | | | | |
| 212 | [oUs\|oGs\|oAp\|oUp\|oCs\|5mCs\|dTs\|dGs\| | 38.73671 | 25.57041 | 2.1198962 | 0.563413 |
| | dTs\|dTs\|5mCs\|dTs\|5mCs\|5mCs\|5mCs\|o | | | | |
| | As\|oGs\|oCs\|oAs\|oAs\|oC] | | | | |
| 213 | [oUs\|oGs\|oAs\|oUs\|oCs\|5mCs\|dTs\|dGs\| | 35.00403 | 27.29374 | 0.97998025 | 2.42817 |
| | dTs\|dTs\|5mCs\|dTs\|5mCs\|5mCs\|5mCs\|o | | | | |
| | As\|oGp\|oCs\|oAs\|oAs\|oC] | | | | |
| 214 | [oUs\|oGs\|oAs\|oUs\|oCs\|5mCs\|dTs\|dGs\| | 35.15246 | 27.73562 | 3.7541248 | 1.408177 |
| | dTs\|dTs\|5mCs\|dTs\|5mCs\|5mCs\|5mCs\|o | | | | |
| | Ap\|oGs\|oCs\|oAs\|oAs\|oC] | | | | |
| 215 | [oUs\|oGs\|oAs\|oUs\|oCs\|5mCs\|dTs\|dGs\| | 39.43029 | 24.36535 | 0.35631305 | 2.03814 |
| | dTs\|dTs\|5mCs\|dTs\|5mCs\|5mCs\|5mCs\|o | | | | |
| | Ap\|oGp\|oCs\|oAs\|oAs\|oC] | | | | |
| 216 | [oUs\|oGs\|oAp\|oUs\|oCs\|5mCs\|dTs\|dGs\| | 38.4821 | 24.80134 | 2.7737859 | 0.915852 |
| | dTs\|dTs\|5mCs\|dTs\|5mCs\|5mCs\|5mCs\|o | | | | |
| | As\|oGp\|oCs\|oAs\|oAs\|oC] | | | | |
| 217 | [oUs\|oGs\|oAp\|oUs\|oCs\|5mCs\|dTs\|dGs\| | 38.86511 | 22.24657 | 1.93345215 | 1.178782 |
| | dTs\|dTs\|5mCs\|dTs\|5mCs\|5mCs\|5mCs\|o | | | | |
| | Ap\|oGs\|oCs\|oAs\|oAs\|oC] | | | | |
| 218 | [oUs\|oGs\|oAs\|oUp\|oCs\|5mCs\|dTs\|dGs\| | 40.69835 | 26.7572 | 2.8425562 | 2.117941 |
| | dTs\|dTs\|5mCs\|dTs\|5mCs\|5mCs\|5mCs\|o | | | | |
| | As\|oGp\|oCs\|oAs\|oAs\|oC] | | | | |
| 219 | [oUs\|oGs\|oAs\|oUp\|oCs\|5mCs\|dTs\|dGs\| | 39.97252 | 26.31039 | 1.18627875 | 1.508108 |
| | dTs\|dTs\|5mCs\|dTs\|5mCs\|5mCs\|5mCs\|o | | | | |
| | Ap\|oGs\|oCs\|oAs\|oAs\|oC] | | | | |
| 220 | [oUs\|oGs\|oAp\|oUs\|oCs\|5mCs\|dTs\|dGs\| | 40.94544 | 24.39298 | 2.69640305 | 1.085099 |
| | dTs\|dTs\|5mCs\|dTs\|5mCs\|5mCs\|5mCs\|o | | | | |
| | Ap\|oGp\|oCs\|oAs\|oAs\|oC] | | | | |
| 221 | [oUs\|oGs\|oAs\|oUp\|oCs\|5mCs\|dTs\|dGs\| | 39.59504 | 26.25572 | 2.87140485 | 1.887767 |
| | dTs\|dTs\|5mCs\|dTs\|5mCs\|5mCs\|5mCs\|o | | | | |
| | Ap\|oGp\|oCs\|oAs\|oAs\|oC] | | | | |
| 222 | [oUs\|oGs\|oAp\|oUp\|oCs\|5mCs\|dTs\|dGs | 34.32736 | 25.9504 | 1.1160027 | 1.377325 |
| | dTs\|dTs\|5mCs\|dTs\|5mCs\|5mCs\|5mCs\|o | | | | |
| | As\|oGp\|oCs\|oAs\|oAs\|oC] | | | | |
| 223 | [oUs\|oGs\|oAp\|oUp\|oCs\|5mCs\|dTs\|dGs\| | 40.65856 | 25.62251 | 1.38290445 | 2.322099 |
| | dTs\|dTs\|5mCs\|dTs\|5mCs\|5mCs\|5mCs\|o | | | | |
| | Ap\|oGs\|oCs\|oAs\|oAs\|oC] | | | | |
| 224 | [oUs\|oGs\|oAp\|oUp\|oCs\|5mCs\|dTs\|dGs\| | 38.35045 | 24.81935 | 1.78788745 | 5.896552 |
| | dTs\|dTs\|5mCs\|dTs\|5mCs\|5mCs\|5mCs\|o | | | | |
| | Ap\|oGp\|oCs\|oAs\|oAs\|oC] | | | | |
| 225 | [oUs\|oGs\|oAs\|oUs\|oCs\|5mCs\|dTs\|dGs\| | 38.20957 | 22.48775 | 0.8970076 | 2.000011 |
| | dTs\|dTs\|5mCs\|dTs\|5mCs\|5mCs\|5mCs\|o | | | | |
| | As\|oGs\|oCs\|oAs\|oAs\|oCs\|oA] | | | | |
| 226 | [oUs\|oGs\|oAp\|oUs\|oCs\|5mCs\|dTs\|dGs\| | 33.35769 | 27.38264 | 1.58936325 | 1.493528 |
| | dTs\|dTs\|5mCs\|dTs\|5mCs\|5mCs\|5mCs\|o | | | | |
| | As\|oGs\|oCs\|oAs\|oAs\|oCs\|oA] | | | | |
| 227 | [oUs\|oGs\|oAs\|oUp\|oCs\|5mCs\|dTs\|dGs\| | 30.72978 | 24.56478 | 1.12605755 | 0.803606 |
| | dTs\|dTs\|5mCs\|dTs\|5mCs\|5mCs\|5mCs\|o | | | | |
| | As\|oGs\|oCs\|oAs\|oAs\|oCs\|oA] | | | | |
| 228 | [oUs\|oGs\|oAp\|oUp\|oCs\|5mCs\|dTs\|dGs\| | 41.09495 | 22.65474 | 2.4141379 | 1.912592 |
| | dTs\|dTs\|5mCs\|dTs\|5mCs\|5mCs\|5mCs\|o | | | | |
| | As\|oGs\|oCs\|oAs\|oAs\|oCs\|oA] | | | | |
| 229 | [oUs\|oGs\|oAs\|oUs\|oCs\|5mCs\|dTs\|dGs\| | 36.5236 | 24.84599 | 1.6011969 | 1.293078 |
| | dTs\|dTs\|5mCs\|dTs\|5mCs\|5mCs\|5mCs\|o | | | | |
| | As\|oGp\|oCs\|oAs\|oAs\|oCs\|oA] | | | | |
| 230 | [oUs\|oGs\|oAs\|oUs\|oCs\|5mCs\|dTs\|dGs\| | 34.54957 | 26.61084 | 1.41653565 | 1.233035 |
| | dTs\|dTs\|5mCs\|dTs\|5mCs\|5mCs\|5mCs\|o | | | | |
| | Ap\|oGs\|oCs\|oAs\|oAs\|oCs\|oA] | | | | |
| 231 | [oUs\|oGs\|oAs\|oUs\|oCs\|5mCs\|dTs\|dGs\| | 46.03036 | 34.13517 | 3.10815325 | 2.788523 |
| | dTs\|dTs\|5mCs\|dTs\|5mCs\|5mCs\|5mCs\|o | | | | |
| | Ap\|oGp\|oCs\|oAs\|oAs\|oCs\|oA] | | | | |
| 232 | [oUs\|oGs\|oAp\|oUs\|oCs\|5mCs\|dTs\|dGs\| | 35.25195 | 23.45108 | 1.14545295 | 0.875176 |
| | dTs\|dTs\|5mCs\|dTs\|5mCs\|5mCs\|5mCs\|o | | | | |
| | As\|oGp\|oCs\|oAs\|oAs\|oCs\|oA] | | | | |
| 233 | [oUs\|oGs\|oAp\|oUs\|oCs\|5mCs\|dTs\|dGs\| | 39.59948 | 27.15972 | 1.1364332 | 1.671372 |
| | dTs\|dTs\|5mCs\|dTs\|5mCs\|5mCs\|5mCs\|o | | | | |
| | Ap\|oGs\|oCs\|oAs\|oAs\|oCs\|oA] | | | | |
| 234 | [oUs\|oGs\|oAs\|oUp\|oCs\|5mCs\|dTs\|dGs\| | 29.84471 | 24.57841 | 1.1966453 | 0.580119 |
| | dTs\|dTs\|5mCs\|dTs\|5mCs\|5mCs\|5mCs\|o | | | | |
| | As\|oGp\|oCs\|oAs\|oAs\|oCs\|oA] | | | | |
| 235 | [oUs\|oGs\|oAs\|oUp\|oCs\|5mCs\|dTs\|dGs\| | 35.45558 | 25.43503 | 3.1614937 | 2.281794 |
| | dTs\|dTs\|5mCs\|dTs\|5mCs\|5mCs\|5mCs\|o | | | | |
| | Ap\|oGs\|oCs\|oAs\|oAs\|oCs\|oA] | | | | |
| 236 | [oUs\|oGs\|oAp\|oUs\|oCs\|5mCs\|dTs\|dGs\| | 33.98368 | 25.36152 | 2.2232316 | 0.753668 |
| | dTs\|dTs\|5mCs\|dTs\|5mCs\|5mCs\|5mCs\|o | | | | |
| | Ap\|oGp\|oCs\|oAs\|oAs\|oCs\|oA] | | | | |
| 237 | [oUs\|oGs\|oAs\|oUp\|oCs\|5mCs\|dTs\|dGs\| | 35.20734 | 26.34259 | 2.31304315 | 1.232096 |
| | dTs\|dTs\|5mCs\|dTs\|5mCs\|5mCs\|5mCs\|o | | | | |
| | Ap\|oGp\|oCs\|oAs\|oAs\|oCs\|oA] | | | | |
| 238 | [oUs\|oGs\|oAp\|oUp\|oCs\|5mCs\|dTs\|dGs\| | 34.15353 | 22.53496 | 1.9007468 | 1.47269 |
| | dTs\|dTs\|5mCs\|dTs\|5mCs\|5mCs\|5mCs\|o | | | | |
| | As\|oGp\|oCs\|oAs\|oAs\|oCs\|oA] | | | | |
| 239 | [oUs\|oGs\|oAp\|oUp\|oCs\|5mCs\|dTs\|dGs\| | 34.30311 | 25.10675 | 3.04473305 | 1.325174 |
| | dTs\|dTs\|5mCs\|dTs\|5mCs\|5mCs\|5mCs\|o | | | | |
| | Ap\|oGs\|oCs\|oAs\|oAs\|oCs\|oA] | | | | |
| 240 | [oUs\|oGs\|oAp\|oUp\|oCs\|5mCs\|dTs\|dGs\| | 32.7831 | 26.79337 | 0.780257 | 1.496388 |
| | dTs\|dTs\|5mCs\|dTs\|5mCs\|5mCs\|5mCs\|o | | | | |
| | Ap\|oGp\|oCs\|oAs\|oAs\|oCs\|oA] | | | | |
| 241 | [oUs\|oGs\|oAs\|oUs\|oCs\|5mCs\|dTs\|dGs\| | 36.83221 | 25.878 | 1.45957465 | 1.202151 |
| | dTs\|dTs\|5mCs\|dTs\|5mCs\|5mCs\|5mCs\|o | | | | |
| | As\|oGs\|oCs\|oAs\|oAs\|oCs\|oAs\|oC] | | | | |
| 242 | [oUs\|oGs\|oAp\|oUs\|oCs\|5mCs\|dTs\|dGs\| | 35.39125 | 26.34255 | 2.0733604 | 1.939231 |
| | dTs\|dTs\|5mCs\|dTs\|5mCs\|5mCs\|5mCs\|o | | | | |
| | As\|oGs\|oCs\|oAs\|oAs\|oCs\|oAs\|oC] | | | | |
| 243 | [oUs\|oGs\|oAs\|oUp\|oCs\|5mCs\|dTs\|dGs\| | 31.25962 | 26.87411 | 1.1648233 | 1.9468 |
| | dTs\|dTs\|5mCs\|dTs\|5mCs\|5mCs\|5mCs\|o | | | | |
| | As\|oGs\|oCs\|oAs\|oAs\|oCs\|oAs\|oC] | | | | |
| 244 | [oUs\|oGs\|oAp\|oUp\|oCs\|5mCs\|dTs\|dGs\| | 33.7126 | 20.71987 | 2.985748 | 2.347571 |
| | dTs\|dTs\|5mCs\|dTs\|5mCs\|5mCs\|5mCs\|o | | | | |
| | As\|oGs\|oCs\|oAs\|oAs\|oCs\|oAs\|oC] | | | | |
| 245 | [oUs\|oGs\|oAs\|oUs\|oCs\|5mCs\|dTs\|dGs\| | 37.45041 | 27.92649 | 2.2914196 | 1.775925 |
| | dTs\|dTs\|5mCs\|dTs\|5mCs\|5mCs\|5mCs\|o | | | | |
| | As\|oGp\|oCs\|oAs\|oAs\|oCs\|oAs\|oC] | | | | |
| 246 | [oUs\|oGs\|oAs\|oUs\|oCs\|5mCs\|dTs\|dGs\| | 37.40703 | 27.91025 | 0.37894275 | 2.801681 |
| | dTs\|dTs\|5mCs\|dTs\|5mCs\|5mCs\|5mCs\|o | | | | |
| | Ap\|oGs\|oCs\|oAs\|oAs\|oCs\|oAs\|oC] | | | | |
| 247 | [oUs\|oGs\|oAs\|oUs\|oCs\|5mCs\|dTs\|dGs\| | 44.1762 | 24.73343 | 3.02169625 | 0.96668 |
| | dTs\|dTs\|5mCs\|dTs\|5mCs\|5mCs\|5mCs\|o | | | | |
| | Ap\|oGp\|oCs\|oAs\|oAs\|oCs\|oAs\|oC] | | | | |
| 248 | [oUs\|oGs\|oAp\|oUs\|oCs\|5mCs\|dTs\|dGs\| | 38.30889 | 23.31712 | 1.4411529 | 1.489325 |
| | dTs\|dTs\|5mCs\|dTs\|5mCs\|5mCs\|5mCs\|o | | | | |
| | As\|oGp\|oCs\|oAs\|oAs\|oCs\|oAs\|oC] | | | | |
| 249 | [oUs\|oGs\|oAp\|oUs\|oCs\|5mCs\|dTs\|dGs\| | 38.31735 | 24.56753 | 2.5346059 | 1.77217 |
| | dTs\|dTs\|5mCs\|dTs\|5mCs\|5mCs\|5mCs\|o | | | | |
| | Ap\|oGs\|oCs\|oAs\|oAs\|oCs\|oAs\|oC] | | | | |
| 250 | [oUs\|oGs\|oAs\|oUp\|oCs\|5mCs\|dTs\|dGs\| | 35.17502 | 26.1372 | 1.8152499 | 2.083902 |
| | dTs\|dTs\|5mCs\|dTs\|5mCs\|5mCs\|5mCs\|o | | | | |
| | As\|oGp\|oCs\|oAs\|oAs\|oCs\|oAs\|oC] | | | | |
| 25 | [oUs\|oGs\|oAs\|oUp\|oCs\|5mCs\|dTs\|dGs\| | 36.24617 | 24.66592 | 1.38376155 | 0.821536 |
| | dTs\|dTs\|5mCs\|dTs\|5mCs\|5mCs\|5mCs\|o | | | | |
| | Ap\|oGs\|oCs\|oAs\|oAs\|oCs\|oAs\|oC] | | | | |
| 252 | [oUs\|oGs\|oAp\|oUs\|oCs\|5mCs\|dTs\|dGs\| | 38.74019 | 26.77353 | 1.6343742 | 1.579313 |
| | dTs\|dTs\|5mCs\|dTs\|5mCs\|5mCs\|5mCs\|o | | | | |
| | Ap\|oGp\|oCs\|oAs\|oAs\|oCs\|oAs\|oC] | | | | |
| 253 | [oUs\|oGs\|oAs\|oUp\|oCs\|5mCs\|dTs\|dGs\| | 38.33121 | 30.17397 | 1.0889853 | 2.797118 |
| | dTs\|dTs\|5mCs\|dTs\|5mCs\|5mCs\|5mCs\|o | | | | |
| | Ap\|oGp\|oCs\|oAs\|oAs\|oCs\|oAs\|oC] | | | | |
| 254 | [oUs\|oGs\|oAp\|oUp\|oCs\|5mCs\|dTs\|dGs\| | 44.32188 | 27.49314 | 2.67802585 | 1.31818 |
| | dTs\|dTs\|5mCs\|dTs\|5mCs\|5mCs\|5mCs\|o | | | | |
| | As\|oGp\|oCs\|oAs\|oAs\|oCs\|oAs\|oC] | | | | |
| 255 | [oUs\|oGs\|oAp\|oUp\|oCs\|5mCs\|dTs\|dGs\| | 38.70488 | 30.24362 | 2.4257059 | 2.598832 |
| | dTs\|dTs\|5mCs\|dTs\|5mCs\|5mCs\|5mCs\|o | | | | |
| | Ap\|oGs\|oCs\|oAs\|oAs\|oCs\|oAs\|oC] | | | | |
| 256 | [oUs\|oGs\|oAp\|oUp\|oCs\|5mCs\|dTs\|dGs\| | 43.28825 | 28.25934 | 3.05476895 | 1.497906 |
| | dTs\|dTs\|5mCs\|dTs\|5mCs\|5mCs\|5mCs\|o | | | | |
| | Ap\|oGp\|oCs\|oAs\|oAs\|oCs\|oAs\|oC] | | | | |
| 257 | [oUs\|oGs\|oAs\|oUs\|oCs\|5mCs\|dTs\|dGs\| | 45.77041 | 30.01057 | 2.41725105 | 2.688685 |
| | dTs\|dTs\|5mCs\|dTs\|5mCs\|5mCs\|5mCs\|d | | | | |
| | As\|oGs\|oCs\|oAs\|oAs\|oC] | | | | |
| 258 | [oUs\|oGs\|oAp\|oUs\|oCs\|5mCs\|dTs\|dGs | 37.50516 | 29.19368 | 3.2609215 | 1.306107 |
| | dTs\|dTs\|5mCs\|dTs\|5mCs\|5mCs\|5mCs\|d | | | | |
| | As\|oGs\|oCs\|oAs\|oAs\|oC] | | | | |
| 259 | [oUs\|oGs\|oAs\|oUp\|oCs\|5mCs\|dTs\|dGs\| | 37.61875 | 28.41851 | 4.79014865 | 1.914106 |
| | dTs\|dTs\|5mCs\|dTs\|5mCs\|5mCs\|5mCs\|d | | | | |
| | As\|oGs\|oCs\|oAs\|oAs\|oC] | | | | |
| 260 | [oUs\|oGs\|oAp\|oUp\|oCs\|5mCs\|dTs\|dGs\| | 39.57219 | 25.95941 | 1.0745636 | 2.611124 |
| | dTs\|dTs\|5mCs\|dTs\|5mCs\|5mCs\|5mCs\|d | | | | |
| | As\|oGs\|oCs\|oAs\|oAs\|oC] | | | | |
| 261 | [oUs\|oGs\|oAs\|oUs\|oCs\|5mCs\|dTs\|dGs\| | 40.85794 | 29.04845 | 2.10254735 | 0.910299 |
| | dTs\|dTs\|5mCs\|dTs\|5mCs\|5mCs\|5mCs\|d | | | | |
| | As\|oGp\|oCs\|oAs\|oAs\|oC] | | | | |
| 262 | [oUs\|oGs\|oAs\|oUs\|oCs\|5mCs\|dTs\|dGs\| | 48.57987 | 31.96517 | 3.30537215 | 3.77167 |
| | dTs\|dTs\|5mCs\|dTs\|5mCs\|5mCs\|5mCs\|d | | | | |
| | Ap\|oGs\|oCs\|oAs\|oAs\|oC] | | | | |
| 263 | [oUs\|oGs\|oAs\|oUs\|oCs\|5mCs\|dTs\|dGs\| | 42.44673 | 32.35736 | 1.9875946 | 2.639212 |
| | dTs\|dTs\|5mCs\|dTs\|5mCs\|5mCs\|5mCs\|d | | | | |
| | Ap\|oGp\|oCs\|oAs\|oAs\|oC] | | | | |
| 264 | [oUs\|oGs\|oAp\|oUs\|oCs\|5mCs\|dTs\|dGs\| | 43.82073 | 27.45321 | 2.0919192 | 1.819505 |
| | dTs\|dTs\|5mCs\|dTs\|5mCs\|5mCs\|5mCs\|d | | | | |
| | As\|oGp\|oCs\|oAs\|oAs\|oC] | | | | |
| 265 | [oUs\|oGs\|oAp\|oUs\|oCs\|5mCs\|dTs\|dGs\| | 40.81426 | 34.44821 | 3.2274531 | 3.246932 |
| | dTs\|dTs\|5mCs\|dTs\|5mCs\|5mCs\|5mCs\|d | | | | |
| | Ap\|oGs\|oCs\|oAs\|oAs\|oC] | | | | |
| 266 | [oUs\|oGs\|oAs\|oUp\|oCs\|5mCs\|dTs\|dGs\| | 44.58453 | 32.33173 | 4.3552897 | 2.179554 |
| | dTs\|dTs\|5mCs\|dTs\|5mCs\|5mCs\|5mCs\|d | | | | |
| | As\|oGp\|oCs\|oAs\|oAs\|oC] | | | | |
| 267 | [oUs\|oGs\|oAs\|oUp\|oCs\|5mCs\|dTs\|dGs\| | 45.58382 | 28.39511 | 0.9009167 | 1.911982 |
| | dTs\|dTs\|5mCs\|dTs\|5mCs\|5mCs\|5mCs\|d | | | | |
| | Ap\|oGs\|oCs\|oAs\|oAs\|oC] | | | | |
| 268 | [oUs\|oGs\|oAp\|oUs\|oCs\|5mCs\|dTs\|dGs\| | 42.5512 | 29.79543 | 1.03173485 | 1.860662 |
| | dTs\|dTs\|5mCs\|dTs\|5mCs\|5mCs\|5mCs\|d | | | | |
| | Ap\|oGp\|oCs\|oAs\|oAs\|oC] | | | | |
| 269 | [oUs\|oGs\|oAs\|oUp\|oCs\|5mCs\|dTs\|dGs\| | 44.6866 | 30.01983 | 2.5231542 | 4.434048 |
| | dTs\|dTs\|5mCs\|dTs\|5mCs\|5mCs\|5mCs\|d | | | | |
| | Ap\|oGp\|oCs\|oAs\|oAs\|oC] | | | | |
| 270 | [oUs\|oGs\|oAp\|oUp\|oCs\|5mCs\|dTs\|dGs\| | 38.02311 | 32.80756 | 3.4793287 | 4.749247 |
| | dTs\|dTs\|5mCs\|dTs\|5mCs\|5mCs\|5mCs\|d | | | | |
| | As\|oGp\|oCs\|oAs\|oAs\|oC] | | | | |
| 271 | [oUs\|oGs\|oAp\|oUp\|oCs\|5mCs\|dTs\|dGs\| | 48.88263 | 27.15217 | 3.5851377 | 1.816405 |
| | dTs\|dTs\|5mCs\|dTs\|5mCs\|5mCs\|5mCs\|d | | | | |
| | Ap\|oGs\|oCs\|oAs\|oAs\|oC] | | | | |
| 272 | [oUs\|oGs\|oAp\|oUp\|oCs\|5mCs\|dTs\|dGs\| | 46.69598 | 30.73665 | 2.0490884 | 2.855452 |
| | dTs\|dTs\|5mCs\|dTs\|5mCs\|5mCs\|5mCs\|d | | | | |
| | Ap\|oGp\|oCs\|oAs\|oAs\|oC] | | | | |
| 273 | [oUs\|oGs\|oAs\|oUs\|oCs\|5mCs\|dTs\|dGs\| | 42.70873 | 30.68745 | 3.65148505 | 3.255575 |
| | dTs\|dTs\|5mCs\|dTs\|5mCs\|5mCs\|5mCs\|d | | | | |
| | As\|dGs\|oCs\|oAs\|oAs\|oCs\|oA] | | | | |
| 274 | [oUs\|oGs\|oAp\|oUs\|oCs\|5mCs\|dTs\|dGs\| | 40.32396 | 26.35517 | 3.335113 | 2.701905 |
| | dTs\|dTs\|5mCs\|dTs\|5mCs\|5mCs\|5mCs\|d | | | | |
| | As\|dGs\|oCs\|oAs\|oAs\|oCs\|oA] | | | | |
| 275 | [oUs\|oGs\|oAs\|oUp\|oCs\|5mCs\|dTs\|dGs\| | 40.74281 | 29.94494 | 0.6334559 | 2.557136 |
| | dTs\|dTs\|5mCs\|dTs\|5mCs\|5mCs\|5mCs\|d | | | | |
| | As\|dGs\|oCs\|oAs\|oAs\|oCs\|oA] | | | | |
| 276 | [oUs\|oGs\|oAp\|oUp\|oCs\|5mCs\|dTs\|dGs\| | 40.29221 | 27.33965 | 0.8763938 | 2.574619 |
| | dTs\|dTs\|5mCs\|dTs\|5mCs\|5mCs\|5mCs\|d | | | | |
| | As\|dGs\|oCs\|oAs\|oAs\|oCs\|oA] | | | | |
| 277 | [oUs\|oGs\|oAs\|oUs\|oCs\|5mCs\|dTs\|dGs\| | 47.44893 | 29.86629 | 2.4738361 | 2.55434 |
| | dTs\|dTs\|5mCs\|dTs\|5mCs\|5mCs\|5mCs\|d | | | | |
| | As\|dGp\|oCs\|oAs\|oAs\|oCs\|oA] | | | | |
| 278 | [oUs\|oGs\|oAs\|oUs\|oCs\|5mCs\|dTs\|dGs\| | 46.17489 | 33.16617 | 3.1173163 | 1.327764 |
| | dTs\|dTs\|5mCs\|dTs\|5mCs\|5mCs\|5mCs\|d | | | | |
| | Ap\|dGs\|oCs\|oAs\|oAs\|oCs\|oA] | | | | |
| 279 | [oUs\|oGs\|oAs\|oUs\|oCs\|5mCs\|dTs\|dGs\| | 46.58545 | 33.39568 | 3.2287529 | 1.864188 |
| | dTs\|dTs\|5mCs\|dTs\|5mCs\|5mCs\|5mCs\|d | | | | |
| | Ap\|dGp\|oCs\|oAs\|oAs\|oCs\|oA] | | | | |
| 280 | [oUs\|oGs\|oAp\|oUs\|oCs\|5mCs\|dTs\|dGs\| | 43.27573 | 27.28916 | 2.31554215 | 1.262593 |
| | dTs\|dTs\|5mCs\|dTs\|5mCs\|5mCs\|5mCs\|d | | | | |
| | As\|dGp\|oCs\|oAs\|oAs\|oCs\|oA] | | | | |
| 281 | [oUs\|oGs\|oAp\|oUs\|oCs\|5mCs\|dTs\|dGs\| | 47.10235 | 32.83978 | 1.53071395 | 4.229922 |
| | dTs\|dTs\|5mCs\|dTs\|5mCs\|5mCs\|5mCs\|d | | | | |
| | Ap\|dGs\|oCs\|oAs\|oAs\|oCs\|oA] | | | | |
| 282 | [oUs\|oGs\|oAs\|oUp\|oCs\|5mCs\|dTs\|dGs\| | 47.70091 | 29.60736 | 3.7604108 | 2.052869 |
| | dTs\|dTs\|5mCs\|dTs\|5mCs\|5mCs\|5mCs\|d | | | | |
| | As\|dGp\|oCs\|oAs\|oAs\|oCs\|oA] | | | | |
| 283 | [oUs\|oGs\|oAs\|oUp\|oCs\|5mCs\|dTs\|dGs\| | 49.28544 | 34.66856 | 1.59176345 | 3.017275 |
| | dTs\|dTs\|5mCs\|dTs\|5mCs\|5mCs\|5mCs\|d | | | | |
| | Ap\|dGs\|oCs\|oAs\|oAs\|oCs\|oA] | | | | |
| 284 | [oUs\|oGs\|oAp\|oUs\|oCs\|5mCs\|dTs\|dGs\| | 49.84421 | 32.28262 | 4.79490775 | 5.858741 |
| | dTs\|dTs\|5mCs\|dTs\|5mCs\|5mCs\|5mCs\|d | | | | |
| | Ap\|dGp\|oCs\|oAs\|oAs\|oCs\|oA] | | | | |
| 285 | [oUs\|oGs\|oAs\|oUp\|oCs\|5mCs\|dTs\|dGs\| | 34.59283 | 22.87145 | 3.0164757 | 2.082604 |
| | dTs\|dTs\|5mCs\|dTs\|5mCs\|5mCs\|5mCs\|d | | | | |
| | Ap\|dGp\|oCs\|oAs\|oAs\|oCs\|oA] | | | | |
| 286 | [oUs\|oGs\|oAp\|oUp\|oCs\|5mCs\|dTs\|dGs\| | 39.38162 | 26.12473 | 1.8902058 | 1.570875 |
| | dTs\|dTs\|5mCs\|dTs\|5mCs\|5mCs\|5mCs\|d | | | | |
| | As\|dGp\|oCs\|oAs\|oAs\|oCs\|oA] | | | | |
| 287 | [oUs\|oGs\|oAp\|oUp\|oCs\|5mCs\|dTs\|dGs\| | 50.21733 | 30.06642 | 3.10898985 | 1.234999 |
| | dTs\|dTs\|5mCs\|dTs\|5mCs\|5mCs\|5mCs\|d | | | | |
| | Ap\|dGs\|oCs\|oAs\|oAs\|oCs\|oA] | | | | |
| 288 | [oUs\|oGs\|oAp\|oUp\|oCs\|5mCs\|dTs\|dGs\| | 50.69014 | 32.61033 | 2.79327395 | 1.497337 |
| | dTs\|dTs\|5mCs\|dTs\|5mCs\|5mCs\|5mCs\|d | | | | |
| | Ap\|dGp\|oCs\|oAs\|oAs\|oCs\|oA] | | | | |
| 289 | [oUs\|oUs\|oGs\|oAs\|oUs\|5mCs\|5mCs\|dT | 65.03387 | 39.71818 | 3.2453458 | 1.232538 |
| | s\|dGs\|dTs\|dTs\|5mCs\|dTs\|5mCs\|5mCs\|o | | | | |
| | Cs\|oAs\|oGs\|oCs\|oA] | | | | |
| 290 | [oUs\|oUs\|oGp\|oAs\|oUs\|5mCs\|5mCs\|dT | 61.89021 | 44.17769 | 3.0819054 | 2.818603 |
| | s\|dGs\|dTs\|dTs\|5mCs\|dTs\|5mCs\|5mCs\|o | | | | |
| | Cs\|oAs\|oGs\|oCs\|oA] | | | | |
| 291 | [oUs\|oUs\|oGs\|oAp\|oUs\|5mCs\|5mCs\|dT | 70.81215 | 39.23055 | 5.9432651 | 2.293842 |
| | s\|dGs\|dTs\|dTs\|5mCs\|dTs\|5mCs\|5mCs\|o | | | | |
| | Cs\|oAs\|oGs\|oCs\|oA] | | | | |
| 292 | [oUs\|oUs\|oGp\|oAp\|oUs\|5mCs\|5mCs\|dT | 64.28917 | 35.27306 | 4.4374966 | 2.084275 |
| | s\|dGs\|dTs\|dTs\|5mCs\|dTs\|5mCs\|5mCs\|o | | | | |
| | Cs\|oAs\|oGs\|oCs\|oA] | | | | |
| 293 | [oUs\|oUs\|oGs\|oAs\|oUs\|5mCs\|5mCs\|dT | 75.30924 | 41.75287 | 6.3677188 | 3.571926 |
| | s\|dGs\|dTs\|dTs\|5mCs\|dTs\|5mCs\|5mCs\|o | | | | |
| | Cs\|oAp\|oGs\|oCs\|oA] | | | | |
| 294 | [oUs\|oUs\|oGs\|oAs\|oUs\|5mCs\|5mCs\|dT | 72.21538 | 40.63977 | 9.45384065 | 2.395694 |
| | s\|dGs\|dTs\|dTs\|5mCs\|dTs\|5mCs\|5mCs\|o | | | | |
| | Cp\|oAs\|oGs\|oCs\|oA] | | | | |
| 295 | [oUs\|oUs\|oGs\|oAs\|oUs\|5mCs\|5mCs\|dT | 65.43466 | 40.60514 | 4.9966345 | 4.010523 |
| | s\|dGs\|dTs\|dTs\|5mCs\|dTs\|5mCs\|5mCs\|o | | | | |
| | Cp\|oAp\|oGs\|oCs\|oA] | | | | |
| 296 | [oUs\|oUs\|oGp\|oAs\|oUs\|5mCs\|5mCs\|dT | 70.68577 | 41.62804 | 4.61698135 | 0.639112 |
| | s\|dGs\|dTs\|dTs\|5mCs\|dTs\|5mCs\|5mCs\|o | | | | |
| | Cs\|oAp\|oGs\|oCs\|oA] | | | | |
| 297 | [oUs\|oUs\|oGp\|oAs\|oUs\|5mCs\|5mCs\|dT | 68.70839 | 43.19171 | 4.0611799 | 2.66127 |
| | s\|dGs\|dTs\|dTs\|5mCs\|dTs\|5mCs\|5mCs\|o | | | | |
| | Cp\|oAs\|oGs\|oCs\|oA] | | | | |
| 298 | [oUs\|oUs\|oGs\|oAp\|oUs\|5mCs\|5mCs\|dT | 68.74235 | 42.8638 | 6.63886525 | 2.69242 |
| | s\|dGs\|dTs\|dTs\|5mCs\|dTs\|5mCs\|5mCs\|o | | | | |
| | Cs\|oAp\|oGs\|oCs\|oA] | | | | |
| 299 | [oUs\|oUs\|oGs\|oAp\|oUs\|5mCs\|5mCs\|dT | 65.56432 | 41.13655 | 2.8508521 | 1.098556 |
| | s\|dGs\|dTs\|dTs\|5mCs\|dTs\|5mCs\|5mCs\|o | | | | |
| | Cp\|oAs\|oGs\|oCs\|oA] | | | | |
| 300 | [oUs\|oUs\|oGp\|oAs\|oUs\|5mCs\|5mCs\|dT | 73.25913 | 44.19704 | 0.3218877 | 3.006132 |
| | s\|dGs\|dTs\|dTs\|5mCs\|dTs\|5mCs\|5mCs\|o | | | | |
| | Cp\|oAp\|oGs\|oCs\|oA] | | | | |
| 301 | [oUs\|oUs\|oGs\|oAp\|oUs\|5mCs\|5mCs\|dT | 66.80031 | 44.33691 | 5.58574735 | 2.190286 |
| | s\|dGs\|dTs\|dTs\|5mCs\|dTs\|5mCs\|5mCs\|o | | | | |
| | Cp\|oAp\|oGs\|oCs\|oA] | | | | |
| 302 | [oUs\|oUs\|oGp\|oAp\|oUs\|5mCs\|5mCs\|dT | 67.79988 | 43.25911 | 3.47248465 | 2.245861 |
| | s\|dGs\|dTs\|dTs\|5mCs\|dTs\|5mCs\|5mCs\|o | | | | |
| | Cs\|oAp\|oGs\|oCs\|oA] | | | | |
| 303 | [oUs\|oUs\|oGp\|oAp\|oUs\|5mCs\|5mCs\|dT | 69.0764 | 39.9134 | 2.2580266 | 2.514186 |
| | s\|dGs\|dTs\|dTs\|5mCs\|dTs\|5mCs\|5mCs\|o | | | | |
| | Cp\|oAs\|oGs\|oCs\|oA] | | | | |
| 304 | [oUs\|oUs\|oGp\|oAp\|oUs\|5mCs\|5mCs\|dT | 68.85722 | 43.2033 | 2.66821415 | 1.577173 |
| | s\|dGs\|dTs\|dTs\|5mCs\|dTs\|5mCs\|5mCs\|o | | | | |
| | Cp\|oAp\|oGs\|oCs\|oA] | | | | |
| 305 | [oUs\|oUs\|oGs\|oAs\|oUs\|5mCs\|5mCs\|dT | 64.32531 | 36.98857 | 6.6048679 | 1.862941 |
| | s\|dGs\|dTs\|dTs\|5mCs\|dTs\|5mCs\|5mCs\|o | | | | |
| | Cs\|oAs\|oGs\|oCs\|oAs\|oA] | | | | |
| 306 | [oUs\|oUs\|oGp\|oAs\|oUs\|5mCs\|5mCs\|dT | 79.43795 | 38.19117 | 10.61625475 | 3.700974 |
| | s\|dGs\|dTs\|dTs\|5mCs\|dTs\|5mCs\|5mCs\|o | | | | |
| | Cs\|oAs\|oGs\|oCs\|oAs\|oA] | | | | |
| 307 | [oUs\|oUs\|oGs\|oAp\|oUs\|5mCs\|5mCs\|dT | 65.05429 | 35.73871 | 5.07137215 | 2.026283 |
| | s\|dGs\|dTs\|dTs\|5mCs\|dTs\|5mCs\|5mCs\|o | | | | |
| | Cs\|oAs\|oGs\|oCs\|oAs\|oA] | | | | |
| 308 | [oUs\|oUs\|oGp\|oAp\|oUs\|5mCs\|5mCs\|dT | 67.69046 | 37.80261 | 3.3970135 | 1.359287 |
| | s\|dGs\|dTs\|dTs\|5mCs\|dTs\|5mCs\|5mCs\|o | | | | |
| | Cs\|oAs\|oGs\|oCs\|oAs\|oA] | | | | |
| 309 | [oUs\|oUs\|oGs\|oAs\|oUs\|5mCs\|5mCs\|dT | 72.9064 | 39.22807 | 2.6173443 | 1.983172 |
| | s\|dGs\|dTs\|dTs\|5mCs\|dTs\|5mCs\|5mCs\|o | | | | |
| | Cs\|oAp\|oGs\|oCs\|oAs\|oA] | | | | |
| 310 | [oUs\|oUs\|oGs\|oAs\|oUs\|5mCs\|5mCs\|dT | 65.42524 | 41.63383 | 1.64477605 | 2.287743 |
| | s\|dGs\|dTs\|dTs\|5mCs\|dTs\|5mCs\|5mCs\|o | | | | |
| | Cp\|oAs\|oGs\|oCs\|oAs\|oA] | | | | |
| 311 | [oUs\|oUs\|oGs\|oAs\|oUs\|5mCs\|5mCs\|dT | 79.57865 | 47.30341 | 3.2793354 | 4.879421 |
| | s\|dGs\|dTs\|dTs\|5mCs\|dTs\|5mCs\|5mCs\|o | | | | |
| | Cp\|oAp\|oGs\|oCs\|oAs\|oA] | | | | |
| 312 | [oUs\|oUs\|oGp\|oAs\|oUs\|5mCs\|5mCs\|dT | 74.90458 | 37.7908 | 7.9156018 | 2.492285 |
| | s\|dGs\|dTs\|dTs\|5mCs\|dTs\|5mCs\|5mCs\|o | | | | |
| | Cs\|oAp\|oGs\|oCs\|oAs\|oA] | | | | |
| 313 | [oUs\|oUs\|oGp\|oAs\|oUs\|5mCs\|5mCs\|dT | 64.17809 | 41.25918 | 3.60546685 | 2.451177 |
| | s\|dGs\|dTs\|dTs\|5mCs\|dTs\|5mCs\|5mCs\|o | | | | |
| | Cp\|oAs\|oGs\|oCs\|oAs\|oA] | | | | |
| 314 | [oUs\|oUs\|oGs\|oAp\|oUs\|5mCs\|5mCs\|dT | 77.79264 | 36.3895 | 5.16839925 | 1.47233 |
| | s\|dGs\|dTs\|dTs\|5mCs\|dTs\|5mCs\|5mCs\|o | | | | |
| | Cs\|oAp\|oGs\|oCs\|oAs\|oA] | | | | |
| 315 | [oUs\|oUs\|oGs\|oAp\|oUs\|5mCs\|5mCs\|dT | 69.27439 | 40.78583 | 0.70050225 | 3.765485 |
| | s\|dGs\|dTs\|dTs\|5mCs\|dTs\|5mCs\|5mCs\|o | | | | |
| | Cp\|oAs\|oGs\|oCs\|oAs\|oA] | | | | |
| 316 | [oUs\|oUs\|oGp\|oAs\|oUs\|5mCs\|5mCs\|dT | 66.79254 | 41.42264 | 4.07791885 | 2.517419 |
| | s\|dGs\|dTs\|dTs\|5mCs\|dTs\|5mCs\|5mCs\|o | | | | |
| | Cp\|oAp\|oGs\|oCs\|oAs\|oA] | | | | |
| 317 | [oUs\|oUs\|oGs\|oAp\|oUs\|5mCs\|5mCs\|dT | 69.95295 | 38.70855 | 5.1765897 | 1.322 |
| | s\|dGs\|dTs\|dTs\|5mCs\|dTs\|5mCs\|5mCs\|o | | | | |
| | Cp\|oAp\|oGs\|oCs\|oAs\|oA] | | | | |
| 318 | [oUs\|oUs\|oGp\|oAp\|oUs\|5mCs\|5mCs\|dT | 64.59882 | 36.2354 | 1.94088075 | 2.245535 |
| | s\|dGs\|dTs\|dTs\|5mCs\|dTs\|5mCs\|5mCs\|o | | | | |
| | Cs\|oAp\|oGs\|oCs\|oAs\|oA] | | | | |
| 319 | [oUs\|oUs\|oGp\|oAp\|oUs\|5mCs\|5mCs\|dT | 71.61855 | 40.11992 | 4.8955589 | 1.16262 |
| | s\|dGs\|dTs\|dTs\|5mCs\|dTs\|5mCs\|5mCs\|o | | | | |
| | Cp\|oAs\|oGs\|oCs\|oAs\|oA] | | | | |
| 320 | [oUs\|oUs\|oGp\|oAp\|oUs\|5mCs\|5mCs\|dT | 73.74875 | 40.87392 | 1.9294505 | 0.764434 |
| | s\|dGs\|dTs\|dTs\|5mCs\|dTs\|5mCs\|5mCs\|o | | | | |
| | Cp\|oAp\|oGs\|oCs\|oAs\|oA] | | | | |
| 321 | [oUs\|oUs\|oGs\|oAs\|oUs\|5mCs\|5mCs\|dT | 69.14922 | 42.64199 | 6.9911749 | 2.990965 |
| | s\|dGs\|dTs\|dTs\|5mCs\|dTs\|5mCs\|5mCs\|o | | | | |
| | Cs\|oAs\|oGs\|oCs\|oAs\|oAs\|oC] | | | | |
| 322 | [oUs\|oUs\|oGp\|oAs\|oUs\|5mCs\|5mCs\|dT | 68.03041 | 40.82452 | 3.81307025 | 3.211419 |
| | s\|dGs\|dTs\|dTs\|5mCs\|dTs\|5mCs\|5mCs\|o | | | | |
| | Cs\|oAs\|oGs\|oCs\|oAs\|oAs\|oC] | | | | |
| 323 | [oUs\|oUs\|oGs\|oAp\|oUs\|5mCs\|5mCs\|dT | 64.06085 | 38.06864 | 3.2673432 | 2.579056 |
| | sdGs\|dTs\|dTs\|5mCs\|dTs\|5mCs\|5mCs\|o | | | | |
| | Cs\|oAs\|oGs\|oCs\|oAs\|oAs\|oC] | | | | |
| 324 | [oUs\|oUs\|oGp\|oAp\|oUs\|5mCs\|5mCs\|dT | 64.06883 | 40.38284 | 2.58838335 | 2.66873 |
| | s\|dGs\|dTs\|dTs\|5mCs\|dTs\|5mCs\|5mCs\|o | | | | |
| | Cs\|oAs\|oGs\|oCs\|oAs\|oAs\|oC] | | | | |
| 325 | [oUs\|oUs\|oGs\|oAs\|oUs\|5mCs\|5mCs\|dT | 73.59074 | 39.51843 | 5.5155268 | 1.608472 |
| | s\|dGs\|dTs\|dTs\|5mCs\|dTs\|5mCs\|5mCs\|o | | | | |
| | Cs\|oAp\|oGs\|oCs\|oAs\|oAs\|oC] | | | | |
| 326 | [oUs\|oUs\|oGs\|oAs\|oUs\|5mCs\|5mCs\|dT | 64.14324 | 38.43417 | 2.9521007 | 1.560155 |
| | s\|dGs\|dTs\|dTs\|5mCs\|dTs\|5mCs\|5mCs\|o | | | | |
| | Cp\|oAs\|oGs\|oCs\|oAs\|oAs\|oC] | | | | |
| 327 | [oUs\|oUs\|oGs\|oAs\|oUs\|5mCs\|5mCs\|dT | 67.68504 | 45.94997 | 5.2482586 | 2.096218 |
| | s\|dGs\|dTs\|dTs\|5mCs\|dTs\|5mCs\|5mCs\|o | | | | |
| | Cp\|oAp\|oGs\|oCs\|oAs\|oAs\|oC] | | | | |
| 328 | [oUs\|oUs\|oGp\|oAs\|oUs\|5mCs\|5mCs\|dT | 74.0999 | 38.3912 | 0.5712221 | 1.732884 |
| | s\|dGs\|dTs\|dTs\|5mCs\|dTs\|5mCs\|5mCs\|o | | | | |
| | Cs\|oAp\|oGs\|oCs\|oAs\|oAs\|oC] | | | | |
| 329 | [oUs\|oUs\|oGp\|oAs\|oUs\|5mCs\|5mCs\|dT | 59.85657 | 40.50332 | 4.4957246 | 1.479709 |
| | s\|dGs\|dTs\|dTs\|5mCs\|dTs\|5mCs\|5mCs\|o | | | | |
| | Cp\|oAs\|oGs\|oCs\|oAs\|oAs\|oC] | | | | |
| 330 | [oUs\|oUs\|oGs\|oAp\|oUs\|5mCs\|5mCs\|dT | 62.98679 | 36.72381 | 9.8570092 | 0.854923 |
| | s\|dGs\|dTs\|dTs\|5mCs\|dTs\|5mCs\|5mCs\|o | | | | |
| | Cs\|oAp\|oGs\|oCs\|oAs\|oAs\|oC] | | | | |
| 331 | [oUs\|oUs\|oGs\|oAp\|oUs\|5mCs\|5mCs\|dT | 66.22289 | 38.29377 | 7.80879995 | 2.702458 |
| | s\|dGs\|dTs\|dTs\|5mCs\|dTs\|5mCs\|5mCs\|o | | | | |
| | Cp\|oAs\|oGs\|oCs\|oAs\|oAs\|oC] | | | | |
| 332 | [oUs\|oUs\|oGp\|oAs\|oUs\|5mCs\|5mCs\|dT | 64.84554 | 38.62897 | 2.9279718 | 1.019158 |
| | s\|dGs\|dTs\|dTs\|5mCs\|dTs\|5mCs\|5mCs\|o | | | | |
| | Cp\|oAp\|oGs\|oCs\|oAs\|oAs\|oC] | | | | |
| 333 | [oUs\|oUs\|oGs\|oAp\|oUs\|5mCs\|5mCs\|dT | 72.02838 | 42.5541 | 4.6483644 | 3.24576 |
| | s\|dGs\|dTs\|dTs\|5mCs\|dTs\|5mCs\|5mCs\|o | | | | |
| | Cp\|oAp\|oGs\|oCs\|oAs\|oAs\|oC] | | | | |
| 334 | [oUs\|oUs\|oGp\|oAp\|oUs\|5mCs\|5mCs\|dT | 73.13125 | 37.42697 | 3.52337675 | 2.273121 |
| | s\|dGs\|dTs\|dTs\|5mCs\|dTs\|5mCs\|5mCs\|o | | | | |
| | Cs\|oAp\|oGs\|oCs\|oAs\|oAs\|oC] | | | | |
| 335 | [oUs\|oUs\|oGp\|oAp\|oUs\|5mCs\|5mCs\|dT | 65.10868 | 38.41509 | 2.69335365 | 3.463081 |
| | s\|dGs\|dTs\|dTs\|5mCs\|dTs\|5mCs\|5mCs\|o | | | | |
| | Cp\|oAs\|oGs\|oCs\|oAs\|oAs\|oC] | | | | |
| 336 | [oUs\|oUs\|oGp\|oAp\|oUs\|5mCs\|5mCs\|dT | 74.0986 | 41.06858 | 5.59980225 | 3.735298 |
| | s\|dGs\|dTs\|dTs\|5mCs\|dTs\|5mCs\|5mCs\|o | | | | |
| | Cp\|oAp\|oGs\|oCs\|oAs\|oAs\|oC] | | | | |
| 337 | [oUs\|oUs\|oGs\|oAs\|oUs\|5mCs\|5mCs\|dT | 68.10988 | 33.22407 | 2.04506795 | 1.219344 |
| | s\|dGs\|dTs\|dTs\|5mCs\|dTs\|5mCs\|5mCs\|o | | | | |
| | Cs\|oAs\|oGs\|oCs\|oAs\|oAs\|oCs\|oA] | | | | |
| 338 | [oUs\|oUs\|oGp\|oAs\|oUs\|5mCs\|5mCs\|dT | 61.51268 | 39.42608 | 3.2210901 | 1.60843 |
| | s\|dGs\|dTs\|dTs\|5mCs\|dTs\|5mCs\|5mCs\|o | | | | |
| | Cs\|oAs\|oGs\|oCs\|oAs\|oAs\|oCs\|oA] | | | | |
| 339 | [oUs\|oUs\|oGs\|oAp\|oUs\|5mCs\|5mCs\|dT | 61.62901 | 38.10779 | 5.0486966 | 1.469955 |
| | s\|dGs\|dTs\|dTs\|5mCs\|dTs\|5mCs\|5mCs\|o | | | | |
| | Cs\|oAs\|oGs\|oCs\|oAs\|oAs\|oCs\|oA] | | | | |
| 340 | [oUs\|oUs\|oGp\|oAp\|oUs\|5mCs\|5mCs\|dT | 68.70971 | 37.9342 | 9.15707545 | 2.987791 |
| | s\|dGs\|dTs\|dTs\|5mCs\|dTs\|5mCs\|5mCs\|o | | | | |
| | Cs\|oAs\|oGs\|oCs\|oAs\|oAs\|oCs\|oA] | | | | |
| 341 | [oUs\|oUs\|oGs\|oAs\|oUs\|5mCs\|5mCs\|dT | 64.63658 | 37.92174 | 8.0800218 | 2.867291 |
| | sdGs\|dTs\|dTs\|5mCs\|dTs\|5mCs\|5mCs\|o | | | | |
| | Cs\|oAp\|oGs\|oCs\|oAs\|oAs\|oCs\|oA] | | | | |
| 342 | [oUs\|oUs\|oGs\|oAs\|oUs\|5mCs\|5mCs\|dT | 71.59294 | 36.10313 | 4.23800025 | 2.212339 |
| | s\|dGs\|dTs\|dTs\|5mCs\|dTs\|5mCs\|5mCs\|o | | | | |
| | Cp\|oAs\|oGs\|oCs\|oAs\|oAs\|oCs\|oA] | | | | |
| 343 | [oUs\|oUs\|oGs\|oAs\|oUs\|5mCs\|5mCs\|dT | 67.77124 | 42.50938 | 4.24974715 | 0.606911 |
| | s\|dGs\|dTs\|dTs\|5mCs\|dTs\|5mCs\|5mCs\|o | | | | |
| | Cp\|oAp\|oGs\|oCs\|oAs\|oAs\|oCs\|oA] | | | | |
| 344 | [oUs\|oUs\|oGp\|oAs\|oUs\|5mCs\|5mCs\|dT | 62.77385 | 38.9199 | 1.20627285 | 2.566854 |
| | s\|dGs\|dTs\|dTs\|5mCs\|dTs\|5mCs\|5mCs\|o | | | | |
| | Cs\|oAp\|oGs\|oCs\|oAs\|oAs\|oCs\|oA] | | | | |
| 345 | [oUs\|oUs\|oGp\|oAs\|oUs\|5mCs\|5mCs\|dT | 65.30274 | 37.50644 | 3.3490132 | 1.120349 |
| | s\|dGs\|dTs\|dTs\|5mCs\|dTs\|5mCs\|5mCs\|o | | | | |
| | Cp\|oAs\|oGs\|oCs\|oAs\|oAs\|oCs\|oA] | | | | |
| 346 | [oUs\|oUs\|oGs\|oAp\|oUs\|5mCs\|5mCs\|dT | 59.99562 | 43.52686 | 2.17603655 | 3.501488 |
| | s\|dGs\|dTs\|dTs\|5mCs\|dTs\|5mCs\|5mCs\|o | | | | |
| | Cs\|oAp\|oGs\|oCs\|oAs\|oAs\|oCs\|oA] | | | | |
| 347 | [oUs\|oUs\|oGs\|oAp\|oUs\|5mCs\|5mCs\|dT | 76.91818 | 36.71682 | 5.11543135 | 1.003503 |
| | s\|dGs\|dTs\|dTs\|5mCs\|dTs\|5mCs\|5mCs\|o | | | | |
| | Cp\|oAs\|oGs\|oCs\|oAs\|oAs\|oCs\|oA] | | | | |
| 348 | [oUs\|oUs\|oGp\|oAs\|oUs\|5mCs\|5mCs\|dT | 74.07843 | 40.52268 | 2.68814825 | 1.670121 |
| | s\|dGs\|dTs\|dTs\|5mCs\|dTs\|5mCs\|5mCs\|o | | | | |
| | Cp\|oAp\|oGs\|oCs\|oAs\|oAs\|oCs\|oA] | | | | |
| 349 | [oUs\|oUs\|oGs\|oAp\|oUs\|5mCs\|5mCs\|dT | 66.80045 | 41.82373 | 5.23486615 | 1.955829 |
| | s\|dGs\|dTs\|dTs\|5mCs\|dTs\|5mCs\|5mCs\|o | | | | |
| | Cp\|oAp\|oGs\|oCs\|oAs\|oAs\|oCs\|oA] | | | | |
| 350 | [oUs\|oUs\|oGp\|oAp\|oUs\|5mCs\|5mCs\|dT | 71.27418 | 43.95148 | 3.022233 | 1.912625 |
| | s\|dGs\|dTs\|dTs\|5mCs\|dTs\|5mCs\|5mCs\|o | | | | |
| | Cs\|oAp\|oGs\|oCs\|oAs\|oAs\|oCs\|oA] | | | | |
| 351 | [oUs\|oUs\|oGp\|oAp\|oUs\|5mCs\|5mCs\|dT | 68.26772 | 40.87145 | 1.67750485 | 1.489503 |
| | s\|dGs\|dTs\|dTs\|5mCs\|dTs\|5mCs\|5mCs\|o | | | | |
| | Cp\|oAs\|oGs\|oCs\|oAs\|oAs\|oCs\|oA] | | | | |
| 352 | [oUs\|oUs\|oGp\|oAp\|oUs\|5mCs\|5mCs\|dT | 74.70355 | 46.98739 | 4.10748375 | 3.361829 |
| | s\|dGs\|dTs\|dTs\|5mCs\|dTs\|5mCs\|5mCs\|o | | | | |
| | Cp\|oAp\|oGs\|oCs\|oAs\|oAs\|oCs\|oA] | | | | |
| 353 | [oUs\|oUs\|oGs\|oAs\|oUs\|5mCs\|5mCs\|dT | 82.69998 | 45.53944 | 2.7748001 | 5.248864 |
| | s\|dGs\|dTs\|dTs\|5mCs\|dTs\|5mCs\|5mCs\|5 | | | | |
| | mCs\|oAs\|oGs\|oCs\|oAs\|oA] | | | | |
| 354 | [oUs\|oUs\|oGp\|oAs\|oUs\|5mCs\|5mCs\|dT | 75.2964 | 41.58329 | 4.28755645 | 1.604165 |
| | s\|dGs\|dTs\|dTs\|5mCs\|dTs\|5mCs\|5mCs\|5 | | | | |
| | mCs\|oAs\|oGs\|oCs\|oAs\|oA] | | | | |
| 355 | [oUs\|oUs\|oGs\|oAp\|oUs\|5mCs\|5mCs\|dT | 78.25078 | 38.04859 | 2.1756527 | 2.812668 |
| | s\|dGs\|dTs\|dTs\|5mCs\|dTs\|5mCs\|5mCs\|5 | | | | |
| | mCs\|oAs\|oGs\|oCs\|oAs\|oA] | | | | |
| 356 | [oUs\|oUs\|oGp\|oAp\|oUs\|5mCs\|5mCs\|dT | 75.66047 | 46.42479 | 5.32824395 | 3.512293 |
| | s\|dGs\|dTs\|dTs\|5mCs\|dTs\|5mCs\|5mCs\|5 | | | | |
| | mCs\|oAs\|oGs\|oCs\|oAs\|oA] | | | | |
| 357 | [oUs\|oUs\|oGs\|oAs\|oUs\|5mCs\|5mCs\|dT | 70.8279 | 39.60585 | 4.17139065 | 1.315399 |
| | s\|dGs\|dTs\|dTs\|5mCs\|dTs\|5mCs\|5mCs\|5 | | | | |
| | mCs\|oAp\|oGs\|oCs\|oAs\|oA] | | | | |
| 358 | [oUs\|oUs\|oGs\|oAs\|oUs\|5mCs\|5mCs\|dT | 88.71474 | 41.43116 | 2.63982225 | 2.837026 |
| | s\|dGs\|dTs\|dTs\|5mCs\|dTs\|5mCs\|5mCs\|5 | | | | |
| | mCp\|oAs\|oGs\|oCs\|oAs\|oA] | | | | |
| 359 | [oUs\|oUs\|oGs\|oAs\|oUs\|5mCs\|5mCs\|dT | 94.02871 | 55.61287 | 3.28254665 | 5.31701 |
| | s\|dGs\|dTs\|dTs\|5mCs\|dTs\|5mCs\|5mCs\|5 | | | | |
| | mCp\|oAp\|oGs\|oCs\|oAs\|oA] | | | | |
| 360 | [oUs\|oUs\|oGp\|oAs\|oUs\|5mCs\|5mCs\|dT | 81.20155 | 48.93426 | 2.9797871 | 3.293206 |
| | s\|dGs\|dTs\|dTs\|5mCs\|dTs\|5mCs\|5mCs\|5 | | | | |
| | mCs\|oAp\|oGs\|oCs\|oAs\|oA] | | | | |
| 361 | [oUs\|oUs\|oGp\|oAs\|oUs\|5mCs\|5mCs\|dT | 81.56161 | 43.33641 | 5.9890201 | 2.745271 |
| | sdGs\|dTs\|dTs\|5mCs\|dTs\|5mCs\|5mCs\|5 | | | | |
| | mCp\|oAs\|oGs\|oCs\|oAs\|oA] | | | | |
| 362 | [oUs\|oUs\|oGs\|oAp\|oUs\|5mCs\|5mCs\|dT | 77.46717 | 40.6332 | 6.53245465 | 2.851351 |
| | s\|dGs\|dTs\|dTs\|5mCs\|dTs\|5mCs\|5mCs\|5 | | | | |
| | mCs\|oAp\|oGs\|oCs\|oAs\|oA] | | | | |
| 363 | [oUs\|oUs\|oGs\|oAp\|oUs\|5mCs\|5mCs\|dT | 91.06332 | 37.09205 | 6.16485235 | 1.515162 |
| | s\|dGs\|dTs\|dTs\|5mCs\|dTs\|5mCs\|5mCs\|5 | | | | |
| | mCp\|oAs\|oGs\|oCs\|oAs\|oA] | | | | |
| 364 | [oUs\|oUs\|oGp\|oAs\|oUs\|5mCs\|5mCs\|dT | 87.55428 | 42.57427 | 3.1016107 | 2.002001 |
| | s\|dGs\|dTs\|dTs\|5mCs\|dTs\|5mCs\|5mCs\|5 | | | | |
| | mCp\|oAp\|oGs\|oCs\|oAs\|oA] | | | | |
| 365 | [oUs\|oUs\|oGs\|oAp\|oUs\|5mCs\|5mCs\|dT | 90.76046 | 46.77878 | 4.93885515 | 3.608661 |
| | s\|dGs\|dTs\|dTs\|5mCs\|dTs\|5mCs\|5mCs\|5 | | | | |
| | mCp\|oAp\|oGs\|oCs\|oAs\|oA] | | | | |
| 366 | [oUs\|oUs\|oGp\|oAp\|oUs\|5mCs\|5mCs\|dT | 76.47815 | 41.28109 | 4.02130685 | 3.175797 |
| | s\|dGs\|dTs\|dTs\|5mCs\|dTs\|5mCs\|5mCs\|5 | | | | |
| | mCs\|oAp\|oGs\|oCs\|oAs\|oA] | | | | |
| 367 | [oUs\|oUs\|oGp\|oAp\|oUs\|5mCs\|5mCs\|dT | 81.30346 | 46.78571 | 5.4938338 | 2.280535 |
| | s\|dGs\|dTs\|dTs\|5mCs\|dTs\|5mCs\|5mCs\|5 | | | | |
| | mCp\|oAs\|oGs\|oCs\|oAs\|oA] | | | | |
| 368 | [oUs\|oUs\|oGp\|oAp\|oUs\|5mCs\|5mCs\|dT | 84.12098 | 44.30416 | 5.38885525 | 2.935461 |
| | s\|dGs\|dTs\|dTs\|5mCs\|dTs\|5mCs\|5mCs\|5 | | | | |
| | mCp\|oAp\|oGs\|oCs\|oAs\|oA] | | | | |
| 369 | [oUs\|oUs\|oGs\|oAs\|oUs\|5mCs\|5mCs\|dT | 70.44063 | 38.60541 | 6.7004305 | 3.620983 |
| | s\|dGs\|dTs\|dTs\|5mCs\|dTs\|5mCs\|5mCs\|5 | | | | |
| | mCs\|dAs\|oGs\|oCs\|oAs\|oAs\|oC] | | | | |
| 370 | [oUs\|oUs\|oGp\|oAs\|oUs\|5mCs\|5mCs\|dT | 74.17394 | 36.98506 | 2.4084315 | 1.467456 |
| | s\|dGs\|dTs\|dTs\|5mCs\|dTs\|5mCs\|5mCs\|5 | | | | |
| | mCs\|dAs\|oGs\|oCs\|oAs\|oAs\|oC] | | | | |
| 371 | [oUs\|oUs\|oGs\|oAp\|oUs\|5mCs\|5mCs\|dT | 74.77409 | 37.54156 | 7.2673398 | 1.490426 |
| | s\|dGs\|dTs\|dTs\|5mCs\|dTs\|5mCs\|5mCs\|5 | | | | |
| | mCs\|dAs\|oGs\|oCs\|oAs\|oAs\|oC] | | | | |
| 372 | [oUs\|oUs\|oGp\|oAp\|oUs\|5mCs\|5mCs\|dT | 78.93023 | 38.38505 | 5.56282815 | 3.943036 |
| | s\|dGs\|dTs\|dTs\|5mCs\|dTs\|5mCs\|5mCs\|5 | | | | |
| | mCs\|dAs\|oGs\|oCs\|oAs\|oAs\|oC] | | | | |
| 373 | [oUs\|oUs\|oGs\|oAs\|oUs\|5mCs\|5mCs\|dT | 71.77732 | 36.26151 | 4.19725075 | 2.479808 |
| | s\|dGs\|dTs\|dTs\|5mCs\|dTs\|5mCs\|5mCs\|5 | | | | |
| | mCs\|dAp\|oGs\|oCs\|oAs\|oAs\|oC] | | | | |
| 374 | [oUs\|oUs\|oGs\|oAs\|oUs\|5mCs\|5mCs\|dT | 75.98736 | 46.94162 | 3.67475295 | 2.387602 |
| | s\|dGs\|dTs\|dTs\|5mCs\|dTs\|5mCs\|5mCs\|5 | | | | |
| | mCp\|dAs\|oGs\|oCs\|oAs\|oAs\|oC] | | | | |
| 375 | [oUs\|oUs\|oGs\|oAs\|oUs\|5mCs\|5mCs\|dT | 83.01221 | 48.4748 | 1.83557915 | 3.954731 |
| | s\|dGs\|dTs\|dTs\|5mCs\|dTs\|5mCs\|5mCs\|5 | | | | |
| | mCp\|dAp\|oGs\|oCs\|oAs\|oAs\|oC] | | | | |
| 376 | [oUs\|oUs\|oGp\|oAs\|oUs\|5mCs\|5mCs\|dT | 73.40898 | 45.08328 | 1.55749415 | 2.879961 |
| | s\|dGs\|dTs\|dTs\|5mCs\|dTs\|5mCs\|5mCs\|5 | | | | |
| | mCs\|dAp\|oGs\|oCs\|oAs\|oAs\|oC] | | | | |
| 377 | [oUs\|oUs\|oGp\|oAs\|oUs\|5mCs\|5mCs\|dT | 81.18265 | 43.25494 | 2.32470145 | 2.898854 |
| | s\|dGs\|dTs\|dTs\|5mCs\|dTs\|5mCs\|5mCs\|5 | | | | |
| | mCp\|dAs\|oGs\|oCs\|oAs\|oAs\|oC] | | | | |
| 378 | [oUs\|oUs\|oGs\|oAp\|oUs\|5mCs\|5mCs\|dT | 85.8249 | 35.81711 | 7.0913574 | 1.999337 |
| | s\|dGs\|dTs\|dTs\|5mCs\|dTs\|5mCs\|5mCs\|5 | | | | |
| | mCs\|dAp\|oGs\|oCs\|oAs\|oAs\|oC] | | | | |
| 379 | [oUs\|oUs\|oGs\|oAp\|oUs\|5mCs\|5mCs\|dT | 85.9207 | 39.07233 | 8.2451204 | 1.716324 |
| | s\|dGs\|dTs\|dTs\|5mCs\|dTs\|5mCs\|5mCs\|5 | | | | |
| | mCp\|dAs\|oGs\|oCs\|oAs\|oAs\|oC] | | | | |
| 380 | [oUs\|oUs\|oGp\|oAs\|oUs\|5mCs\|5mCs\|dT | 89.28203 | 44.69979 | 3.57026875 | 2.48225 |
| | s\|dGs\|dTs\|dTs\|5mCs\|dTs\|5mCs\|5mCs\|5 | | | | |
| | mCp\|dAp\|oGs\|oCs\|oAs\|oAs\|oC] | | | | |
| 381 | [oUs\|oUs\|oGs\|oAp\|oUs\|5mCs\|5mCs\|dT | 80.47781 | 43.39312 | 1.49203405 | 1.667053 |
| | sdGs\|dTs\|dTs\|5mCs\|dTs\|5mCs\|5mCs\|5 | | | | |
| | mCp\|dAp\|oGs\|oCs\|oAs\|oAs\|oC] | | | | |
| 382 | [oUs\|oUs\|oGp\|oAp\|oUs\|5mCs\|5mCs\|dT | 84.37027 | 35.88873 | 4.3911855 | 0.578314 |
| | s\|dGs\|dTs\|dTs\|5mCs\|dTs\|5mCs\|5mCs\|5 | | | | |
| | mCs\|dAp\|oGs\|oCs\|oAs\|oAs\|oC] | | | | |
| 383 | [oUs\|oUs\|oGp\|oAp\|oUs\|5mCs\|5mCs\|dT | 78.04549 | 35.55957 | 3.76452225 | 3.138921 |
| | s\|dGs\|dTs\|dTs\|5mCs\|dTs\|5mCs\|5mCs\|5 | | | | |
| | mCp\|dAs\|oGs\|oCs\|oAs\|oAs\|oC] | | | | |
| 384 | [oUs\|oUs\|oGp\|oAp\|oUs\|5mCs\|5mCs\|dT | 86.50257 | 42.97401 | 7.1309543 | 0.436476 |
| | sdGs\|dTs\|dTs\|5mCs\|dTs\|5mCs\|5mCs\|5 | | | | |
| | mCp\|dAp\|oGs\|oCs\|oAs\|oAs\|oC] | | | | |

TABLE 4

| Antisense | |
|---|---|
| Oligo No./ | |
| SEQ ID NO: | Variant Sequence |
| 390 | [oUs\|LGs\|o5mCs\|oUs\|oAs\|oGs\|oGs\|oUs\|dGs\|dAs\|dTs\|dGs\|5mCs\|dAs\|5mCs\|dTs\|dG |
| | s\|5mCs\|oUs\|oU] |
| 391 | [oUs\|oGs\|L5mCs\|oUs\|oAs\|oGs\|oGs\|oUs\|dGs\|dAs\|dTs\|dGs\|5mCs\|dAs\|5mCs\|dTs\|dG |
| | s\|5mCs\|oUs\|oU] |
| 392 | [oUs\|oGs\|o5mCs\|LTs\|oAs\|oGs\|oGs\|oUs\|dGs\|dAs\|dTs\|dGs\|5mCs\|dAs\|5mCs\|dTs\|dG |
| | s\|5mCs\|oUs\|oU] |
| 393 | [oUs\|oGs\|o5mCs\|oUs\|LAs\|oGs\|oGs\|oUs\|dGs\|dAs\|dTs\|dGs\|5mCs\|dAs\|5mCs\|dTs\|dG |
| | s\|5mCs\|oUs\|oU] |
| 394 | [oUs\|oGs\|o5mCs\|oUs\|oAs\|LGs\|oGs\|oUs\|dGs\|dAs\|dTs\|dGs\|5mCs\|dAs\|5mCs\|dTs\|dG |
| | s\|5mCs\|oUs\|oU] |
| 395 | [oUs\|oGs\|o5mCs\|oUs\|oAs\|oGs\|LGs\|oUs\|dGs\|dAs\|dTs\|dGs\|5mCs\|dAs\|5mCs\|dTs\|dG |
| | s\|5mCs\|oUs\|oU] |
| 396 | [oUs\|oGs\|5mCs\|dTs\|dAs\|dGs\|dGs\|dTs\|dGs\|dAs\|dTs\|dGs\|o5mCs\|oAs\|o5mCs\|oUs\|o |
| | Gs\|o5mCs\|LTs\|oU] |
| 397 | [oUs\|oGs\|5mCs\|dTs\|dAs\|dGs\|dGs\|dTs\|dGs\|dAs\|dTs\|dGs\|o5mCs\|oAs\|o5mCs\|oUs\|o |
| | Gs\|L5mCs\|oUs\|oU] |
| 398 | [oUs\|oGs\|5mCs\|dTs\|dAs\|dGs\|dGs\|dTs\|dGs\|dAs\|dTs\|dGs\|o5mCs\|oAs\|o5mCs\|oUs\|L |
| | Gs\|o5mCs\|oUs\|oU] |
| 399 | [oUs\|oGs\|5mCs\|dTs\|dAs\|dGs\|dGs\|dTs\|dGs\|dAs\|dTs\|dGs\|o5mCs\|oAs\|o5mCs\|LTs\|o |
| | Gs\|o5mCs\|oUs\|oU] |
| 400 | [oUs\|oGs\|5mCs\|dTs\|dAs\|dGs\|dGs\|dTs\|dGs\|dAs\|dTs\|dGs\|o5mCs\|oAs\|L5mCs\|oUs\|o |
| | Gs\|o5mCs\|oUs\|oU] |
| 401 | [oUs\|oGs\|5mCs\|dTs\|dAs\|dGs\|dGs\|dTs\|dGs\|dAs\|dTs\|dGs\|o5mCs\|LAs\|o5mCs\|oUs\|o |
| | Gs\|o5mCs\|oUs\|oU] |
| 402 | [oUs\|LGs\|o5mCs\|oUs\|oAs\|oGs\|oGs\|dTs\|dGs\|dAs\|dTs\|dGs\|5mCs\|dAs\|5mCs\|dTs\|dG |
| | s\|o5mCs\|oUs\|oU] |
| 403 | [oUs\|oGs\|L5mCs\|oUs\|oAs\|oGs\|oGs\|dTs\|dGs\|dAs\|dTs\|dGs\|5mCs\|dAs\|5mCs\|dTs\|dG |
| | s\|o5mCs\|oUs\|oU] |
| 404 | [oUs\|oGs\|o5mCs\|LTs\|oAs\|oGs\|oGs\|dTs\|dGs\|dAs\|dTs\|dGs\|5mCs\|dAs\|5mCs\|dTs\|dG |
| | s\|o5mCs\|oUs\|oU] |
| 405 | [oUs\|oGs\|o5mCs\|oUs\|LAs\|oGs\|oGs\|dTs\|dGs\|dAs\|dTs\|dGs\|5mCs\|dAs\|5mCs\|dTs\|dG |
| | s\|o5mCs\|oUs\|oU] |
| 406 | [oUs\|oGs\|o5mCs\|oUs\|oAs\|LGs\|oGs\|dTs\|dGs\|dAs\|dTs\|dGs\|5mCs\|dAs\|5mCs\|dTs\|dG |
| | s\|o5mCs\|oUs\|oU] |
| 407 | [oUs\|oGs\|o5mCs\|dTs\|dAs\|dGs\|dGs\|dTs\|dGs\|dAs\|dTs\|dGs\|5mCs\|oAs\|o5mCs\|oUs\|o |
| | Gs\|o5mCs\|LTs\|oU] |
| 408 | [oUs\|oGs\|o5mCs\|dTs\|dAs\|dGs\|dGs\|dTs\|dGs\|dAs\|dTs\|dGs\|5mCs\|oAs\|o5mCs\|oUs\|o |
| | Gs\|L5mCs\|oUs\|oU] |
| 409 | [oUs\|oGs\|o5mCs\|dTs\|dAs\|dGs\|dGs\|dTs\|dGs\|dAs\|dTs\|dGs\|5mCs\|oAs\|o5mCs\|oUs\|L |
| | Gs\|o5mCs\|oUs\|oU] |
| 410 | [oUs\|oGs\|o5mCs\|dTs\|dAs\|dGs\|dGs\|dTs\|dGs\|dAs\|dTs\|dGs\|5mCs\|oAs\|o5mCs\|LTs\|o |
| | Gs\|o5mCs\|oUs\|oU] |
| 411 | [oUs\|oGs\|o5mCs\|dTs\|dAs\|dGs\|dGs\|dTs\|dGs\|dAs\|dTs\|dGs\|5mCs\|oAs\|L5mCs\|oUs\|o |
| | Gs\|o5mCs\|oUs\|oU] |
| 412 | [oUs\|LGs\|o5mCs\|oUs\|oAs\|oGs\|oGs\|dTs\|dGs\|dAs\|dTs\|dGs\|5mCs\|dAs\|5mCs\|dTs\|dG |
| | s\|5mCs\|oUs\|oU] |
| 413 | [oUs\|oGs\|L5mCs\|oUs\|oAs\|oGs\|oGs\|dTs\|dGs\|dAs\|dTs\|dGs\|5mCs\|dAs\|5mCs\|dTs\|dG |
| | s\|5mCs\|oUs\|oU] |
| 414 | [oUs\|oGs\|o5mCs\|LTs\|oAs\|oGs\|oGs\|dTs\|dGs\|dAs\|dTs\|dGs\|5mCs\|dAs\|5mCs\|dTs\|dG |
| | s\|5mCs\|oUs\|oU] |
| 415 | [oUs\|oGs\|o5mCs\|oUs\|LAs\|oGs\|oGs\|dTs\|dGs\|dAs\|dTs\|dGs\|5mCs\|dAs\|5mCs\|dTs\|dG |
| | s\|5mCs\|oUs\|oU] |
| 416 | [oUs\|oGs\|o5mCs\|oUs\|oAs\|LGs\|oGs\|dTs\|dGs\|dAs\|dTs\|dGs\|5mCs\|dAs\|5mCs\|dTs\|dG |
| | s\|5mCs\|oUs\|oU] |
| 417 | [oGs\|o5mCs\|dTs\|dAs\|dGs\|dGs\|dTs\|dGs\|dAs\|dTs\|dGs\|5mCs\|oAs\|o5mCs\|oUs\|oGs\|o5 |
| | mCs\|LTs\|oU] |
| 418 | [oGs\|o5mCs\|dTs\|dAs\|dGs\|dGs\|dTs\|dGs\|dAs\|dTs\|dGs\|5mCs\|oAs\|o5mCs\|oUs\|oGs\|L |
| | 5mCs\|oUs\|oU] |
| 419 | [oGs\|o5mCs\|dTs\|dAs\|dGs\|dGs\|dTs\|dGs\|dAs\|dTs\|dGs\|5mCs\|oAs\|o5mCs\|oUs\|LGs\|o |
| | 5mCs\|oUs\|oU] |
| 420 | [oGs\|o5mCs\|dTs\|dAs\|dGs\|dGs\|dTs\|dGs\|dAs\|dTs\|dGs\|5mCs\|oAs\|o5mCs\|LTs\|oGs\|o5 |
| | mCs\|oUs\|oU] |
| 421 | [oGs\|o5mCs\|dTs\|dAs\|dGs\|dGs\|dTs\|dGs\|dAs\|dTs\|dGs\|5mCs\|oAs\|L5mCs\|oUs\|oGs\|o |
| | 5mCs\|oUs\|oU] |
| 422 | [oUs\|LGs\|o5mCs\|oUs\|oAs\|oGs\|dGs\|dTs\|dGs\|dAs\|dTs\|dGs\|5mCs\|dAs\|5mCs\|dTs\|oG |
| | s\|o5mCs\|oUs\|oU] |
| 423 | [oUs\|oGs\|L5mCs\|oUs\|oAs\|oGs\|dGs\|dTs\|dGs\|dAs\|dTs\|dGs\|5mCs\|dAs\|5mCs\|dTs\|oG |
| | s\|o5mCs\|oUs\|oU] |
| 424 | [oUs\|oGs\|o5mCs\|LTs\|oAs\|oGs\|dGs\|dTs\|dGs\|dAs\|dTs\|dGs\|5mCs\|dAs\|5mCs\|dTs\|oG |
| | s\|o5mCs\|oUs\|oU] |
| 425 | [oUs\|oGs\|o5mCs\|oUs\|LAs\|oGs\|dGs\|dTs\|dGs\|dAs\|dTs\|dGs\|5mCs\|dAs\|5mCs\|dTs\|oG |
| | s\|o5mCs\|oUs\|oU] |
| 426 | [oUs\|oGs\|o5mCs\|oUs\|dAs\|dGs\|dGs\|dTs\|dGs\|dAs\|dTs\|dGs\|5mCs\|dAs\|o5mCs\|oUs\|o |
| | Gs\|o5mCs\|LTs\|oU] |
| 427 | [oUs\|oGs\|o5mCs\|oUs\|dAs\|dGs\|dGs\|dTs\|dGs\|dAs\|dTs\|dGs\|5mCs\|dAs\|o5mCs\|oUs\|o |
| | Gs\|L5mCs\|oUs\|oU] |
| 428 | [oUs\|oGs\|o5mCs\|oUs\|dAs\|dGs\|dGs\|dTs\|dGs\|dAs\|dTs\|dGs\|5mCs\|dAs\|o5mCs\|oUs\|L |
| | Gs\|o5mCs\|oUs\|oU] |
| 429 | [oUs\|oGs\|o5mCs\|oUs\|dAs\|dGs\|dGs\|dTs\|dGs\|dAs\|dTs\|dGs\|5mCs\|dAs\|o5mCs\|LTs\|o |
| | Gs\|o5mCs\|oUs\|oU] |
| 430 | [oUs\|LGs\|o5mCs\|oUs\|oAs\|oGs\|dGs\|dTs\|dGs\|dAs\|dTs\|dGs\|5mCs\|dAs\|5mCs\|dTs\|oG |
| | s\|o5mCs\|oU] |
| 431 | [oUs\|oGs\|L5mCs\|oUs\|oAs\|oGs\|dGs\|dTs\|dGs\|dAs\|dTs\|dGs\|5mCs\|dAs\|5mCs\|dTs\|oG |
| | s\|o5mCs\|oU] |
| 432 | [oUs\|oGs\|o5mCs\|LTs\|oAs\|oGs\|dGs\|dTs\|dGs\|dAs\|dTs\|dGs\|5mCs\|dAs\|5mCs\|dTs\|oG |
| | s\|o5mCs\|oU] |
| 433 | [oUs\|oGs\|o5mCs\|oUs\|LAs\|oGs\|dGs\|dTs\|dGs\|dAs\|dTs\|dGs\|5mCs\|dAs\|5mCs\|dTs\|oG |
| | s\|o5mCs\|oU] |
| 434 | [oGs\|o5mCs\|oUs\|dAs\|dGs\|dGs\|dTs\|dGs\|dAs\|dTs\|dGs\|5mCs\|dAs\|o5mCs\|oUs\|oGs\|o |
| | 5mCs\|LTs\|oU] |
| 435 | [oGs\|o5mCs\|oUs\|dAs\|dGs\|dGs\|dTs\|dGs\|dAs\|dTs\|dGs\|5mCs\|dAs\|o5mCs\|oUs\|oGs\|L |
| | 5mCs\|oUs\|oU] |
| 436 | [oGs\|o5mCs\|oUs\|dAs\|dGs\|dGs\|dTs\|dGs\|dAs\|dTs\|dGs\|5mCs\|dAs\|o5mCs\|oUs\|LGs\|o |
| | 5mCs\|oUs\|oU] |
| 437 | [oGs\|o5mCs\|oUs\|dAs\|dGs\|dGs\|dTs\|dGs\|dAs\|dTs\|dGs\|5mCs\|dAs\|o5mCs\|LTs\|oGs\|o |
| | 5mCs\|oUs\|oU] |
| 438 | [oUs\|LGs\|o5mCs\|oUs\|oAs\|oGs\|dGs\|dTs\|dGs\|dAs\|dTs\|dGs\|5mCs\|dAs\|5mCs\|dTs\|oG |
| | s\|o5mC] |
| 439 | [oUs\|oGs\|L5mCs\|oUs\|oAs\|oGs\|dGs\|dTs\|dGs\|dAs\|dTs\|dGs\|5mCs\|dAs\|5mCs\|dTs\|oG |
| | s\|o5mC] |
| 440 | [oUs\|oGs\|o5mCs\|LTs\|oAs\|oGs\|dGs\|dTs\|dGs\|dAs\|dTs\|dGs\|5mCs\|dAs\|5mCs\|dTs\|oG |
| | s\|o5mC] |
| 441 | [oUs\|oGs\|o5mCs\|oUs\|LAs\|oGs\|dGs\|dTs\|dGs\|dAs\|dTs\|dGs\|5mCs\|dAs\|5mCs\|dTs\|oG |
| | s\|o5mC] |
| 442 | [o5mCs\|oUs\|dAs\|dGs\|dGs\|dTs\|dGs\|dAs\|dTs\|dGs\|5mCs\|dAs\|o5mCs\|oUs\|oGs\|o5mC |
| | s\|LTs\|oU] |
| 443 | [o5mCs\|oUs\|dAs\|dGs\|dGs\|dTs\|dGs\|dAs\|dTs\|dGs\|5mCs\|dAs\|o5mCs\|oUs\|oGs\|L5mC |
| | s\|oUs\|oU] |
| 444 | [o5mCs\|oUs\|dAs\|dGs\|dGs\|dTs\|dGs\|dAs\|dTs\|dGs\|5mCs\|dAs\|o5mCs\|oUs\|LGs\|o5mC |
| | s\|oUs\|oU] |
| 445 | [o5mCs\|oUs\|dAs\|dGs\|dGs\|dTs\|dGs\|dAs\|dTs\|dGs\|5mCs\|dAs\|o5mCs\|LTs\|oGs\|o5mC |
| | s\|oUs\|oU] |
| 446 | [oUs\|LGs\|o5mCs\|oUs\|oAs\|dGs\|dGs\|dTs\|dGs\|dAs\|dTs\|dGs\|5mCs\|dAs\|5mCs\|oUs\|oG |
| | s\|o5mCs\|oUs\|oU |
| 447 | [oUs\|oGs\|L5mCs\|oUs\|oAs\|dGs\|dGs\|dTs\|dGs\|dAs\|dTs\|dGs\|5mCs\|dAs\|5mCs\|oUs\|oG |
| | s\|o5mCs\|oUs\|oU] |
| 448 | [oUs\|oGs\|o5mCs\|LTs\|oAs\|dGs\|dGs\|dTs\|dGs\|dAs\|dTs\|dGs\|5mCs\|dAs\|5mCs\|oUs\|oG |
| | s\|o5mCs\|oUs\|oU] |
| 449 | [oUs\|oGs\|o5mCs\|oUs\|oAs\|dGs\|dGs\|dTs\|dGs\|dAs\|dTs\|dGs\|5mCs\|dAs\|5mCs\|oUs\|oG |
| | s\|o5mCs\|LTs\|oU] |
| 450 | [oUs\|oGs\|o5mCs\|oUs\|oAs\|dGs\|dGs\|dTs\|dGs\|dAs\|dTs\|dGs\|5mCs\|dAs\|5mCs\|oUs\|oG |
| | s\|L5mCs\|oUs\|oU] |
| 45 | [oUs\|oGs\|o5mCs\|oUs\|oAs\|dGs\|dGs\|dTs\|dGs\|dAs\|dTs\|dGs\|5mCs\|dAs\|5mCs\|oUs\|LG |
| | s\|o5mCs\|oUs\|oU] |
| 452 | [oUs\|LGs\|o5mCs\|oUs\|oAs\|dGs\|dGs\|dTs\|dGs\|dAs\|dTs\|dGs\|5mCs\|dAs\|5mCs\|oUs\|oG |
| | s\|o5mCs\|oU] |
| 453 | [oUs\|oGs\|L5mCs\|oUs\|oAs\|dGs\|dGs\|dTs\|dGs\|dAs\|dTs\|dGs\|5mCs\|dAs\|5mCs\|oUs\|oG |
| | s\|o5mCs\|oU] |
| 454 | [oUs\|oGs\|o5mCs\|LTs\|oAs\|dGs\|dGs\|dTs\|dGs\|dAs\|dTs\|dGs\|5mCs\|dAs\|5mCs\|oUs\|oG |
| | s\|o5mCs\|oU] |
| 455 | [oGs\|o5mCs\|oUs\|oAs\|dGs\|dGs\|dTs\|dGs\|dAs\|dTs\|dGs\|5mCs\|dAs\|5mCs\|oUs\|oGs\|o5 |
| | mCs\|LTs\|oU] |
| 456 | [oGs\|o5mCs\|oUs\|oAs\|dGs\|dGs\|dTs\|dGs\|dAs\|dTs\|dGs\|5mCs\|dAs\|5mCs\|oUs\|oGs\|L5 |
| | mCs\|oUs\|oU] |
| 457 | [oGs\|o5mCs\|oUs\|oAs\|dGs\|dGs\|dTs\|dGs\|dAs\|dTs\|dGs\|5mCs\|dAs\|5mCs\|oUs\|LGs\|o5 |
| | mCs\|oUs\|oU] |
| 458 | [oUs\|LGs\|o5mCs\|oUs\|oAs\|dGs\|dGs\|dTs\|dGs\|dAs\|dTs\|dGs\|5mCs\|dAs\|5mCs\|oUs\|oG |
| | s\|o5mC] |
| 459 | [oUs\|oGs\|L5mCs\|oUs\|oAs\|dGs\|dGs\|dTs\|dGs\|dAs\|dTs\|dGs\|5mCs\|dAs\|5mCs\|oUs\|oG |
| | s\|o5mC] |
| 460 | [oUs\|oGs\|o5mCs\|LTs\|oAs\|dGs\|dGs\|dTs\|dGs\|dAs\|dTs\|dGs\|5mCs\|dAs\|5mCs\|oUs\|oG |
| | s\|o5mC] |
| 461 | [o5mCs\|oUs\|oAs\|dGs\|dGs\|dTs\|dGs\|dAs\|dTs\|dGs\|5mCs\|dAs\|5mCs\|oUs\|oGs\|o5mCs\| |
| | LTs\|oU] |
| 462 | [o5mCs\|oUs\|oAs\|dGs\|dGs\|dTs\|dGs\|dAs\|dTs\|dGs\|5mCs\|dAs\|5mCs\|oUs\|oGs\|L5mCs\| |
| | oUs\|oU] |
| 463 | [o5mCs\|oUs\|oAs\|dGs\|dGs\|dTs\|dGs\|dAs\|dTs\|dGs\|5mCs\|dAs\|5mCs\|oUs\|LGs\|o5mCs\| |
| | oUs\|oU] |
| 464 | [oUs\|LGs\|o5mCs\|oUs\|oAs\|dGs\|dGs\|dTs\|dGs\|dAs\|dTs\|dGs\|5mCs\|dAs\|5mCs\|oUs\|oG] |
| 465 | [oUs\|oGs\|L5mCs\|oUs\|oAs\|dGs\|dGs\|dTs\|dGs\|dAs\|dTs\|dGs\|5mCs\|dAs\|5mCs\|oUs\|oG] |
| 466 | [oUs\|oGs\|o5mCs\|LTs\|oAs\|dGs\|dGs\|dTs\|dGs\|dAs\|dTs\|dGs\|5mCs\|dAs\|5mCs\|oUs\|oG] |
| 467 | [oUs\|oAs\|dGs\|dGs\|dTs\|dGs\|dAs\|dTs\|dGs\|5mCs\|dAs\|5mCs\|oUs\|oGs\|o5mCs\|LTs\|oU] |
| 468 | [oUs\|oAs\|dGs\|dGs\|dTs\|dGs\|dAs\|dTs\|dGs\|5mCs\|dAs\|5mCs\|oUs\|oGs\|L5mCs\|oUs\|oU] |
| 469 | [oUs\|oAs\|dGs\|dGs\|dTs\|dGs\|dAs\|dTs\|dGs\|5mCs\|dAs\|5mCs\|oUs\|LGs\|o5mCs\|oUs\|oU] |
| 470 | [oGs\|L5mCs\|oUs\|oAs\|dGs\|dGs\|dTs\|dGs\|dAs\|dTs\|dGs\|5mCs\|dAs\|5mCs\|oUs\|oGs\|o5 |
| | mCs\|oUs\|oU] |
| 471 | [oGs\|o5mCs\|LTs\|oAs\|dGs\|dGs\|dTs\|dGs\|dAs\|dTs\|dGs\|5mCs\|dAs\|5mCs\|oUs\|oGs\|o5 |
| | mCs\|oUs\|oU] |
| 472 | [oUs\|oGs\|o5mCs\|oUs\|oAs\|dGs\|dGs\|dTs\|dGs\|dAs\|dTs\|dGs\|5mCs\|dAs\|5mCs\|oUs\|oG |
| | s\|L5mCs\|oU] |
| 473 | [oUs\|oGs\|o5mCs\|oUs\|oAs\|dGs\|dGs\|dTs\|dGs\|dAs\|dTs\|dGs\|5mCs\|dAs\|5mCs\|oUs\|LG |
| | s\|o5mCs\|oU] |
| 474 | [oGs\|L5mCs\|oUs\|oAs\|dGs\|dGs\|dTs\|dGs\|dAs\|dTs\|dGs\|5mCs\|dAs\|5mCs\|oUs\|oGs\|o5 |
| | mCs\|oU] |
| 475 | [oGs\|o5mCs\|LTs\|oAs\|dGs\|dGs\|dTs\|dGs\|dAs\|dTs\|dGs\|5mCs\|dAs\|5mCs\|oUs\|oGs\|o5 |
| | mCs\|oU] |
| 476 | [oGs\|o5mCs\|oUs\|oAs\|dGs\|dGs\|dTs\|dGs\|dAs\|dTs\|dGs\|5mCs\|dAs\|5mCs\|oUs\|oGs\|L5 |
| | mCs\|oU] |
| 477 | [oGs\|o5mCs\|oUs\|oAs\|dGs\|dGs\|dTs\|dGs\|dAs\|dTs\|dGs\|5mCs\|dAs\|5mCs\|oUs\|LGs\|o5 |
| | mCs\|oU] |
| 478 | [oGs\|L5mCs\|oUs\|oAs\|dGs\|dGs\|dTs\|dGs\|dAs\|dTs\|dGs\|5mCs\|dAs\|5mCs\|oUs\|oGs\|o5 |
| | mC] |
| 479 | [oGs\|o5mCs\|LTs\|oAs\|dGs\|dGs\|dTs\|dGs\|dAs\|dTs\|dGs\|5mCs\|dAs\|5mCs\|oUs\|oGs\|o5 |
| | mC] |
| 480 | [oGs\|o5mCs\|oUs\|oAs\|dGs\|dGs\|dTs\|dGs\|dAs\|dTs\|dGs\|5mCs\|dAs\|5mCs\|oUs\|LGs\|o5 |
| | mC] |
| 481 | [oUs\|oGs\|o5mCs\|oUs\|oAs\|oGs\|oGs\|oUs\|dGs\|dAs\|dTs\|dGs\|5mCs\|dAs\|5mCs\|dTs\|dG |
| | s\|5mCs\|oUs\|oU] |
| 482 | [oUs\|oGs\|5mCs\|dTs\|dAs\|dGs\|dGs\|dTs\|dGs\|dAs\|dTs\|dGs\|o5mCs\|oAs\|o5mCs\|oUs\|o |
| | Gs\|o5mCs\|oUs\|oU] |
| 483 | [oUs\|oGs\|o5mCs\|oUs\|oAs\|oGs\|oGs\|dTs\|dGs\|dAs\|dTs\|dGs\|5mCs\|dAs\|5mCs\|dTs\|dG |
| | s\|o5mCs\|oUs\|oU] |
| 484 | [oUs\|oGs\|o5mCs\|dTs\|dAs\|dGs\|dGs\|dTs\|dGs\|dAs\|dTs\|dGs\|5mCs\|oAs\|o5mCs\|oUs\|o |
| | Gs\|o5mCs\|oUs\|oU] |
| 485 | [oUs\|oGs\|o5mCs\|oUs\|oAs\|oGs\|oGs\|dTs\|dGs\|dAs\|dTs\|dGs\|5mCs\|dAs\|5mCs\|dTs\|dG |
| | s\|5mCs\|oUs\|oU] |
| 486 | [oGs\|o5mCs\|dTs\|dAs\|dGs\|dGs\|dTs\|dGs\|dAs\|dTs\|dGs\|5mCs\|oAs\|o5mCs\|oUs\|oGs\|o5 |
| | mCs\|oUs\|oU] |
| 487 | [oUs\|oGs\|o5mCs\|oUs\|oAs\|oGs\|dGs\|dTs\|dGs\|dAs\|dTs\|dGs\|5mCs\|dAs\|5mCs\|dTs\|oG |
| | s\|o5mCs\|oUs\|oU] |
| 488 | [oUs\|oGs\|o5mCs\|oUs\|dAs\|dGs\|dGs\|dTs\|dGs\|dAs\|dTs\|dGs\|5mCs\|dAs\|o5mCs\|oUs\|o |
| | Gs\|o5mCs\|oUs\|oU] |
| 489 | [oUs\|oGs\|o5mCs\|oUs\|oAs\|oGs\|dGs\|dTs\|dGs\|dAs\|dTs\|dGs\|5mCs\|dAs\|5mCs\|dTs\|oG |
| | s\|o5mCs\|oU] |
| 490 | [oGs\|o5mCs\|oUs\|dAs\|dGs\|dGs\|dTs\|dGs\|dAs\|dTs\|dGs\|5mCs\|dAs\|o5mCs\|oUs\|oGs\|o |
| | 5mCs\|oUs\|oU] |
| 49 | [oUs\|oGs\|o5mCs\|oUs\|oAs\|oGs\|dGs\|dTs\|dGs\|dAs\|dTs\|dGs\|5mCs\|dAs\|5mCs\|dTs\|oG |
| | s\|o5mC] |
| 492 | [o5mCs\|oUs\|dAs\|dGs\|dGs\|dTs\|dGs\|dAs\|dTs\|dGs\|5mCs\|dAs\|o5mCs\|oUs\|oGs\|o5mC |
| | s\|oUs\|oU] |
| 493 | [oUs\|oGs\|o5mCs\|oUs\|oAs\|dGs\|dGs\|dTs\|dGs\|dAs\|dTs\|dGs\|5mCs\|dAs\|5mCs\|oUs\|oG |
| | s\|o5mCs\|oUs\|oU] |
| 494 | [oUs\|oGs\|o5mCs\|oUs\|oAs\|dGs\|dGs\|dTs\|dGs\|dAs\|dTs\|dGs\|5mCs\|dAs\|5mCs\|oUs\|oG |
| | s\|o5mCs\|oU] |
| 495 | [oUs\|oGs\|o5mCs\|oUs\|oAs\|dGs\|dGs\|dTs\|dGs\|dAs\|dTs\|dGs\|5mCs\|dAs\|5mCs\|oUs\|oG |
| | s\|o5mC] |
| 496 | [o5mCs\|oUs\|oAs\|dGs\|dGs\|dTs\|dGs\|dAs\|dTs\|dGs\|5mCs\|dAs\|5mCs\|oUs\|oGs\|o5mCs\| |
| | oUs\|oU] |
| 497 | [oUs\|oGs\|o5mCs\|oUs\|oAs\|dGs\|dGs\|dTs\|dGs\|dAs\|dTs\|dGs\|5mCs\|dAs\|5mCs\|oUs\|oG] |
| 498 | [oUs\|oAs\|dGs\|dGs\|dTs\|dGs\|dAs\|dTs\|dGs\|5mCs\|dAs\|5mCs\|oUs\|oGs\|o5mCs\|oUs\|oU] |
| 499 | [oGs\|o5mCs\|oUs\|oAs\|dGs\|dGs\|dTs\|dGs\|dAs\|dTs\|dGs\|5mCs\|dAs\|5mCs\|oUs\|oGs\|o5 |
| | mCs\|oUs\|oU] |
| 500 | [oGs\|o5mCs\|oUs\|oAs\|dGs\|dGs\|dTs\|dGs\|dAs\|dTs\|dGs\|5mCs\|dAs\|5mCs\|oUs\|oGs\|o5 |
| | mCs\|oU] |
| 501 | [oGs\|o5mCs\|oUs\|oAs\|dGs\|dGs\|dTs\|dGs\|dAs\|dTs\|dGs\|5mCs\|dAs\|5mCs\|oUs\|oGs\|o5 |
| | mC] |
| 502 | [oUs\|LTs\|oGs\|oAs\|oUs\|o5mCs\|o5mCs\|oUs\|dGs\|dTs\|dTs\|5mCs\|dTs\|5mCs\|5mCs\|5m |
| | Cs\|dAs\|dGs\|o5mCs\|oA] |
| 503 | [oUs\|oUs\|LGs\|oAs\|oUs\|o5mCs\|o5mCs\|oUs\|dGs\|dTs\|dTs\|5mCs\|dTs\|5mCs\|5mCs\|5m |
| | Cs\|dAs\|dGs\|o5mCs\|oA] |
| 504 | [oUs\|oUs\|oGs\|LAs\|oUs\|o5mCs\|o5mCs\|oUs\|dGs\|dTs\|dTs\|5mCs\|dTs\|5mCs\|5mCs\|5m |
| | Cs\|dAs\|dGs\|o5mCs\|oA] |
| 505 | [oUs\|oUs\|oGs\|oAs\|LTs\|o5mCs\|o5mCs\|oUs\|dGs\|dTs\|dTs\|5mCs\|dTs\|5mCs\|5mCs\|5m |
| | Cs\|dAs\|dGs\|o5mCs\|oA] |
| 506 | [oUs\|oUs\|oGs\|oAs\|oUs\|L5mCs\|o5mCs\|oUs\|dGs\|dTs\|dTs\|5mCs\|dTs\|5mCs\|5mCs\|5m |
| | Cs\|dAs\|dGs\|o5mCs\|oA] |
| 507 | [oUs\|oUs\|oGs\|oAs\|oUs\|o5mCs\|L5mCs\|oUs\|dGs\|dTs\|dTs\|5mCs\|dTs\|5mCs\|5mCs\|5m |
| | Cs\|dAs\|dGs\|o5mCs\|oA] |
| 508 | [oUs\|oUs\|dGs\|dAs\|dTs\|5mCs\|5mCs\|dTs\|dGs\|dTs\|dTs\|5mCs\|oUs\|o5mCs\|o5mCs\|o5 |
| | mCs\|oAs\|oGs\|L5mCs\|oA] |
| 509 | [oUs\|oUs\|dGs\|dAs\|dTs\|5mCs\|5mCs\|dTs\|dGs\|dTs\|dTs\|5mCs\|oUs\|o5mCs\|o5mCs\|o5 |
| | mCs\|oAs\|LGs\|o5mCs\|oA] |
| 510 | [oUs\|oUs\|dGs\|dAs\|dTs\|5mCs\|5mCs\|dTs\|dGs\|dTs\|dTs\|5mCs\|oUs\|o5mCs\|o5mCs\|o5 |
| | mCs\|LAs\|oGs\|o5mCs\|oA] |
| 511 | [oUs\|oUs\|dGs\|dAs\|dTs\|5mCs\|5mCs\|dTs\|dGs\|dTs\|dTs\|5mCs\|oUs\|o5mCs\|o5mCs\|L5 |
| | mCs\|oAs\|oGs\|o5mCs\|oA] |
| 512 | [oUs\|oUs\|dGs\|dAs\|dTs\|5mCs\|5mCs\|dTs\|dGs\|dTs\|dTs\|5mCs\|oUs\|o5mCs\|L5mCs\|o5 |
| | mCs\|oAs\|oGs\|o5mCs\|oA] |
| 513 | [oUs\|oUs\|dGs\|dAs\|dTs\|5mCs\|5mCs\|dTs\|dGs\|dTs\|dTs\|5mCs\|oUs\|L5mCs\|o5mCs\|o5 |
| | mCs\|oAs\|oGs\|o5mCs\|oA] |
| 514 | [oUs\|LTs\|oGs\|oAs\|oUs\|o5mCs\|o5mCs\|dTs\|dGs\|dTs\|dTs\|5mCs\|dTs\|5mCs\|5mCs\|5m |
| | Cs\|dAs\|oGs\|o5mCs\|oA] |
| 515 | [oUs\|oUs\|LGs\|oAs\|oUs\|o5mCs\|o5mCs\|dTs\|dGs\|dTs\|dTs\|5mCs\|dTs\|5mCs\|5mCs\|5m |
| | Cs\|dAs\|oGs\|o5mCs\|oA] |
| 516 | [oUs\|oUs\|oGs\|LAs\|oUs\|o5mCs\|o5mCs\|dTs\|dGs\|dTs\|dTs\|5mCs\|dTs\|5mCs\|5mCs\|5m |
| | Cs\|dAs\|oGs\|o5mCs\|oA] |
| 517 | [oUs\|oUs\|oGs\|oAs\|LTs\|o5mCs\|o5mCs\|dTs\|dGs\|dTs\|dTs\|5mCs\|dTs\|5mCs\|5mCs\|5m |
| | Cs\|dAs\|oGs\|o5mCs\|oA] |
| 518 | [oUs\|oUs\|oGs\|oAs\|oUs\|L5mCs\|o5mCs\|dTs\|dGs\|dTs\|dTs\|5mCs\|dTs\|5mCs\|5mCs\|5m |
| | Cs\|dAs\|oGs\|o5mCs\|oA] |
| 519 | [oUs\|oUs\|oGs\|dAs\|dTs\|5mCs\|5mCs\|dTs\|dGs\|dTs\|dTs\|5mCs\|dTs\|o5mCs\|o5mCs\|o5 |
| | mCs\|oAs\|oGs\|L5mCs\|oA] |
| 520 | [oUs\|oUs\|oGs\|dAs\|dTs\|5mCs\|5mCs\|dTs\|dGs\|dTs\|dTs\|5mCs\|dTs\|o5mCs\|o5mCs\|o5 |
| | mCs\|oAs\|LGs\|o5mCs\|oA] |
| 521 | [oUs\|oUs\|oGs\|dAs\|dTs\|5mCs\|5mCs\|dTs\|dGs\|dTs\|dTs\|5mCs\|dTs\|o5mCs\|o5mCs\|o5 |
| | mCs\|LAs\|oGs\|o5mCs\|oA] |
| 522 | [oUs\|oUs\|oGs\|dAs\|dTs\|5mCs\|5mCs\|dTs\|dGs\|dTs\|dTs\|5mCs\|dTs\|o5mCs\|o5mCs\|L5 |
| | mCs\|oAs\|oGs\|o5mCs\|oA] |
| 523 | [oUs\|oUs\|oGs\|dAs\|dTs\|5mCs\|5mCs\|dTs\|dGs\|dTs\|dTs\|5mCs\|dTs\|o5mCs\|L5mCs\|o5 |
| | mCs\|oAs\|oGs\|o5mCs\|oA] |
| 524 | [oUs\|LTs\|oGs\|oAs\|oUs\|o5mCs\|o5mCs\|dTs\|dGs\|dTs\|dTs\|5mCs\|dTs\|5mCs\|5mCs\|5m |
| | Cs\|dAs\|oGs\|o5mC] |
| 525 | [oUs\|oUs\|LGs\|oAs\|oUs\|o5mCs\|o5mCs\|dTs\|dGs\|dTs\|dTs\|5mCs\|dTs\|5mCs\|5mCs\|5m |
| | Cs\|dAs\|oGs\|o5mC] |
| 526 | [oUs\|oUs\|oGs\|LAs\|oUs\|o5mCs\|o5mCs\|dTs\|dGs\|dTs\|dTs\|5mCs\|dTs\|5mCs\|5mCs\|5m |
| | Cs\|dAs\|oGs\|o5mC] |
| 527 | [oUs\|oUs\|oGs\|oAs\|LTs\|o5mCs\|o5mCs\|dTs\|dGs\|dTs\|dTs\|5mCs\|dTs\|5mCs\|5mCs\|5m |
| | Cs\|dAs\|oGs\|o5mC] |
| 528 | [oUs\|oUs\|oGs\|oAs\|oUs\|L5mCs\|o5mCs\|dTs\|dGs\|dTs\|dTs\|5mCs\|dTs\|5mCs\|5mCs\|5m |
| | Cs\|dAs\|oGs\|o5mC] |
| 529 | [oUs\|oGs\|dAs\|dTs\|5mCs\|5mCs\|dTs\|dGs\|dTs\|dTs\|5mCs\|dTs\|o5mCs\|o5mCs\|o5mCs\| |
| | oAs\|oGs\|L5mCs\|oA] |
| 530 | [oUs\|oGs\|dAs\|dTs\|5mCs\|5mCs\|dTs\|dGs\|dTs\|dTs\|5mCs\|dTs\|o5mCs\|o5mCs\|o5mCs\| |
| | oAs\|LGs\|o5mCs\|oA] |
| 531 | [oUs\|oGs\|dAs\|dTs\|5mCs\|5mCs\|dTs\|dGs\|dTs\|dTs\|5mCs\|dTs\|o5mCs\|o5mCs\|o5mCs\| |
| | LAs\|oGs\|o5mCs\|oA] |
| 532 | [oUs\|oGs\|dAs\|dTs\|5mCs\|5mCs\|dTs\|dGs\|dTs\|dTs\|5mCs\|dTs\|o5mCs\|o5mCs\|L5mCs\| |
| | oAs\|oGs\|o5mCs\|oA] |
| 533 | [oUs\|oGs\|dAs\|dTs\|5mCs\|5mCs\|dTs\|dGs\|dTs\|dTs\|5mCs\|dTs\|o5mCs\|L5mCs\|o5mCs\| |
| | oAs\|oGs\|o5mCs\|oA] |
| 534 | [oUs\|LTs\|oGs\|oAs\|oUs\|o5mCs\|5mCs\|dTs\|dGs\|dTs\|dTs\|5mCs\|dTs\|5mCs\|5mCs\|5mC |
| | s\|oAs\|oGs\|o5mCs\|oA] |
| 535 | [oUs\|oUs\|LGs\|oAs\|oUs\|o5mCs\|5mCs\|dTs\|dGs\|dTs\|dTs\|5mCs\|dTs\|5mCs\|5mCs\|5m |
| | Cs\|oAs\|oGs\|o5mCs\|oA] |
| 536 | [oUs\|oUs\|oGs\|LAs\|oUs\|o5mCs\|5mCs\|dTs\|dGs\|dTs\|dTs\|5mCs\|dTs\|5mCs\|5mCs\|5m |
| | Cs\|oAs\|oGs\|o5mCs\|oA] |
| 537 | [oUs\|oUs\|oGs\|oAs\|LTs\|o5mCs\|5mCs\|dTs\|dGs\|dTs\|dTs\|5mCs\|dTs\|5mCs\|5mCs\|5mC |
| | s\|oAs\|oGs\|o5mCs\|oA] |
| 538 | [oUs\|oUs\|oGs\|oAs\|dTs\|5mCs\|5mCs\|dTs\|dGs\|dTs\|dTs\|5mCs\|dTs\|5mCs\|o5mCs\|o5m |
| | Cs\|oAs\|oGs\|L5mCs\|oA] |
| 539 | [oUs\|oUs\|oGs\|oAs\|dTs\|5mCs\|5mCs\|dTs\|dGs\|dTs\|dTs\|5mCs\|dTs\|5mCs\|o5mCs\|o5m |
| | Cs\|oAs\|LGs\|o5mCs\|oA] |
| 540 | [oUs\|oUs\|oGs\|oAs\|dTs\|5mCs\|5mCs\|dTs\|dGs\|dTs\|dTs\|5mCs\|dTs\|5mCs\|o5mCs\|o5m |
| | Cs\|LAs\|oGs\|o5mCs\|oA] |
| 541 | [oUs\|oUs\|oGs\|oAs\|dTs\|5mCs\|5mCs\|dTs\|dGs\|dTs\|dTs\|5mCs\|dTs\|5mCs\|o5mCs\|L5m |
| | Cs\|oAs\|oGs\|o5mCs\|oA] |
| 542 | [oUs\|LTs\|oGs\|oAs\|oUs\|o5mCs\|5mCs\|dTs\|dGs\|dTs\|dTs\|5mCs\|dTs\|5mCs\|5mCs\|5mC |
| | s\|oAs\|oGs\|o5mC] |
| 543 | [oUs\|oUs\|LGs\|oAs\|oUs\|o5mCs\|5mCs\|dTs\|dGs\|dTs\|dTs\|5mCs\|dTs\|5mCs\|5mCs\|5m |
| | Cs\|oAs\|oGs\|o5mC] |
| 544 | [oUs\|oUs\|oGs\|LAs\|oUs\|o5mCs\|5mCs\|dTs\|dGs\|dTs\|dTs\|5mCs\|dTs\|5mCs\|5mCs\|5m |
| | Cs\|oAs\|oGs\|o5mC] |
| 545 | [oUs\|oUs\|oGs\|oAs\|LTs\|o5mCs\|5mCs\|dTs\|dGs\|dTs\|dTs\|5mCs\|dTs\|5mCs\|5mCs\|5mC |
| | s\|oAs\|oGs\|o5mC] |
| 546 | [oUs\|oUs\|oGs\|dAs\|dTs\|5mCs\|5mCs\|dTs\|dGs\|dTs\|dTs\|5mCs\|dTs\|5mCs\|o5mCs\|o5m |
| | Cs\|oAs\|oGs\|L5mCs\|oA] |
| 547 | [oUs\|oUs\|oGs\|dAs\|dTs\|5mCs\|5mCs\|dTs\|dGs\|dTs\|dTs\|5mCs\|dTs\|5mCs\|o5mCs\|o5m |
| | Cs\|oAs\|LGs\|o5mCs\|oA] |
| 548 | [oUs\|oUs\|oGs\|dAs\|dTs\|5mCs\|5mCs\|dTs\|dGs\|dTs\|dTs\|5mCs\|dTs\|5mCs\|o5mCs\|o5m |
| | Cs\|LAs\|oGs\|o5mCs\|oA] |
| 549 | [oUs\|oUs\|oGs\|oAs\|dTs\|5mCs\|5mCs\|dTs\|dGs\|dTs\|dTs\|5mCs\|dTs\|5mCs\|o5mCs\|L5m |
| | Cs\|oAs\|oGs\|o5mCs\|oA] |
| 550 | [oUs\|LTs\|oGs\|oAs\|oUs\|o5mCs\|5mCs\|dTs\|dGs\|dTs\|dTs\|5mCs\|dTs\|5mCs\|5mCs\|5mC |
| | s\|oAs\|oG] |
| 551 | [oUs\|oUs\|LGs\|oAs\|oUs\|o5mCs\|5mCs\|dTs\|dGs\|dTs\|dTs\|5mCs\|dTs\|5mCs\|5mCs\|5m |
| | Cs\|oAs\|oG] |
| 552 | [oUs\|oUs\|oGs\|LAs\|oUs\|o5mCs\|5mCs\|dTs\|dGs\|dTs\|dTs\|5mCs\|dTs\|5mCs\|5mCs\|5m |
| | Cs\|oAs\|oG] |
| 553 | [oUs\|oUs\|oGs\|oAs\|LTs\|o5mCs\|5mCs\|dTs\|dGs\|dTs\|dTs\|5mCs\|dTs\|5mCs\|5mCs\|5mC |
| | s\|oAs\|oG] |
| 554 | [oGs\|oAs\|dTs\|5mCs\|5mCs\|dTs\|dGs\|dTs\|dTs\|5mCs\|dTs\|5mCs\|o5mCs\|o5mCs\|oAs\|o |
| | Gs\|L5mCs\|oA] |
| 555 | [oGs\|oAs\|dTs\|5mCs\|5mCs\|dTs\|dGs\|dTs\|dTs\|5mCs\|dTs\|5mCs\|o5mCs\|o5mCs\|oAs\|L |
| | Gs\|o5mCs\|oA] |
| 556 | [oGs\|oAs\|dTs\|5mCs\|5mCs\|dTs\|dGs\|dTs\|dTs\|5mCs\|dTs\|5mCs\|o5mCs\|o5mCs\|LAs\|o |
| | Gs\|o5mCs\|oA] |
| 557 | [oGs\|oAs\|dTs\|5mCs\|5mCs\|dTs\|dGs\|dTs\|dTs\|5mCs\|dTs\|5mCs\|o5mCs\|L5mCs\|oAs\|o |
| | Gs\|o5mCs\|oA] |
| 558 | [oUs\|LTs\|oGs\|oAs\|oUs\|5mCs\|5mCs\|dTs\|dGs\|dTs\|dTs\|5mCs\|dTs\|5mCs\|5mCs\|o5mC |
| | s\|oAs\|oGs\|o5mCs\|oA] |
| 559 | [oUs\|oUs\|LGs\|oAs\|oUs\|5mCs\|5mCs\|dTs\|dGs\|dTs\|dTs\|5mCs\|dTs\|5mCs\|5mCs\|o5m |
| | Cs\|oAs\|oGs\|o5mCs\|oA] |
| 560 | [oUs\|oUs\|oGs\|LAs\|oUs\|5mCs\|5mCs\|dTs\|dGs\|dTs\|dTs\|5mCs\|dTs\|5mCs\|5mCs\|o5m |
| | Cs\|oAs\|oGs\|o5mCs\|oA] |
| 561 | [oUs\|oUs\|oGs\|oAs\|oUs\|5mCs\|5mCs\|dTs\|dGs\|dTs\|dTs\|5mCs\|dTs\|5mCs\|5mCs\|o5mC |
| | s\|oAs\|oGs\|L5mCs\|oA] |
| 562 | [oUs\|oUs\|oGs\|oAs\|oUs\|5mCs\|5mCs\|dTs\|dGs\|dTs\|dTs\|5mCs\|dTs\|5mCs\|5mCs\|o5mC |
| | s\|oAs\|LGs\|o5mCs\|oA] |
| 563 | [oUs\|oUs\|oGs\|oAs\|oUs\|5mCs\|5mCs\|dTs\|dGs\|dTs\|dTs\|5mCs\|dTs\|5mCs\|5mCs\|o5mC |
| | s\|LAs\|oGs\|o5mCs\|oA] |
| 564 | [oUs\|LTs\|oGs\|oAs\|oUs\|5mCs\|5mCs\|dTs\|dGs\|dTs\|dTs\|5mCs\|dTs\|5mCs\|5mCs\|o5mC |
| | s\|oAs\|oGs\|o5mC] |
| 565 | [oUs\|oUs\|LGs\|oAs\|oUs\|5mCs\|5mCs\|dTs\|dGs\|dTs\|dTs\|5mCs\|dTs\|5mCs\|5mCs\|o5m |
| | Cs\|oAs\|oGs\|o5mC] |
| 566 | [oUs\|oUs\|oGs\|LAs\|oUs\|5mCs\|5mCs\|dTs\|dGs\|dTs\|dTs\|5mCs\|dTs\|5mCs\|5mCs\|o5m |
| | Cs\|oAs\|oGs\|o5mC] |
| 567 | [oUs\|oGs\|oAs\|oUs\|5mCs\|5mCs\|dTs\|dGs\|dTs\|dTs\|5mCs\|dTs\|5mCs\|5mCs\|o5mCs\|oA |
| | s\|oGs\|L5mCs\|oA] |
| 568 | [oUs\|oGs\|oAs\|oUs\|5mCs\|5mCs\|dTs\|dGs\|dTs\|dTs\|5mCs\|dTs\|5mCs\|5mCs\|o5mCs\|oA |
| | s\|LGs\|o5mCs\|oA] |
| 569 | [oUs\|oGs\|oAs\|oUs\|5mCs\|5mCs\|dTs\|dGs\|dTs\|dTs\|5mCs\|dTs\|5mCs\|5mCs\|o5mCs\|L |
| | As\|oGs\|o5mCs\|oA] |
| 570 | [oUs\|LTs\|oGs\|oAs\|oUs\|5mCs\|5mCs\|dTs\|dGs\|dTs\|dTs\|5mCs\|dTs\|5mCs\|5mCs\|o5mC |
| | s\|oAs\|oG] |
| 571 | [oUs\|oUs\|LGs\|oAs\|oUs\|5mCs\|5mCs\|dTs\|dGs\|dTs\|dTs\|5mCs\|dTs\|5mCs\|5mCs\|o5m |
| | Cs\|oAs\|oG] |
| 572 | [oUs\|oUs\|oGs\|LAs\|oUs\|5mCs\|5mCs\|dTs\|dGs\|dTs\|dTs\|5mCs\|dTs\|5mCs\|5mCs\|o5m |
| | Cs\|oAs\|oG] |
| 573 | [oGs\|oAs\|oUs\|5mCs\|5mCs\|dTs\|dGs\|dTs\|dTs\|5mCs\|dTs\|5mCs\|5mCs\|o5mCs\|oAs\|oG |
| | s\|L5mCs\|oA] |
| 574 | [oGs\|oAs\|oUs\|5mCs\|5mCs\|dTs\|dGs\|dTs\|dTs\|5mCs\|dTs\|5mCs\|5mCs\|o5mCs\|oAs\|L |
| | Gs\|o5mCs\|oA] |
| 575 | [oGs\|oAs\|oUs\|5mCs\|5mCs\|dTs\|dGs\|dTs\|dTs\|5mCs\|dTs\|5mCs\|5mCs\|o5mCs\|LAs\|o |
| | Gs\|o5mCs\|oA] |
| 576 | [oUs\|LTs\|oGs\|oAs\|oUs\|5mCs\|5mCs\|dTs\|dGs\|dTs\|dTs\|5mCs\|dTs\|5mCs\|5mCs\|o5mC |
| | s\|oA] |
| 577 | [oUs\|oUs\|LGs\|oAs\|oUs\|5mCs\|5mCs\|dTs\|dGs\|dTs\|dTs\|5mCs\|dTs\|5mCs\|5mCs\|o5m |
| | Cs\|oA] |
| 578 | [oUs\|oUs\|oGs\|LAs\|oUs\|5mCs\|5mCs\|dTs\|dGs\|dTs\|dTs\|5mCs\|dTs\|5mCs\|5mCs\|o5m |
| | Cs\|oA] |
| 579 | [oAs\|oUs\|5mCs\|5mCs\|dTs\|dGs\|dTs\|dTs\|5mCs\|dTs\|5mCs\|5mCs\|o5mCs\|oAs\|oGs\|L5 |
| | mCs\|oA] |
| 580 | [oAs\|oUs\|5mCs\|5mCs\|dTs\|dGs\|dTs\|dTs\|5mCs\|dTs\|5mCs\|5mCs\|o5mCs\|oAs\|LGs\|o5 |
| | mCs\|oA] |
| 581 | [oAs\|oUs\|5mCs\|5mCs\|dTs\|dGs\|dTs\|dTs\|5mCs\|dTs\|5mCs\|5mCs\|o5mCs\|LAs\|oGs\|o5 |
| | mCs\|oA] |
| 582 | [oUs\|LGs\|oAs\|oUs\|5mCs\|5mCs\|dTs\|dGs\|dTs\|dTs\|5mCs\|dTs\|5mCs\|5mCs\|o5mCs\|o |
| | As\|oGs\|o5mCs\|oA] |
| 583 | [oUs\|oGs\|LAs\|oUs\|5mCs\|5mCs\|dTs\|dGs\|dTs\|dTs\|5mCs\|dTs\|5mCs\|5mCs\|o5mCs\|o |
| | As\|oGs\|o5mCs\|oA] |
| 584 | [oUs\|oUs\|oGs\|oAs\|oUs\|5mCs\|5mCs\|dTs\|dGs\|dTs\|dTs\|5mCs\|dTs\|5mCs\|5mCs\|o5mC |
| | s\|oAs\|LGs\|o5mC] |
| 585 | [oUs\|oUs\|oGs\|oAs\|oUs\|5mCs\|5mCs\|dTs\|dGs\|dTs\|dTs\|5mCs\|dTs\|5mCs\|5mCs\|o5mC |
| | s\|LAs\|oGs\|o5mC] |
| 586 | [oUs\|LGs\|oAs\|oUs\|5mCs\|5mCs\|dTs\|dGs\|dTs\|dTs\|5mCs\|dTs\|5mCs\|5mCs\|o5mCs\|o |
| | As\|oGs\|o5mC] |
| 587 | [oUs\|oGs\|LAs\|oUs\|5mCs\|5mCs\|dTs\|dGs\|dTs\|dTs\|5mCs\|dTs\|5mCs\|5mCs\|o5mCs\|o |
| | As\|oGs\|o5mC] |
| 588 | [oUs\|oGs\|oAs\|oUs\|5mCs\|5mCs\|dTs\|dGs\|dTs\|dTs\|5mCs\|dTs\|5mCs\|5mCs\|o5mCs\|oA |
| | s\|LGs\|o5mC] |
| 589 | [oUs\|oGs\|oAs\|oUs\|5mCs\|5mCs\|dTs\|dGs\|dTs\|dTs\|5mCs\|dTs\|5mCs\|5mCs\|o5mCs\|L |
| | As\|oGs\|o5mC] |
| 590 | [oUs\|LGs\|oAs\|oUs\|5mCs\|5mCs\|dTs\|dGs\|dTs\|dTs\|5mCs\|dTs\|5mCs\|5mCs\|o5mCs\|o |
| | As\|oG] |
| 591 | [oUs\|oGs\|LAs\|oUs\|5mCs\|5mCs\|dTs\|dGs\|dTs\|dTs\|5mCs\|dTs\|5mCs\|5mCs\|o5mCs\|o |
| | As\|oG] |
| 592 | [oUs\|oGs\|oAs\|oUs\|5mCs\|5mCs\|dTs\|dGs\|dTs\|dTs\|5mCs\|dTs\|5mCs\|5mCs\|o5mCs\|L |
| | As\|oG] |
| 593 | [oUs\|oUs\|oGs\|oAs\|oUs\|o5mCs\|o5mCs\|oUs\|dGs\|dTs\|dTs\|5mCs\|dTs\|5mCs\|5mCs\|5m |
| | Cs\|dAs\|dGs\|o5mCs\|oA] |
| 594 | [oUs\|oUs\|dGs\|dAs\|dTs\|5mCs\|5mCs\|dTs\|dGs\|dTs\|dTs\|5mCs\|oUs\|o5mCs\|o5mCs\|o5 |
| | mCs\|oAs\|oGs\|o5mCs\|oA] |
| 595 | [oUs\|oUs\|oGs\|oAs\|oUs\|o5mCs\|o5mCs\|dTs\|dGs\|dTs\|dTs\|5mCs\|dTs\|5mCs\|5mCs\|5m |
| | Cs\|dAs\|oGs\|o5mCs\|oA] |
| 596 | [oUs\|oUs\|oGs\|dAs\|dTs\|5mCs\|5mCs\|dTs\|dGs\|dTs\|dTs\|5mCs\|dTs\|o5mCs\|o5mCs\|o5 |
| | mCs\|oAs\|oGs\|o5mCs\|oA] |
| 597 | [oUs\|oUs\|oGs\|oAs\|oUs\|o5mCs\|o5mCs\|dTs\|dGs\|dTs\|dTs\|5mCs\|dTs\|5mCs\|5mCs\|5m |
| | Cs\|dAs\|oGs\|o5mC] |
| 598 | [oUs\|oGs\|dAs\|dTs\|5mCs\|5mCs\|dTs\|dGs\|dTs\|dTs\|5mCs\|dTs\|o5mCs\|o5mCs\|o5mCs\| |
| | oAs\|oGs\|o5mCs\|oA] |
| 599 | [oUs\|oUs\|oGs\|oAs\|oUs\|o5mCs\|5mCs\|dTs\|dGs\|dTs\|dTs\|5mCs\|dTs\|5mCs\|5mCs\|5mC |
| | s\|oAs\|oGs\|o5mCs\|oA] |
| 600 | [oUs\|oUs\|oGs\|oAs\|dTs\|5mCs\|5mCs\|dTs\|dGs\|dTs\|dTs\|5mCs\|dTs\|5mCs\|o5mCs\|o5m |
| | Cs\|oAs\|oGs\|o5mCs\|oA] |
| 601 | [oUs\|oUs\|oGs\|oAs\|oUs\|o5mCs\|5mCs\|dTs\|dGs\|dTs\|dTs\|5mCs\|dTs\|5mCs\|5mCs\|5mC |
| | s\|oAs\|oGs\|o5mC] |
| 602 | [oUs\|oUs\|oGs\|dAs\|dTs\|5mCs\|5mCs\|dTs\|dGs\|dTs\|dTs\|5mCs\|dTs\|5mCs\|o5mCs\|o5m |
| | Cs\|oAs\|oGs\|o5mCs\|oA] |
| 603 | [oUs\|oUs\|oGs\|oAs\|oUs\|o5mCs\|5mCs\|dTs\|dGs\|dTs\|dTs\|5mCs\|dTs\|5mCs\|5mCs\|5mC |
| | s\|oAs\|oG] |
| 604 | [oGs\|oAs\|dTs\|5mCs\|5mCs\|dTs\|dGs\|dTs\|dTs\|5mCs\|dTs\|5mCs\|o5mCs\|o5mCs\|oAs\|o |
| | Gs\|o5mCs\|oA] |
| 605 | [oUs\|oUs\|oGs\|oAs\|oUs\|5mCs\|5mCs\|dTs\|dGs\|dTs\|dTs\|5mCs\|dTs\|5mCs\|5mCs\|o5mC |
| | s\|oAs\|oGs\|o5mCs\|oA] |
| 606 | [oUs\|oUs\|oGs\|oAs\|oUs\|5mCs\|5mCs\|dTs\|dGs\|dTs\|dTs\|5mCs\|dTs\|5mCs\|5mCs\|o5mC |
| | s\|oAs\|oGs\|o5mC] |
| 607 | [oUs\|oUs\|oGs\|oAs\|oUs\|5mCs\|5mCs\|dTs\|dGs\|dTs\|dTs\|5mCs\|dTs\|5mCs\|5mCs\|o5mC |
| | s\|oAs\|oG] |
| 608 | [oGs\|oAs\|oUs\|5mCs\|5mCs\|dTs\|dGs\|dTs\|dTs\|5mCs\|dTs\|5mCs\|5mCs\|o5mCs\|oAs\|oG |
| | s\|o5mCs\|oA] |
| 609 | [oUs\|oUs\|oGs\|oAs\|oUs\|5mCs\|5mCs\|dTs\|dGs\|dTs\|dTs\|5mCs\|dTs\|5mCs\|5mCs\|o5mC |
| | s\|oA] |
| 610 | [oAs\|oUs\|5mCs\|5mCs\|dTs\|dGs\|dTs\|dTs\|5mCs\|dTs\|5mCs\|5mCs\|o5mCs\|oAs\|oGs\|o5 |
| | mCs\|oA] |
| 611 | [oUs\|oGs\|oAs\|oUs\|5mCs\|5mCs\|dTs\|dGs\|dTs\|dTs\|5mCs\|dTs\|5mCs\|5mCs\|o5mCs\|oA |
| | s\|oGs\|o5mCs\|oA] |
| 612 | [oUs\|oGs\|oAs\|oUs\|5mCs\|5mCs\|dTs\|dGs\|dTs\|dTs\|5mCs\|dTs\|5mCs\|5mCs\|o5mCs\|oA |
| | s\|oGs\|o5mC] |
| 613 | [oUs\|oGs\|oAs\|oUs\|5mCs\|5mCs\|dTs\|dGs\|dTs\|dTs\|5mCs\|dTs\|5mCs\|5mCs\|o5mCs\|oA |
| | s\|oG] |
| 617 | [oCs\|oGs\|oGs\|oGs\|oCs\|dAs\|dTs\|dTs\|dTs\|dAs\|dTs\|dTs\|dTs\|5mCs\|5mCs\|oCs\|oUs\|oU |
| | s\|oAs\|oA] |
| 618 | [oGs\|oAs\|oUs\|oCs\|oAs\|5mCs\|dAs\|dGs\|dGs\|5mCs\|dTs\|dTs\|5mCs\|dTs\|5mCs\|oCs\|oA |
| | s\|oAs\|oGs\|oU] |
| 619 | [oUs\|oAs\|oAs\|oAs\|oUs\|dAs\|dTs\|dTs\|dGs\|dAs\|dGs\|dTs\|dTs\|5mCs\|5mCs\|oCs\|oGs\|o |
| | Gs\|oGs\|oC] |
| 620 | [oUs\|oGs\|oUs\|oCs\|oUs\|dTs\|dTs\|5mCs\|dTs\|dGs\|dAs\|dTs\|dTs\|dAs\|dGs\|oCs\|oCs\|oUs\| |
| | oGs\|oA] |
| 621 | [oUs\|oGs\|oGs\|oUs\|oCs\|dTs\|dTs\|5mCs\|dAs\|dTs\|dAs\|dTs\|5mCs\|dAs\|dGs\|oUs\|oCs\|o |
| | Us\|oGs\|oA] |

The mRNA sequence of reference MSH3 mRNA NM_002439.4 (SEQ ID NO: 614) (www.ncbi.nlm.nih.gov/nuccore/NM_002439.4, incorporated herein by reference), is provided below.

| | |
|---|---|
| 1 | ccgcagacgc ctgggaactg cggccgcggg ctcgcgctcc tcgccaggcc ctgccgccgg |
| 61 | gctgccatcc ttgccctgcc atgtctcgcc ggaagcctgc gtcgggcggc ctcgctgcct |
| 121 | ccagctcagc ccctgcgagg caagcggttt tgagccgatt cttccagtct acgggaagcc |
| 181 | tgaaatccac ctcctcctcc acaggtgcag ccgaccaggt ggaccctggc gctgcagcgg |
| 241 | ctgcagcggc cgcagcggcc gcagcgcccc cagcgccccc agctcccgcc ttcccgcccc |
| 301 | agctgccgcc gcacatagct acagaaattg acagaagaaa gaagagacca ttggaaaatg |
| 361 | atgggcctgt taaaaagaaa gtaaagaaag tccaacaaaa ggaaggagga agtgatctgg |
| 421 | gaatgtctgg caactctgag ccaaagaaat gtctgaggac caggaatgtt tcaaagtctc |
| 481 | tggaaaaatt gaaagaattc tgctgcgatt ctgcccttcc tcaaagtaga gtccagacag |
| 541 | aatctctgca ggagagattt gcagttctgc caaaatgtac tgattttgat gatatcagtc |
| 601 | ttctacacgc aaagaatgca gtttcttctg aagattcgaa acgtcaaatt aatcaaaagg |
| 661 | acacaacact ttttgatctc agtcagtttg gatcatcaaa tacaagtcat gaaaatttac |
| 721 | agaaaactgc ttccaaatca gctaacaaac ggtccaaaag catctatacg ccgctagaat |
| 781 | tacaatacat agaaatgaag cagcagcaca aagatgcagt tttgtgtgtg gaatgtggat |
| 841 | ataagtatag attctttggg gaagatgcag agattgcagc ccgagagctc aatatttatt |
| 901 | gccatttaga tcacaacttt atgacagcaa gtatacctac tcacagactg tttgttcatg |
| 961 | tacgccgcct ggtggcaaaa ggatataagg tgggagttgt gaagcaaact gaaactgcag |
| 1021 | cattaaaggc cattggagac aacagaagtt cactcttttc ccggaaattg actgcccttt |
| 1081 | atacaaaatc tacacttatt ggagaagatg tgaatcccct aatcaagctg gatgatgctg |
| 1141 | taaatgttga tgagataatg actgatactt ctaccagcta tcttctgtgc atctctgaaa |
| 1201 | ataaggaaaa tgttagggac aaaaaaaagg gcaacatttt tattggcatt gtgggagtgc |
| 1261 | agcctgccac aggcgaggtt gtgtttgata gtttccagga ctctgcttct cgttcagagc |
| 1321 | tagaaacccg gatgtcaagc ctgcagccag tagagctgct gcttccttcg gccttgtccg |
| 1381 | agcaaacaga ggcgctcatc cacagagcca catctgttag tgtgcaggat gacagaattc |
| 1441 | gagtcgaaag gatggataac atttattttg aatacagcca tgctttccag gcagttacag |
| 1501 | agttttatgc aaaagataca gttgacatca aaggttctca aattatttct ggcattgtta |
| 1561 | acttagagaa gcctgtgatt tgctctttgg ctgccatcat aaaatacctc aaagaattca |
| 1621 | acttggaaaa gatgctctcc aaacctgaga attttaaaca gctatcaagt aaaatggaat |
| 1681 | ttatgacaat taatggaaca acattaagga atctggaaat cctacagaat cagactgata |
| 1741 | tgaaaaccaa aggaagtttg ctgtgggttt tagaccacac taaaacttca tttgggagac |
| 1801 | ggaagttaaa gaagtgggtg acccagccac tccttaaatt aagggaaata aatgcccggc |
| 1861 | ttgatgctgt atcggaagtt ctccattcag aatctagtgt gtttggtcag atagaaaatc |
| 1921 | atctacgtaa attgcccgac atagagaggg gactctgtag catttatcac aaaaaatgtt |
| 1981 | ctacccaaga gttcttcttg attgtcaaaa ctttatatca cctaaagtca gaatttcaag |
| 2041 | caataatacc tgctgttaat tcccacattc agtcagactt gctccggacc gttattttag |
| 2101 | aaattcctga actcctcagt ccagtggagc attacttaaa gatactcaat gaacaagctg |
| 2161 | ccaaagttgg ggataaaact gaattattta aagacctttc tgacttccct ttaataaaaa |
| 2221 | agaggaagga tgaaattcaa ggtgttattg acgagatccg aatgcatttg caagaaatac |
| 2281 | gaaaaatact aaaaaatcct tctgcacaat atgtgacagt atcaggacag gagtttatga |
| 2341 | tagaaataaa gaactctgct gtatcttgta taccaactga ttgggtaaag gttggaagca |
| 2401 | caaaagctgt gagccgcttt cactctcctt ttattgtaga aaattacaga catctgaatc |
| 2461 | agctccggga gcagctagtc cttgactgca gtgctgaatg gcttgatttt ctagagaaat |
| 2521 | tcagtgaaca ttatcactcc ttgtgtaaag cagtgcatca cctagcaact gttgactgca |
| 2581 | ttttctccct ggccaaggtc gctaagcaag gagattactg cagaccaact gtacaagaag |
| 2641 | aaagaaaaat tgtaataaaa aatggaaggc accctgtgat tgatgtgttg ctgggagaac |
| 2701 | aggatcaata tgtcccaaat aatacagatt tatcagagga ctcagagaga gtaatgataa |
| 2761 | ttaccggacc aaacatgggt ggaaagagct cctacataaa acaagttgca ttgattacca |
| 2821 | tcatggctca gattggctcc tatgttcctg cagaagaagc gacaattggg attgtggatg |
| 2881 | gcattttcac aaggatgggt gctgcagaca atatatataa aggacagagt acatttatgg |
| 2941 | aagaactgac tgacacagca gaaataatca gaaaagcaac atcacagtcc ttggttatct |
| 3001 | tggatgaact aggaagaggg acgagcactc atgatggaat tgccattgcc tatgctacac |
| 3061 | ttgagtattt catcagagat gtgaaatcct taaccctgtt tgtcacccat tatccgccag |
| 3121 | tttgtgaact agaaaaaaat tactcacacc aggtggggaa ttaccacatg ggattcttgg |
| 3181 | tcagtgagga tgaaagcaaa ctggatccag gcgcagcaga acaagtccct gattttgtca |
| 3241 | ccttccttta ccaaataact agaggaattg cagcaaggag ttatggatta aatgtggcta |
| 3301 | aactagcaga tgttcctgga gaaattttga agaaagcagc tcacaagtca aaagagctgg |
| 3361 | aaggattaat aaatacgaaa agaaagagac tcaagtattt tgcaaagtta tggacgatgc |
| 3421 | ataatgcaca agacctgcag aagtggacag aggagttcaa catggaagaa acacagactt |
| 3481 | ctcttcttca ttaaaatgaa gactacattt gtgaacaaaa aatggagaat taaaaatacc |
| 3541 | aactgtacaa aataactctc cagtaacagc ctatctttgt gtgacatgtg agcataaaat |
| 3601 | tatgaccatg gtatattcct attggaaaca gagaggtttt tctgaagaca gtctttttca |
| 3661 | agtttctgtc ttcctaactt ttctacgtat aaacactctt gaatagactt ccactttgta |
| 3721 | attagaaaat tttatggaca gtaagtccag taaagcctta agtggcagaa tataattccc |
| 3781 | aagcttttgg agggtgatat aaaaatttac ttgatatttt tatttgtttc agttcagata |
| 3841 | attggcaact gggtgaatct ggcaggaatc tatccattga actaaaataa ttttattatg |
| 3901 | caaccagttt atccaccaag aacataagaa ttttttataa gtagaaagaa ttggccaggc |
| 3961 | atggtggctc atgcctgtaa tcccagcact ttgggaggcc aaggtaggca gatcacctga |
| 4021 | ggtcaggagt tcaagaccag cctggccaac atggcaaaac cccatcttta ctaaaaatat |
| 4081 | aaagtacatc tctactaaaa atacgaaaaa attagctggg catggtggcg cacacctgta |
| 4141 | gtcccagcta ctccggaggc tgaggcagga gaatctcttg aacctgggag gcggaggttg |
| 4201 | caatgagccg agatcacgtc actgcactcc agcttgggca acagagcaag actccatctc |
| 4261 | aaaaaaaaaa aaagaaaaaa gaaaagaaat agaattatca agcttttaaa aactagagca |
| 4321 | cagaaggaat aaggtcatga aatttaaaag gttaaatatt gtcataggat taagcagttt |
| 4381 | aaagattgtt ggatgaaatt atttgtcatt cattcaagta ataaatattt aatgaatact |
| 4441 | tgctataaaa aaaaaaaaaa aaaaaaaaaa aa |

### Example 2. Pharmacokinetic Profile of and Tolerability

### Objective(s)

The objective of this study is to evaluate the pharmacokinetic profile of the administration of Antisense Oligo No. 289 greater than 10 mg dose level and its tolerability and effect in silencing MSH3 mRNA following a single intracerebroventricular (ICV) injection. The data obtained will be also used to conduct pharmacokinetic and pharmacodynamic (PK/PD) modeling and simulation to predict dosing regimen to achieve >40% and >50% MSH3 mRNA silencing in the caudate nucleus at trough levels within 12 weeks of dosing in a first in human (FIH) trial.

### Experimental Design

Sixteen female Cynomolgus monkeys (NHP) were randomized in this study. Necropsy was planned at 2 h, 8 h, 24 h, 48 h, Day 8, Day 15 and Day 29 post dosing (N=2 animals per timepoint). In order to monitor the tolerability of the dose level, dose escalation was initiated at 15 mg as the starting dose level. 2 mL of Antisense Oligo No. 289 via a single ICV injection at infusion rate of 0.1 mL/min was delivered. Up to two sentinel animals were to be dosed at each dose level and monitored for clinical signs over the course of 2-24 hours. If 15 mg dose level is not well tolerated, monkeys will be dosed at 12 mg via single ICV injection and monitored for clinical signs over the course of 2-24 hours.

Tissues from various central nerve system (CNS) and peripheral organs are to be collected at each necropsy timepoint. CSF and plasma samples to be collected from surviving animals and at necropsy. The concentrations of Antisense Oligo No. 289 in CSF, plasma, brain tissues (cerebral cortex, caudate and putamen, nucleus accumbens), liver and kidney will be measured using LC-MS/MS. MSH3 mRNA and protein knockdown in cerebral cortex, caudate, putamen and nucleus accumbens will be measured using RT-qPCR, and western blot or ELISA.

| | | | Dose | Dose | | | |
|---|---|---|---|---|---|---|---|
| Group | Test | Dose Level | Volume | Concentration | No. of | Necropsy | |
| No. | Material | (mg)^{a} | (mL) | (mg/mL)^{a} | Animals | Timepoint | |
| 1 | Antisense Oligo | TBD | 2 | TBD | 2 | 2 | hr |
| | No. 289 | (up to 25 mg) | | | | | |
| 2 | Antisense Oligo | TBD | 2 | TBD | 2 | 8 | hr |
| | No. 289 | (up to 25 mg) | | | | | |
| 3 | Antisense Oligo | TBD | 2 | TBD | 2 | 24 | hr |
| | No. 289 | (up to 25 mg) | | | | | |
| 4 | Antisense Oligo | TBD | 2 | TBD | 2 | 48 | hr |
| | No. 289 | (up to 25 mg) | | | | | |

| | | | | | | |
|---|---|---|---|---|---|---|
| 5 | Antisense Oligo | TBD | 2 | TBD | 2 | Day 8 |
| | No. 289 | (up to 25 mg) | | | | |
| 6 | Antisense Oligo | TBD | 2 | TBD | 2 | Day 15 |
| | No. 289 | (up to 25 mg) | | | | |
| 7 | Antisense Oligo | 15 | 2 | 7.5 | 2 | Day 29 |
| | No. 289 | | | | | |
| 8 | Vehicle (aCSF) | 0 | 2 | 0 | 2 | Day 27 |

No. = Number, h/hr = hours
^{a}If the initial dose level of 15 mg is acutely well tolerated in Group 7, the dose level will be increased to 20 mg for Group 6. If 20 mg is acutely well tolerated in Group 6, the dose level will be increased to 25 mg for the first Group 5 animal. If 25 mg is well tolerated in that animal, the remaining animals on study will be dosed at 25 mg.
If the 15 mg dose is not well tolerated (e.g., neurological or other clinical signs occur prior to dosing subsequent groups that are severe, require intervention, worsen, or are not typical transient procedure-related findings), the dose for all subsequent animals will be lowered to 12 mg.

### Interim Results of Clinical Observation

Two female cynomolgus monkeys (Day 29) were dosed at 15 mg via single ICV injection. The first monkey became ataxic and began vomiting, with hunched posture around 3 hours postdose. She stabilized after receiving a nausea suppressant (cerenia). She recovered after 24 hours and will remain on the study for Day 29 necropsy. The second monkey became ataxic, hunched, with severely decreased activity and decreased pupillary response, and was laterally recumbent approximately 4 hours post dose. After consultation with the veterinarian this animal was euthanized approximately 4-5 hours post dose.

A third monkey was dosed at 12 mg single injection ICV and the dosing was well tolerated. Additional 5 NHPs were dosed at 12 mg single injection ICV for PK/PD timepoints of 24 hrs (n=2), 48 hrs (n=2), Day 8 (n=1), Day 15 (n=1). In total, eight NHPs were dosed, 2 with 15 mg, 6 with 12 mg, and 2 NHPs are for vehicle control. Three out of five NHP experienced ataxia, impaired righting reflex, tremors, and are intermittently laterally recumbent during recovery period. However, all animals recovered to normal condition after 24-36 hours without any intervention.

### Example 3. Pharmacokinetics Pharmacodynamics (PK/PD) Investigation in Non-Human Primate (NHP)

Investigation of Antisense Oligo No. 289 PK/PD in NHP using intrathecal (IT) injection route

### Experimental Design

Twelve male Cynomolgus monkeys were administered 10 mg Antisense Oligo No. 289 (2 mL)(in artificial CSF) via a single IT injection. Necropsy was performed at 2 h, 8 h, 24 h, 48 h, 168 h (7 days) and 672 h (28 days) post dosing (N=2 animals per timepoint). Cerebrospinal fluid (CSF) and tissues from various central nerve system (CNS) regions and peripheral organs were collected at each necropsy timepoint. Plasma samples were collected from surviving animals and at necropsy. The concentrations of Antisense Oligo No. 289 in CSF, plasma, brain tissues (cerebral cortex, caudate and putamen), liver and kidney were measured using liquid chromatography coupled with tandem mass spectrometry (LC-MS/MS). The mRNA levels of MSH3 in cerebral cortex, caudate, putamen, as well as lumbar spinal cord were measured using real-time quantitative polymerase chain reaction (RT-qPCR).

### Experimental Results

The concentration-time profile of Antisense Oligo No. 289 in CSF after a single IT injection was apparently multiple-phasic with fast initial distribution. A similar concentration-time profile was also observed in plasma. Brain tissues (cerebral cortex, caudate and putamen) and peripheral tissues (liver and kidney) showed various exposure levels of Antisense Oligo No. 289, and slow apparent elimination phases. A multi-compartment PK model was developed and adequately fit the observed concentration-time profiles in all tissues measured.

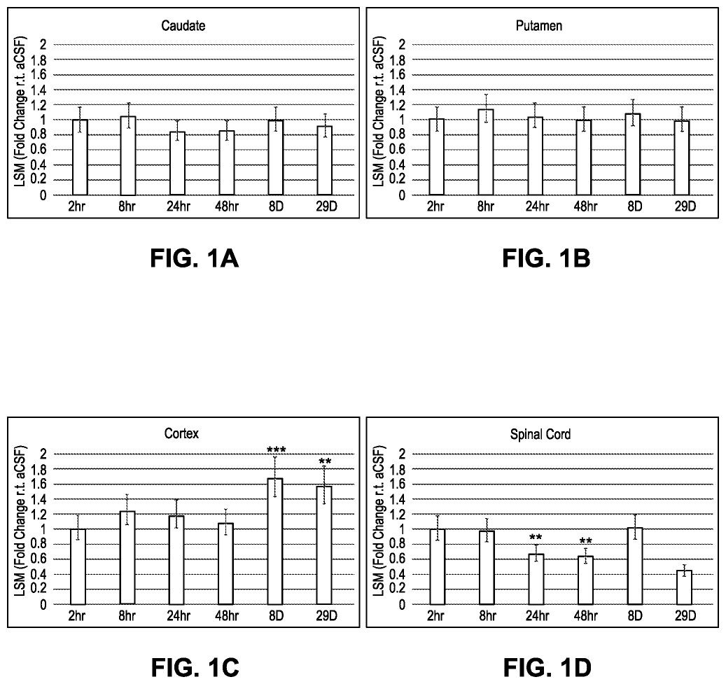

Comparing to the CNS tissues obtained from vehicle-dosed (artificial CSF) control animals from a separate study or tissues collected at an early timepoint (2 h) of the current study, significant decrease of mRNA levels of MSH3 were only observed in lumbar spinal cord; no significant decrease of mRNA levels of MSH3 were observed in cerebral cortex, caudate and putamen (FIGS. 1A-1D).

### Investigation of Antisense Oligo No. 289 PK/PD in NHP Using Intracerebroventricular (ICV) Injection Route

### Experimental Design

Fifteen female Cynomolgus monkeys were administered 10 mg Antisense Oligo No. 289 (2 mL, in artificial CSF) via a single ICV injection. Antisense Oligo No. 289 was infused into the occipital horn of the lateral ventricle. Necropsy was performed at 2 h, 8 h, 24 h, 48 h, 168 h (7 days), 336 h (14 days) and 672 h (28 days) post dosing (N=2 animals per timepoint except N=3 for 336 h (14 days). Two naïve animals that did not receive any injection, were sacrificed and served as controls. The duration of action was further investigated up to day 84. Six female cynomolgus monkeys were dosed with 10 mg Antisense Oligo No. 289 via a single ICV injection. Necropsy was performed on Day 42, Day 56, and Day 84 (N=2 per time point).

Tissues from various CNS regions and peripheral organs were collected at each necropsy timepoint. CSF and plasma samples were collected from selective surviving animals and at necropsy. The concentrations of Antisense Oligo No. 289 in CSF, plasma, brain tissues (cerebral cortex, caudate and putamen), liver and kidney were measured using LC-MS/MS. The mRNA levels of MSH3 in cerebral cortex, caudate, putamen, as well as lumbar spinal cord were measured using RT-qPCR.

### Experimental Results

The concentration-time profile of Antisense Oligo No. 289 in CSF after a single ICV injection was apparently multiple-phasic with fast initial distribution. A similar concentration-time profile was also observed in plasma. Brain tissues (cerebral cortex, caudate and putamen) and peripheral tissues (liver and kidney) showed various exposure levels of Antisense Oligo No. 289, and slow apparent elimination phases. Significantly higher Antisense Oligo No. 289 concentrations were observed in brain tissues than those observed after IT injection at the same dose (10 mg). A multi-compartment PK model was developed and adequately fit the observed concentration-time profiles in all tissues measured.

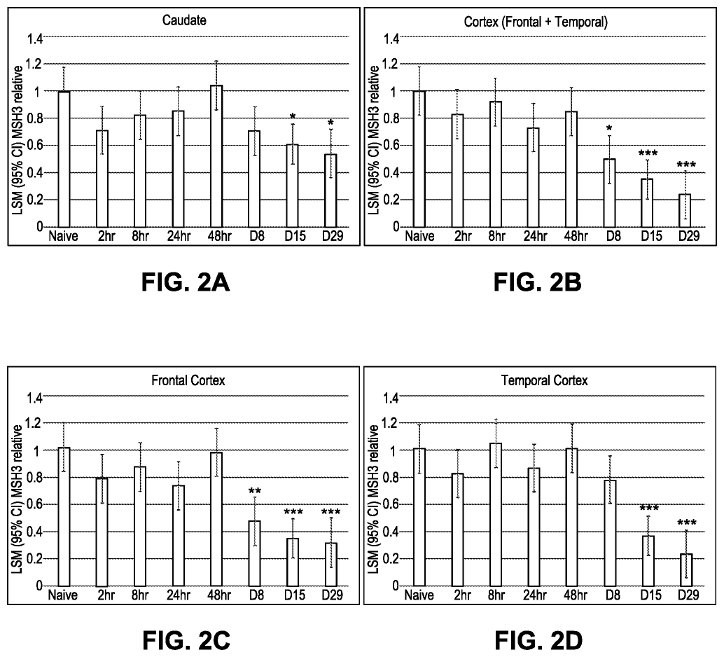

Comparing to the CNS tissues obtained from naïve control animals of the current study, significant time-dependent decrease of mRNA levels of MSH3 were observed in cerebral cortex and caudate, with 76% and 46% knockdown (KD) respectively on day 29 (28 days post dose, FIGS. 2A-2D); no significant MSH3 mRNA KD was observed in putamen.

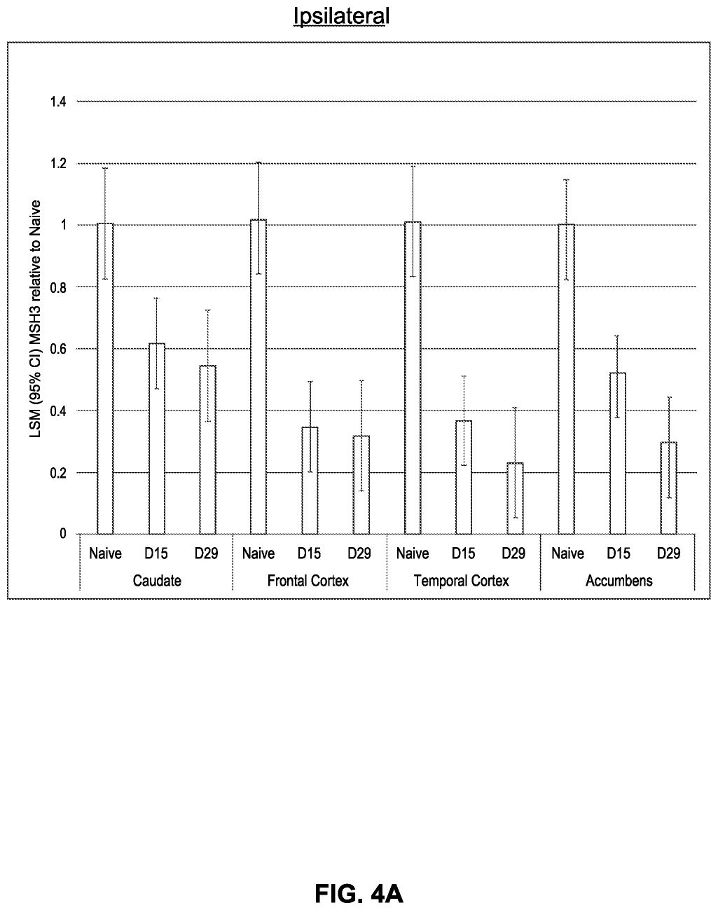

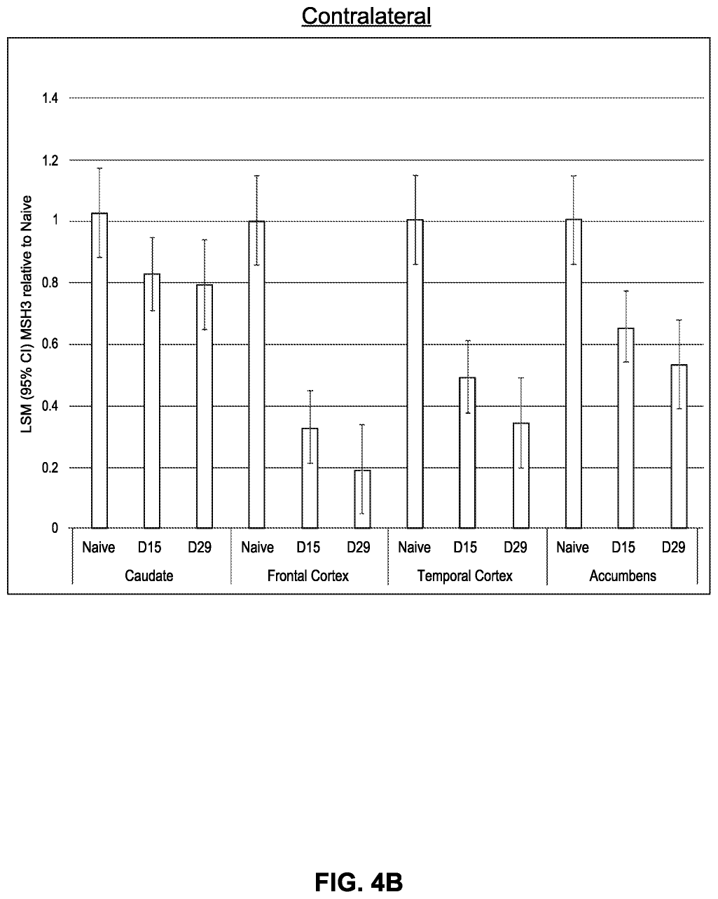

Significant KD of MSH3 mRNA in both ipsilateral and contralateral cortex (frontal and temporal cortex) and nucleus accumbens was observed (see FIGS. 4A-4B). The percent KD on days 8, 15, and 29, respectively, is shown below:

### Frontal Cortex:

Ipsilateral: 53 (**), 65 (***) and 68% (***)
Contralateral: 63, 67 (***) and 81% (***)

### Temporal Cortex

Ipsilateral: 23, 64 (***) and 77% (***)
Contralateral: 36 (*), 51 (***) and 66% (***)

### Nucleus Accumbens

Ipsilateral: 43 (**), 48 (***) and 70% (***)
Contralateral: 20, 36 (*) and 48% (**)

Approximately equal or greater than 40% KD of MSH3 mRNA was observed in ipsilateral caudate at day 15 (39% *) and Day 29 (46% *); No significant KD was detected in contralateral caudate.

No significant MSH3 mRNA KD was observed in putamen.

MSH3 mRNA was significantly knocked down in ipsilateral cerebellum by 47% (***) at Day 15 and 39% (**) at Day 29.

P values from pairwise comparisons vs. Naïve under linear mixed effects model: MSH3 Fold Change ˜(1|Animal)+Housekeeper+Tissue*Time. NOTE: 2 hr, 8 hr, 24 hr, 48 hr timepoints (no effect) were included in analysis. (* P<0.05, ** P<0.01, *** P<0.001)

These results demonstrate that administration of Antisense Oligo No. 289 at 10 mg dose level results in significant and widespread knockdown of MSH3 mRNA after a single ICV injection into the occipital horn of lateral ventricle in NHPs. Several brain regions, regardless of hemisphere, had similar mRNA knockdown effects.

An integrated PK/PD model was developed based on multiple-compartment tissue PK and indirect response PD (Sharma & Jusko, Br J Clin Pharmacol 45:229-239 (1998)). The PK/PD model sufficiently fit to the observed time-dependent MSH3 mRNA responses observed in cerebral cortex, caudate and putamen. Based on the current data with duration of action up to day-84, the PK/PD model estimated the in vivo potency of Antisense Oligo No. 289 for stimulation of MSH3 mRNA degradation in NHP, with Sₘₐₓ=4.7 (Emax˜18%), SC₅₀=8.6 μg/g, and Hill coefficient γ=1.8.

Conclusions on Comparing PK/PD after a Single ICV Vs IT 10 mg Injection of Antisense Oligo No. 289 in NHPs

In NHPs at the same dose (10 mg), administration of Antisense Oligo No. 289 by ICV route demonstrated significantly higher concentrations in cerebral cortex, caudate and putamen than IT route. Upon ICV administration, significant and time-dependent knockdown levels of MSH3 mRNA were observed in cerebral cortex and caudate, but not in putamen. Based on the current data, a PK/PD model could be developed to adequately describe the observed data and estimate the in vivo potency of Antisense Oligo No. 289 in stimulation of MSH3 mRNA degradation.

### Justification and Design for Dose Escalation PK/PD Study by a Single ICV Injection of Antisense Oligo No. 289 in NHPs

To further explore dose-dependency of PK/PD response and tolerability, a dose escalation study in NHP is ongoing. Fourteen female Cynomolgus monkeys will be administered Antisense Oligo No. 289 (2 mL)(in artificial CSF) via a single ICV injection starting at 15 mg. The dose level will be adjusted based on clinical observation of tolerability. Necropsy will be performed at 2 h, 8 h, 24 h, 48 h, 168 h (7 days), 336 h (14 days) and 672 h (28 days) post dosing (N=2 animals per timepoint). Tissues from various CNS regions and peripheral organs will be collected at each necropsy timepoint. CSF and plasma samples will be collected from selective surviving animals and at necropsy. All relevant brain and peripheral tissues as well as CSF and plasma will be analyzed for Antisense Oligo No. 289 concentrations and MSH3 mRNA level as described above.

### Example 4. Investigation of Biodistribution for Three Routes of Administration (RoA) Using SEQ ID NO: 617

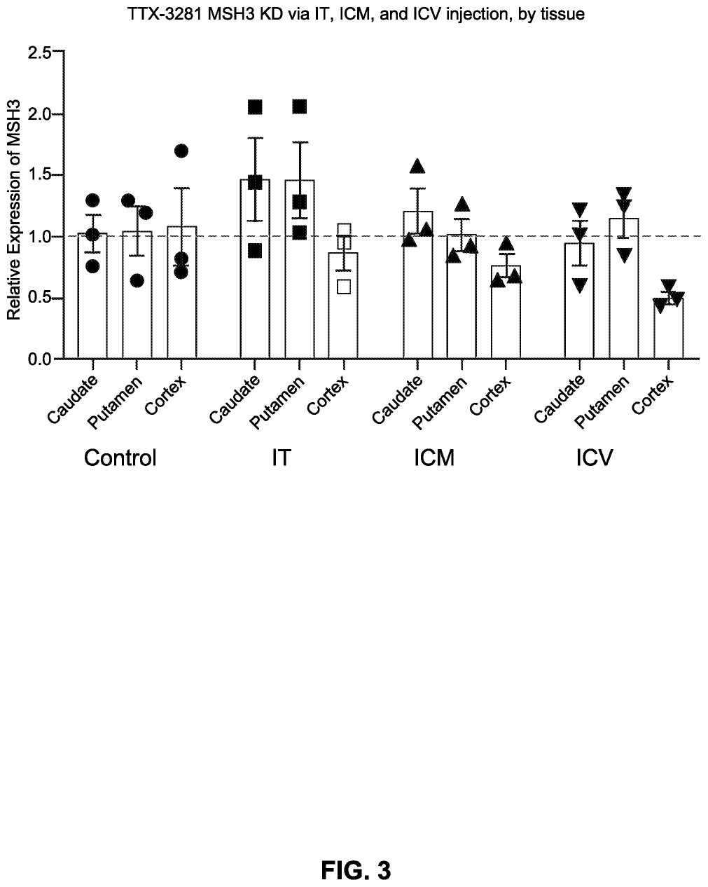

A pharmaceutical composition comprising the oligonucleotide of SEQ ID NO: 617 was used for this investigation. The oligonucleotide of SEQ ID NO: 617 was formulated in CSF as a 7 mg bolus. The composition was used to assess brain biodistribution for IT, intra-cisterna magna (“ICM”), and ICV injection. The same dose was injected for all RoAs and the KD was evaluated after 15 days. As shown in FIG. 3, ICV injection achieved the greatest overall KD across all RoA.

### Example 5. Dose Escalation Study—ICV Administration of Antisense Oligo No. 289

The effect of Antisense Oligo No. 289 on lowering MSH3 mRNA following a single ICV injection in non-human primates was investigated.

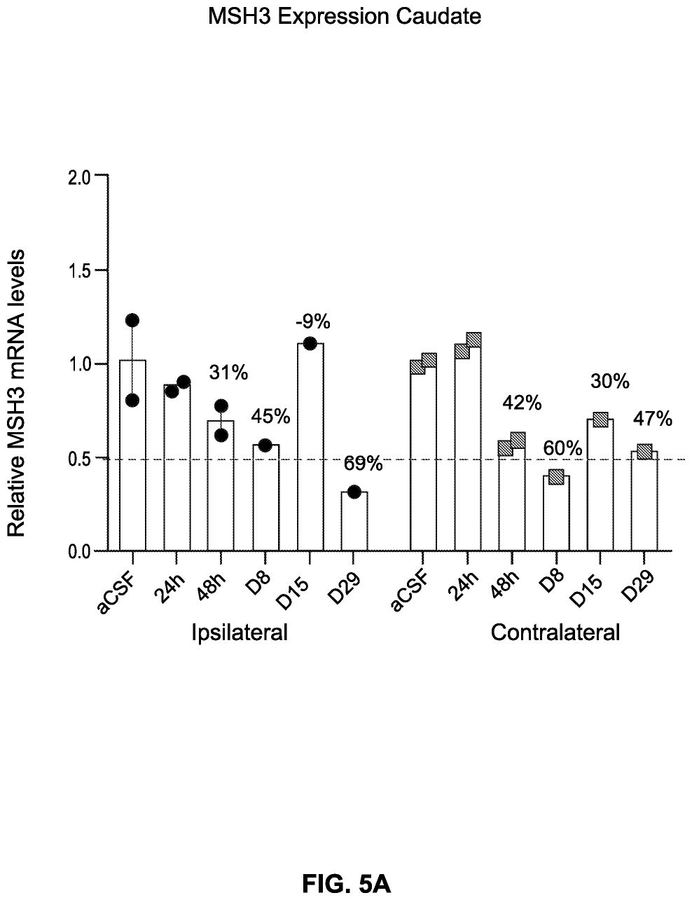

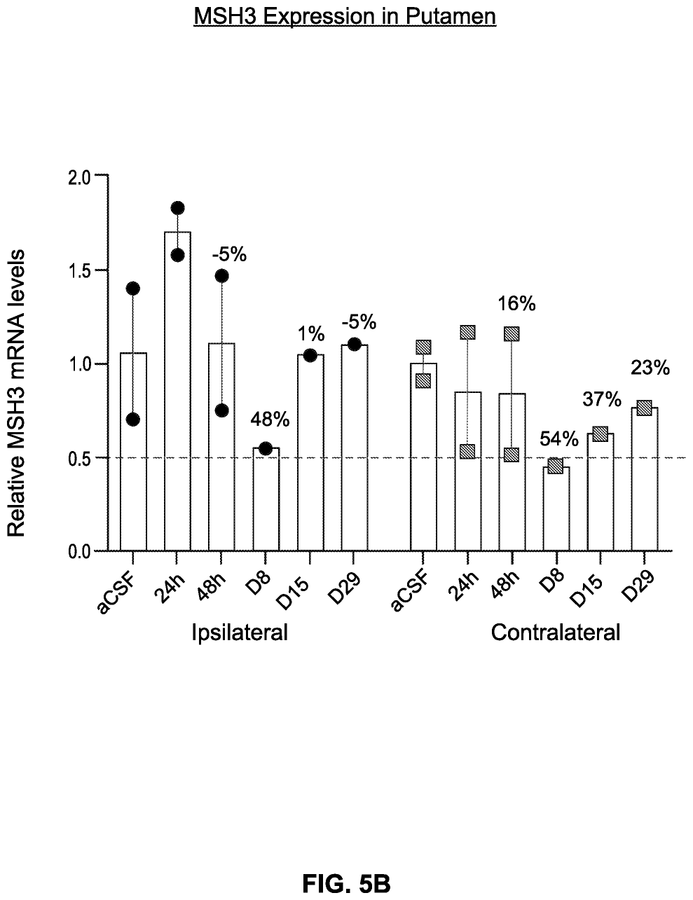

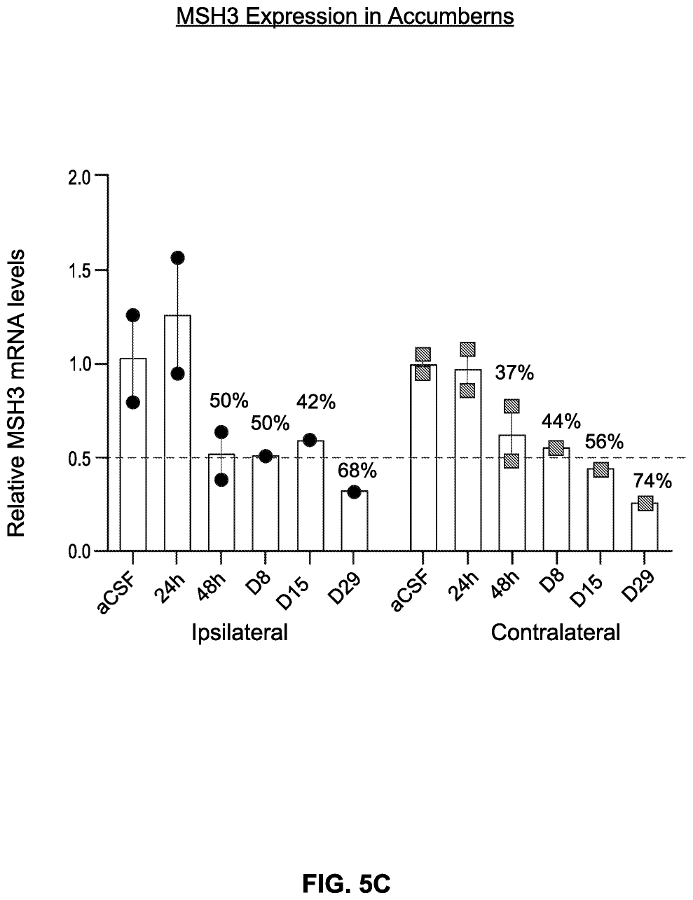

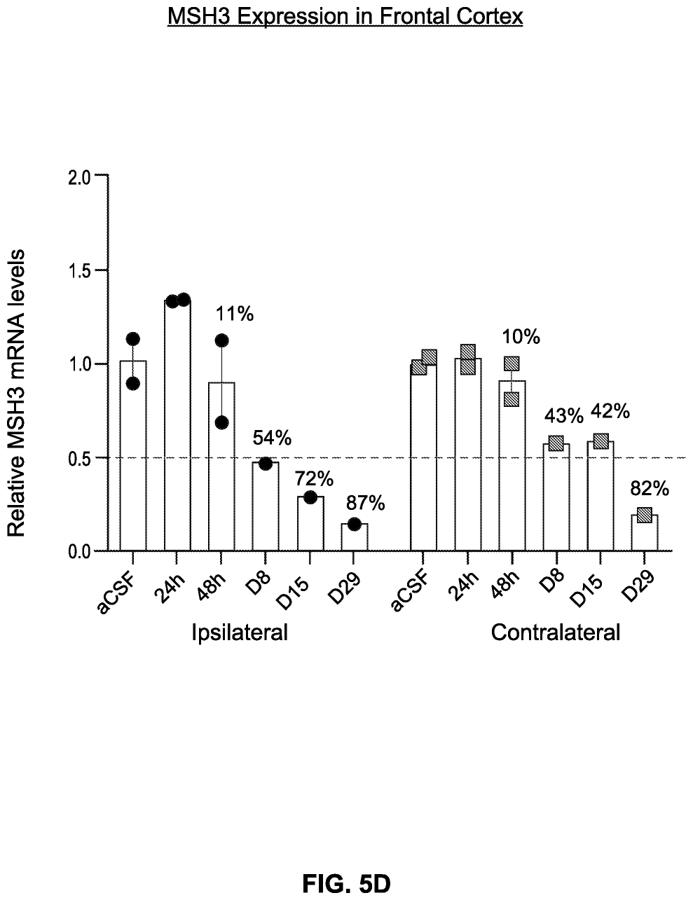

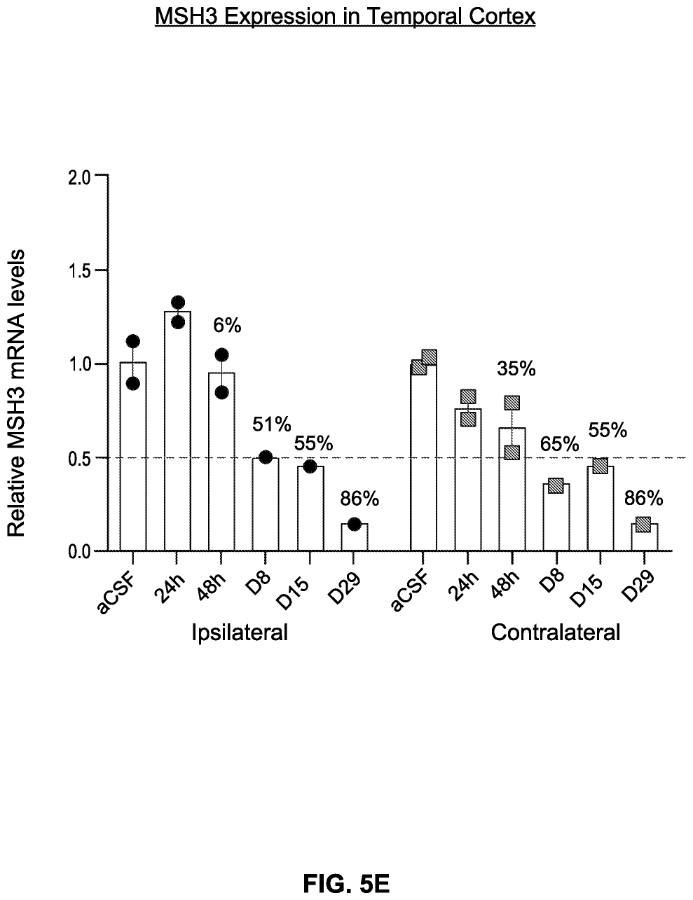

An initial dose of 15 mg given to two animals resulted in adverse clinical signs necessitating early euthanasia of one animal. Thereafter, six animals were dosed at a tolerable dose of 12 mg. Four of six animals showed transient adverse clinical signs. Another group of 6 animals were then given a 10 mg ICV dose to assess duration of action of Antisense Oligo No. 289 by measuring MSH3 mRNA at 6, 8, and 12 weeks post injection (2 animals per timepoint). MSH3 mRNA expression in various areas of the brain showed a dose-dependent decline after a single dose (12 mg and 15 mg) ICV injection (FIGS. 5A-5E).

The percent knockdown of MSH3 mRNA after a single ICV injection of Antisense Oligo No. 289 at Day 8 (10 mg dose and 12 mg dose) and Day 29 (10 mg dose and 15 mg dose) is shown below in Tables 5 and 6. These results demonstrate that MSH3 mRNA expression declines following a single ICV dose of Antisense Oligo No. 289.

TABLE 5

| | KD % at Day 8 after 10 mg ICV | | KD % at Day 8 after 12 mg ICV | |
|---|---|---|---|---|
| | (n = 2) | | (n = 1) | |
| Brain tissue | Ipsilateral | Contralateral | Ipsilateral | Contralateral |
| Caudate | 30 | −4 | 45 | 60 |
| Putamen | −11 | n/a | 48 | 54 |
| Accumbens | 43 | 31 | 50 | 44 |
| Frontal cortex | 54 | 63 | 54 | 43 |
| Temporal cortex | 23 | 36 | 51 | 65 |

TABLE 6

| | KD % at Day 29 after 10 mg ICV | | KD % at Day 29 after 15 mg ICV | |
|---|---|---|---|---|
| | (n = 2) | | (n = 1) | |
| Brain tissue | Ipsilateral | Contralateral | Ipsilateral | Contralateral |
| Caudate | 46 | 24 | 69 | 47 |
| Putamen | −5 | n/a | −5 | 23 |
| Accumbens | 70 | 47 | 68 | 77 |
| Frontal cortex | 69 | 81 | 87 | 82 |
| Temporal cortex | 77 | 66 | 86 | 86 |

### Example 6. Investigation of MSH3 mRNA and Protein Knockdown Following IT Administration of Antisense Oligo No. 289

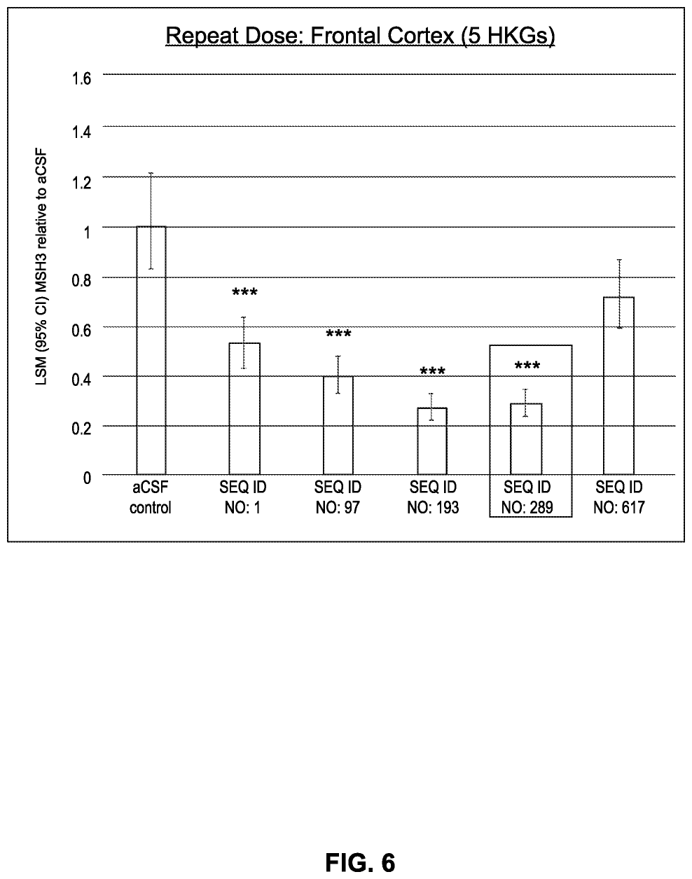

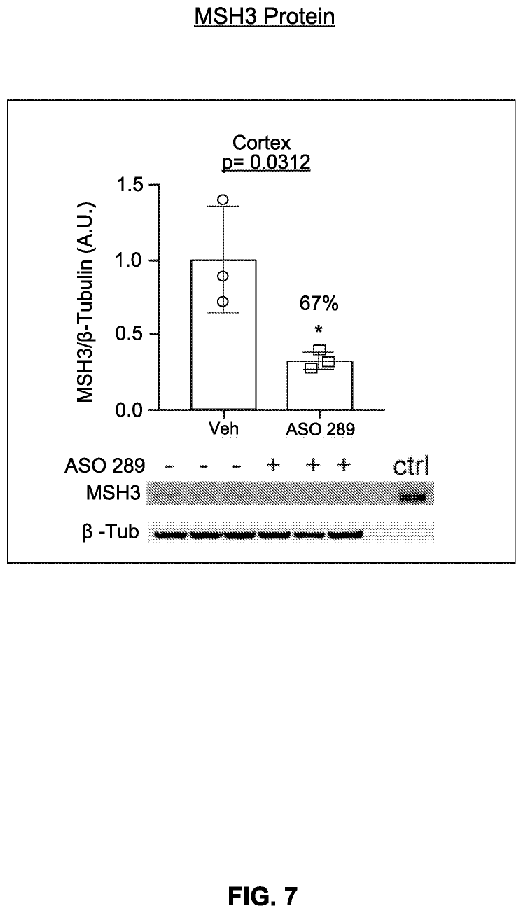

Antisense Oligo No. 289 was intrathecally administered to NHPs and the result on MSH3 mRNA and protein knockdown in the Frontal Cortex was investigated. Antisense Oligo No. 289 was injected twice, on day 1 and day 15. Three animals per group were sacrificed on day 30 (29 days after the 1^{st} IT injection). Brains were removed and punches from regions of interest, including cortex, caudate and putamen, were processed for RNA and protein assays. MSH3 mRNA in animals administered Antisense Oligo No. 289 was suppressed as compared to animals administered other antisense oligos or a control (aCSF) (FIG. 6). Animals administered Antisense Oligo No. 289 produced about 67% of the MSH3 protein, as compared to animals administered the other antisense oligos (FIG. 7).

### Example 7: Dosing Strategies—Safety Margin Assessment

Various dosing strategies to keep the predicted Cₘₐₓ of Antisense Oligo No. 289 in cerebrospinal fluid (CSF) below the threshold for preclinical observations while maintaining the desired MSH3 mRNA knockdown (KD) in key brain tissues was investigated.

Dosing strategies are related to the the following clinical observations in NHPs:
CSF C₀ of 696 μg/mL was reached upon a single ICV dose of 10 mg; dose was well tolerated.
Assuming linear PK, CSF Co of 835 μg/mL was extrapolated upon a single ICV dose of 12 mg; clinical adverse observations at this dose were reversible.

For desired KD of over 50% in human caudate and cortex, and approximately 30% KD in putamen.
CSF Cₘₐₓ of 610 μg/mL is projected upon a dosage regimen of 150 mg Q28D×2 (two ICV doses of 150 mg every 28 days) followed by 150 mg Q168 D×n (#n maintance ICV doses of 150 mg every 168 days); 1.1× and 1.2× below CSF C₀ values from NHP studies.
CSF Cₘₐₓ of 300 μg/mL is projected upon a dosage regimen of 75 mg Q14D×2 (two ICV doses of 75 mg every 14 days) followed by 75 mg Q84D×n (#n maintance ICV doses of 75 mg every 84 days); 2.3× and 2.8× below CSF C₀ values from NHP studies.

### Example 8: Tolerability and Efficacy of a Single Intravitreal (IVT) Injection in Mice Retinas

The purpose of this study was to evaluate Antisense Oligo No. 617 at different doses for tolerability and efficacy in wildtype C57BL6 mice retinas following a single intravitreal (IVT) injection. The protocol was reviewed and approved by the Institutional Animal Care and Use Committee (IACUC), and all animal welfare concerns have been addressed and documented.

### Experimental Groups:

PBS 1 μl: n=5
Antisense Oligo No. 617 50 μg/1 μl: n=5
Antisense Oligo No. 617 100 μg/1 μl: n=5

### Study Design

### Treatment Solutions:

Antisense Oligo No. 617 stock solutions (100 μl, 100 mg/ml) were received on dry ice and stored at −80° C. The stock solution was diluted 1:1 (e.g. 10 μl (stock): 10 μl (PBS) with PBS (Gibco 10010-031) to make a final solution of 50 mg/ml for the 50 g group; and stock solution was used for the 100 g group. One micro liter (1 μl) was given to each animal for a final dose of 0 μg, 50 μg, or 100 μg.

### Intravitreal Injection (IVT)

Animals were anesthetized with 2-3% isoflurane in 100% 02. Once anesthetized, one drop of 0.5% tropicamide was used for dilation of the pupil. Proparacaine was applied to the mouse eyes as a topical anesthetic before IVT injection. The mouse was positioned under a surgical scope to expose the superior temporal sclera region of the eye. A sterile 30-gauge needle was used to puncture the superior temporal sclera at the approximate level of the par plana. The ocular muscles and vessels were avoided. The tip of the prepared Hamilton syringe with a 32-gauge blunt needle containing the solution was inserted through the puncture hole in the sclera at an angle of approximately 45°, into the vitreous body. One microliter of solution was slowly injected into the vitreous humour and the needle was left to dwell in place for a few seconds prior to withdrawal. Particular attention was paid towards avoiding any contact with the lens and the retina. After the full withdrawal of the needle, a tobramycin ophthalmic antibiotic ointment was applied to the injection site to further prevent infection. The procedure was done on both eyes. The animals were then placed in the recovery chamber, on a heating pad, until recovery from anesthesia, and then returned to home cages.

### Animal Weight and Observations

At Day 14 after the IVT injection, mice were euthanized by CO₂. The whole eyes were collected and briefly frozen by dry ice. The eyes were then partially thawed for removal of the anterior chamber of the eye. For technical reasons, to prevent loss of vitreous, the lens was included in the collected samples of vitreous humour. The retina (including the RPE (retinal pigment epithelium) cells) were collected and frozen individually.

### QRT-PCR

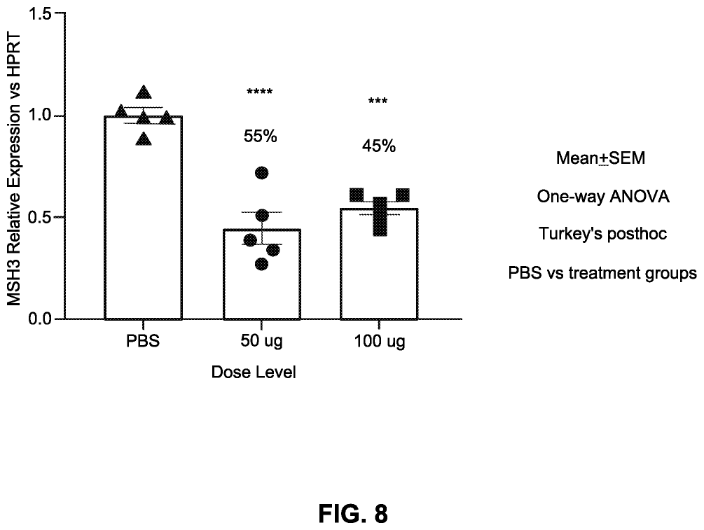

Left and right retina including RPE were pooled for mRNA analysis due to small tissue size. QRT-PCR was performed to assess mRNA of the mouse MSH3 gene in the retina from the 3 experimental groups. The HPRT gene was used as house-keeping gene for normalization of data. The results are shown in FIG. 8. As shown in FIG. 8, there was a significant MSH3 mRNA knockdown in wildtype mouse retinas following a single IVT injection.

### Example 9: Duration of Action after a Single ICV Administration of Antisense Oligo No. 289 (10 Mg)

The duration of action for mRNA knockdown at 6, 8, and 12 weeks following a single ICV administration of Antisense Oligo No. 289 in NHPs was investigated. Two artificial CSF treated NHPs and two naive NHPs were used as controls. Day 29 tissue samples from the PK/PD ICV modeling study (Example 3 above; N=2) were used as a comparator across all different time points. In this study, 2 mL of Antisense Oligo No. 289 in artificial cerebrospinal fluid (not isovolumetric) were administered into the occipital horn of the lateral ventricle. The statistical analysis pairwise comparison for this experiment was compared between the treated groups and an artificial CSF treated control.

### Results

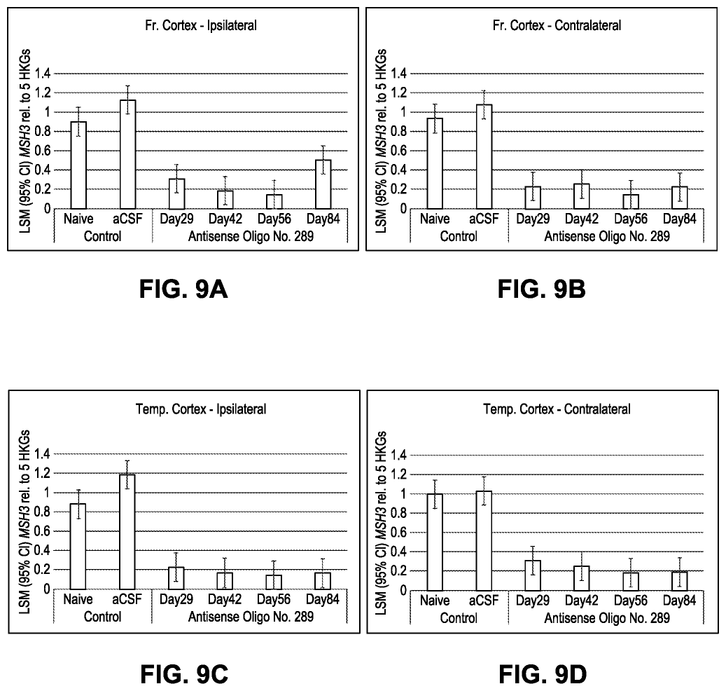

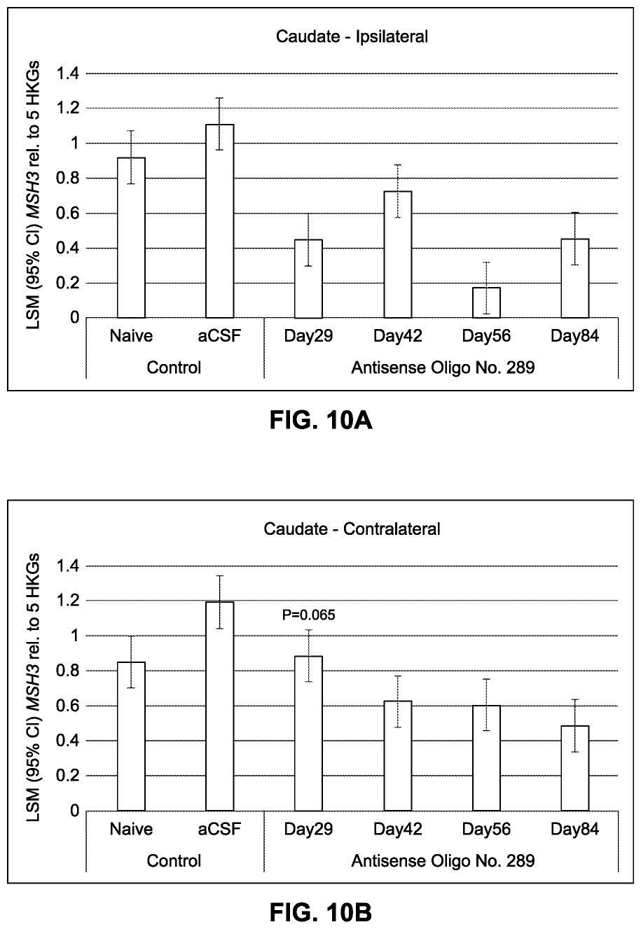

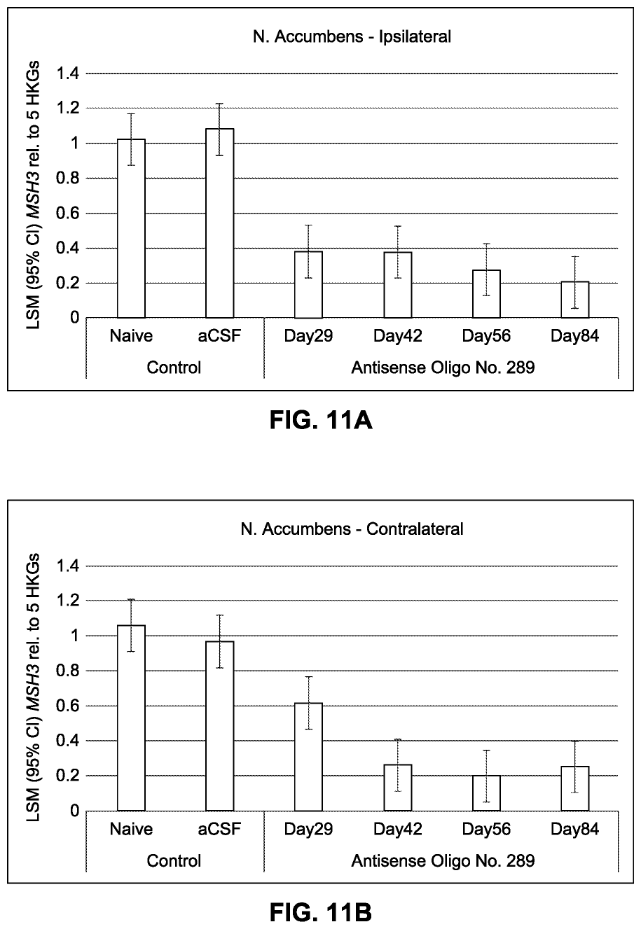

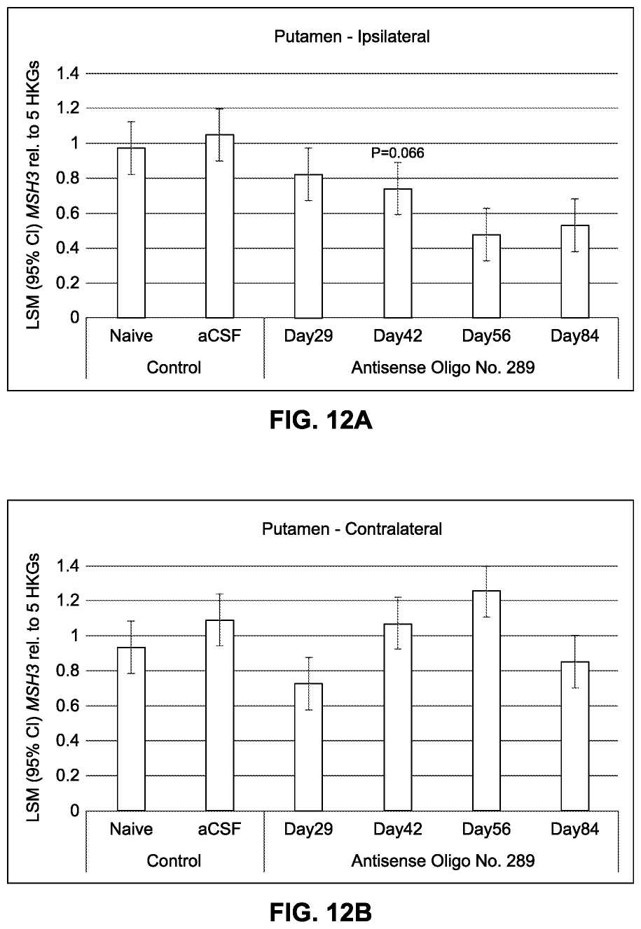

A single 10 mg ICV dose of Antisense Oligo No. 289 resulted in sustained, significant MSH3 mRNA knockdown for up to 12 weeks. In particular, sustained, significant MSH3 mRNA knockdown for up to 12 weeks was achieved in the cortex (FIGS. 9A-9D), the caudate (FIGS. 10A-10B), and the n. accumbens (FIGS. 11A-11B). Significant MSH3 mRNA knockdown was observed in the ipsilateral putamen at 8 and 12 weeks (FIGS. 12A-12B). No significant knockdown was detected in the contralateral putamen.

### Example 10: PK/PD Modeling of Single ICV Administration of Antisense Oligo No. 289

Single ICV PK/PD modeling was used to predict a dosing regimen for >50% mRNA KD in the caudate nucleus within 12 weeks.

### Study Design:

ICV Injection: occipital horn (posterior lateral ventricle)
Necropsy timepoints: 2, 8, 24, 48 h, 8, 15 and 29 days

Endpoint analysis was to determine mRNA KD in brain regions, PK in biofluids, brain regions, the liver and the kidney, and gather results from PK/PD model simulation/prediction.

### Results

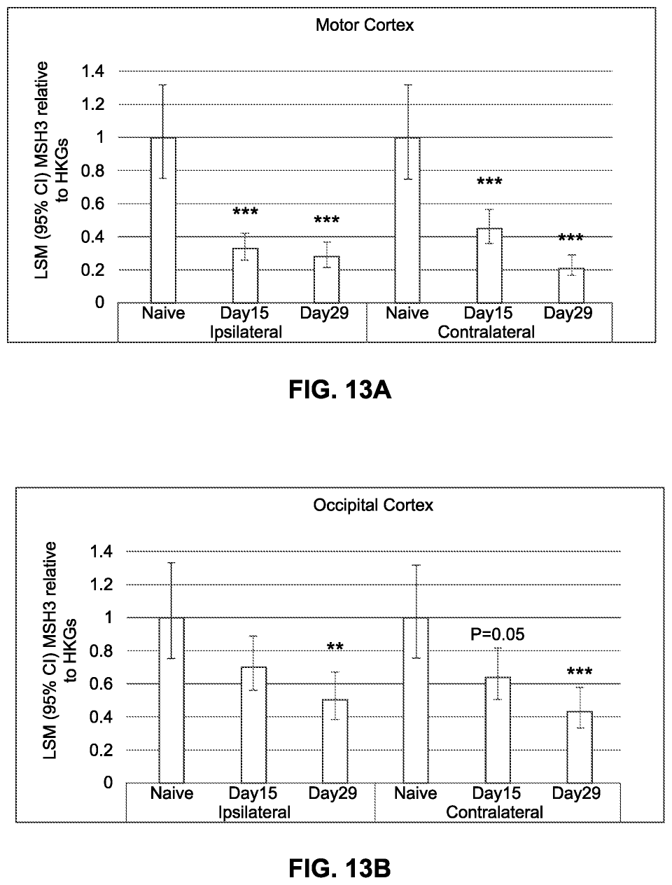

Significant mRNA KD was achieved in the ipsilateral and contralateral cortex for up to 4 weeks (FIGS. 13A and 13B).

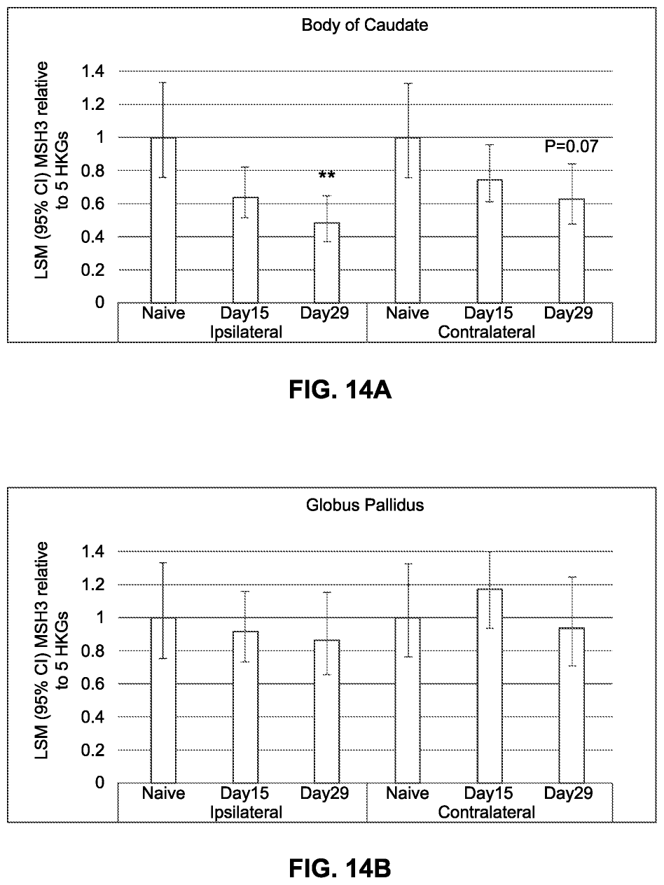

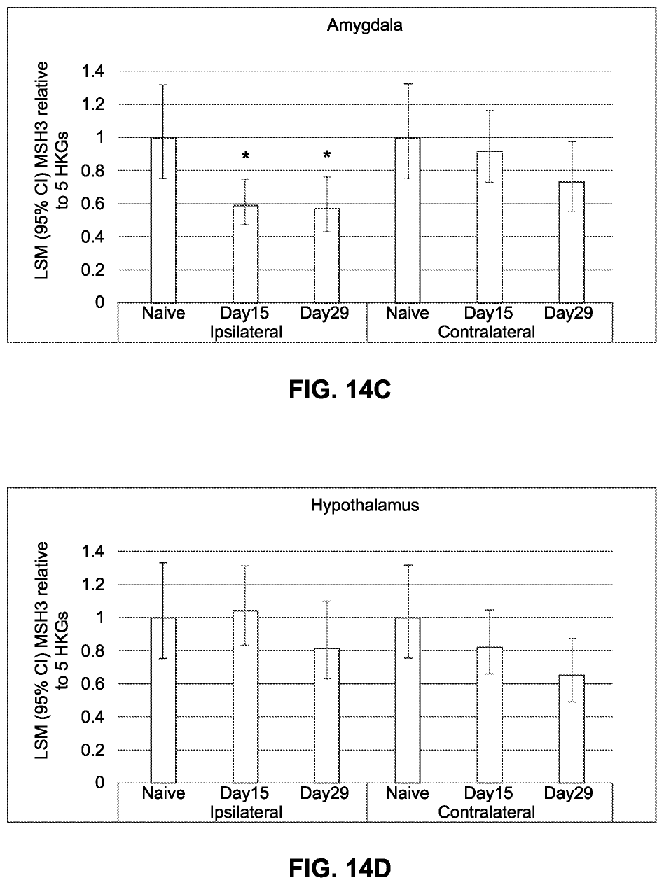

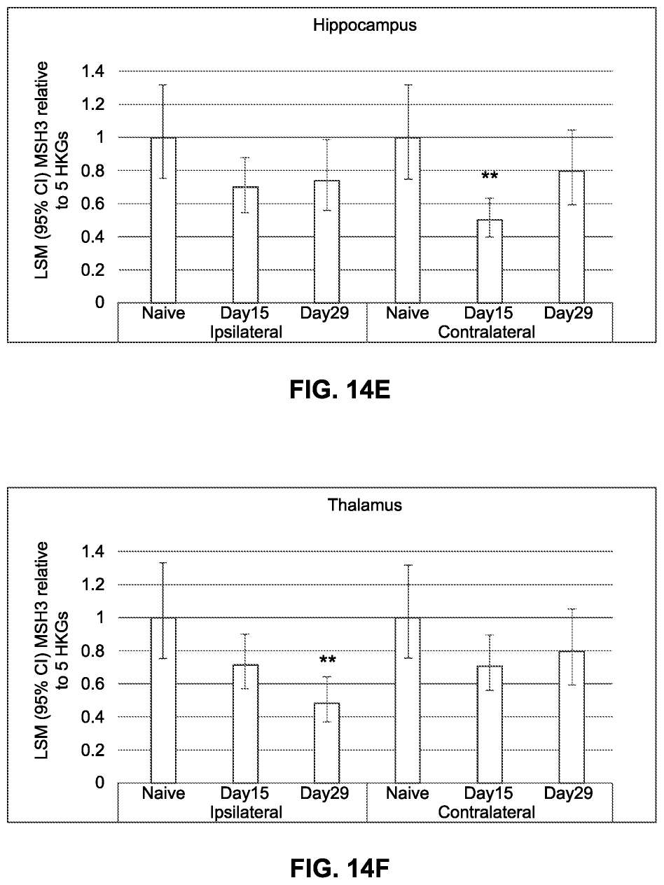

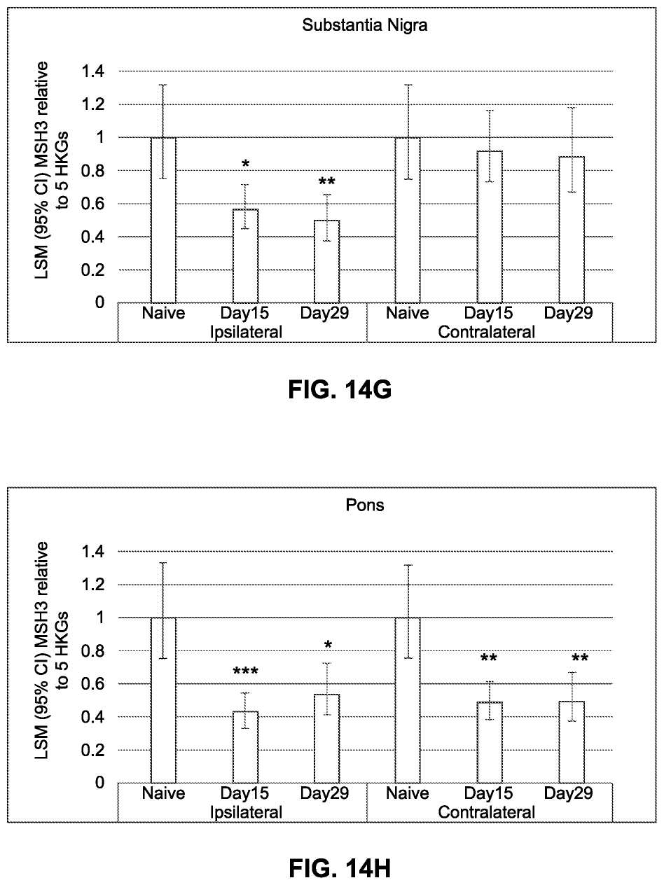

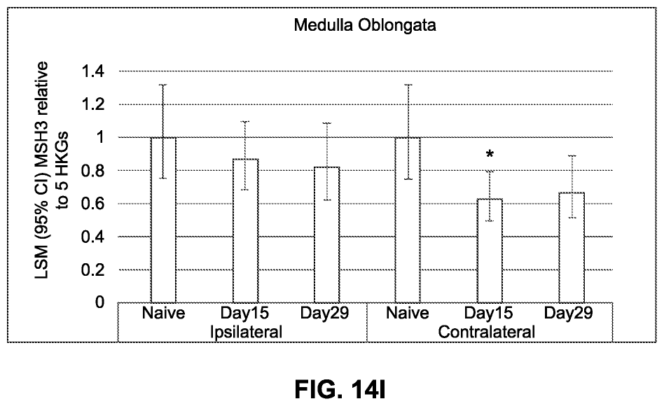

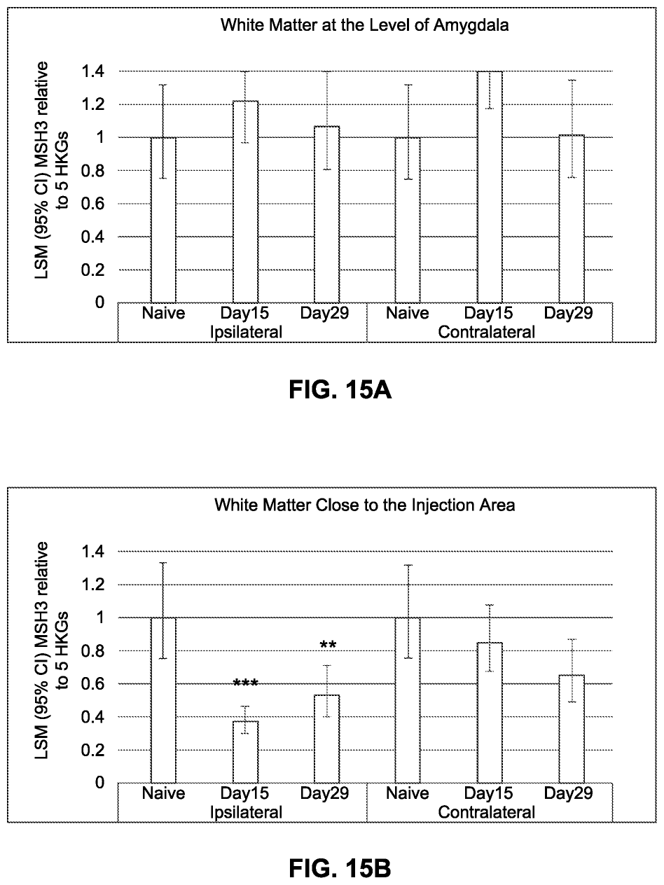

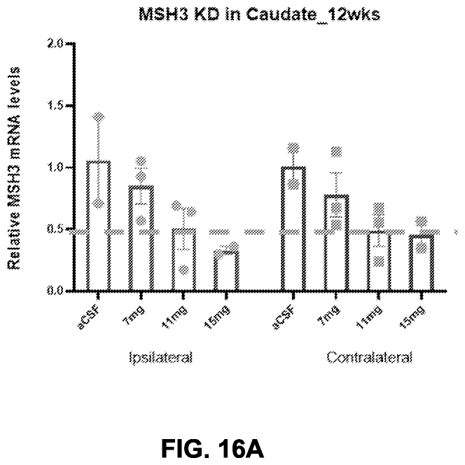

Significant mRNA KD was achieved in the ipsilateral head and body of caudate, amygdala, hypothalamus, hippocampus, thalamus, substantia nigra, pons, medulla oblongata (FIGS. 14A-14I), and ipsilateral white matter near the injection site for up to 4 weeks. (FIGS. 15A-15B). Significant mRNA KD was also achieved in both ipsilateral and contralateral pons.

These results demonstrate that single ICV administration of Antisense Oligo No. 289 at a well tolerated dose level achieves significant and widespread mRNA KD in NHPs.

### Example 11: Effect of Administration of 500 μg on Nuclear Inclusions in Q111 Mice

ICV administration of 500 μg and 300 μg doses of Antisense Oligo No. 617 in Htt^{Q111/+} mice was investigated. Age-matched 5.5 month old wild-type (C57BL6) or HttQ^{Q111/+} mice received initial ICV injections of either sterile PBS or Antisense Oligo No. 617 (300 μg or 500 μg dose levels) into the right ventricle (n=15/group). Mice in all groups were scheduled to receive three additional ICV injections, once every 8 weeks until the end of the study (n=4 ICV injections), rotating between the left and right ventricles. Plasma and CSF were collected from each animal at necropsy. Whole brains were isolated, divided into hemispheres and striatum and cortex were flash frozen separately for either histological (e.g. nuclear inclusions) or molecular analyses (MSH3 mRNA and somatic expansion).

In the 500 μg dose group, adverse clinical observations occurred in all animals following third 500 μg ICV injection of Antisense Oligo No. 617. Elevated NfL and GFAP levels in plasma were observed, suggesting sustained axonal damage and immune activation, likely in the brain. However, a 55-70% MSH3 knockdown was observed in the striatum and cortex, which resulted in a block of further somatic expansion. A mild increase in nuclear inclusion size was observed in treated mice compared to controls (phosphate buffered saline), but no statistical difference was observed between treated and control mice in the total number of nuclear inclusions counted in the striatum.

In the 300 μg dose group, no adverse clinical observations were observed during the entire study. However, Htt^{Q111/+} and wild-type mice treated with 300ug of Antisense Oligo No. 617 took longer to cross the balance beam than control mice. The number and size of the nuclear inclusions were similar between the treated group and the controls.

Nuclear inclusions (NI) reflect the accumulation of mutant HTT protein which can be detected with antibodies that recognized the mutant HTT protein. The appearance of NI in neuronal tissues are a recognized part of HD neuropathology although the formation, exact composition and biological consequence of NI are not fully understood. Genetic analyses in mice suggest that the formation of NI are linked to somatic expansion in the neuronal tissues. Antisense oligo No. 617 was not able to reduce NI in this study, despite a halt in further somatic expansion. There are several potential explanations:

Neuronal damage, as indicated by elevated NfL and GFAP, may have contributed to the lack of reduction.

Initiating therapeutic treatment at 5.5 months of age was too late since somatic expansion was already significantly increased.

It is necessary to achieve greater than 55% MSH3 mRNA reduction in striatum tissues.

### Example 12: NfL and GFAP Plasma Levels in Mice Treated with 500 μg of Antisense Oligo No. 617

The effect of ICV administration of 500 μg of Antisense Oligo No. 617 in Htt^{Q111/+} and wild-type mice was investigated.

Briefly, NfL, GFAP, UCHL1 and Tau were measured on the Quanterix 4-Plex human assay panel (Tau was not detected due to poor conservation between human and mouse). All treated animals were euthanized approximately 6 wks after their third 500 μg dose. Cerebrospinal fluid was not collected after euthanization.

Elevated NfL and GFAP plasma levels were observed. These findings suggest:
Elevated NfL levels in plasma suggest persistent axonal damage.
Elevated GFAP levels in plasma suggest microglia activation in the brain.
The increase in NfL is likely due to very high levels of antisense oligonucleotide treatment to the brain.

Wild type and Htt^{Q111/+} mice which received ICV injections of PBS did not show elevations of either NfL or GFAP. To investigate alternative tool compounds, additional ASOs were tested as described in Example 12.

### Example 13: Screening of New MSH3 ASOs for Tolerability and Activity in WT Mice

Four MSH3 ASOs were chosen with greatest MSH3 KD from an initial Axolab screen. Antisense Oligo No. 617 was included as the control. C57BL6 mice (N=6/group) received a single ICV injection at 2 dose levels, 100ug and 300ug. The control group was administered PBS control group. Mice were euthanized at 4 wks post administration and the striatum, cortex, and the rest of brains were collected. MSH3 mRNA knockdown was measured. Plasma was collected at baseline, 7 days and at 28 days post administration and CSF was collected at necropsy (28 days). The plasma and CSF were sent to Quanterix Corp. (Billerica, MA) for analyses of NfL, Tau, GFAP, and UCHL1 using their Human Neuro 4-Plex assay. Note: Tau levels were undetected in all samples, likely due to poor conservation between human and mouse

The four ASOs tested were Antisense Oligo No: 618, Antisense Oligo No. 619, Antisense Oligo No. 620, and Antisense Oligo No. 621.

### Results

Antisense Oligo No. 619 was well tolerated, showed potent MSH3 knockdown, and showed no clinical observations following ICV administration. Antisense Oligo No. 619 showed similar MSH3 knockdown in the cortex, as compared to Antisense Oligo No. 617. However, administration of each of Antisense Oligo No. 618, Antisense Oligo No. 620, and Antisense Oligo No. 621 caused negative clinical outcomes including seizures and death.

These results demonstrated that:
Tolerability of ASOs are not related to potency (i.e. MSH3 KD);
Tolerability of ASOs are not linked to elevations in NfL;
NfL elevations appear to be ASO specific and dose-dependent; and
Plasma NfL elevations are sustained, suggesting prolonged axonal damage.

None of the ASOs tested tested increased GFAP CSF or plasma levels following a single ICV at either 100 mg or 300 mg. This suggests that either higher and/or multiple doses may be required to increase GFAP and replicate the effects observed in the 500 mg SEQ ID NO: 617 groups in the HTTQ111/+ study (Example 11).

### Example 14: Antisense Oligo No. 289—ICV Delivery Protocol to Determine Tolerability

Alternative ICV delivery protocols for non-human primates that enable the assessment of a maximum tolerated dose were investigated. The initial ICV injection protocol was compared with a pilot dose escalation protocol. The findings are provided below.

The initial NHP dosing protocol was a 2 mL injection (no CSF removed) into the occipital horn of the lateral ventricle. At this dosing, Antisense Oligo No. 289 was well tolerated. Investigators observed significant knockdown in the caudate (>45%) and cortex (>75%) at day 29.

The pilot NHP dosing protocol was a 2 mL injection (no CSF removed) into the occipital horn of the lateral ventricle. A 20% dose increase resulted in transient ataxia in 4 of 6 NHPs, and a 50% dose increase resulted in adversity requiring euthanasia in one of two NHPs; the other NHP exhibited transient symptoms.

The planned NHP dosing protocol is a 1 mL injection (CSF to be removed) into the anterior horn of the lateral ventricle.

### Example 15: Antisense Oligo No. 289 Administration in Non-Human Primate (NHP) Studies

Over 125 cynomolgus monkeys have been dosed with Antisense Oligo No. 289. These studies have shown that:
It takes about 4 weeks to reach robust knockdown of MSH3 in deep brain regions, with a sustained duration of action of ≥24 weeks.
There is a predictable and linear PK/PD relationship in the therapeutic dose range.
There is good overall clinical safety and tolerability up to 20 mg (>2-fold above projected top clinical dose level.
Clinical pathology and histopathology was generally benign, except for minimal/mild adversity.

The clinical safety and tolerability of Antisense Oligo No. 289 was identified to be up to 20 mg. Tolerability was improved by lowering the dosing volumes of 1 mL vs. 2 mL, and isovolumetric injections (i.e. a 20 mg isovolumetric 1 mL repeat dose is tolerated, while a 15 mg single dose with 2 mL non-isovolumetric was not well tolerated). The study details are below in Table 7.

TABLE 7
Summary of Completed Antisense Oligo No. 289 NHP studies

| | | Results | |
|---|---|---|---|
| | Dosing | | Tolerability / |
| Study | Paradigm | Activity | Histopathology |
| PD effect, safety and | RoA: IT | MSH3 mRNA KD | Well-tolerated by all |
| tolerability of Antisense | | Cortex: ~40% | animals; |
| Oligo No. 289 and other | QD*1:2 W | Caudate: inconclusive | Histology findings |
| MSH3 ASOs after a | 10 mg | results Putamen: no | (related to IT dosing |
| single IT administration | N = 3/group | KD observed Spinal | procedure) include |
| Single IT 2 mL lumbar | (Total = 3) | Cord: ~50% | minimal |
| injection at 10 mg | | | degenerative |
| Assessed tolerability | | | changes in the spinal |
| (incl. histology), mRNA | | | cord tract in 1 |
| KD in brain tissues | | | animal and slightly |
| | | | increased incidence |
| | | | of minimal tomild |
| | | | perivascular |
| | | | mononuclear cell |
| | | | infiltrates |
| PD effect, safety and | RoA: IT | MSH3 KD | Well-tolerated by all |
| tolerability of Antisense | Q2W*2: 2 W | Cortex: ~70% | animals; |
| Oligo No. 289 and other | 10 mg | Caudate: no KD | Histology findings |
| MSH3 ASOs after repeat | N = 3/group | observed Putamen: no | (related to IT dosing |
| IT administration | (Total = 3) | KD observed Spinal | procedure) include |
| Repeat IT 2 mL lumbar | | Cord: ~40% | slightly increased |
| injection at 10 mg. | | | incidence of |
| Assessed tolerability | | | minimal perivascular |
| (incl. histology), mRNA | | | mononuclear cell |
| KD in brain tissues | | | infiltrates |
| PK/PD and modeling of | RoA: IT | MSH3 KD (Day 29 | Well-tolerated by all |
| Antisense Oligo No. 289 | QD*1:4 W | samples) Cortex: no | animals |
| after a single IT | 10 mg | KD observed Caudate: | |
| administration | N = 2/group | no KD observed | |
| Single IT 2 mL lumbar | (Total = 12) | Putamen: no KD | |
| injection at 10 mg | | observed Spinal | |
| Assessed mRNA KD and | | Cord: ~40% | |
| ASO levels in brain | | | |
| tissues Samples collected | | | |
| at 2 h, 8 h, 24 h, 48 h, Day | | | |
| 8, Day 29 | | | |
| PK/PD effect, safety and | RoA: ICV | MSH3 KD (Day 29 | Well-tolerated by all |
| tolerability of Antisense | QD*1:4W | samples) | animals. |
| Oligo No. 289 dose | 10 mg | Cortex: ~ 70% | |
| finding after a single ICV | N = 2/group | (bilateral) | |
| administration | (Total = 15) | Caudate: ~ 45% | |
| Single ICV 2 mL non- | | (ipsi), ~20% | |
| isovolumetric injection | | (contra) | |
| via posterior horn at 10 mg | | Putamen: no KD | |
| Assessed tolerability, | | observed | |
| mRNA KD and ASO | | | |
| levels in brain tissues | | | |
| Samples collected at 2 h, | | | |
| 8 h, 24 h, 48 h, Day 8, Day | | | |
| 15, Day 29 | | | |
| PK/PD duration of action | RoA: ICV | MSH3 KD (across | Well-tolerated by all |
| of Antisense Oligo No. | QD*1: 6 W, | 6 W to 12 W) | animals. |
| 289 at 6, 8 and 12 weeks | 8 W, 12 W | Cortex: ~ 80% | |
| after a single ICV | 10 mg | (bilateral) | |
| administration | N = 2/group | Caudate: ~ 50% | |
| Single ICV 2 mL non- | (Total = 6) | (ipsi), ~40% (contra) | |
| isovolumetric injection | | Putamen: ~ 40% (ipsi), | |
| via posterior horn at 10 | | no KD (6-8 W | |
| mg Assessed tolerability, | | contra), ~30% | |
| mRNA KD and ASO | | (12 W contra) | |
| levels in brain tissues | | | |
| PD effect, safety and | RoA: ICV | MSH3 KD @ Day 29 | Moderate (12 mg) to |
| tolerability of Antisense | QD*1:4W | (similar for 12 mg + | severe (15 mg) |
| Oligo No. 289 dose | 12 mg, 15 mg | 15 mg) | clinical signs; |
| finding after a single ICV | N = 1-2/group | Cortex: >80% | Clinical signs were |
| administration at 24 h, | (Total = 8) | (bilateral) | transient (resolved |
| 48h, Day 8, Day 15, Day | | Caudate: ~70% | by 24-48 hours) at |
| 29 | | (ipsi), ~50% (contra) | 12 mg |
| Single ICV 2 mL non- | | Putamen: no KD | Early euthanasia for |
| isovolumetric injection | | observed (ipsi), ~20% | 1 of 2 animals at 15 |
| via posterior horn at 12 | | (contra) | mg due to severity of |
| mg and 15 mg | | | clinical signs; no |
| Assessed tolerability and | | | microscopic findings |
| mRNA KD in brain | | | were observed |
| tissues | | | |

The clinical pathology and histopathology of NHP Antisense Oligo No. 289 dosing studies were benign. However, single sparse eosinophilic neurons in the temporal cortex after two injections two weeks apart (Q2W*2) was interpreted as necrosis based on the hematoxylin and eosin (H&E) stain by a trained pathologist. The Fluoro-Jade B (FIB) stain did not show neuronal necrosis in selected groups.

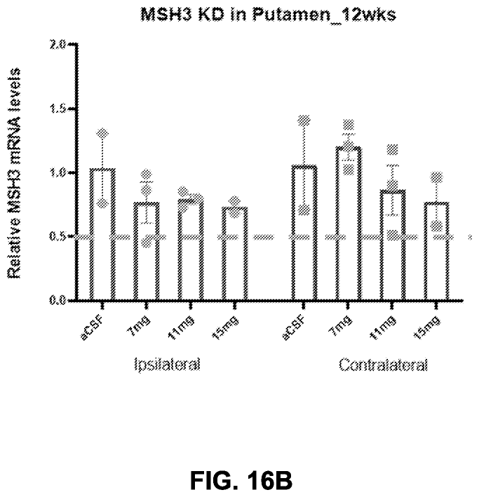

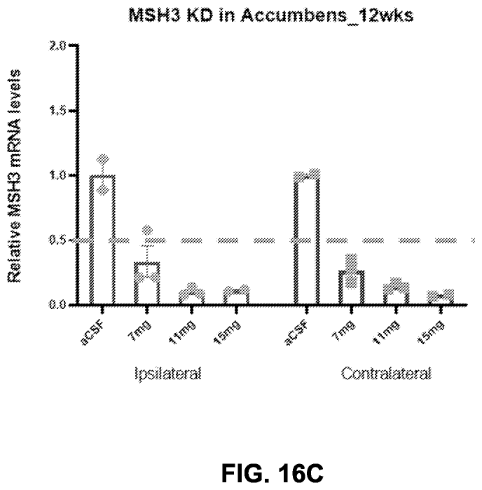

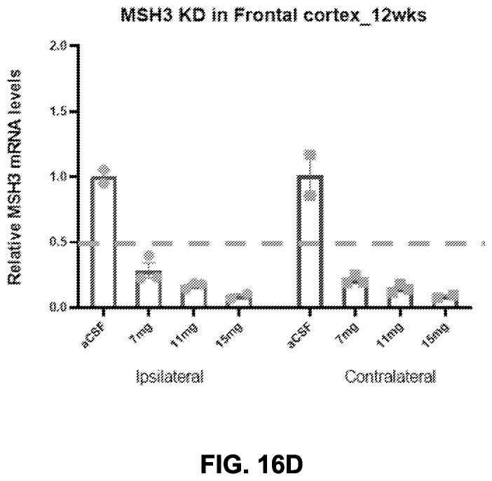

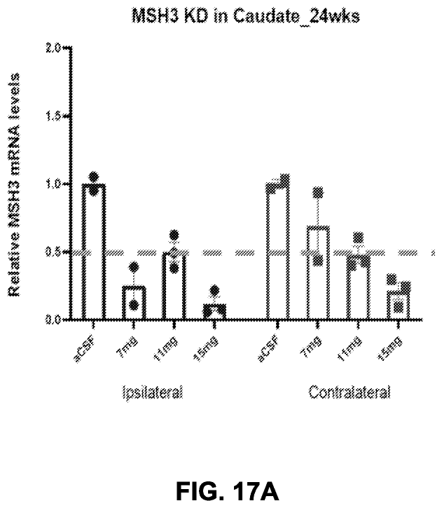

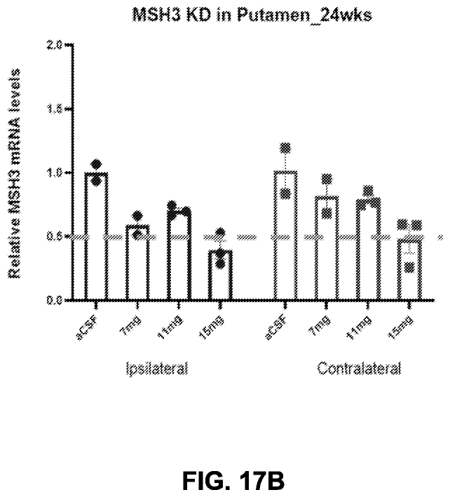

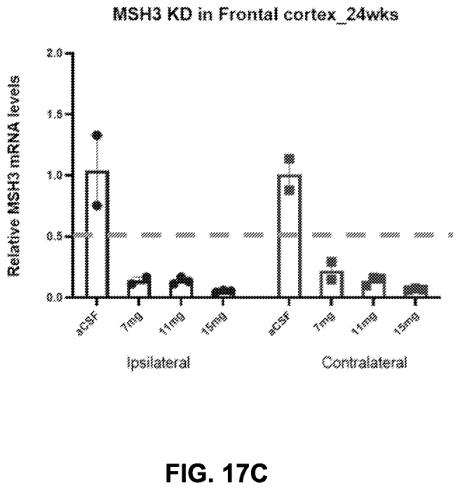

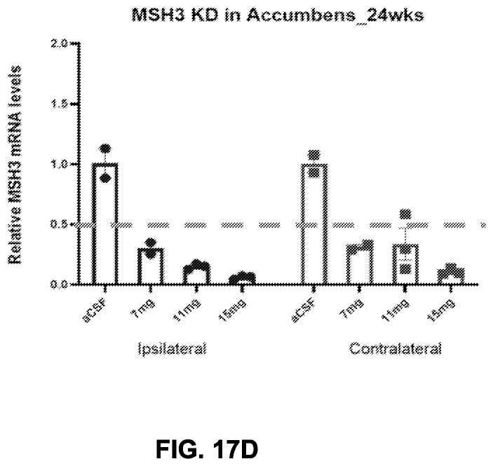

As noted above, sustained durability of MSH3 KD was observed over 24 weeks, and may be sustained to 12 months. Study details are found below in Table 8, as well as in FIGS. 16A-16D and 17A-17D.

TABLE 8
Summary of Completed and Ongoing Antisense Oligo No. 289 NHP studies

| | | | Results | |
|---|---|---|---|---|
| | Dosing | | | Tolerability / |
| Study | Paradigm | Status | Activity | Histopathology |
| Repeat ICV DRF | RoA: ICV | Completed | MSH3 KD | No clear |
| and dosing | Q2 W*2 + 2 W/ | | Cortex: | association of |
| frequency | 8 W Rec | | 60-80% across all | histopathology |
| assessment of | 5 mg, 10 mg, | | doses | findings with |
| Antisense Oligo | 12 mg, 15 mg, | | Caudate: | clinical signs |
| No. 289 with 2- | 19 mg | | 50-70% across all | No test article (TA) |
| week recovery | N = 3/group | | doses (ipsi) | related effects were |
| Repeat ICV 1 mL | (Total = 20) | | 30-60% across all | observed in serum |
| isovolumetric | | | doses (contra) | cytokines and |
| injections via | | | Putamen: ~30% | complement |
| anterior horn at | | | at 5 mg at | products after 1^{st} or |
| ascending dose | | | Wk8 (contra) | 2^{nd} doses (4 and |
| levels | | | 10-20% at 10 mg | 24 hr post-dose) |
| Assessed | | | (bilateral) ~20% | Procedure-related |
| tolerability (incl. | | | at 12 mg | increase in CSF NfL |
| histology), mRNA | | | (bilateral) | levels but Antisense |
| KD and ASO | | | 20-30% at 15 mg | Oligo No. 289 did |
| levels in brain | | | (bilateral) ~50% | not affect rate of |
| tissues | | | at 19 mg | change |
| | | | (bilateral) | |
| Antisense Oligo | RoA: ICV | Ongoing | Cortex MSH3 KD | 12 wk DoA |
| No. 289 | QD*1:12 W | | 12 wk: 70-90% | Histopathology |
| Single/Repeat ICV | Rec | | across all doses | (incl. FJB): |
| 7 mg, 11 mg, 15 | Q2 W*2: 6 & | | 24 wk: 80-95% | Non-adverse, |
| mg for PD and TE | 12 mo Rec | | across all doses | minimal to mild TA- |
| Biomarker | 7 mg, 11 mg, | | Caudate MSH3 | related findings at |
| Duration of Action | 15 mg | | KD | all dose levels |
| Single / Repeat | N = 1-3/group | | 12 wk: 20% (7 mg), | |
| ICV 1 mL | (Total = 22) | | 50% (11 mg), 55- | |
| isovolumetric | | | 70% (15 mg) | |
| injection via | | | 24 wk: 30~70% | |
| anterior horn at | | | (7 mg + 11 mg), 75- | |
| ascending dose | | | 90% (15 mg) | |
| levels | | | Putamen MSH3 | |
| Assessed | | | KD | |
| tolerability (incl. | | | 12 wk: 0-30% | |
| histology for | | | across all doses | |
| single-dose 12wk | | | 24 wk: 20-50% | |
| arm), mRNA KD | | | (7 mg + 11 mg) 50- | |
| and ASO levels in | | | 70% (15 mg) | |
| brain tissues | | | | |
| GLP 13-week | RoA: ICV | Ongoing | PD readout is not | Histopathology (incl. |
| toxicology study | Q12 W*2 + 8 W | | prepared | FJB) report is |
| (repeat dosed | Rec | | | prepared |
| Q12 W*2 via ICV | 2.5 mg, 7.5 mg, | | | |
| with 8-week | 20 mg | | | |
| recovery) | N = 4/sex/group | | | |
| Repeat ICV 1 mL | (Main), | | | |
| isovolumetric | 2/sex/group | | | |
| injections via | (Rec) | | | |
| anterior horn at | (Total = 36) | | | |
| ascending dose | | | | |
| levels | | | | |

The whole brains of NHPs were collected at necropsy after each dosing study of Antisense Oligo No. 289. NHP whole brains were placed in a chilled matrix, sliced coronally at approximately 3 mm of thickness, and stored at −80° C. A punch of tissue at approximately 2 mm in diameter was collected from various regions (i. e. cortex, caudate, putamen etc.). To quantify MSH3 mRNA from brain tissue:
Total RNA was extracted from tissue punches.
cDNA was synthesized from a determined amount of total extracted RNA.
MSH3 mRNA was quantified against reference genes GUSB, TBP, HPRT, ARL1, and RPL13A by RT-PCR.
Relative MSH3 mRNA was normalized with reference genes to calculate mRNA levels.

### Example 16: Expression Analysis for Genes of Interest from Cerebrospinal Fluid (CSF)-Derived RNAs Using qPCR

Successful analysis of MSH3 knockdown by Antisense Oligo No. 289 was performed in Glutaneuron cells and Hela cells by isolating extracellular vesicles (EV).

The analysis procedure consisted of the following steps:
1. EV isolation by ultrafiltration (UF): a. In a Amicon® filter device (Ultra-4-100KDa), 2-3 mL of cleared CSF sample was added. b. The CSF sample was spun at 4,000×g using a swinging bucket centrifuge until the original volume CSF was concentrated 20-30 times. c. The concentrated sample was transferred from the centrifuge to a new 1.5 mL tube.
2. RNA purification by QIAzol® followed by RNeasy column purification: a. After EV isolation by UF, QIAzol® was added to the tube containing the concentrated EV to perform cell lysis. b. Chloroform was added to the lysate, vortexed and centrifuged at 12,000×g for 15 min at 4° C. c. After centrifugation, the upper aqueous phase was transferred to a new 1.5 mL tube. Ethanol of 100% was added, mixed with the sample, and transfered into an RNeasy MinElute® spin column. d. After multiple wash steps, RNAs was eluted from the spin column with ˜20 ul RNase-free water. 3. Target analysis by multiplex qPCR: a. Purified RNA was reversed transcribed into cDNA using the SuperScript® IV VILO kit. b. A multiplex qPCR reaction was carried out with four different assay probes or primers (consted of one target, two housekeeping genes, and one spike-in control), a TaqPath polymerase, the cDNA, and nuclease-free water. c. After completion of PCR reaction, QuantStudio® 7 pro was used to analyze the PCR data.

### Other Aspects

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific aspects thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and can be applied to the essential features hereinbefore set forth, and follows in the scope of the claimed.

In addition to the various aspects described herein, the present disclosure includes the following aspects numbered E1 through E220. This list of aspects is presented as an exemplary list and the application is not limited to these particular aspects.

E1. A method of treating, preventing, or delaying the onset and/or progression of a nucleotide repeat expansion disorder in a subject in need thereof, the method comprising intracerebroventricularly administering a single-stranded oligonucleotide that targets MSH3, or a pharmaceutically acceptable salt thereof, wherein the single-stranded oligonucleotide, or a pharmaceutically acceptable salt thereof, is administered at a dose of about 2 mg to about 300 mg.

E2. The method of E1, wherein the oligonucleotide, or a portion thereof, is at least 95% complementary to at least 15 contiguous nucleobase at positions 2543-2573 of SEQ ID NO: 614, or a pharmaceutically acceptable salt thereof, wherein the single-stranded oligonucleotide, or a pharmaceutically acceptable salt thereof, is administered at a dose of about 2 mg to about 300 mg.

E3. The method of E1 or E2, wherein the oligonucleotide, or a portion thereof, is at least 98% complementary to at least 15 contiguous nucleobase at positions 2543-2573 of SEQ ID NO: 614, or a pharmaceutically acceptable salt thereof.

E4. The method of E1 or E2, wherein the oligonucleotide, or a portion thereof, is at least 99% complementary to at least 15 contiguous nucleobase at positions 2543-2573 of SEQ ID NO: 614, or a pharmaceutically acceptable salt thereof.

E5. The method of E1 or E2, wherein the oligonucleotide, or a portion thereof, is 100% complementary to at least 15 contiguous nucleobase at positions 2543-2573 of SEQ ID NO: 614, or a pharmaceutically acceptable salt thereof.

E6. The method of any one of E1-E5, wherein the oligonucleotide, or a portion thereof, is complementary to 17-23 contiguous nucleobase at positions 2543-2573 of SEQ ID NO: 614, or a pharmaceutically acceptable salt thereof.

E7. The method of any one of E1-E6, wherein the oligonucleotide is complementary to 17-20 contiguous nucleobase at positions 2543-2573 of SEQ ID NO: 614, or a pharmaceutically acceptable salt thereof.

E8. The method of E7, wherein the 17-20 contiguous nucleobase begin at position 2543, 2544, 2545, 2546, 2547, 2548, 2549, 2550, 2551, 2552, 2553, 2554, 2555, 2556, or 2557 of SEQ ID NO: 614, or a pharmaceutically acceptable salt thereof.

E9. The method of any one of E1-E8, wherein the oligonucleotide is 17-20 linked nucleotide in length, or a pharmaceutically acceptable salt thereof.

E10. The method of any one of E1-E8, wherein the oligonucleotide, or a portion thereof, is complementary to 20-23 contiguous nucleobase at positions 2543-2573 of SEQ ID NO: 614, or a pharmaceutically acceptable salt thereof.

E11. The method of E10, wherein the 20-23 contiguous nucleobase begin at position 2543, 2544, 2545, 2546, 2547, 2548, 2549, 2550, 2551, 2552, 2553, or 2554 of SEQ ID NO: 614, or a pharmaceutically acceptable salt thereof.

E12. The method of any one of E1-E11, wherein the oligonucleotide is 20-23 linked nucleotide in length, or a pharmaceutically acceptable salt thereof.

E13. The method of any one of E1-E12, wherein the oligonucleotide, or a portion thereof, is complementary to positions 2543-2570 of SEQ ID NO: 614, or a pharmaceutically acceptable salt thereof.

E14. A method of treating, preventing, or delaying the onset and/or progression of a nucleotide repeat expansion disorder in a subject in need thereof, the method comprising intracerebroventricularly administering a single-stranded oligonucleotide of 15-30 linked nucleotide in length, wherein the oligonucleotide, or a portion thereof, is at least 95% complementary to at least 15 contiguous nucleobase at positions 2685-2714 of SEQ ID NO: 614, or a pharmaceutically acceptable salt thereof, wherein the single-stranded oligonucleotide, or a pharmaceutically acceptable salt thereof, is administered at a dose of about 2 mg to about 300 m g.

E15. The method of E14, wherein the oligonucleotide, or a portion thereof, is at least 98% complementary to at least 15 contiguous nucleobase at positions 2685-2714 of SEQ ID NO: 614, or a pharmaceutically acceptable salt thereof.

E16. The method of E14, wherein the oligonucleotide, or a portion thereof, is at least 99% complementary to at least 15 contiguous nucleobase at positions 2685-2714 of SEQ ID NO: 614, or a pharmaceutically acceptable salt thereof.

E17. The method of E14, wherein the oligonucleotide or a portion thereof, is 100% complementary to at least 15 contiguous nucleobase at positions 2685-2714 of SEQ ID NO: 614, or a pharmaceutically acceptable salt thereof.

E18. The method of any one of E14-E17, wherein the oligonucleotide, or a portion thereof is complementary to 17-23 contiguous nucleobase at positions 2685-2714 of SEQ ID NO: 614, or a pharmaceutically acceptable salt thereof.

E19. The method of any one of E14-E18, wherein the oligonucleotide, or a portion thereof, is complementary to 17-20 contiguous nucleobase at positions 2685-2714 of SEQ ID NO: 614, or a pharmaceutically acceptable salt thereof.

E20. The method of E19, wherein the oligonucleotide, or a portion thereof, is complementary to 17-20 contiguous nucleobase beginning at position 2685, 2686, 2687, 2688, 2689, 2690, 2691, 2692, 2693, 2694, 2695, 2696, 2697, or 2698 of SEQ ID NO: 614, or a pharmaceutically acceptable salt thereof.

E21. The method of any one of E14-E20, wherein the oligonucleotide is 17-20 linked nucleotide in length, or a pharmaceutically acceptable salt thereof.

E22. The method of any one of E14-E21, wherein the oligonucleotide, or a portion thereof, is complementary to 20-23 contiguous nucleobase at positions 2685-2714 of SEQ ID NO: 614, or a pharmaceutically acceptable salt thereof.

E23. The method of E22, wherein the oligonucleotide is complementary to 20-23 contiguous nucleobase beginning at position 2685, 2686, 2687, 2688, 2689, 2690, 2691, 2692, 2693, 2694, or 2695 of SEQ ID NO: 614, or a pharmaceutically acceptable salt thereof.

E24. The method of any one of E14-E23, wherein the oligonucleotide is 20-23 linked nucleotide in length, or a pharmaceutically acceptable salt thereof.

E25. The method of any one of E14-E24, wherein the oligonucleotide, or a portion thereof, is complementary to positions 2685-2714 of SEQ ID NO: 614, or a pharmaceutically acceptable salt thereof.

E26. The method of any one of E14-E25, wherein the oligonucleotide is not any one of Antisense Oligo Nos. 1, 97, 193, or 289 of Table 3.

E27. The method of any one of E14-E26, wherein the oligonucleotide doE not have a nucleobase sequence consisting of any one of SEQ ID NOs: 1, 97, 193, or 289.

E28. The method of any one of E14-E27, wherein the oligonucleotide comprise:
(a) a DNA core sequence comprising linked deoxyribonucleoside;
(b) a 5′ flanking sequence comprising linked nucleoside; and
(c) a 3′ flanking sequence comprising linked nucleoside;
wherein the DNA core comprise a region of at least 10 contiguous nucleobase positioned between the 5′ flanking sequence and the 3′ flanking sequence; wherein the 5′ flanking sequence and the 3′ flanking sequence each comprise at least two linked nucleosides; and wherein at least one nucleoside of each flanking sequence comprises an alternative nucleoside, or a pharmaceutically acceptable salt thereof.

E29. The method of any one of E14-E28, wherein the oligonucleotide comprises at least one alternative internucleoside linkage, or a pharmaceutically acceptable salt thereof.

E30. The method of E29, wherein the at least one alternative internucleoside linkage is a phosphorothioate internucleoside linkage.

E31. The method of E29, wherein the at least one alternative internucleoside linkage is a 2′-alkoxy internucleoside linkage.

E32. The method of E29, wherein the at least one alternative internucleoside linkage is an alkyl phosphate internucleoside linkage.

E33. The method of any one of E14-E32, wherein the oligonucleotide comprises at least one alternative nucleobase, or a pharmaceutically acceptable salt thereof.

E34. The method of E33, wherein the alternative nucleobase is 5′-methylcytosine, pseudouridine, or 5-methoxyuridine.

E35. The method of any one of E14-E34, wherein the oligonucleotide comprises at least one alternative sugar moiety, or a pharmaceutically acceptable salt thereof.

E36. The method of E35, wherein the alternative sugar moiety is 2′—OMe or a bicyclic nucleic acid.

E37. The method of any one of E14-E36, wherein the oligonucleotide further comprises a ligand conjugated to the 5′ end or the 3′ end of the oligonucleotide through a monovalent or branched bivalent or trivalent linker, or a pharmaceutically acceptable salt thereof.

E38. The method of any one of E14-E37, wherein the oligonucleotide consists of a nucleobase sequence selected from the group consisting of any of SEQ ID NOs: 1-384 and 390-613, or a pharmaceutically acceptable salt thereof.

E39. The method of E38, wherein the oligonucleotide consists of a nucleobase sequence selected from the group consisting of any of SEQ ID NOs: 2-96, 98-192, 194-288, 290-384, and 390-613, or a pharmaceutically acceptable salt thereof.

E40. The method of E38, wherein the oligonucleotide consists of a nucleobase sequence selected from the group consisting of any of SEQ ID NOs: 1-384, or a pharmaceutically acceptable salt thereof.

E41. The method of E38, wherein the oligonucleotide consists of a nucleobase sequence selected from the group consisting of any of SEQ ID NOs: 2-96, 98-192, 194-288, and 290-384, or a pharmaceutically acceptable salt thereof.

E42. The method of E38, wherein the oligonucleotide consists of a nucleobase sequence selected from the group consisting of any of SEQ ID NOs: 1-96, or a pharmaceutically acceptable salt thereof.

E43. The method of E38, wherein the oligonucleotide consists of a nucleobase sequence selected from the group consisting of any of SEQ ID NOs: 2-96, or a pharmaceutically acceptable salt thereof.

E44. The method of E38, wherein the oligonucleotide consists of a nucleobase sequence selected from the group consisting of any of SEQ ID NOs: 97-192, or a pharmaceutically acceptable salt thereof.

E45. The method of E38, wherein the oligonucleotide consists of a nucleobase sequence selected from the group consisting of any of SEQ ID NOs: 98-192, or a pharmaceutically acceptable salt thereof.

E46. The method of E38, wherein the oligonucleotide consists of a nucleobase sequence selected from the group consisting of any of SEQ ID NOs: 193-288, or a pharmaceutically acceptable salt thereof.

E47. The method of E38, wherein the oligonucleotide consists of a nucleobase sequence selected from the group consisting of any of SEQ ID NOs: 194-288, or a pharmaceutically acceptable salt thereof.

E48. The method of E38, wherein the oligonucleotide consists of a nucleobase sequence selected from the group consisting of any of SEQ ID NOs: 289-384, or a pharmaceutically acceptable salt thereof.

E49. The method of E38, wherein the oligonucleotide consists of a nucleobase sequence selected from the group consisting of any of SEQ ID NOs: 288-384, or a pharmaceutically acceptable salt thereof.

E50. The method of E38, wherein the oligonucleotide consists of a nucleobase sequence selected from the group consisting of any of SEQ ID NOs: 390-613, or a pharmaceutically acceptable salt thereof.

E51. The method of E38, wherein the oligonucleotide consists of a nucleobase sequence selected from the group consisting of any of SEQ ID NOs: 390-480, or a pharmaceutically acceptable salt thereof.

E52. The method of E38, wherein the oligonucleotide consists of a nucleobase sequence selected from the group consisting of any of SEQ ID NOs: 481-571, or a pharmaceutically acceptable salt thereof.

E53. The method of E38, wherein the oligonucleotide consists of a nucleobase sequence selected from the group consisting of any of SEQ ID NOs: 572-662, or a pharmaceutically acceptable salt thereof.

E54. The method of E38, wherein the oligonucleotide consists of a nucleobase sequence selected from the group consisting of any of SEQ ID NOs: 663-613, or a pharmaceutically acceptable salt thereof.

E55. The method of E38, wherein the oligonucleotide consists of a nucleobase sequence selected from the group consisting of any of SEQ ID NOs: 1, 6, 13, 17, 21, 24, 26, 29, 33-34, 37, 44, 49-55, 57, 60-73, 75-76, 79-82, 84-86, 88-92, or 94-96, or a pharmaceutically acceptable salt thereof.

E56. The method of E38, wherein the oligonucleotide consists of a nucleobase sequence selected from the group consisting of any of SEQ ID NOs: 6, 13, 17, 21, 24, 26, 29, 33-34, 37, 44, 49-55, 57, 60-73, 75-76, 79-82, 84-86, 88-92, or 94-96, or a pharmaceutically acceptable salt thereof.

E57. The method of E38, wherein the oligonucleotide consists of a nucleobase sequence that is SEQ ID NO: 1, or a pharmaceutically acceptable salt thereof.

E58. The method of E38, wherein the oligonucleotide consists of a nucleobase sequence that is SEQ ID NO: 6, or a pharmaceutically acceptable salt thereof.

E59. The method of E38, wherein the oligonucleotide consists of a nucleobase sequence selected from the group consisting of any of SEQ ID NOs: 97, 100, 103, 105, 108, 110-111, 113-117, 122-123, 127, 129-130, 133-136, 138-139, 141, 143-145, 147-148, 154-155, 157-165, 168-170, 172, 174-180, 184, 187, or 191, or a pharmaceutically acceptable salt thereof.

E60. The method of E38, wherein the oligonucleotide consists of a nucleobase sequence selected from the group consisting of any of SEQ ID NOs: 100, 103, 105, 108, 110-111, 113-117, 122-123, 127, 129-130, 133-136, 138-139, 141, 143-145, 147-148, 154-155, 157-165, 168-170, 172, 174-180, 184, 187, or 191, or a pharmaceutically acceptable salt thereof.

E61. The method of E38, wherein the oligonucleotide consists of a nucleobase sequence that is SEQ ID NO: 97, or a pharmaceutically acceptable salt thereof.

E62. The method of E38, wherein the oligonucleotide consists of a nucleobase sequence that is SEQ ID NO: 145, or a pharmaceutically acceptable salt thereof.

E 63. The method of E38, wherein the oligonucleotide consists of a nucleobase sequence that is SEQ ID NO: 163, or a pharmaceutically acceptable salt thereof.

E64. The method of E38, wherein the oligonucleotide consists of a nucleobase sequence selected from the group consisting of any of SEQ ID NOs: 193-200, 202-230, 232-246, 248-253, 255, 258-261, 265, 270, 274-276, or 285-286, or a pharmaceutically acceptable salt thereof.

E65. The method of E38, wherein the oligonucleotide consists of a nucleobase sequence selected from the group consisting of any of SEQ ID NOs: 194-200, 202-230, 232-246, 248-253, 255, 258-261, 265, 270, 274-276, or 285-286, or a pharmaceutically acceptable salt thereof.

E66. The method of E38, wherein the oligonucleotide consists of a nucleobase sequence that is SEQ ID NO: 193, or a pharmaceutically acceptable salt thereof.

E67. The method of E38, wherein the oligonucleotide consists of a nucleobase sequence selected from the group consisting of any of SEQ ID NOs: 226-227, 234, 240, or 243-244, or a pharmaceutically acceptable salt thereof.

E68. The method of E38, wherein the oligonucleotide consists of a nucleobase sequence selected from the group consisting of any of SEQ ID NOs: 227, 234, 240, or 243-244, or a pharmaceutically acceptable salt thereof.

E69. The method of E38, wherein oligonucleotide consists of the nucleobase sequence that is SEQ ID NO: 226, or a pharmaceutically acceptable salt thereof.

E70. The method of E38, wherein the oligonucleotide consists of a nucleobase sequence selected from the group consisting of any of SEQ ID NOs: 289-290, 292, 305, 307, 313, 318, 323-324, 326, 329-330, 332, 338-339, 341, 344, or 346, or a pharmaceutically acceptable salt thereof.

E71. The method of E38, wherein the oligonucleotide consists of a nucleobase sequence selected from the group consisting of any of SEQ ID NOs: 290, 292, 305, 307, 313, 318, 323-324, 326, 329-330, 332, 338-339, 341, 344, or 346, or a pharmaceutically acceptable salt thereof.

E72. The method of E38, wherein the oligonucleotide consists of a nucleobase sequence that is SEQ ID NO: 289, or a pharmaceutically acceptable salt thereof.

E73. The method of E38, wherein the oligonucleotide consists of a nucleobase sequence that is SEQ ID NO: 329, or a pharmaceutically acceptable salt thereof.

E74. The method of E38, wherein the oligonucleotide consists of a nucleobase sequence that is SEQ ID NO: 346, or a pharmaceutically acceptable salt thereof.

E75. The method of E1, wherein the nucleobase sequence of the oligonucleotide consists of any one of SEQ ID NOs: 1-384 and 390-613, or a pharmaceutically acceptable salt thereof, wherein the single-stranded oligonucleotide, or a pharmaceutically acceptable salt thereof, is administered at a dose of about 2 mg to about 300 mg.

E76. The method of E75, wherein the nucleobase sequence of the oligonucleotide consists of any one of SEQ ID NOs: 2-96, 98-192, 194-288, 290-384, and 390-613, or a pharmaceutically acceptable salt thereof.

E77. The method of E75, wherein the nucleobase sequence of the oligonucleotide consists of any one of SEQ ID NOs: 1-384, or a pharmaceutically acceptable salt thereof.

E78. The method of E75, wherein the nucleobase sequence of the oligonucleotide consists of any one of SEQ ID NOs: 2-96, 98-192, 194-288, or 290-384, or a pharmaceutically acceptable salt thereof.

E79. The method of E75, wherein the oligonucleotide consists of the nucleobase sequence of any one of SEQ ID NOs: 1-96, or a pharmaceutically acceptable salt thereof.

E80. The method of E75, wherein the oligonucleotide consists of the nucleobase sequence of any one of SEQ ID NOs: 2-96, or a pharmaceutically acceptable salt thereof.

E81. The method of E75, wherein the oligonucleotide consists of the nucleobase sequence of any one of SEQ ID NOs: 97-192, or a pharmaceutically acceptable salt thereof.

E82. The method of E75, wherein the oligonucleotide consists of the nucleobase sequence of any one of SEQ ID NOs: 96-192, or a pharmaceutically acceptable salt thereof.

E83. The method of E75, wherein the oligonucleotide consists of the nucleobase sequence of any one of SEQ ID NOs: 193-288, or a pharmaceutically acceptable salt thereof.

E84. The method of E75, wherein the oligonucleotide consists of the nucleobase sequence of any one of SEQ ID NOs: 194-288, or a pharmaceutically acceptable salt thereof.

E85. The method of E75, wherein the oligonucleotide consists of the nucleobase sequence of any one of SEQ ID NOs: 289-384, or a pharmaceutically acceptable salt thereof.

E86. The method of E75, wherein the oligonucleotide consists of the nucleobase sequence of any one of SEQ ID NOs: 290-384, or a pharmaceutically acceptable salt thereof.

E87. The method of E75, wherein the oligonucleotide consists of the nucleobase sequence of any one of SEQ ID NOs: 390-613, or a pharmaceutically acceptable salt thereof.

E88. The method of E75, wherein the oligonucleotide consists of the nucleobase sequence of any one of SEQ ID NOs: 390-480, or a pharmaceutically acceptable salt thereof.

E89. The method of E75, wherein the oligonucleotide consists of the nucleobase sequence of any one of SEQ ID NOs: 481-571, or a pharmaceutically acceptable salt thereof.

E90. The method of E75, wherein the oligonucleotide consists of the nucleobase sequence of any one of SEQ ID NOs: 572-662, or a pharmaceutically acceptable salt thereof.

E91. The method of E75, wherein the oligonucleotide consists of the nucleobase sequence of any one of SEQ ID NOs: 663-613, or a pharmaceutically acceptable salt thereof.

E92. The method of E75, wherein the oligonucleotide consists of the nucleobase sequence of any one of SEQ ID NOs: 1, 6, 13, 17, 21, 24, 26, 29, 33-34, 37, 44, 49-55, 57, 60-73, 75-76, 79-82, 84-86, 88-92, or 94-96, or a pharmaceutically acceptable salt thereof.

E93. The method of E75, wherein the oligonucleotide consists of the nucleobase sequence of any one of SEQ ID NOs: 6, 13, 17, 21, 24, 26, 29, 33-34, 37, 44, 49-55, 57, 60-73, 75-76, 79-82, 84-86, 88-92, or 94-96, or a pharmaceutically acceptable salt thereof.

E94. The method of E75, wherein the oligonucleotide consists of the nucleobase sequence of SEQ ID NO: 1, or a pharmaceutically acceptable salt thereof.

E95. The method of E75, wherein the oligonucleotide consists of the nucleobase sequence of SEQ ID NO: 6, or a pharmaceutically acceptable salt thereof.

E96. The method of E75, wherein the oligonucleotide consists of the nucleobase sequence of any one of SEQ ID NOs: 97, 100, 103, 105, 108, 110-111, 113-117, 122-123, 127, 129-130, 133-136, 138-139, 141, 143-145, 147-148, 154-155, 157-165, 168-170, 172, 174-180, 184, 187, or 191, or a pharmaceutically acceptable salt thereof.

E97. The method of E75, wherein the oligonucleotide consists of the nucleobase sequence of any one of SEQ ID NOs: 100, 103, 105, 108, 110-111, 113-117, 122-123, 127, 129-130, 133-136, 138-139, 141, 143-145, 147-148, 154-155, 157-165, 168-170, 172, 174-180, 184, 187, or 191, or a pharmaceutically acceptable salt thereof.

E98. The method of E75, wherein the oligonucleotide consists of the nucleobase sequence of SEQ ID NO: 97, or a pharmaceutically acceptable salt thereof.

E99. The method of E75, wherein the oligonucleotide consists of a nucleobase sequence that is SEQ ID NO: 145, or a pharmaceutically acceptable salt thereof.

E100. The method of E75, wherein the oligonucleotide consists of a nucleobase sequence that is SEQ ID NO: 163, or a pharmaceutically acceptable salt thereof.

E101. The method of E75, wherein the oligonucleotide consists of the nucleobase sequence of any one of SEQ ID NOs: 193-200, 202-230, 232-246, 248-253, 255, 258-261, 265, 270, 274-276, or 285-286, or a pharmaceutically acceptable salt thereof.

E102. The method of E75, wherein the oligonucleotide consists of the nucleobase sequence of any one of SEQ ID NOs: 194-200, 202-230, 232-246, 248-253, 255, 258-261, 265, 270, 274-276, or 285-286, or a pharmaceutically acceptable salt thereof.

E103. The method of E75, wherein the oligonucleotide consists of the nucleobase sequence of any one of SEQ ID NO: 193, or a pharmaceutically acceptable salt thereof.

E104. The method of E75, wherein the oligonucleotide consists of the nucleobase sequence of any one of SEQ ID NOs: 226-227, 234, 240, or 243-244, or a pharmaceutically acceptable salt thereof.

E105. The method of E75, wherein the oligonucleotide consists of the nucleobase sequence of any one of SEQ ID NOs: 227, 234, 240, or 243-244, or a pharmaceutically acceptable salt thereof.

E106. The method of E75, wherein the oligonucleotide consists of the nucleobase sequence of SEQ ID NO: 226, or a pharmaceutically acceptable salt thereof.

E107. The method of E75, wherein the oligonucleotide consists of the nucleobase sequence of any one of SEQ ID NOs: 289-290, 292, 305, 307, 313, 318, 323-324, 326, 329-330, 332, 338-339, 341, 344, or 346, or a pharmaceutically acceptable salt thereof.

E108. The method of E75, wherein the oligonucleotide consists of the nucleobase sequence of any one of SEQ ID NOs: 290, 292, 305, 307, 313, 318, 323-324, 326, 329-330, 332, 338-339, 341, 344, or 346, or a pharmaceutically acceptable salt thereof.

E109. The method of E75, wherein the oligonucleotide consists of the nucleobase sequence of SEQ ID NO: 289, or a pharmaceutically acceptable salt thereof.

E110. The method of E75, wherein the oligonucleotide consists of the nucleobase sequence of SEQ ID NO: 329, or a pharmaceutically acceptable salt thereof.

E111. The method of E75, wherein the oligonucleotide consists of the nucleobase sequence of SEQ ID NO: 346, or a pharmaceutically acceptable salt thereof.

E112. A method of treating, preventing, or delaying the onset and/or progression of a nucleotide repeat expansion disorder in a subject in need thereof, the method comprising intracerebroventricularly administering an oligonucleotide selected from the group consisting of Antisense Oligo Nos. 1-384 of Table 3 or 390-613 of Table 4, or a pharmaceutically acceptable salt thereof, wherein the single-stranded oligonucleotide, or a pharmaceutically acceptable salt thereof, is administered at a dose of about 2 mg to about 300 mg.

E113. The method of E112, wherein the oligonucleotide is selected from the group consisting of Antisense Oligo Nos. 2-96, 98-192, 194-288, 290-384 of Table 3 and 390-613 of Table 4, or a pharmaceutically acceptable salt thereof.

E114. The method of E112, wherein the oligonucleotide is selected from the group consisting of Antisense Oligo Nos. 1-384 of Table 3, or a pharmaceutically acceptable salt thereof.

E115. The method of E112, wherein the oligonucleotide is selected from the group consisting of Antisense Oligo Nos. 2-96, 98-192, 194-288, and 290-384 of Table 3, or a pharmaceutically acceptable salt thereof.

E116. The method of E112, wherein the oligonucleotide is selected from the group consisting of Antisense Oligo Nos. 1-96 of Table 3, or a pharmaceutically acceptable salt thereof.

E117. The method of E112, wherein the oligonucleotide is selected from the group consisting of Antisense Oligo Nos. 2-96 of Table 3, or a pharmaceutically acceptable salt thereof.

E118. The method of E112, wherein the oligonucleotide is selected from the group consisting of Antisense Oligo Nos. 97-192 of Table 3, or a pharmaceutically acceptable salt thereof.

E119. The method of E112, wherein the oligonucleotide is selected from the group consisting of Antisense Oligo Nos. 98-192 of Table 3, or a pharmaceutically acceptable salt thereof.

E120. The method of E112, wherein the oligonucleotide is selected from the group consisting of Antisense Oligo Nos. 193-288 of Table 3, or a pharmaceutically acceptable salt thereof.

E121. The method of E112, wherein the oligonucleotide is selected from the group consisting of Antisense Oligo Nos. 194-288 of Table 3, or a pharmaceutically acceptable salt thereof.

122. The method of E112, wherein the oligonucleotide is selected from the group consisting of Antisense Oligo Nos. 289-384 of Table 3, or a pharmaceutically acceptable salt thereof.

E123. The method of E112, wherein the oligonucleotide is selected from the group consisting of Antisense Oligo Nos. 290-384 of Table 3, or a pharmaceutically acceptable salt thereof.

E124. The method of E112, wherein the oligonucleotide is selected from the group consisting of Antisense Oligo Nos. 390-613 of Table 4, or a pharmaceutically acceptable salt thereof.

E125. The method of E112, wherein the oligonucleotide is selected from the group consisting of Antisense Oligo Nos. 390-480 of Table 4, or a pharmaceutically acceptable salt thereof.

E126. The method of E112, wherein the oligonucleotide is selected from the group consisting of Antisense Oligo Nos. 481-571 of Table 4, or a pharmaceutically acceptable salt thereof.

E127. The method of E112, wherein the oligonucleotide is selected from the group consisting of Antisense Oligo Nos. 1, 6, 13, 17, 21, 24, 26, 29, 33-34, 37, 44, 49-55, 57, 60-73, 75-76, 79-82, 84-86, 88-92, or 94-96 of Table 3, or a pharmaceutically acceptable salt thereof.

E128. The method of E112, wherein the oligonucleotide is selected from the group consisting of Antisense Oligo Nos. 6, 13, 17, 21, 24, 26, 29, 33-34, 37, 44, 49-55, 57, 60-73, 75-76, 79-82, 84-86, 88-92, or 94-96 of Table 3, or a pharmaceutically acceptable salt thereof.

E129. The method of E112, wherein the oligonucleotide is Antisense Oligo No. 1 of Table 3, or a pharmaceutically acceptable salt thereof.

E130. The method of E112, wherein the oligonucleotide is Antisense Oligo No. 6 of Table 3, or a pharmaceutically acceptable salt thereof.

E131. The method of E112, wherein the oligonucleotide is selected from the group consisting of Antisense Oligo Nos. 97, 100, 103, 105, 108, 110-111, 113-117, 122-123, 127, 129-130, 133-136, 138-139, 141, 143-145, 147-148, 154-155, 157-165, 168-170, 172, 174-180, 184, 187, or 191 of Table 3, or a pharmaceutically acceptable salt thereof.

E132. The method of E112, wherein the oligonucleotide is selected from the group consisting of Antisense Oligo Nos. 100, 103, 105, 108, 110-111, 113-117, 122-123, 127, 129-130, 133-136, 138-139, 141, 143-145, 147-148, 154-155, 157-165, 168-170, 172, 174-180, 184, 187, or 191 of Table 3, or a pharmaceutically acceptable salt thereof.

E133. The method of E112, wherein the oligonucleotide is Antisense Oligo No. 97 of Table 3, or a pharmaceutically acceptable salt thereof.

E134. The method of E112, wherein the oligonucleotide is Antisense Oligo No. 145 of Table 3, or a pharmaceutically acceptable salt thereof.

E135. The method of E112, wherein the oligonucleotide is Antisense Oligo No. 163 of Table 3, or a pharmaceutically acceptable salt thereof.

E136. The method of E112, wherein the oligonucleotide is selected from the group consisting of Antisense Oligo Nos. 193-200, 202-230, 232-246, 248-253, 255, 258-261, 265, 270, 274-276, or 285-286 of Table 3, or a pharmaceutically acceptable salt thereof.

E137. The method of E112, wherein the oligonucleotide is selected from the group consisting of Antisense Oligo Nos. 194-200, 202-230, 232-246, 248-253, 255, 258-261, 265, 270, 274-276, or 285-286 of Table 3, or a pharmaceutically acceptable salt thereof.

E138. The method of E112, wherein the oligonucleotide is Antisense Oligo No. 193 of Table 3, or a pharmaceutically acceptable salt thereof.

E139. The method of E112, wherein the oligonucleotide is selected from the group consisting of Antisense Oligo Nos. 226-227, 234, 240, or 243-244 of Table 3, or a pharmaceutically acceptable salt thereof.

E140. The method of E112, wherein the oligonucleotide is selected from the group consisting of Antisense Oligo Nos. 227, 234, 240, or 243-244 of Table 3, or a pharmaceutically acceptable salt thereof.

E141. The method of E112, wherein the oligonucleotide is Antisense Oligo No. 226 of Table 3, or a pharmaceutically acceptable salt thereof.

E142. The method of E112, wherein the oligonucleotide is selected from the group consisting of Antisense Oligo Nos. 289-290, 292, 305, 307, 313, 318, 323-324, 326, 329-330, 332, 338-339, 341, 344, or 346 of Table 3, or a pharmaceutically acceptable salt thereof.

E143. The method of E112, wherein the oligonucleotide is selected from the group consisting of Antisense Oligo Nos. 290, 292, 305, 307, 313, 318, 323-324, 326, 329-330, 332, 338-339, 341, 344, or 346 of Table 3, or a pharmaceutically acceptable salt thereof.

E144. The method of E112, wherein the oligonucleotide is Antisense Oligo No. 289 of Table 3, or a pharmaceutically acceptable salt thereof.

E145. The method of E112, wherein the oligonucleotide is Antisense Oligo No. 329 of Table 3, or a pharmaceutically acceptable salt thereof.

E146. The method of E112, wherein the oligonucleotide is Antisense Oligo No. 346 of Table 3, or a pharmaceutically acceptable salt thereof.

E147. The method of any one of E1-E146, wherein the oligonucleotide, or a pharmaceutically acceptable salt thereof, cause at least a 50% reduction in MSH3 mRNA expression at an oligonucleotide concentration of 10 nM.

E148. The method of any one of E1-E146, wherein the oligonucleotide, or a pharmaceutically acceptable salt thereof, cause at least a 60% reduction in MSH3 mRNA expression at an oligonucleotide concentration of 10 nM.

E149. The method of any one of E1-E146, wherein the oligonucleotide, or a pharmaceutically acceptable salt thereof, cause at least a 70% reduction in MSH3 mRNA expression at an oligonucleotide concentration of 10 nM.

E150. The method of any one of E1-E146, wherein the oligonucleotide, or a pharmaceutically acceptable salt thereof, cause at least an 80% reduction in MSH3 mRNA expression at an oligonucleotide concentration of 10 nM.

E151. The method of any one of E1-E146, wherein the oligonucleotide, or a pharmaceutically acceptable salt thereof, cause at least a 50% reduction in MSH3 mRNA expression at an oligonucleotide concentration of 1 nM.

E152. The method of any one of E1-E146, wherein the oligonucleotide, or a pharmaceutically acceptable salt thereof, cause at least a 60% reduction in MSH3 mRNA expression at an oligonucleotide concentration of 1 nM.

E153. The method of any one of E1-E146, wherein the oligonucleotide, or a pharmaceutically acceptable salt thereof, cause at least a 70% reduction in MSH3 mRNA expression at an oligonucleotide concentration of 1 nM.

E154. The method of any one of E147-E153, wherein the MSH3 mRNA expression is evaluated in vitro.

E155. The method of E154, wherein the MSH3 mRNA expression is evaluated in a cell based assay.

E156. The method of E155, wherein the MSH3 mRNA expression is evaluated in HeLa cells.

E157. The method of any one of E147-E156, wherein the MSH3 mRNA expression is determined by the quantitative reverse transcription polymerase chain reaction (RT-qPCR).

E158. The method of any one of E147-E156, wherein the MSH3 mRNA is expression is normalized to the mRNA expression of a reference gene.

E159. The method of E158, wherein the MSH3 mRNA expression is normalized to the mRNA expression of beta-glucuronidase (GUSB).

E160. The method of any one of E147-E159, wherein the reduction in MSH3 mRNA expression is relative to a control.

E161. The method of E160, wherein the control is the MSH3 mRNA expression in the absence of the oligonucleotide, or pharmaceutically acceptable salt thereof.

E162. The method of E161, wherein the control is the MSH3 mRNA expression in the absence of the oligonucleotide, or pharmaceutically acceptable salt thereof, but in the prEence of a control oligonucleotide, or salt thereof.

E163. The method of E162, wherein the control oligonucleotide, or salt thereof, is a scrambled luciferase targeting oligonucleotide.

E164. The method of any one of E147-E163, wherein the reduction in MSH3 mRNA expression is calculated by a delta-delta Ct (ΔΔCT) method.

E165. The method of E164, wherein the delta-delta Ct (ΔΔCT) method comprising the normalization of the MSH3 mRNA expression to the mRNA expression of a reference gene and to the MSH3 mRNA expression in the absence of the oligonucleotide, or pharmaceutically acceptable salt thereof but in the prEence of a control oligonucleotide, or salt thereof.

E166. The method of E165, wherein the reference gene is beta-glucuronidase (GUSB) and/or the control oligonucleotide, or salt thereof, is a scrambled luciferase targeting oligonucleotide.

E167. The method of any one of E147-E166, wherein the reduction in MSH3 mRNA expression is determined by the method of Example 1.

E168. The method of any one of E147-E150 and 152-E167, wherein in the same assay, Antisense Oligo No. 1 cause approximately a 58% reduction in MSH3 mRNA expression at an oligonucleotide concentration of 10 nM.

E169. The method of any one of E154-E168, wherein in the same assay, Antisense Oligo No. 1 cause approximately a 14% reduction in MSH3 mRNA expression at an oligonucleotide concentration of 1 nM.

E170. The method of any one of E1-E169, wherein the oligonucleotide is in the free base form.

E171. The method of any one of E1-E169, wherein the oligonucleotide is a pharmaceutically acceptable salt thereof.

E172. The method of E171, wherein the oligonucleotide is a sodium salt.

E173. The method of any one of E1-E172, wherein the one or more oligonucleotide, or pharmaceutically acceptable salts thereof, are intracerebroventricularly administered as a pharmaceutical composition that comprises one or more of the oligonucleotide, or pharmaceutically acceptable salts thereof, and a pharmaceutically acceptable carrier or excipient.

E174. The method of E173, wherein the pharmaceutical composition further comprises artificial cerebrospinal fluid.

E175. The method of any one of E1-E174, wherein the subject is a primate.

E176. The method of E175, wherein the primate is a human.

E177. The method of E175, wherein the primate is a nonhuman primate.

E178. The method of any one of E1-E177, wherein the nucleotide repeat expansion disorder is spinocerebellar ataxia type 36 or frontotemporal dementia.

E179. The method of any one of E1-E177, wherein the nucleotide repeat expansion disorder is a trinucleotide repeat expansion disorder.

E180. The method of E179, wherein the trinucleotide repeat expansion disorder is a polyglutamine disease.

E181. The method of E180, wherein the polyglutamine disease is selected from the group consisting of dentatorubropallidoluysian atrophy, Huntington's disease, spinal and bulbar muscular atrophy, spinocerebellar ataxia type 1, spinocerebellar ataxia type 2, spinocerebellar ataxia type 3, spinocerebellar ataxia type 6, spinocerebellar ataxia type 7, spinocerebellar ataxia type 17, and Huntington's disease-like 2.

E182. The method of E179, wherein the trinucleotide repeat expansion disorder is a non-polyglutamine disease.

E183. The method of E182, wherein the non-polyglutamine disease is selected from the group consisting of fragile X syndrome, fragile X-associated tremor/ataxia syndrome, fragile XE mental retardation, Friedreich's ataxia, myotonic dystrophy type 1, spinocerebellar ataxia type 8, spinocerebellar ataxia type 12, oculopharyngeal muscular dystrophy, Fragile X-associated premature ovarian failure, FRA2A syndrome, FRA7A syndrome, and early infantile epileptic encephalopathy.

E184. The method of any one of E1-E183, further comprising administering an additional therapeutic agent.

E185. The method of E184, wherein the additional therapeutic agent is another oligonucleotide that hybridizE to an mRNA encoding the Huntingtin gene.

E186. The method of any one of E1-E185, wherein the single-stranded oligonucleotide, or a pharmaceutically acceptable salt thereof, is administered at a dose of about 10 mg to about 250 mg.

E187. The method of E186, wherein the single-stranded oligonucleotide, or a pharmaceutically acceptable salt thereof, is administered at a dose of about 15 mg to about 200 mg.

E188. The method of E186, wherein the single-stranded oligonucleotide, or a pharmaceutically acceptable salt thereof, is administered at a dose of about 25 mg to about 200 mg.

E189. The method of E186, wherein the single-stranded oligonucleotide, or a pharmaceutically acceptable salt thereof, is administered at a dose of about 50 mg to about 200 mg.

E190. The method of E186, wherein the single-stranded oligonucleotide, or a pharmaceutically acceptable salt thereof, is administered at a dose of about 100 mg to about 150 mg.

E191. The method of any one of E1-E190, wherein the single-stranded oligonucleotide, or a pharmaceutically acceptable salt thereof, is administered once weekly.

E192. The method of any one of E1-E190, wherein the single-stranded oligonucleotide, or a pharmaceutically acceptable salt thereof, is administered once every two weeks.

E193. The method of any one of E1-E190, wherein the single-stranded oligonucleotide, or a pharmaceutically acceptable salt thereof, is administered once every three weeks.

E194. The method of any one of E1-E190, wherein the single-stranded oligonucleotide, or a pharmaceutically acceptable salt thereof, is administered once every four weeks.

E195. The method of any one of E1-E190, wherein the single-stranded oligonucleotide, or a pharmaceutically acceptable salt thereof, is administered once every month.

E196. The method of any one of E1-E190, wherein the single-stranded oligonucleotide, or a pharmaceutically acceptable salt thereof, is administered once every six weeks.

E197. The method of any one of E1-E190, wherein the single-stranded oligonucleotide, or a pharmaceutically acceptable salt thereof, is administered once every eight weeks.

E198. The method of any one of E1-E190, wherein the single-stranded oligonucleotide, or a pharmaceutically acceptable salt thereof, is administered once every two months.

E199. The method of any one of E1-E190, wherein the single-stranded oligonucleotide, or a pharmaceutically acceptable salt thereof, is administered once every ten weeks.

E200. The method of any one of E1-E190, wherein the single-stranded oligonucleotide, or a pharmaceutically acceptable salt thereof, is administered once every twelve weeks.

E201. The method of any one of E1-E190, wherein the single-stranded oligonucleotide, or a pharmaceutically acceptable salt thereof, is administered once every three months.

E202. The method of any one of E1-E190, wherein the single-stranded oligonucleotide, or a pharmaceutically acceptable salt thereof, is administered once every sixteen weeks.

E203. The method of any one of E1-E190, wherein the single-stranded oligonucleotide, or a pharmaceutically acceptable salt thereof, is administered once every four months.

E204. The method of any one of E1-E190, wherein the single-stranded oligonucleotide, or a pharmaceutically acceptable salt thereof, is administered once every twenty weeks.

E205. The method of any one of E1-E190, wherein the single-stranded oligonucleotide, or a pharmaceutically acceptable salt thereof, is administered once every five months.

E206. The method of any one of E1-E190, wherein the single-stranded oligonucleotide, or a pharmaceutically acceptable salt thereof, is administered once every twenty-four weeks.

E207. The method of any one of E1-E190, wherein the single-stranded oligonucleotide, or a pharmaceutically acceptable salt thereof, is administered once every six months.

E208. The method of any one of E1-E207, wherein administration of the single-stranded oligonucleotide, or a pharmaceutically acceptable salt thereof delays the onset and/or progression of the nucleotide repeat expansion disorder by at least 120 days, at least 6 months, at least 12 months, at least 2 years, at least 3 years, at least 4 years, at least 5 years, at least 10 years or more, when compared with a predicted onset and/or progression.

E209. The method of any one of E1-E208, further comprising administering an additional therapeutic agent.

E210. The method of E209, wherein the additional therapeutic agent is another oligonucleotide that hybridize to an mRNA encoding the Huntingtin gene.

E211. The method of any one of E75-E110, wherein the oligonucleotide comprises:
(a) a DNA core sequence comprising linked deoxyribonucleosides;
(b) a 5′ flanking sequence comprising linked nucleosides; and
(c) a 3′ flanking sequence comprising linked nucleosides;
wherein the DNA core comprises a region of at least 10 contiguous nucleobases positioned between the 5′ flanking sequence and the 3′ flanking sequence; wherein the 5′ flanking sequence and the 3′ flanking sequence each comprises at least two linked nucleosides; and wherein at least one nucleoside of each flanking sequence comprises an alternative nucleoside, or a pharmaceutically acceptable salt thereof.

E212. The method of any one of E75-E111, wherein the oligonucleotide comprises at least one alternative internucleoside linkage, or a pharmaceutically acceptable salt thereof.

E213. The method of E212, wherein the at least one alternative internucleoside linkage is a phosphorothioate internucleoside linkage.

E214. The method of E212, wherein the at least one alternative internucleoside linkage is a 2′-alkoxy internucleoside linkage.

E215. The method of E212, wherein the at least one alternative internucleoside linkage is an alkyl phosphate internucleoside linkage.

E216. The method of any one of E211-E215, wherein the oligonucleotide comprises at least one alternative nucleobase, or a pharmaceutically acceptable salt thereof.

E217. The method of E216, wherein the alternative nucleobase is 5′-methylcytosine, pseudouridine, or 5-methoxyuridine.

E218. The method of any one of E211-E217, wherein the oligonucleotide comprises at least one alternative sugar moiety, or a pharmaceutically acceptable salt thereof.

E219. The method of E218, wherein the alternative sugar moiety is 2′—OMe or a bicyclic nucleic acid.

E220. The method of any one of E211-E219, wherein the oligonucleotide further comprises a ligand conjugated to the 5′ end or the 3′ end of the oligonucleotide through a monovalent or branched bivalent or trivalent linker, or a pharmaceutically acceptable salt thereof.

We claim:

1. A method of treating, preventing, or delaying the onset and/or progression of a nucleotide repeat expansion disorder in a subject in need thereof, the method comprising intracerebroventricularly administering a single-stranded oligonucleotide that targets MSH3, or a pharmaceutically acceptable salt thereof, wherein the single-stranded oligonucleotide, or a pharmaceutically acceptable salt thereof, is administered at a dose of about 2 mg to about 300 mg.

2. The method of claim 1, wherein the oligonucleotide, or a portion thereof, is at least 95% complementary to at least 15 contiguous nucleobases at positions 2543-2573 of SEQ ID NO: 614, or a pharmaceutically acceptable salt thereof, wherein the single-stranded oligonucleotide, or a pharmaceutically acceptable salt thereof, is administered at a dose of about 2 mg to about 300 mg.

3. The method of claim 1 or 2, wherein the oligonucleotide, or a portion thereof, is at least 98% complementary to at least 15 contiguous nucleobases at positions 2543-2573 of SEQ ID NO: 614, or a pharmaceutically acceptable salt thereof.

4. The method of claim 1 or 2, wherein the oligonucleotide, or a portion thereof, is at least 99% complementary to at least 15 contiguous nucleobases at positions 2543-2573 of SEQ ID NO: 614, or a pharmaceutically acceptable salt thereof.

5. The method of claim 1 or 22, wherein the oligonucleotide, or a portion thereof, is 100% complementary to at least 15 contiguous nucleobases at positions 2543-2573 of SEQ ID NO: 614, or a pharmaceutically acceptable salt thereof.

6. The method of any one of claims 1-5, wherein the oligonucleotide, or a portion thereof, is complementary to 17-23 contiguous nucleobases at positions 2543-2573 of SEQ ID NO: 614, or a pharmaceutically acceptable salt thereof.

7. The method of any one of claims 1-6, wherein the oligonucleotide is complementary to 17-20 contiguous nucleobases at positions 2543-2573 of SEQ ID NO: 614, or a pharmaceutically acceptable salt thereof.

8. The method of claim 7, wherein the 17-20 contiguous nucleobases begin at position 2543, 2544, 2545, 2546, 2547, 2548, 2549, 2550, 2551, 2552, 2553, 2554, 2555, 2556, or 2557 of SEQ ID NO: 614, or a pharmaceutically acceptable salt thereof.

9. The method of any one of claims 1-8, wherein the oligonucleotide is 17-20 linked nucleotides in length, or a pharmaceutically acceptable salt thereof.

10. The method of any one of claims 1-8, wherein the oligonucleotide, or a portion thereof, is complementary to 20-23 contiguous nucleobases at positions 2543-2573 of SEQ ID NO: 614, or a pharmaceutically acceptable salt thereof.

11. The method of claim 10, wherein the 20-23 contiguous nucleobases begin at position 2543, 2544, 2545, 2546, 2547, 2548, 2549, 2550, 2551, 2552, 2553, or 2554 of SEQ ID NO: 614, or a pharmaceutically acceptable salt thereof.

12. The method of any one of claims 1-11, wherein the oligonucleotide is 20-23 linked nucleotides in length, or a pharmaceutically acceptable salt thereof.

13. The method of any one of claims 1-12, wherein the oligonucleotide, or a portion thereof, is complementary to positions 2543-2570 of SEQ ID NO: 614, or a pharmaceutically acceptable salt thereof.

14. A method of treating, preventing, or delaying the onset and/or progression of a nucleotide repeat expansion disorder in a subject in need thereof, the method comprising intracerebroventricularly administering a single-stranded oligonucleotide of 15-30 linked nucleotides in length, wherein the oligonucleotide, or a portion thereof, is at least 95% complementary to at least 15 contiguous nucleobases at positions 2685-2714 of SEQ ID NO: 614, or a pharmaceutically acceptable salt thereof, wherein the single-stranded oligonucleotide, or a pharmaceutically acceptable salt thereof, is administered at a dose of about 2 mg to about 300 mg.

15. The method of claim 14, wherein the oligonucleotide, or a portion thereof, is at least 98% complementary to at least 15 contiguous nucleobases at positions 2685-2714 of SEQ ID NO: 614, or a pharmaceutically acceptable salt thereof.

16. The method of claim 14, wherein the oligonucleotide, or a portion thereof, is at least 99% complementary to at least 15 contiguous nucleobases at positions 2685-2714 of SEQ ID NO: 614, or a pharmaceutically acceptable salt thereof.

17. The method of claim 14, wherein the oligonucleotide or a portion thereof, is 100% complementary to at least 15 contiguous nucleobases at positions 2685-2714 of SEQ ID NO: 614, or a pharmaceutically acceptable salt thereof.

18. The method of any one of claims 14-17, wherein the oligonucleotide, or a portion thereof is complementary to 17-23 contiguous nucleobases at positions 2685-2714 of SEQ ID NO: 614, or a pharmaceutically acceptable salt thereof.

19. The method of any one of claims 14-17, wherein the oligonucleotide, or a portion thereof, is complementary to 17-20 contiguous nucleobases at positions 2685-2714 of SEQ ID NO: 614, or a pharmaceutically acceptable salt thereof.

20. The method of claim 19, wherein the oligonucleotide, or a portion thereof, is complementary to 17-20 contiguous nucleobases beginning at position 2685, 2686, 2687, 2688, 2689, 2690, 2691, 2692, 2693, 2694, 2695, 2696, 2697, or 2698 of SEQ ID NO: 614, or a pharmaceutically acceptable salt thereof.

21. The method of any one of claims 14-20, wherein the oligonucleotide is 17-20 linked nucleotides in length, or a pharmaceutically acceptable salt thereof.

22. The method of any one of claims 14-21, wherein the oligonucleotide, or a portion thereof, is complementary to 20-23 contiguous nucleobases at positions 2685-2714 of SEQ ID NO: 614, or a pharmaceutically acceptable salt thereof.

23. The method of claim 21, wherein the oligonucleotide is complementary to 20-23 contiguous nucleobases beginning at position 2685, 2686, 2687, 2688, 2689, 2690, 2691, 2692, 2693, 2694, or 2695 of SEQ ID NO: 614, or a pharmaceutically acceptable salt thereof.

24. The method of any one of claims 14-23, wherein the oligonucleotide is 20-23 linked nucleotides in length, or a pharmaceutically acceptable salt thereof.

25. The method of any one of claims 14-24, wherein the oligonucleotide, or a portion thereof, is complementary to positions 2685-2714 of SEQ ID NO: 614, or a pharmaceutically acceptable salt thereof.

26. The method of any one of claims 14-25, wherein the oligonucleotide is not any one of Antisense Oligo Nos. 1, 97, 193, or 289 of Table 3.

27. The method of any one of claims 14-26, wherein the oligonucleotide does not have a nucleobase sequence consisting of any one of SEQ ID NOs: 1, 97, 193, or 289.

28. The method of any one of claims 431-27, wherein the oligonucleotide comprises:
(a) a DNA core sequence comprising linked deoxyribonucleosides;
(b) a 5′ flanking sequence comprising linked nucleosides; and
(c) a 3′ flanking sequence comprising linked nucleosides;
wherein the DNA core comprises a region of at least 10 contiguous nucleobases positioned between the 5′ flanking sequence and the 3′ flanking sequence; wherein the 5′ flanking sequence and the 3′ flanking sequence each comprises at least two linked nucleosides; and wherein at least one nucleoside of each flanking sequence comprises an alternative nucleoside, or a pharmaceutically acceptable salt thereof.

29. The method of any one of claims 1-28, wherein the oligonucleotide comprises at least one alternative internucleoside linkage, or a pharmaceutically acceptable salt thereof.

30. The method of claim 29, wherein the at least one alternative internucleoside linkage is a phosphorothioate internucleoside linkage.

31. The method of claim 29, wherein the at least one alternative internucleoside linkage is a 2′-alkoxy internucleoside linkage.

32. The method of claim 29, wherein the at least one alternative internucleoside linkage is an alkyl phosphate internucleoside linkage.

33. The method of any one of claims 14-32, wherein the oligonucleotide comprises at least one alternative nucleobase, or a pharmaceutically acceptable salt thereof.

34. The method of claim 33, wherein the alternative nucleobase is 5′-methylcytosine, pseudouridine, or 5-methoxyuridine.

35. The method of any one of claims 14-34, wherein the oligonucleotide comprises at least one alternative sugar moiety, or a pharmaceutically acceptable salt thereof.

36. The method of claim 35, wherein the alternative sugar moiety is 2′—OMe or a bicyclic nucleic acid.

37. The method of any one of claims 14-36, wherein the oligonucleotide further comprises a ligand conjugated to the 5′ end or the 3′ end of the oligonucleotide through a monovalent or branched bivalent or trivalent linker, or a pharmaceutically acceptable salt thereof.

38. The method of any one of claims 14-37, wherein the oligonucleotide consists of a nucleobase sequence selected from the group consisting of any of SEQ ID NOs: 1-384 and 390-613, or a pharmaceutically acceptable salt thereof.

39. The method of claim 38, wherein the oligonucleotide consists of a nucleobase sequence selected from the group consisting of any of SEQ ID NOs: 2-96, 98-192, 194-288, 290-384, and 390-613, or a pharmaceutically acceptable salt thereof.

40. The method of claim 38, wherein the oligonucleotide consists of a nucleobase sequence selected from the group consisting of any of SEQ ID NOs: 1-384, or a pharmaceutically acceptable salt thereof.

41. The method of claim 38, wherein the oligonucleotide consists of a nucleobase sequence selected from the group consisting of any of SEQ ID NOs: 2-96, 98-192, 194-288, and 290-384, or a pharmaceutically acceptable salt thereof.

42. The method of claim 38, wherein the oligonucleotide consists of a nucleobase sequence selected from the group consisting of any of SEQ ID NOs: 1-96, or a pharmaceutically acceptable salt thereof.

43. The method of claim 38, wherein the oligonucleotide consists of a nucleobase sequence selected from the group consisting of any of SEQ ID NOs: 2-96, or a pharmaceutically acceptable salt thereof.

44. The method of claim 38, wherein the oligonucleotide consists of a nucleobase sequence selected from the group consisting of any of SEQ ID NOs: 97-192, or a pharmaceutically acceptable salt thereof.

45. The method of claim 38, wherein the oligonucleotide consists of a nucleobase sequence selected from the group consisting of any of SEQ ID NOs: 98-192, or a pharmaceutically acceptable salt thereof.

46. The method of claim 38, wherein the oligonucleotide consists of a nucleobase sequence selected from the group consisting of any of SEQ ID NOs: 193-288, or a pharmaceutically acceptable salt thereof.

47. The method of claim 38, wherein the oligonucleotide consists of a nucleobase sequence selected from the group consisting of any of SEQ ID NOs: 194-288, or a pharmaceutically acceptable salt thereof.

48. The method of claim 38, wherein the oligonucleotide consists of a nucleobase sequence selected from the group consisting of any of SEQ ID NOs: 289-384, or a pharmaceutically acceptable salt thereof.

49. The method of claim 38, wherein the oligonucleotide consists of a nucleobase sequence selected from the group consisting of any of SEQ ID NOs: 288-384, or a pharmaceutically acceptable salt thereof.

50. The method of claim 38, wherein the oligonucleotide consists of a nucleobase sequence selected from the group consisting of any of SEQ ID NOs: 390-613, or a pharmaceutically acceptable salt thereof.

51. The method of claim 38, wherein the oligonucleotide consists of a nucleobase sequence selected from the group consisting of any of SEQ ID NOs: 390-480, or a pharmaceutically acceptable salt thereof.

52. The method of claim 38, wherein the oligonucleotide consists of a nucleobase sequence selected from the group consisting of any of SEQ ID NOs: 481-571, or a pharmaceutically acceptable salt thereof.

53. The method of claim 38, wherein the oligonucleotide consists of a nucleobase sequence selected from the group consisting of any of SEQ ID NOs: 572-662, or a pharmaceutically acceptable salt thereof.

54. The method of claim 38, wherein the oligonucleotide consists of a nucleobase sequence selected from the group consisting of any of SEQ ID NOs: 663-613, or a pharmaceutically acceptable salt thereof.

55. The method of claim 38, wherein the oligonucleotide consists of a nucleobase sequence selected from the group consisting of any of SEQ ID NOs: 1, 6, 13, 17, 21, 24, 26, 29, 33-34, 37, 44, 49-55, 57, 60-73, 75-76, 79-82, 84-86, 88-92, or 94-96, or a pharmaceutically acceptable salt thereof.

56. The method of claim 38, wherein the oligonucleotide consists of a nucleobase sequence selected from the group consisting of any of SEQ ID NOs: 6, 13, 17, 21, 24, 26, 29, 33-34, 37, 44, 49-55, 57, 60-73, 75-76, 79-82, 84-86, 88-92, or 94-96, or a pharmaceutically acceptable salt thereof.

57. The method of claim 38, wherein the oligonucleotide consists of a nucleobase sequence that is SEQ ID NO: 1, or a pharmaceutically acceptable salt thereof.

58. The method of claim 38, wherein the oligonucleotide consists of a nucleobase sequence that is SEQ ID NO: 6, or a pharmaceutically acceptable salt thereof.

59. The method of claim 38, wherein the oligonucleotide consists of a nucleobase sequence selected from the group consisting of any of SEQ ID NOs: 97, 100, 103, 105, 108, 110-111, 113-117, 122-123, 127, 129-130, 133-136, 138-139, 141, 143-145, 147-148, 154-155, 157-165, 168-170, 172, 174-180, 184, 187, or 191, or a pharmaceutically acceptable salt thereof.

60. The method of claim 38, wherein the oligonucleotide consists of a nucleobase sequence selected from the group consisting of any of SEQ ID NOs: 100, 103, 105, 108, 110-111, 113-117, 122-123, 127, 129-130, 133-136, 138-139, 141, 143-145, 147-148, 154-155, 157-165, 168-170, 172, 174-180, 184, 187, or 191, or a pharmaceutically acceptable salt thereof.

61. The method of claim 38, wherein the oligonucleotide consists of a nucleobase sequence that is SEQ ID NO: 97, or a pharmaceutically acceptable salt thereof.

62. The method of claim 38, wherein the oligonucleotide consists of a nucleobase sequence that is SEQ ID NO: 145, or a pharmaceutically acceptable salt thereof.

63. The method of claim 38, wherein the oligonucleotide consists of a nucleobase sequence that is SEQ ID NO: 163, or a pharmaceutically acceptable salt thereof.

64. The method of claim 38, wherein the oligonucleotide consists of a nucleobase sequence selected from the group consisting of any of SEQ ID NOs: 193-200, 202-230, 232-246, 248-253, 255, 258-261, 265, 270, 274-276, or 285-286, or a pharmaceutically acceptable salt thereof.

65. The method of claim 38, wherein the oligonucleotide consists of a nucleobase sequence selected from the group consisting of any of SEQ ID NOs: 194-200, 202-230, 232-246, 248-253, 255, 258-261, 265, 270, 274-276, or 285-286, or a pharmaceutically acceptable salt thereof.

66. The method of claim 38, wherein the oligonucleotide consists of a nucleobase sequence that is SEQ ID NO: 193, or a pharmaceutically acceptable salt thereof.

67. The method of claim 38, wherein the oligonucleotide consists of a nucleobase sequence selected from the group consisting of any of SEQ ID NOs: 226-227, 234, 240, or 243-244, or a pharmaceutically acceptable salt thereof.

68. The method of claim 38, wherein the oligonucleotide consists of a nucleobase sequence selected from the group consisting of any of SEQ ID NOs: 227, 234, 240, or 243-244, or a pharmaceutically acceptable salt thereof.

69. The method of claim 38, wherein oligonucleotide consists of the nucleobase sequence that is SEQ ID NO: 226, or a pharmaceutically acceptable salt thereof.

70. The method of claim 38, wherein the oligonucleotide consists of a nucleobase sequence selected from the group consisting of any of SEQ ID NOs: 289-290, 292, 305, 307, 313, 318, 323-324, 326, 329-330, 332, 338-339, 341, 344, or 346, or a pharmaceutically acceptable salt thereof.

71. The method of claim 38, wherein the oligonucleotide consists of a nucleobase sequence selected from the group consisting of any of SEQ ID NOs: 290, 292, 305, 307, 313, 318, 323-324, 326, 329-330, 332, 338-339, 341, 344, or 346, or a pharmaceutically acceptable salt thereof.

72. The method of claim 38, wherein the oligonucleotide consists of a nucleobase sequence that is SEQ ID NO: 289, or a pharmaceutically acceptable salt thereof.

73. The method of claim 38, wherein the oligonucleotide consists of a nucleobase sequence that is SEQ ID NO: 329, or a pharmaceutically acceptable salt thereof.

74. The method of claim 38, wherein the oligonucleotide consists of a nucleobase sequence that is SEQ ID NO: 346, or a pharmaceutically acceptable salt thereof.

75. The method of claim 1, wherein the nucleobase sequence of the oligonucleotide consists of any one of SEQ ID NOs: 1-384 and 390-613, or a pharmaceutically acceptable salt thereof, wherein the single-stranded oligonucleotide, or a pharmaceutically acceptable salt thereof, is administered at a dose of about 2 mg to about 300 mg.

76. The method of claim 75, wherein the nucleobase sequence of the oligonucleotide consists of any one of SEQ ID NOs: 2-96, 98-192, 194-288, 290-384, and 390-613, or a pharmaceutically acceptable salt thereof.

77. The method of claim 75, wherein the nucleobase sequence of the oligonucleotide consists of any one of SEQ ID NOs: 1-384, or a pharmaceutically acceptable salt thereof.

78. The method of claim 75, wherein the nucleobase sequence of the oligonucleotide consists of any one of SEQ ID NOs: 2-96, 98-192, 194-288, or 290-384, or a pharmaceutically acceptable salt thereof.

79. The method of claim 75, wherein the oligonucleotide consists of the nucleobase sequence of any one of SEQ ID NOs: 1-96, or a pharmaceutically acceptable salt thereof.

80. The method of claim 75, wherein the oligonucleotide consists of the nucleobase sequence of any one of SEQ ID NOs: 2-96, or a pharmaceutically acceptable salt thereof.

81. The method of claim 75, wherein the oligonucleotide consists of the nucleobase sequence of any one of SEQ ID NOs: 97-192, or a pharmaceutically acceptable salt thereof.

82. The method of claim 75, wherein the oligonucleotide consists of the nucleobase sequence of any one of SEQ ID NOs: 96-192, or a pharmaceutically acceptable salt thereof.

83. The method of claim 75, wherein the oligonucleotide consists of the nucleobase sequence of any one of SEQ ID NOs: 193-288, or a pharmaceutically acceptable salt thereof.

84. The method of claim 75, wherein the oligonucleotide consists of the nucleobase sequence of any one of SEQ ID NOs: 194-288, or a pharmaceutically acceptable salt thereof.

85. The method of claim 75, wherein the oligonucleotide consists of the nucleobase sequence of any one of SEQ ID NOs: 289-384, or a pharmaceutically acceptable salt thereof.

86. The method of claim 75, wherein the oligonucleotide consists of the nucleobase sequence of any one of SEQ ID NOs: 290-384, or a pharmaceutically acceptable salt thereof.

87. The method of claim 75, wherein the oligonucleotide consists of the nucleobase sequence of any one of SEQ ID NOs: 390-613, or a pharmaceutically acceptable salt thereof.

88. The method of claim 75, wherein the oligonucleotide consists of the nucleobase sequence of any one of SEQ ID NOs: 390-480, or a pharmaceutically acceptable salt thereof.

89. The method of claim 75, wherein the oligonucleotide consists of the nucleobase sequence of any one of SEQ ID NOs: 481-571, or a pharmaceutically acceptable salt thereof.

90. The method of claim 75, wherein the oligonucleotide consists of the nucleobase sequence of any one of SEQ ID NOs: 572-662, or a pharmaceutically acceptable salt thereof.

91. The method of claim 75, wherein the oligonucleotide consists of the nucleobase sequence of any one of SEQ ID NOs: 663-613, or a pharmaceutically acceptable salt thereof.

92. The method of claim 75, wherein the oligonucleotide consists of the nucleobase sequence of any one of SEQ ID NOs: 1, 6, 13, 17, 21, 24, 26, 29, 33-34, 37, 44, 49-55, 57, 60-73, 75-76, 79-82, 84-86, 88-92, or 94-96, or a pharmaceutically acceptable salt thereof.

93. The method of claim 75, wherein the oligonucleotide consists of the nucleobase sequence of any one of SEQ ID NOs: 6, 13, 17, 21, 24, 26, 29, 33-34, 37, 44, 49-55, 57, 60-73, 75-76, 79-82, 84-86, 88-92, or 94-96, or a pharmaceutically acceptable salt thereof.

94. The method of claim 75, wherein the oligonucleotide consists of the nucleobase sequence of SEQ ID NO: 1, or a pharmaceutically acceptable salt thereof.

95. The method of claim 75, wherein the oligonucleotide consists of the nucleobase sequence of SEQ ID NO: 6, or a pharmaceutically acceptable salt thereof.

96. The method of claim 75, wherein the oligonucleotide consists of the nucleobase sequence of any one of SEQ ID NOs: 97, 100, 103, 105, 108, 110-111, 113-117, 122-123, 127, 129-130, 133-136, 138-139, 141, 143-145, 147-148, 154-155, 157-165, 168-170, 172, 174-180, 184, 187, or 191, or a pharmaceutically acceptable salt thereof.

97. The method of claim 75 wherein the oligonucleotide consists of the nucleobase sequence of any one of SEQ ID NOs: 100, 103, 105, 108, 110-111, 113-117, 122-123, 127, 129-130, 133-136, 138-139, 141, 143-145, 147-148, 154-155, 157-165, 168-170, 172, 174-180, 184, 187, or 191, or a pharmaceutically acceptable salt thereof.

98. The method of claim 75, wherein the oligonucleotide consists of the nucleobase sequence of SEQ ID NO: 97, or a pharmaceutically acceptable salt thereof.

99. The method of claim 75, wherein the oligonucleotide consists of a nucleobase sequence that is SEQ ID NO: 145, or a pharmaceutically acceptable salt thereof.

100. The method of claim 75, wherein the oligonucleotide consists of a nucleobase sequence that is SEQ ID NO: 163, or a pharmaceutically acceptable salt thereof.

101. The method of claim 75, wherein the oligonucleotide consists of the nucleobase sequence of any one of SEQ ID NOs: 193-200, 202-230, 232-246, 248-253, 255, 258-261, 265, 270, 274-276, or 285-286, or a pharmaceutically acceptable salt thereof.

102. The method of claim 75, wherein the oligonucleotide consists of the nucleobase sequence of any one of SEQ ID NOs: 194-200, 202-230, 232-246, 248-253, 255, 258-261, 265, 270, 274-276, or 285-286, or a pharmaceutically acceptable salt thereof.

103. The method of claim 75, wherein the oligonucleotide consists of the nucleobase sequence of any one of SEQ ID NO: 193, or a pharmaceutically acceptable salt thereof.

104. The method of claim 75, wherein the oligonucleotide consists of the nucleobase sequence of any one of SEQ ID NOs: 226-227, 234, 240, or 243-244, or a pharmaceutically acceptable salt thereof.

105. The method of claim 75, wherein the oligonucleotide consists of the nucleobase sequence of any one of SEQ ID NOs: 227, 234, 240, or 243-244, or a pharmaceutically acceptable salt thereof.

106. The method of claim 75, wherein the oligonucleotide consists of the nucleobase sequence of SEQ ID NO: 226, or a pharmaceutically acceptable salt thereof.

107. The method of claim 75, wherein the oligonucleotide consists of the nucleobase sequence of any one of SEQ ID NOs: 289-290, 292, 305, 307, 313, 318, 323-324, 326, 329-330, 332, 338-339, 341, 344, or 346, or a pharmaceutically acceptable salt thereof.

108. The method of claim 75, wherein the oligonucleotide consists of the nucleobase sequence of any one of SEQ ID NOs: 290, 292, 305, 307, 313, 318, 323-324, 326, 329-330, 332, 338-339, 341, 344, or 346, or a pharmaceutically acceptable salt thereof.

109. The method of claim 75, wherein the oligonucleotide consists of the nucleobase sequence of SEQ ID NO: 289, or a pharmaceutically acceptable salt thereof.

110. The method of claim 75, wherein the oligonucleotide consists of the nucleobase sequence of SEQ ID NO: 329, or a pharmaceutically acceptable salt thereof.

111. The method of claim 75, wherein the oligonucleotide consists of the nucleobase sequence of SEQ ID NO: 346, or a pharmaceutically acceptable salt thereof.

112. A method of treating, preventing, or delaying the onset and/or progression of a nucleotide repeat expansion disorder in a subject in need thereof, the method comprising intracerebroventricularly administering an oligonucleotide selected from the group consisting of Antisense Oligo Nos. 1-384 of Table 3 or 390-613 of Table 4, or a pharmaceutically acceptable salt thereof, wherein the single-stranded oligonucleotide, or a pharmaceutically acceptable salt thereof, is administered at a dose of about 2 mg to about 300 mg.

113. The method of claim 112, wherein the oligonucleotide is selected from the group consisting of Antisense Oligo Nos. 2-96, 98-192, 194-288, 290-384 of Table 3 and 390-613 of Table 4, or a pharmaceutically acceptable salt thereof.

114. The method of claim 112, wherein the oligonucleotide is selected from the group consisting of Antisense Oligo Nos. 1-384 of Table 3, or a pharmaceutically acceptable salt thereof.

115. The method of claim 112, wherein the oligonucleotide is selected from the group consisting of Antisense Oligo Nos. 2-96, 98-192, 194-288, and 290-384 of Table 3, or a pharmaceutically acceptable salt thereof.

116. The method of claim 112, wherein the oligonucleotide is selected from the group consisting of Antisense Oligo Nos. 1-96 of Table 3, or a pharmaceutically acceptable salt thereof.

117. The method of claim 112, wherein the oligonucleotide is selected from the group consisting of Antisense Oligo Nos. 2-96 of Table 3, or a pharmaceutically acceptable salt thereof.

118. The method of claim 112, wherein the oligonucleotide is selected from the group consisting of Antisense Oligo Nos. 97-192 of Table 3, or a pharmaceutically acceptable salt thereof.

119. The method of claim 112, wherein the oligonucleotide is selected from the group consisting of Antisense Oligo Nos. 98-192 of Table 3, or a pharmaceutically acceptable salt thereof.

120. The method of claim 112, wherein the oligonucleotide is selected from the group consisting of Antisense Oligo Nos. 193-288 of Table 3, or a pharmaceutically acceptable salt thereof.

121. The method of claim 112, wherein the oligonucleotide is selected from the group consisting of Antisense Oligo Nos. 194-288 of Table 3, or a pharmaceutically acceptable salt thereof.

122. The method of claim 112, wherein the oligonucleotide is selected from the group consisting of Antisense Oligo Nos. 289-384 of Table 3, or a pharmaceutically acceptable salt thereof.

123. The method of claim 112, wherein the oligonucleotide is selected from the group consisting of Antisense Oligo Nos. 290-384 of Table 3, or a pharmaceutically acceptable salt thereof.

124. The method of claim 112, wherein the oligonucleotide is selected from the group consisting of Antisense Oligo Nos. 390-613 of Table 4, or a pharmaceutically acceptable salt thereof.

125. The method of claim 112, wherein the oligonucleotide is selected from the group consisting of Antisense Oligo Nos. 390-480 of Table 4, or a pharmaceutically acceptable salt thereof.

126. The method of claim 112, wherein the oligonucleotide is selected from the group consisting of Antisense Oligo Nos. 481-571 of Table 4, or a pharmaceutically acceptable salt thereof.

127. The method of claim 112, wherein the oligonucleotide is selected from the group consisting of Antisense Oligo Nos. 1, 6, 13, 17, 21, 24, 26, 29, 33-34, 37, 44, 49-55, 57, 60-73, 75-76, 79-82, 84-86, 88-92, or 94-96 of Table 3, or a pharmaceutically acceptable salt thereof.

128. The method of claim 112, wherein the oligonucleotide is selected from the group consisting of Antisense Oligo Nos. 6, 13, 17, 21, 24, 26, 29, 33-34, 37, 44, 49-55, 57, 60-73, 75-76, 79-82, 84-86, 88-92, or 94-96 of Table 3, or a pharmaceutically acceptable salt thereof.

129. The method of claim 112, wherein the oligonucleotide is Antisense Oligo No. 1 of Table 3, or a pharmaceutically acceptable salt thereof.

130. The method of claim 112, wherein the oligonucleotide is Antisense Oligo No. 6 of Table 3, or a pharmaceutically acceptable salt thereof.

131. The method of claim 112, wherein the oligonucleotide is selected from the group consisting of Antisense Oligo Nos. 97, 100, 103, 105, 108, 110-111, 113-117, 122-123, 127, 129-130, 133-136, 138-139, 141, 143-145, 147-148, 154-155, 157-165, 168-170, 172, 174-180, 184, 187, or 191 of Table 3, or a pharmaceutically acceptable salt thereof.

132. The method of claim 112, wherein the oligonucleotide is selected from the group consisting of Antisense Oligo Nos. 100, 103, 105, 108, 110-111, 113-117, 122-123, 127, 129-130, 133-136, 138-139, 141, 143-145, 147-148, 154-155, 157-165, 168-170, 172, 174-180, 184, 187, or 191 of Table 3, or a pharmaceutically acceptable salt thereof.

133. The method of claim 112, wherein the oligonucleotide is Antisense Oligo No. 97 of Table 3, or a pharmaceutically acceptable salt thereof.

134. The method of claim 112, wherein the oligonucleotide is Antisense Oligo No. 145 of Table 3, or a pharmaceutically acceptable salt thereof.

135. The method of claim 112, wherein the oligonucleotide is Antisense Oligo No. 163 of Table 3, or a pharmaceutically acceptable salt thereof.

136. The method of claim 112, wherein the oligonucleotide is selected from the group consisting of Antisense Oligo Nos. 193-200, 202-230, 232-246, 248-253, 255, 258-261, 265, 270, 274-276, or 285-286 of Table 3, or a pharmaceutically acceptable salt thereof.

137. The method of claim 112, wherein the oligonucleotide is selected from the group consisting of Antisense Oligo Nos. 194-200, 202-230, 232-246, 248-253, 255, 258-261, 265, 270, 274-276, or 285-286 of Table 3, or a pharmaceutically acceptable salt thereof.

138. The method of claim 112, wherein the oligonucleotide is Antisense Oligo No. 193 of Table 3, or a pharmaceutically acceptable salt thereof.

139. The method of claim 112, wherein the oligonucleotide is selected from the group consisting of Antisense Oligo Nos. 226-227, 234, 240, or 243-244 of Table 3, or a pharmaceutically acceptable salt thereof.

140. The method of claim 112, wherein the oligonucleotide is selected from the group consisting of Antisense Oligo Nos. 227, 234, 240, or 243-244 of Table 3, or a pharmaceutically acceptable salt thereof.

141. The method of claim 112, wherein the oligonucleotide is Antisense Oligo No. 226 of Table 3, or a pharmaceutically acceptable salt thereof.

142. The method of claim 112, wherein the oligonucleotide is selected from the group consisting of Antisense Oligo Nos. 289-290, 292, 305, 307, 313, 318, 323-324, 326, 329-330, 332, 338-339, 341, 344, or 346 of Table 3, or a pharmaceutically acceptable salt thereof.

143. The method of claim 112, wherein the oligonucleotide is selected from the group consisting of Antisense Oligo Nos. 290, 292, 305, 307, 313, 318, 323-324, 326, 329-330, 332, 338-339, 341, 344, or 346 of Table 3, or a pharmaceutically acceptable salt thereof.

144. The method of claim 112, wherein the oligonucleotide is Antisense Oligo No. 289 of Table 3, or a pharmaceutically acceptable salt thereof.

145. The method of claim 112, wherein the oligonucleotide is Antisense Oligo No. 329 of Table 3, or a pharmaceutically acceptable salt thereof.

146. The method of claim 112, wherein the oligonucleotide is Antisense Oligo No. 346 of Table 3, or a pharmaceutically acceptable salt thereof.

147. The method of any one of claims 1-146, wherein the oligonucleotide, or a pharmaceutically acceptable salt thereof, causes at least a 50% reduction in MSH3 mRNA expression at an oligonucleotide concentration of 10 nM.

148. The method of any one of claims 1-146, wherein the oligonucleotide, or a pharmaceutically acceptable salt thereof, causes at least a 60% reduction in MSH3 mRNA expression at an oligonucleotide concentration of 10 nM.

149. The method of any one of claims 1-146, wherein the oligonucleotide, or a pharmaceutically acceptable salt thereof, causes at least a 70% reduction in MSH3 mRNA expression at an oligonucleotide concentration of 10 nM.

150. The method of any one of claims 1-146, wherein the oligonucleotide, or a pharmaceutically acceptable salt thereof, causes at least an 80% reduction in MSH3 mRNA expression at an oligonucleotide concentration of 10 nM.

151. The method of any one of claims 1-146, wherein the oligonucleotide, or a pharmaceutically acceptable salt thereof, causes at least a 50% reduction in MSH3 mRNA expression at an oligonucleotide concentration of 1 nM.

152. The method of any one of claims 1-146, wherein the oligonucleotide, or a pharmaceutically acceptable salt thereof, causes at least a 60% reduction in MSH3 mRNA expression at an oligonucleotide concentration of 1 nM.

153. The method of any one of claims 1-146, wherein the oligonucleotide, or a pharmaceutically acceptable salt thereof, causes at least a 70% reduction in MSH3 mRNA expression at an oligonucleotide concentration of 1 nM.

154. The method of any one of claims 147-153, wherein the MSH3 mRNA expression is evaluated in vitro.

155. The method of claim 154, wherein the MSH3 mRNA expression is evaluated in a cell based assay.

156. The method of claim 155, wherein the MSH3 mRNA expression is evaluated in HeLa cells.

157. The method of any one of claims 147-156, wherein the MSH3 mRNA expression is determined by the quantitative reverse transcription polymerase chain reaction (RT-qPCR).

158. The method of any one of claims 147-156, wherein the MSH3 mRNA expression is normalized to the mRNA expression of a reference gene.

159. The method of claim 158, wherein the MSH3 mRNA expression is normalized to the mRNA expression of beta-glucuronidase (GUSB).

160. The method of any one of claims 147-159, wherein the reduction in MSH3 mRNA expression is relative to a control.

161. The method of claim 160, wherein the control is the MSH3 mRNA expression in the absence of the oligonucleotide, or pharmaceutically acceptable salt thereof.

162. The method of claim 161, wherein the control is the MSH3 mRNA expression in the absence of the oligonucleotide, or pharmaceutically acceptable salt thereof, but in the presence of a control oligonucleotide, or salt thereof.

163. The method of claim 162, wherein the control oligonucleotide, or salt thereof, is a scrambled luciferase targeting oligonucleotide.

164. The method of any one of claims 147-163, wherein the reduction in MSH3 mRNA expression is calculated by a delta-delta Ct (ΔΔCT) method.

165. The method of claim 164, wherein the delta-delta Ct (ΔΔCT) method comprises the normalization of the MSH3 mRNA expression to the mRNA expression of a reference gene and to the MSH3 mRNA expression in the absence of the oligonucleotide, or pharmaceutically acceptable salt thereof but in the presence of a control oligonucleotide, or salt thereof.

166. The method of claim 165, wherein the reference gene is beta-glucuronidase (GUSB) and/or the control oligonucleotide, or salt thereof, is a scrambled luciferase targeting oligonucleotide.

167. The method of any one of claims 147-166, wherein the reduction in MSH3 mRNA expression is determined by the method of Example 1.

168. The method of any one of claims 147-150 and 152-167, wherein in the same assay, Antisense Oligo No. 1 causes approximately a 58% reduction in MSH3 mRNA expression at an oligonucleotide concentration of 10 nM.

169. The method of any one of claims 154-168, wherein in the same assay, Antisense Oligo No. 1 causes approximately a 14% reduction in MSH3 mRNA expression at an oligonucleotide concentration of 1 nM.

170. The method of any one of claims 1-169, wherein the oligonucleotide is in the free base form.

171. The method of any one of claims 1-169, wherein the oligonucleotide is a pharmaceutically acceptable salt thereof.

172. The method of claim 171, wherein the oligonucleotide is a sodium salt.

173. The method of any one of claims 1-172, wherein the one or more oligonucleotides, or pharmaceutically acceptable salts thereof, are intracerebroventricularly administered as a pharmaceutical composition that comprises one or more of the oligonucleotides, or pharmaceutically acceptable salts thereof, and a pharmaceutically acceptable carrier or excipient.

174. The method of claim 173, wherein the pharmaceutical composition further comprises artificial cerebrospinal fluid.

175. The method of any one of claims 1-174, wherein the subject is a primate.

176. The method of claim 175, wherein the primate is a human.

177. The method of claim 175, wherein the primate is a nonhuman primate.

178. The method of any one of claims 1-177, wherein the nucleotide repeat expansion disorder is spinocerebellar ataxia type 36 or frontotemporal dementia.

179. The method of any one of claims 1-177, wherein the nucleotide repeat expansion disorder is a trinucleotide repeat expansion disorder.

180. The method of claim 179, wherein the trinucleotide repeat expansion disorder is a polyglutamine disease.

181. The method of claim 180, wherein the polyglutamine disease is selected from the group consisting of dentatorubropallidoluysian atrophy, Huntington's disease, spinal and bulbar muscular atrophy, spinocerebellar ataxia type 1, spinocerebellar ataxia type 2, spinocerebellar ataxia type 3, spinocerebellar ataxia type 6, spinocerebellar ataxia type 7, spinocerebellar ataxia type 17, and Huntington's disease-like 2.

182. The method of claim 179, wherein the trinucleotide repeat expansion disorder is a non-polyglutamine disease.

183. The method of claim 182, wherein the non-polyglutamine disease is selected from the group consisting of fragile X syndrome, fragile X-associated tremor/ataxia syndrome, fragile XE mental retardation, Friedreich's ataxia, myotonic dystrophy type 1, spinocerebellar ataxia type 8, spinocerebellar ataxia type 12, oculopharyngeal muscular dystrophy, Fragile X-associated premature ovarian failure, FRA2A syndrome, FRA7A syndrome, and early infantile epileptic encephalopathy.

184. The method of any one of claims 1-183, further comprising administering an additional therapeutic agent.

185. The method of claim 184, wherein the additional therapeutic agent is another oligonucleotide that hybridizes to an mRNA encoding the Huntingtin gene.

186. The method of any one of claims 1-185, wherein the single-stranded oligonucleotide, or a pharmaceutically acceptable salt thereof, is administered at a dose of about 10 mg to about 250 mg.

187. The method of claim 186, wherein the single-stranded oligonucleotide, or a pharmaceutically acceptable salt thereof, is administered at a dose of about 15 mg to about 200 mg.

188. The method of claim 186, wherein the single-stranded oligonucleotide, or a pharmaceutically acceptable salt thereof, is administered at a dose of about 25 mg to about 200 mg.

189. The method of claim 186, wherein the single-stranded oligonucleotide, or a pharmaceutically acceptable salt thereof, is administered at a dose of about 50 mg to about 200 mg.

190. The method of claim 186, wherein the single-stranded oligonucleotide, or a pharmaceutically acceptable salt thereof, is administered at a dose of about 100 mg to about 150 mg.

191. The method of any one of claims 1-190, wherein the single-stranded oligonucleotide, or a pharmaceutically acceptable salt thereof, is administered once weekly.

192. The method of any one of claims 1-190, wherein the single-stranded oligonucleotide, or a pharmaceutically acceptable salt thereof, is administered once every two weeks.

193. The method of any one of claims 1-190, wherein the single-stranded oligonucleotide, or a pharmaceutically acceptable salt thereof, is administered once every three weeks.

194. The method of any one of claims 1-190, wherein the single-stranded oligonucleotide, or a pharmaceutically acceptable salt thereof, is administered once every four weeks.

195. The method of any one of claims 1-190, wherein the single-stranded oligonucleotide, or a pharmaceutically acceptable salt thereof, is administered once every month.

196. The method of any one of claims 1-190, wherein the single-stranded oligonucleotide, or a pharmaceutically acceptable salt thereof, is administered once every six weeks.

197. The method of any one of claims 1-190, wherein the single-stranded oligonucleotide, or a pharmaceutically acceptable salt thereof, is administered once every eight weeks.

198. The method of any one of claims 1-190, wherein the single-stranded oligonucleotide, or a pharmaceutically acceptable salt thereof, is administered once every two months.

199. The method of any one of claims 1-190, wherein the single-stranded oligonucleotide, or a pharmaceutically acceptable salt thereof, is administered once every ten weeks.

200. The method of any one of claims 1-190, wherein the single-stranded oligonucleotide, or a pharmaceutically acceptable salt thereof, is administered once every twelve weeks.

201. The method of any one of claims 1-190, wherein the single-stranded oligonucleotide, or a pharmaceutically acceptable salt thereof, is administered once every three months.

202. The method of any one of claims 1-190, wherein the single-stranded oligonucleotide, or a pharmaceutically acceptable salt thereof, is administered once every sixteen weeks.

203. The method of any one of claims 1-190, wherein the single-stranded oligonucleotide, or a pharmaceutically acceptable salt thereof, is administered once every four months.

204. The method of any one of claims 1-190, wherein the single-stranded oligonucleotide, or a pharmaceutically acceptable salt thereof, is administered once every twenty weeks.

205. The method of any one of claims 1-190, wherein the single-stranded oligonucleotide, or a pharmaceutically acceptable salt thereof, is administered once every five months.

206. The method of any one of claims 1-190, wherein the single-stranded oligonucleotide, or a pharmaceutically acceptable salt thereof, is administered once every twenty-four weeks.

207. The method of any one of claims 1-190, wherein the single-stranded oligonucleotide, or a pharmaceutically acceptable salt thereof, is administered once every six months.

208. The method of any one of claims 1-207, wherein administration of the single-stranded oligonucleotide, or a pharmaceutically acceptable salt thereof delays the onset and/or progression of the nucleotide repeat expansion disorder by at least 120 days, at least 6 months, at least 12 months, at least 2 years, at least 3 years, at least 4 years, at least 5 years, at least 10 years or more, when compared with a predicted onset and/or progression.

209. The method of any one of claims 1-208, further comprising administering an additional therapeutic agent.

210. The method of claim 209, wherein the additional therapeutic agent is another oligonucleotide that hybridizes to an mRNA encoding the Huntingtin gene.

211. The method of any one of claims 75-110, wherein the oligonucleotide comprises:
(a) a DNA core sequence comprising linked deoxyribonucleosides;
(b) a 5′ flanking sequence comprising linked nucleosides; and
(c) a 3′ flanking sequence comprising linked nucleosides;
wherein the DNA core comprises a region of at least 10 contiguous nucleobases positioned between the 5′ flanking sequence and the 3′ flanking sequence; wherein the 5′ flanking sequence and the 3′ flanking sequence each comprises at least two linked nucleosides; and wherein at least one nucleoside of each flanking sequence comprises an alternative nucleoside, or a pharmaceutically acceptable salt thereof.

212. The method of any one of claims 75-111, wherein the oligonucleotide comprises at least one alternative internucleoside linkage, or a pharmaceutically acceptable salt thereof.

213. The method of claim 212, wherein the at least one alternative internucleoside linkage is a phosphorothioate internucleoside linkage.

214. The method of claim 212, wherein the at least one alternative internucleoside linkage is a 2′-alkoxy internucleoside linkage.

215. The method of claim 212, wherein the at least one alternative internucleoside linkage is an alkyl phosphate internucleoside linkage.

216. The method of any one of claims 211-215, wherein the oligonucleotide comprises at least one alternative nucleobase, or a pharmaceutically acceptable salt thereof.

217. The method of claim 216, wherein the alternative nucleobase is 5′-methylcytosine, pseudouridine, or 5-methoxyuridine.

218. The method of any one of claims 211-217, wherein the oligonucleotide comprises at least one alternative sugar moiety, or a pharmaceutically acceptable salt thereof.

219. The method of claim 218, wherein the alternative sugar moiety is 2′—OMe or a bicyclic nucleic acid.

220. The method of any one of claims 211-219, wherein the oligonucleotide further comprises a ligand conjugated to the 5′ end or the 3′ end of the oligonucleotide through a monovalent or branched bivalent or trivalent linker, or a pharmaceutically acceptable salt thereof.
